(19) United States
(12) Patent Application Publication
(10) Pub. No.: US 2024/0108006 A1
(43) Pub. Date: Apr. 4, 2024

(54) PESTICIDALLY ACTIVE HETEROAROMATIC COMPOUNDS

(71) Applicant: SYNGENTA CROP PROTECTION AG, Basel (CH)

(72) Inventors: Jagadeesh Prathap KILARU, Corlim, Ilhas (IN); Mangala PHADTE, Corlim, Ilhas (IN); Simone BERARDOZZI, Stein (CH); Roger Graham HALL, Stein (CH); Viorel Andrei IOSUB, Stein (CH); André JEANGUENAT, Stein (CH); Thomas PITTERNA, Stein (CH); Vincent QUETGLAS, Stein (CH); Matthias WEISS, Stein (CH)

(73) Assignee: SYNGENTA CROP PROTECTION AG, Basel (CH)

(21) Appl. No.: 18/262,474

(22) PCT Filed: Jan. 19, 2022
(86) PCT No.: PCT/EP2022/051111

§ 371 Date: Jul. 21, 2023

(30) Foreign Application Priority Data
Jan. 23, 2021 (IN) 202111003249
Apr. 12, 2021 (IN) 202111017068
Nov. 19, 2021 (IN) 202111053250

(51) Int. Cl. A01N 43/653; A01N 43/60; A01N 43/78; A01N 43/90; A01P 7/00

(52) U.S. Cl. CPC A01N 43/653; A01N 43/60; A01N 43/78; A01N 43/90; A01P 7/00

(57) ABSTRACT

Compounds of formula (I) wherein the substituents are as defined in claim 1, and the agrochemically acceptable salts, stereoisomers, enantiomers, tautomers and N-oxides of those compounds, can be used as insecticides.

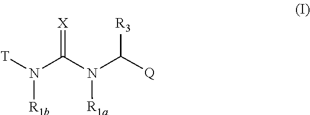

The present invention relates to pesticidally active, in particular insecticidally active compounds, to processes for their preparation, to compositions comprising those compounds, and to their use for controlling animal pests, including arthropods and in particular insects or representatives of the order Acarina.

There have now been found novel pesticidally active compounds.

The present invention accordingly relates, in a first aspect, to a compound of the formula I

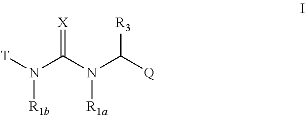

wherein:
X is O or S;

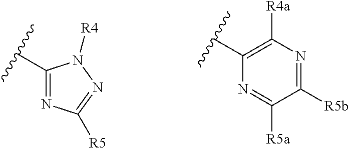

Q is Q^{a} or Q^{b};
T is phenyl, pyridine, pyrimidine, pyrazine, pyridazine or a five or nine membered heteroaromatic ring; or
T is phenyl, pyridine, pyrimidine, pyrazine, pyridazine or a five or nine membered heteroaromatic ring, each of which, independent of each other, is substituted with one to three substituents independently selected from R₂;
R₁ₐ and R_{1b} are independently selected from hydrogen, C₁-C₆alkyl, C₁-C₆alkyl substituted with one substituent independently selected from CN, C₁-C₃alkoxy, C(O)NH₂, C(O)OH, NO2 and —Si(CH₃)₃, C₁-C₆haloalkyl, C₂-C₆alkenyl, C₂-C₆haloalkenyl, C₂-C₆alkynyl, C₂-C₆haloalkynyl, C₃-C₄cycloalkyl-C₁-C₂alkyl, C₃-C₄cycloalkyl-C₁-C₂alkyl substituted with 1 or 2 halo atoms, oxetan-3-yl-CH₂—, C₁-C₆alkylcarbonyl, C₁-C₆alkoxycarbonyl, phenyloxycarbonyl, benzyloxycarbonyl, benzyl and benzyl substituted with 1 to 3 substituents independently selected from halogen, C₁-C₆alkoxy and C₁-C₆haloalkyl; or
R₁ₐ and R_{1b} together is selected from —(CH₂)₂—, —(CH₂)₃—, —CH₂—O—CH₂—, —CH₂—S—CH₂—, —CH₂—S(═O)—CH₂—, —CH₂—SO₂—CH₂— and —CH₂—N(R_{Y})—CH₂—, to form a 5- or 6-membered ring with the nitrogen atoms to which they are connected;
R_{1b} and T together with the nitrogen atom form a nine or ten membered bicyclic heterocyclic ring, which can be substituted with one to three substituents independently selected from C₁-C₃alkyl, C₁-C₃haloalkyl, C₁-C₃alkoxy, C₃-C₄cycloalkyl, halogen, hydroxyl, cyano and C₁-C₃haloalkoxy;
R₂ is independently selected from C₁-C₃alkyl, C₁-C₃haloalkyl, C₁-C₃haloalkylthio, C₁-C₃alkoxy, C₁-C₃haloalkoxy, halo, NO₂, SF₅, CN, C(O)NR₇R₈, C(O)OH, C(S)NH₂, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, C₃-C₆cycloalkyl, C₃-C₆cycloalkyl substituted with one to three substituents independently selected from Rₓ, C₃-C₆cycloalkylcarbonyl, phenyl, phenyl substituted with one to three substituents independently selected from Rₓ, heteroaryl, heteroaryl substituted with one to three substituents independently selected from Rₓ, OR₆, piperidin-2-on-1-yl, piperidin-2-on-1-yl substituted with one to two substituents independently selected from Rₓ, pyridin-2-on-1-yl, pyridin-2-on-1-yl substituted with one to two substituents independently selected from Rₓ, azetidin-1-yl, azetidin-1-yl substituted with one to two substituents independently selected from Rₓ, pyrrolidin-1-yl, pyrrolidin-1-yl substituted with one to two substituents independently selected from Rₓ, C₃-C₆cycloalkylC₁-C₄alkyl, C₃-C₆cycloalkylC₁-C₄alkyl substituted with one to two substituents independently selected from R_{z}, C₃-C₆cycloalkylC₁-C₃alkoxy, C₃-C₆cycloalkylC₁-C₃alkoxy substituted with one to two substituents independently selected from Rₓ, C₁-C₅cyanoalkyl, C₁-C₅cyanoalkoxy, C₁-C₄alkylsulfonyl, C₁-C₄alkylsulfonyl substituted with one to two substituents independently selected from Rₓ, C₁-C₄alkylsulfinyl, C₁-C₄alkylsulfinyl substituted with one to two substituents independently selected from Rₓ, C₃-C₆cycloalkylsulfanyl, C₃-C₆cycloalkylsulfinyl, C₃-C₆cycloalkylsulfonyl; a divalent group selected from —O—C₁-C₂haloalkanediyl-O—, —CH₂—C(CH₃)₂—O—, —CH₂—C(CH₃)₂—S—, —CH₂—C(CH₃)₂—SO₂—, wherein said divalent group is connected to T via two adjacent ring members of T; and eight to twelve membered spiroheterocyclic ring, which ring can be substituted by one or two substituents selected from halo, C₁-C₃alkyl, C₁-C₃haloalkyl, and C₁-C₃alkoxy; or
R₃ is C₁-C₃alkyl or C₁-C₃haloalkyl;
R₄ is pyridine, pyrimidine, pyrazine or pyridazine; or
R₄ is pyridine, pyrimidine, pyrazine or pyridazine each of which, independently of each other, is substituted with one to two substituents independently selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, NH₂C(O)—, NH₂C(S)—, (OH)N═C(NH₂)— and a 5-membered heteroaryl ring optionally substituted by 1 to 3 substituents independently selected from halogen, C₁-C₃alkyl, C₁-C₃haloalkyl, C₁-C₃alkoxy and C₁-C₃haloalkoxy;
R₄ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl; or
R₄ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl each of which, independently of each other, is substituted with one to two substituents independently selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₅-C₄halocycloalkoxy, NH₂C(O)—, NH₂C(S)—, (OH)N═C(NH₂)— and a 5-membered heteroaryl ring optionally substituted by 1 to 3 substituents independently selected from halogen, C₁-C₃alkyl, C₁-C₃haloalkyl, C₁-C₃alkoxy and C₁-C₃haloalkoxy;
R₄ₐ is pyridine, pyrimidine, pyrazine, or pyridazine; or
R₄ₐ is pyridine, pyrimidine, pyrazine or pyridazine each of which, independently of each other, is substituted with one to three substituents independently selected from C₁-C₃alkyl, C₁-C₃haloalkyl, C₁-C₃alkoxy, C₃-C₄cycloalkyl, halogen, hydroxyl, cyano and C₁-C₃haloalkoxy; or
R₄ₐ is Y1, Y2, Y3 or Y4

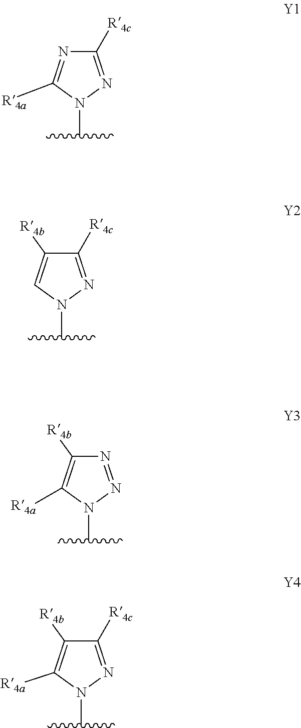

wherein, R′₄ₐ, R′_{4b} and R′_{4c}, independently of each other and independently of Y1 to Y4, are selected from hydrogen, halogen, CN, C₁-C₃alkyl, C₁-C₃haloalkyl, C₃-C₄cycloalkyl, C₁-C₃alkoxy and C₁-C₃haloalkoxy;
R₄ₐ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl; or
R₄ₐ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl each of which, independently of each other, is substituted with one to two substituents independently selected from C₁-C₃alkyl, C₁-C₃haloalkyl, C₁-C₃alkoxy, C₃-C₄cycloalkyl, halogen, hydroxyl, cyano, and C₁-C₃haloalkoxy; R₅ is hydrogen, C₁-C₃alkyl, C₁-C₃haloalkyl, C₃-C₄cycloalkyl, C₁-C₃alkoxy, C₃-C₄alkoxyC(O)—, (C₁-C₃alkoxy)₂CH—, halogen, CN, NH₂C(O), amino (i.e. NH₂), (C₁-C₃alkyl)amino, di(C₁-C₃alkyl)amino, hydroxy, C₃-C₄halocycloalkyl, C₃-C₄cyanocycloalkyl, C₂-C₆alkenyl, C₂-C₆haloalkenyl, C₂-C₆alkynyl, C₂-C₆haloalkynyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, C₁-C₃alkoxy-C₁-C₃alkyl, C₁-C₃alkoxy-C₁-C₃alkoxy-C₁-C₃alkyl, (C₁-C₃alkyl)sulfonylamino, (C₁-C₃alkyl)sulfonyl(C₁-C₃alkyl)amino, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), (C₁-C₃alkyl)C(O)(C₁-C₃alkyl)N, (C₁-C₃alkyl)C(O)NH, (C₁-C₃alkyl)C(O), (C₁-C₃alkoxy)C(O), HC(O), diphenylmethanimine, C₁-C₃haloalkoxy, phenyl, or a 5-membered heteroaromatic ring; or
R₅ is phenyl substituted with one to three substituents selected from C₁-C₃alkyl, C₁-C₃haloalkyl, C₁-C₃alkoxy, C₃-C₄cycloalkyl, halogen, CN, and hydroxyl; or
R₅ is a 5-membered heteroaromatic ring substituted with one to three substituents selected from C₁-C₃alkyl, C₁-C₃haloalkyl, C₁-C₃alkoxy, C₃-C₄cycloalkyl, halogen, CN, and hydroxyl;
R₅ₐ and R_{5b} are, independently of each other, selected from hydrogen, halogen, CN, C₁-C₃alkyl, C₁-C₃haloalkyl, C₃-C₄cycloalkyl, C₁-C₃alkoxy and C₁-C₃haloalkoxy;
R₆ is phenyl, benzyl, heteroaryl, C₃-C₆cycloalkyl, or C₃-C₆cycloalkylC₁-C₃alkyl; or
R₆ is phenyl, benzyl, heteroaryl, C₃-C₆ cycloalkyl, or C₃-C₆cycloalkylC₁-C₃alkyl, each of which, independent of each other, is substituted with one to three substituents independently selected from Rₓ;
R₇ is hydrogen, C₁-C₃alkyl or C₃-C₆cycloalkyl; or
R₇ is C₁-C₃alkyl or C₃-C₆cycloalkyl, each of which, independent of each other, is substituted with one to three substituents independently selected from R_{z};
R₈ is hydrogen, C₁-C₃alkyl or C₃-C₆cycloalkyl; or
R₈ is C₁-C₃alkyl or C₃-C₆cycloalkyl, each of which, independent of each other, is substituted with one to three substituents independently selected from R_{z}; or
R₇ and R₈ together is —(CH₂)₂—O—(CH₂)₂— and form 6-membered ring with the nitrogen atom;
Rₓ is independently selected from halogen, C₁-C₃alkyl, C₁-C₃haloalkyl, C₁-C₃alkoxy, C₁-C₃haloalkoxy, NO₂, SF₅, CN, C(O)NH₂, C(S)NH₂, C₃-C₆cycloalkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl;
R_{Y} is C₁-C₄ alkyl, C₃-C₅ alkenyl, C₃-C₄ cycloalkyl, C₂-C₅ alkynyl, or benzyl;
R_{z} is selected from oxo, halogen, C₁-C₃ alkyl, C₁-C₃haloalkyl, C₁-C₃alkoxy, C₁-C₃haloalkoxy and CN; or an agrochemically acceptable salt, stereoisomer, enantiomer, tautomer, and N-oxide of the compound of formula 1.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula I according to the invention are in free form, in oxidized form as a N-oxide or in salt form, e.g., an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula I according to the invention also include hydrates which may be formed during the salt formation.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂fluoroalkyl” would refer to a C₁-C₂alkyl radical which carries 1, 2, 3, 4, or 5 fluorine atoms, for example, any one of difluoromethyl, trifluoromethyl, 1-fluoroethyl, 2-fluoroethyl, 2,2-difluoroethyl, 2,2,2-trifluoroethyl, 1,1,2,2-tetrafluoroethyl or pentafluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of the radicals methoxy, ethoxy, n-propoxy, 1-methylethoxy, n-butoxy, 1-methylpropoxy, 2-methylpropoxy or 1,1-dimethylethoxy. The term “haloC₁-Cₙalkoxy” as used herein refers to a C₁-Cₙalkoxy radical where one or more hydrogen atoms on the alkyl radical is replaced by the same or different halo atom(s)—examples include trifluoromethoxy, 2-fluoroethoxy, 3-fluoropropoxy, 3,3,3-trifluoropropoxy, 4-chlorobutoxy.

The term “C₁-Cₙcyanoalkyl” as used herein refers to a straight chain or branched saturated C₁-Cₙalkyl radical having 1 to n carbon atoms (as mentioned above), where one of the hydrogen atoms in these radicals is be replaced by a cyano group: for example, cyanomethyl, 2-cyanoethyl, 2-cyanopropyl, 3-cyanopropyl, 1-(cyanomethyl)-2-ethyl, 1-(methyl)-2-cyanoethyl, 4-cyanobutyl, and the like.

The term “C₃-Cₙcycloalkyl” as used herein refers to 3-n membered cycloalkyl groups such as cyclopropane, cyclobutane, cyclopentane and cyclohexane.

The term “C₃-Cₙcycloalkylcarbonyl” as used herein refers to a 3-n membered cycloalkyl group attached to a carbonyl (C═O) group, which carbonyl group is connected to the rest of the molecule. Similarly, the terms “C₁-Cₙalkylcarbony”, “C₁-Cₙalkoxycarbonyl”, “phenyloxycarbonyl” and “benzyloxycarbonyl” as used herein refers to an alkyl, alkoxy, phenyloxy and benzyloxy group attached to a carbonyl (C═O) group, which carbonyl group is connected to the rest of the molecule.

The term “C₃-C₄cycloalkyl-C₁-C₂alkyl” as used herein refers to 3 or 4 membered cycloalkyl group with either a methylene or ethylene group, which methylene or ethylene group is connected to the rest of the molecule. In the instance, the C₃-C₄cycloalkyl-C₁-C₂alkyl- group is substituted, the substituent(s) can be on the cycloalkyl group and/or on the alkyl group.

The term “C₃-C₆cycloalkylC₁-C₄haloalkoxy” as used herein refers to a 3 to 6 membered cycloalkyl group connected to a 1 to 4 membered haloalkoxy, which haloalkoxy group is connected to the rest of the molecule.

The term “aminocarbonylC₁-Cₙalkyl” as used herein refers to an alkyl radical where one of the hydrogen atoms in the radical is replaced by CONH₂ group.

The term “hydroxycarbonylC₁-Cₙalkyl” as used herein refers to an alkyl radical where one of the hydrogen atoms in the radical is replaced by COOH group.

The term “C₁-Cₙalkylsulfanyl” as used herein refers to a C₁-Cₙalkyl moiety linked through a sulfur atom. Similarly, the term “C₁-Cₙhaloalkylthio” or “C₁-Cₙhaloalkylsulfanyl” as used herein refers to a C₁-Cₙhaloalkyl moiety linked through a sulfur atom. Similarly, the term “C₃-Cₙcycloalkylsulfanyl” refers to 3-n membered cycloalkyl moiety linked through a sulfur atom.

The term “C₁-Cₙalkylsulfinyl” as used herein refers to a C₁-Cₙalkyl moiety linked through the sulfur atom of the S(═O) group. Similarly, the term “C₁-Cₙhaloalkylsulfinyl” or “C₁-Cₙhaloalkylsulfinyl” as used herein refers to a C₁-Cₙhaloalkyl moiety linked through the sulfur atom of the S(═O) group.

Similarly, the term “C₃-Cₙcycloalkylsulfinyl” refers to 3-n membered cycloalkyl moiety linked through the sulfur atom of the S(═O) group.

The term “C₁-Cₙalkylsulfonyl” as used herein refers to a C₁-Cₙalkyl moiety linked through the sulfur atom of the S(═O)₂ group. Similarly, the term “C₁-Cₙhaloalkylsulfonyl” or “C₁-Cₙhaloalkylsulfonyl” as used herein refers to a C₁-Cₙhaloalkyl moiety linked through the sulfur atom of the S(═O)₂ group.

Similarly, the term “C₃-Cₙcycloalkylsulfonyl” refers to 3-n membered cycloalkyl moiety linked through the sulfur atom of the S(═O)₂ group The term “trimethylsilaneC₁-Cₙalkyl” as used herein refers to an C₁-Cₙalkyl radical where one of the hydrogen atoms in the radical is replaced by a —Si(CH₃)₃ group.

The term “C₁-C₄alkylsulfonylamino” as used herein refers to C₁-Cₙalkylsulfonyl moiety linked through an amino (or NH) group; examples are methylsulfonyl amino (MeS(O)₂NH—).

The term “aminosulfonyl” as used herein refers to an amino moiety linked through a sulfonyl group, i.e. NH₂S(O)₂—.

The term “C₁-C₄alkylaminosulfonyl” as used herein refers to a C₁-Cₙalkylamino moiety linked through a sulfonyl group; examples are, i.e. MeNHS(O)₂—, EtNHS(O)₂.

The term “di(C₁-C₄alkyl)aminosulfonyl” as used herein refers to a di(C₁-C₄alkyl)amino moiety linked through a sulfonyl group; examples are, i.e. Me₂NS(O)₂—, Et(Me)NS(O)₂.

The term “C₃-C₆cycloalkylaminosulfonyl” as used herein refers to a C₃-C₆cycloalkylamino moiety linked through a sulfonyl group; examples are, i.e. cyclopropylNHS(O)₂—.

The term “—O—C₁-C₂haloalkanediyl-O—” as used herein refers to a straight-chain saturated halogenated divalent group attached via each of the oxygen atoms and having 1 or 2 carbon atoms between the oxygen atoms. When referring to the T ring, the —O—C₁-C₂haloalkanediyl-O— group is connected to adjacent ring members of the T ring (i.e. phenyl, pyridine, pyrimidine, pyrazine, pyridazine or a five or nine membered heteroaromatic ring) via the oxygen atoms of the —O—C₁-C₂haloalkanediyl-O—group and the oxygen atoms are linked by 1 to 2 carbon atoms substituted with one or more halogen atoms. Examples include —OCF₂O— and —OCF₂CF₂O—. Together with the two adjacent ring members of the T ring to which it is attached, the —O—C₁-C₂haloalkanediyl-O— group thus forms a 5- or 6-membered ring.

The term “C₂-Cₙalkenyl” as used herein refers to a straight or branched alkenyl chain having from two to n carbon atoms and one or two double bonds, for example, ethenyl, prop-1-enyl, but-2-enyl.

The term “C₂-Cₙhaloalkenyl” as used herein refers to a C₂-Cₙalkenyl moiety substituted with one or more halo atoms which may be the same or different.

The term “C₂-Cₙalkynyl” as used herein refers to a straight or branched alkynyl chain having from two to n carbon atoms and one triple bond, for example, ethynyl, prop-2-ynyl, but-3-ynyl, The term “C₂-Cₙhaloalkynyl” as used herein refers to a C₂-Cₙalkynyl moiety substituted with one or more halo atoms which may be the same or different.

Halogen or “halo” is generally fluorine, chlorine, bromine, or iodine. This also applies, correspondingly, to halogen in combination with other meanings, such as haloalkyl.

The term “heteroaryl” as used herein refers to a 5- or 6-membered aromatic monocyclic ring having 1 to 3 heteroatoms independently selected from N, O and S. Examples are heteroaryls J-1 to J-41 shown in Scheme A below. Preferred heteroaryl is pyridyl, pyrimidinyl and pyrazolyl.

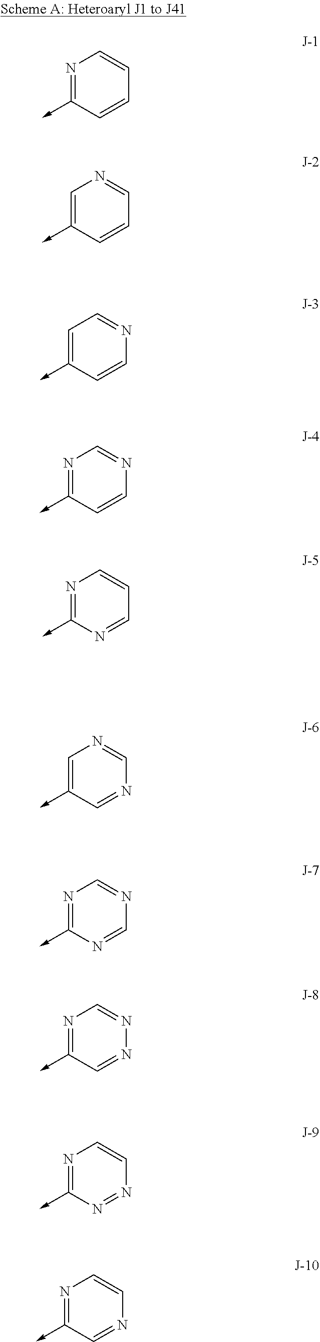

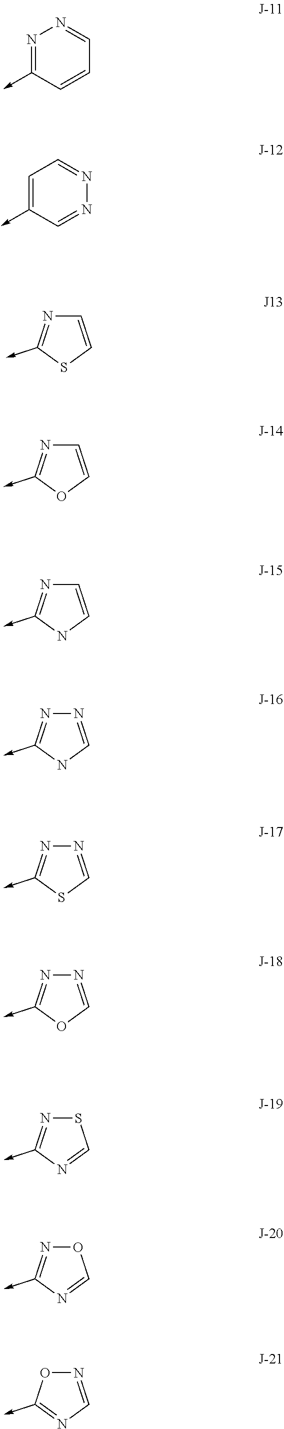

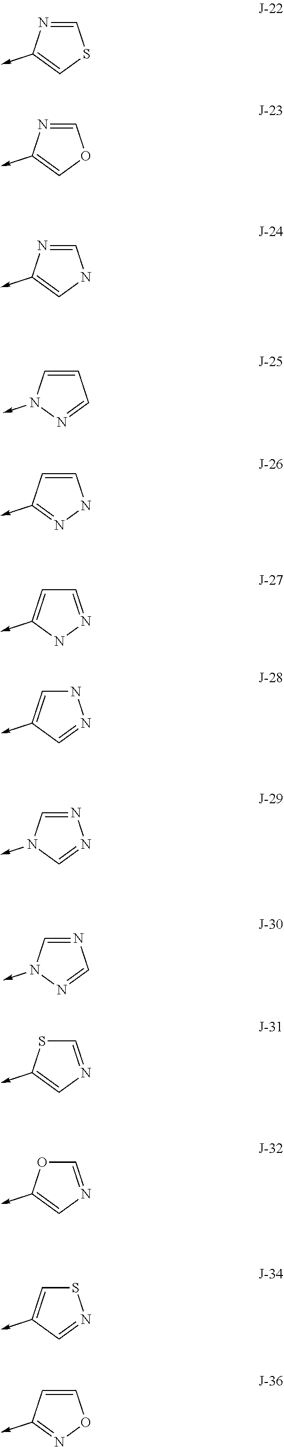

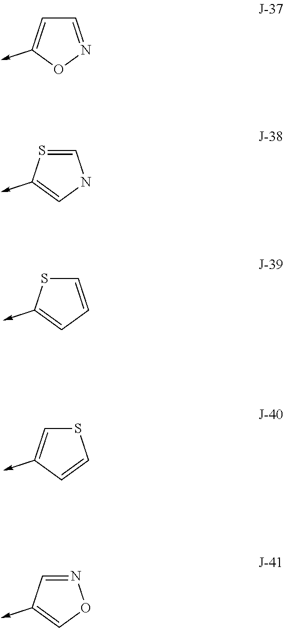

Examples of nine membered heteroaromatic rings for T are heteroaromatics made up of two rings, having 1 to 3 carbon atoms replaced independently by nitrogen, sulfur, or oxygen (the heteroatoms can be in one ring or distributed amongst the two). Examples are purinyl, thieno[2,3-d]pyrimidinyl, [1,2,4]triazolo[1,5-a]pyrimidinyl, quinolinyl, cinnolinyl, quinoxalinyl, indolyl, indazolyl, benzimidazolyl, benzothiophenyl and benzothiazolyl.

The term “nine or ten membered bicyclic heterocyclic ring” as used herein in context of the ring formed by T and R_{1b} with the N atom refers to a partially or fully saturated ring; having in addition to the N atom, optionally 1 or 2 more heteroatoms selected from N and O. An example of a 9-membered heterocyclic ring formed by R_{1b} and T together is indolinyl.

The term “spiroheterocyclic ring” as used herein refers to an 8- to 12-membered spiro ring, preferably a ten membered spiro ring, having 3 to 4 heteroatoms selected from N and O, which ring is partially or fully saturated. If substituted, the substituent can be on a carbon atom or a heteroatom. An example of spiroheterocyclic ring is oxa-2,8-diazaspiro[4.5]dec-2-en-3-yl.

The pyridine, pyrimidine, pyrazine and pyridazine groups (unsubstituted or substituted) for R₄ and R₄ₐ are each connected via a carbon atom on the respective ring to the rest of the compound.

As used herein, the term “controlling” refers to reducing the number of pests, eliminating pests and/or preventing further pest damage such that damage to a plant or to a plant derived product is reduced.

The staggered line as used herein, for example, in Q^{a} and Y1, and the arrow used, for example, in scheme A, represent the point of connection or attachment to the rest of the compound.

As used herein, the term “pest” refers to insects and molluscs that are found in agriculture, horticulture, forestry, the storage of products of vegetable origin (such as fruit, grain, and timber); and those pests associated with the damage of man-made structures. The term pest encompasses all stages in the life cycle of the pest.

As used herein, the term “effective amount” refers to the amount of the compound, or a salt thereof, which, upon single or multiple applications provides the desired effect.

An effective amount is readily determined by the skilled person in the art, by the use of known techniques and by observing results obtained under analogous circumstances. In determining the effective amount, a number of factors are considered including, but not limited to: the type of plant or derived product to be applied; the pest to be controlled & its lifecycle; the particular compound applied; the type of application; and other relevant circumstances.

As one of ordinary skill in the art will appreciate, compounds of formula I contain a stereogenic centre which is indicated with an asterisk in the structure below:

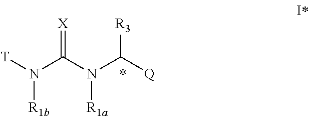

where T, X, R₁ₐ, R_{1b}, R₃, and Q are as defined in the first aspect.

The present invention contemplates both racemates and individual enantiomers. Compounds having preferred stereochemistry are set out below.

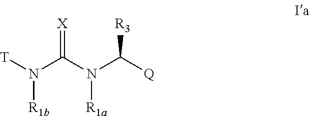

Particularly preferred compounds of the present invention are compounds of formula I′a where T, X, R₁ₐ, R_{1b}, R₃, and Q are as defined in the first aspect, and stereoisomers, enantiomers, tautomers, and N-oxides of the compounds of formula (I′a), and agrochemically acceptable salts thereof.

The term “optionally substituted” as used herein means that the group referenced is either unsubstituted or is substituted by a designated substituent. For example, “C₃-C₄cycloalkyl is optionally substituted with 1 or 2 halo atoms” means C₃-C₄cycloalkyl, C₃-C₄cycloalkyl substituted with 1 halo atom and C₃-C₄cycloalkyl substituted with 2 halo atoms.

Embodiments according to the invention are provided as set out below.

In an embodiment of each aspect of the invention,
A. X is oxygen; or
B. X is sulfur.

In an embodiment of each aspect of the invention, R₁ₐ is
A. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, C₃-C₄cycloalkylC₁-C₂alkyl- wherein the C₃-C₄cycloalkyl group is substituted with 1 or 2 halogen atoms, oxetan-3-yl-CH₂—, C₁-C₃alkylcarbonyl, C₁-C₃alkoxycarbonyl, phenyloxycarbonyl, benzyloxycarbonyl, or benzyl; or
B. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
C. hydrogen, C₁-C₆alkyl, C₁-C₆cyanoalkyl, aminocarbonylC₁-C₆alkyl, hydroxycarbonylC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
D. hydrogen, C₁-C₆alkyl, C₁-C₆cyanoalkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
E. hydrogen, C₁-C₄alkyl, C₁-C₄cyanoalkyl, C₁-C₄alkoxy-C₁-C₃alkyl, C₁-C₄haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
F. hydrogen, C₁-C₃alkyl, C₁-C₃cyanoalkyl, C₁-C₃alkoxy-C₁-C₃alkyl, C₁-C₃haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
G. hydrogen, methyl, ethyl, cyanomethyl, methoxymethyl, cyclopropyl-methyl, allyl, propargyl, benzyloxycarbonyl, or benzyl; or
H. hydrogen, methyl, ethyl, allyl, propargyl, or cyclopropyl-methyl; or
I. hydrogen, methyl, propargyl, or cyclopropyl-methyl;
J. hydrogen, or methyl; or
K. hydrogen.

In an embodiment of each aspect of the invention, R_{1b} is
A. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, C₃-C₄cycloalkylC₁-C₂alkyl- wherein the C₃-C₄cycloalkyl group is substituted with 1 or 2 halogen atoms, oxetan-3-yl-CH₂—, C₁-C₃alkylcarbonyl, C₁-C₃alkoxycarbonyl, phenyloxycarbonyl, benzyloxycarbonyl, or benzyl; or
B. hydrogen, C₁-C₆alkyl, C₁-C₆cyanoalkyl, aminocarbonylC₁-C₆alkyl, hydroxycarbonylC₁-C₆alkyl, C₁-C₆nitroalkyl, trimethylsilaneC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
C. hydrogen, C₁-C₆alkyl, C₁-C₆cyanoalkyl, aminocarbonylC₁-C₆alkyl, hydroxycarbonylC₁-C₆alkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
D. hydrogen, C₁-C₆alkyl, C₁-C₆cyanoalkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl; or
E. hydrogen, C₁-C₄alkyl, C₁-C₄cyanoalkyl, C₁-C₄alkoxy-C₁-C₃alkyl, C₁-C₄haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
F. hydrogen, C₁-C₃alkyl, C₁-C₃cyanoalkyl, C₁-C₃alkoxy-C₁-C₃alkyl, C₁-C₃haloalkyl, C₂-C₄alkenyl, C₂-C₄haloalkenyl, C₂-C₄alkynyl, C₂-C₄haloalkynyl, C₃-C₄cycloalkylC₁-C₂alkyl-, benzyloxycarbonyl, or benzyl; or
G. hydrogen, methyl, ethyl, cyanomethyl, methoxymethyl, cyclopropyl-methyl, allyl, propargyl, benzyloxycarbonyl, or benzyl; or
H. hydrogen, methyl, ethyl, allyl, propargyl, or cyclopropyl-methyl; or
I. hydrogen, methyl, propargyl, or cyclopropyl-methyl; or
J. hydrogen, or methyl;
K. hydrogen.

In an embodiment of each aspect of the invention, R₁ₐ and R_{1b} together is
A. selected from —(CH₂)₂—, —(CH₂)₃—, —CH₂—O—CH₂—, —CH₂—S—CH₂—, —CH₂—S(═O)—CH₂—, and —CH₂—SO₂—CH₂—; or
B. selected from —(CH₂)₃—, —CH₂—O—CH₂—, —CH₂—S—CH₂—, —CH₂—S(═O)—CH₂—, and —CH₂—SO₂—CH₂—; or
C. selected from —CH₂—O—CH₂—, —CH₂—S—CH₂—, —CH₂—S(═O)—CH₂—, and —CH₂—SO₂—CH₂—; or
D. —CH₂—O—CH₂—.

In an embodiment of each aspect of the invention, T is
A. phenyl, pyridine, pyrimidine, pyrazine, pyridazine or a heteroaromatic ring selected from J-13, J-16, J-22, J-25, J-26, J-27, J-28, J-31, J-36, J-37, J-38, J-39, J-40, J-41, thieno[2,3-d]pyrimidinyl, [1,2,4]triazolo[1,5-a]pyrimidinyl, and indazolyl, which are, independently of each other, unsubstituted or substituted with one to three substituents independently selected from R₂;
B. phenyl, pyridine, pyrimidine, pyrazine, pyridazine or a heteroaromatic ring selected from J-13, J-16, J-22, J-25, J-26, J-27, J-28, J-31, J-36, J-37, J-38, J-39, J-40, J-41, thieno[2,3-d]pyrimidinyl, [1,2,4]triazolo[1,5-a]pyrimidinyl, and indazolyl, each of which, independent of each other, is substituted with one to three substituents independently selected from R₂; or
C. phenyl, pyridine, pyrimidine, or a heteroaromatic ring selected from J-13, J-16, J-22, J-25, J-26, J-27, J-28, J-31, J-36, J-37, J-38, J-39, J-40, J-41, thieno[2,3-d]pyrimidinyl, [1,2,4]triazolo[1,5-a]pyrimidinyl, and indazolyl, each of which, independent of each other, is substituted with one to three substituents independently selected from R₂; or
D. phenyl, pyridine, pyrimidine or a heteroaromatic ring selected from J-16, J-26, J-27 and J-28, J-41, thieno[2,3-d]pyrimidinyl, [1,2,4]triazolo[1,5-a]pyrimidinyl, and indazolyl, each of which, independent of each other, is substituted with one to three substituents independently selected from R₂; or
E. phenyl, pyrid-2-yl (or J-1), J-2, J-3, J-4. J-5, J-6, J-16, J-26, J-27, J-28, J-41, thieno[2,3-d]pyrimidinyl, [1,2,4]triazolo[1,5-a]pyrimidinyl, or indazolyl each of which, independent of each other, is substituted with one to three substituents independently selected from R₂; or
F. phenyl, pyrid-2-yl (or J-1), or J-2, each of which, independent of each other, is substituted with one to three substituents independently selected from R₂; or
G. phenyl, pyrid-2-yl (or J-1), or J-2, each of which, independent of each other, is substituted with one to three substituents independently selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ, OR₆, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring; or
H. phenyl, pyrid-2-yl (or J-1), or J-2, each of which, independent of each other, is substituted with one to three substituents independently selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1, J-13 and J-25, each of C₃-C₄cycloalkyl, phenyl or heteroaryl, independent of each other, being substituted with one to three substituents Rₓ, ORB, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered spiroheterocyclic ring substituted with a C₁-C₃alkoxy; or
I. phenyl, pyrid-2-yl (or J-1), or J-2, each of which, independent of each other, is substituted with one to three substituents independently selected from halogen, C₁-C₃alkyl, C₁-C₃haloalkyl, C₁-C₃haloalkoxy, methoxy, CN, OR₆ (where RB is phenyl, J-1 or J-17, each of which can be substituted by one to three substituents independently selected from Rₓ), and —O—C₁-C₂haloalkanediyl-O—; or
J. phenyl, pyrid-2-yl (or J-1), or J-2, each of which, independent of each other, is substituted with one to three substituents independently selected from chlorine, fluorine, bromine, iodine, methyl, difluoromethyl, trifluoromethyl, difluoromethoxy, trifluoromethoxy, 1,1,2,2-tetrafluoroethoxy, 1,1,1,2,2-pentafluoroethoxy, cyano, O—CF₂CF₂—O—, and O—CF₂—O—;
K. phenyl, pyrid-2-yl (or J-1), or J-2, each of which, independent of each other, is substituted with one to three substituents independently selected from chlorine, fluorine, bromine, iodine, methyl, trifluoromethyl, difluoromethoxy, and 1,1,2,2-tetrafluoroethoxy; or
L. 2,4-bis(trifluoromethyl)phenyl, 2,4-chloro-5-(1,1,2,2-tetrafluoroethoxy)phenyl, 2-bromo-4-chloro-5-(trifluoromethyl)phenyl, 4-bromo-2-(difluoromethoxy)-5-fluoro-phenyl, 2-bromo-4-(difluoromethoxy)phenyl, 2-iodo-4-(trifluoromethyl)phenyl, 3,5-bis(trifluoromethyl)phenyl, 2-chloro-4-(trifluoromethyl)phenyl, 4-chloro-2-(trifluoromethyl)phenyl, 2,4-dibromophenyl, 5-chloro-2-(trifluoromethyl)phenyl, 2-chloro-5-(trifluoromethyl)phenyl, 4-chloro-3-(trifluoromethyl)phenyl, 3-chloro-4-(trifluoromethyl)phenyl, 4-chloro-5-cyano-2-fluoro-phenyl, 2-methyl-4-(trifluoromethyl)phenyl, 4-chloro-6-(trifluoromethyl)-3-pyridyl, 4,6-dibromo-3-pyridyl, 4-bromo-6-(trifluoromethyl)-3-pyridyl, 4-methyl-6-(trifluoromethyl)-3-pyridyl, 4-methoxy-6-(trifluoromethyl)-3-pyridyl, 3-chloro-5-(trifluoromethyl)-2-pyridyl, 5-chloro-4-(trifluoromethyl)thiazol-2-yl, 4-methyl-6-(trifluoromethyl)-3-pyridyl, or 4-methoxy-6-(trifluoromethyl)-3-pyridyl; or
M. 2,4-bis(trifluoromethyl)phenyl, 2,4-chloro-5-(1,1,2,2-tetrafluoroethoxy)phenyl, 2-bromo-4-chloro-5-(trifluoromethyl)phenyl, 2-bromo-4-(difluoromethoxy)phenyl, 2-iodo-4-(trifluoromethyl)phenyl, 3,5-bis(trifluoromethyl)phenyl, 2-chloro-4-(trifluoromethyl)phenyl, 4-chloro-2-(trifluoromethyl)phenyl, 2,4-dibromophenyl, 5-chloro-2-(trifluoromethyl)phenyl, 2-chloro-5-(trifluoromethyl)phenyl, 4-chloro-3-(trifluoromethyl)phenyl, 3-chloro-4-(trifluoromethyl)phenyl, 4-chloro-6-(trifluoromethyl)-3-pyridyl, 4,6-dibromo-3-pyridyl, 3-chloro-5-(trifluoromethyl)-2-pyridyl, 5-chloro-4-(trifluoromethyl)thiazol-2-yl, 4-methyl-6-(trifluoromethyl)-3-pyridyl, or 4-methoxy-6-(trifluoromethyl)-3-pyridyl; or
N. 4-bromo-2-(difluoromethoxy)-5-fluoro-phenyl, 4-chloro-5-cyano-2-fluoro-phenyl, 2-methyl-4-(trifluoromethyl)phenyl, 4-chloro-6-(trifluoromethyl)-3-pyridyl, 4,6-dibromo-3-pyridyl, 4-bromo-6-(trifluoromethyl)-3-pyridyl, 4-methyl-6-(trifluoromethyl)-3-pyridyl, 4-methoxy-6-(trifluoromethyl)-3-pyridyl, 4-methyl-6-(trifluoromethyl)-3-pyridyl, or 4-methoxy-6-(trifluoromethyl)-3-pyridyl; or
O. 2,4-(trifluoromethyl)phenyl, 2,4-chloro-5-(1,1,2,2-tetrafluoroethoxy)phenyl, 2-bromo-4-chloro-5-(trifluoromethyl)phenyl, 2-bromo-4-(difluoromethoxy)phenyl, 2-iodo-4-(trifluoromethyl)phenyl, 4-chloro-6-(trifluoromethyl)-3-pyridyl, 4,6-dibromo-3-pyridyl, 4-methyl-6-(trifluoromethyl)-3-pyridyl, or 4-methoxy-6-(trifluoromethyl)-3-pyridyl.

In an embodiment of each aspect of the invention, R_{1b} and T together with the nitrogen atom form
A. a nine membered bicyclic heterocyclic ring, which can be substituted with one to three substituents independently selected from C₁-C₃haloalkyl, C₁-C₃alkoxy, C₃-C₄cycloalkyl, halogen, and C₁-C₃haloalkoxy; or
B. a nine membered bicyclic heterocyclic ring, which can be substituted with one to three substituents independently selected from trifluoromethyl, methoxy, trifluoromethoxy, cyclopropyl, chloro, fluoro, and bromo; or
C. an indoline ring, which can be substituted with one to three substituents independently selected from trifluoromethyl, methoxy, trifluoromethoxy, cyclopropyl, chloro, fluoro, and bromo; or
D. an indoline ring, which can be substituted with one to three substituents independently selected from trifluoromethyl, chloro, fluoro, and bromo; or
E. an indoline ring, substituted with one to three substituents independently selected from trifluoromethyl, chloro, fluoro, and bromo.

In an embodiment of each aspect of the invention, R₂ is, independent of each other, selected from
A. halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ, OR₆, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring; or
B. halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1, J-13 and J-25. pyrazolyl, each of C₃-C₄cycloalkyl, phenyl, heteroaryl and pyrazolyl, independent of each other, is substituted with one to three substituents Rₓ, OR₆, piperidin-2-one-1-yl, pyridin-2-one-1-yl, azetidin-1-yl optionally substituted with Rₓ, pyrrolidin-1-yl, C₃-CcycloalkylC₁-C₄alkyl optionally substituted with one or two substituents R_{z}, C₃-CcycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ and C₁-C₄alkylsulfinyl optionally substituted by one to three substituents Rₓ, C(O)NR₇R₈, C₁-C₄alkylsulfonylamino, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered spiroheterocyclic ring substituted with a C₁-C₃alkoxy; or
C. halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl or pyrazolyl, each of C₃-C₄cycloalkyl, phenyl, pyrazolyl, independent of each other, is substituted with one to two substituents Rₓ, ORB, azetidin-1-yl optionally substituted with Rₓ, C₃-C₆cycloalkylC₁-C₄alkyl optionally substituted with one or two substituents R_{z}, C₃-C₆cycloalkylC₁-C₃alkoxy optionally substituted with Rₓ, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
D. halogen, C₁-C₃alkyl, C₁-C₃haloalkyl, C₁-C₃alkoxy, C₁-C₃haloalkoxy, CN, C₃-C₄cycloalkyl, C₃-C₄cycloalkyl substituted with one to two substituents Rₓ, C₃-C₆cycloalkylcarbonyl, ORB, C₃-C₆cycloalkylC₁-C₄alkyl, C₃-C₆cycloalkylC₁-C₄alkyl substituted with one or two substituents R_{z}, C₁-C₄alkylsulfanyl, C₁-C₄alkylsulfanyl substituted by one to three substituents Rₓ, C₁-C₄alkylsulfonyl, C₁-C₄alkylsulfonyl substituted by one to three substituents Rₓ, C₁-C₄alkylsulfinyl, C₁-C₄alkylsulfinyl substituted by one to three substituents Rₓ, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
E. halogen, C₁-C₃alkyl, C₁-C₃haloalkyl, C₁-C₃alkoxy, C₁-C₃haloalkoxy, CN, C₃-C₄cycloalkyl, C₃-C₄cycloalkyl substituted with one to two substituents independently selected from halogen, C₁-C₃alkyl and C₁-C₃haloalkyl, C₃-C₄cycloalkylcarbonyl, ORB, C₃-C₄cycloalkylmethyl, C₃-C₄cycloalkylmethyl substituted with one to two substituents independently selected from oxo, halogen, C₁-C₃alkyl and C₁-C₃haloalkyl, C₁-C₂alkylsulfanyl substituted with one to three halogens, C₁-C₂alkylsulfonyl substituted with one to three halogens, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
F. halogen, C₁-C₃alkyl, C₁-C₃haloalkyl, C₁-C₃alkoxy, C₁-C₃haloalkoxy, CN, cyclopropyl, cyclopropyl substituted with one to two substituents independently selected from halogen, methyl and trifluoromethyl, cyclopropylcarbonyl, ORB, cyclopropylmethyl substituted with one to two substituents independently selected from oxo, halogen and trifluoromethyl, C₁-C₂alkylsulfanyl substituted with one to three halogens, C₁-C₂alkylsulfonyl substituted with one to three halogens, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
G. halogen, C₁-C₃alkyl, C₁-C₃haloalkyl, C₁-C₃haloalkylsulfanyl, C₁-C₃alkoxy, C₁-C₃haloalkoxy, CN, C₃-C₆cycloalkyl, C₃-C_{B}cycloalkyl substituted with one to three substituents independently selected from C₁-C₃alkyl, C₁-C₃haloalkyl, cyano and halogen, cyclopropylcarbonyl, OR₆, C₃-C₆cycloalkylC₁-C₄alkyl, C₃-C₆cycloalkylC₁-C₄alkyl substituted with one to five substituents independently selected from oxo, C₁-C₃alkyl, C₁-C₃haloalkyl, cyano and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, C₃-C₆cycloalkylsulfonyl, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
H. halogen, C₁-C₃alkyl, C₁-C₃haloalkyl, C₁-C₃haloalkylsulfanyl, C₁-C₃alkoxy, C₁-C₃haloalkoxy, CN, C₃-C₆cycloalkyl, C₃-C₆cycloalkyl substituted with one or two substituents independently selected from C₁-C₃haloalkyl, cyano and halogen, C₃-C₄cycloalkylcarbonyl, ORB, C₃-C₆cycloalkylC₁-C₄alkyl, C₃-C₆cycloalkylC₁-C₄alkyl substituted with one to three substituents independently selected from oxo, C₁-C₃haloalkyl, cyano and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, C₃-C₆cycloalkylsulfonyl, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
I. halogen, C₁-C₃haloalkyl, C₁-C₃haloalkylsulfanyl, C₁-C₃haloalkoxy, CN, C₃-C₆cycloalkyl, C₃-C₆cycloalkyl substituted with one or two substituents independently selected from C₁-C₃haloalkyl, cyano and halogen, C₃-C₄cycloalkylcarbonyl, ORB, C₃-C₆cycloalkylC₁-C₄alkyl, C₃-C₆cycloalkylC₁-C₄alkyl substituted with one to three substituents independently selected from oxo, C₁-C₃haloalkyl, cyano and halogen, C₁-C₅cyanoalkyl, C₁-C₄alkylsulfonyl, C₁-C₄haloalkylsulfonyl, C₁-C₄alkylsulfinyl, C₁-C₄haloalkylsulfinyl, C₃-C₆cycloalkylsulfanyl, C₃-C₆cycloalkylsulfinyl, C₃-C₆cycloalkylsulfonyl, C(O)NH(C₁-C₄alkyl), C₁-C₄alkylsulfonylamino, C₁-C₄alkylaminosulfonyl, C₃-C₆cycloalkylaminosulfonyl, and —O—C₁-C₂haloalkanediyl-O—; or
J. halogen, C₃-C₄cycloalkyl, C₃-C₄cycloalkylcarbonyl, C₃-C₄cycloalkyl-C₁-C₂alkyl optionally substituted with one to two substituents selected from oxo, halogen, C₁-C₃alkyl and C₁-C₃haloalkyl, C₁-C₃haloalkyl, OR₆ (where RB is phenyl, J-1 or J-17, each of which can be substituted by one to three substituents independently selected from Rₓ), C₁-C₃haloalkylsulfanyl, C₁-C₃haloalkysulfonyl, C₁-C₃alkoxy, C₁-C₃haloalkoxy, CN, and —O—C₁-C₂haloalkanediyl-O—; or
K. halogen, C₃-C₄cycloalkyl, C₁-C₃haloalkyl, OR₆ (where RB is phenyl, J-1 or J-17, each of which can be substituted by one to three substituents independently selected from Rₓ), C₁-C₃haloalkylsulfanyl, C₁-C₃haloalkysulfonyl, —O—CF₂—O—; —OCF₂CF₂O—, and C₁-C₃haloalkoxy; or
L. halogen, C₁-C₂haloalkyl, C₁-C₂haloalkylsulfanyl, C₁-C₂haloalkysulfonyl, —O—CF₂—O—; —OCF₂CF₂O—, and C₁-C₂haloalkoxy; or
M. chlorine, fluorine, bromine, iodine, cyclopropyl, difluoromethyl, trifluoromethyl, trifluoromethylsulfanyl, trifluoromethylsulfonyl, difluoromethoxy, trifluoromethoxy, 1,1,2,2-tetrafluoroethoxy, 1,1,1,2,2-pentafluoroethoxy, —O—CF₂CF₂—O—, and —O—CF₂—O—; or
N. fluorine, chlorine, bromine, iodine, difluoromethyl, trifluoromethyl, difluoromethoxy, trifluoromethoxy, 1,1,2,2-tetrafluoroethoxy, 1,1,1,2,2-pentafluoroethoxy, —O—CF₂CF₂—O—, and —O—CF₂—O—; or
O. fluorine, chlorine, bromine, iodine, trifluoromethyl, difluoromethoxy, 1,1,2,2-tetrafluoroethoxy, —O—CF₂CF₂—O—, and —O—CF₂—O—; or
P. fluorine, chlorine, bromine, iodine, trifluoromethyl, difluoromethoxy, and 1,1,2,2-tetrafluoroethoxy.

When R₂ is a divalent group, T carries only one such divalent group as R₂ and said divalent group is connected to T via two adjacent ring members of T. Examples of divalent groups include —O—C₁-C₂haloalkanediyl-O—, —CH₂—C(CH₃)₂—O—, —CH₂—C(CH₃)₂—S—, —CH₂—C(CH₃)₂—SO₂. Examples of —O—C₁-C₂haloalkanediyl-O— include —O—CF₂—CF₂—O—, —O—CFH—CF₂—O—, —O—CFH—CFH—O—, —OCH₂—CHF—O—, —OCH₂—CF₂—O—, —O—CF₂—O—, and —O—CFH—O—; preferably —O—CF₂CF₂—O—, and —O—CF₂—O—. In an embodiment, T is phenyl, pyridine, pyrimidine, pyrazine, pyridazine or a five or nine membered heteroaromatic ring, substituted via two adjacent ring members of the phenyl, pyridine, pyrimidine, pyrazine, pyridazine or five or nine membered heteroaromatic ring, with a divalent groups selected from —O—C₁-C₂haloalkanediyl-O—, —CH₂—C(CH₃)₂—O—, —CH₂—C(CH₃)₂—S—, —CH₂—C(CH₃)₂—SO₂, preferably selected from —O—CF₂CF₂—O—, and —O—CF₂—O—.

In an embodiment of each aspect of the invention, R₃ is
A. C₁-C₂alkyl or C₁-C₂haloalkyl; or
B. methyl or trifluoromethyl; or
C. methyl.

In an embodiment of each aspect of the invention, Q is
A. Q^{a}; or
B. Q^{b}; or
C. selected from Q^{a}-1 to Q^{a}-16, or selected from Q^{b}-1 to Q^{b}-13; or
D. selected from Q^{a}-1, Q^{a}-6, Q^{a}-7, Q^{a}-10, Q^{a}-15 and Q^{b}-1; or
E. Q^{a}-1, Q^{a}-15, or Q^{b}-1; or
F. Q^{a}-1, or Q^{b}-1;
G. Q^{a}-1; or
H. Q^{b}-1.

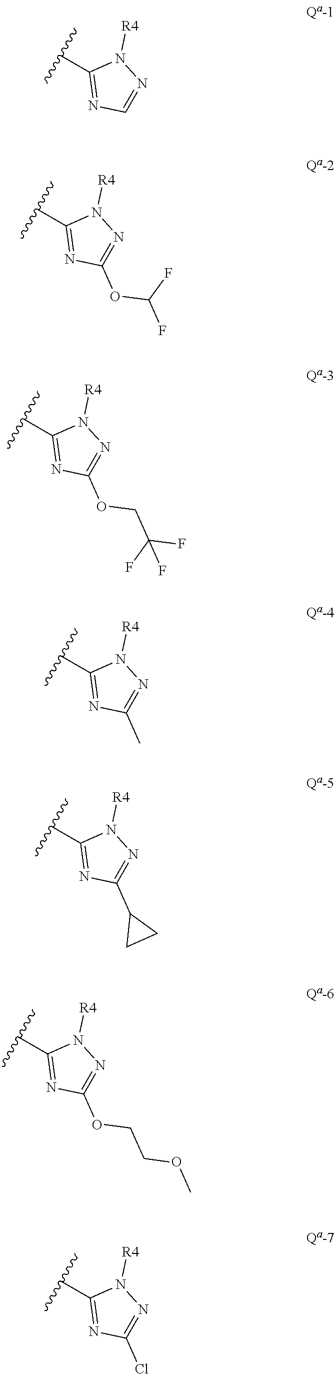

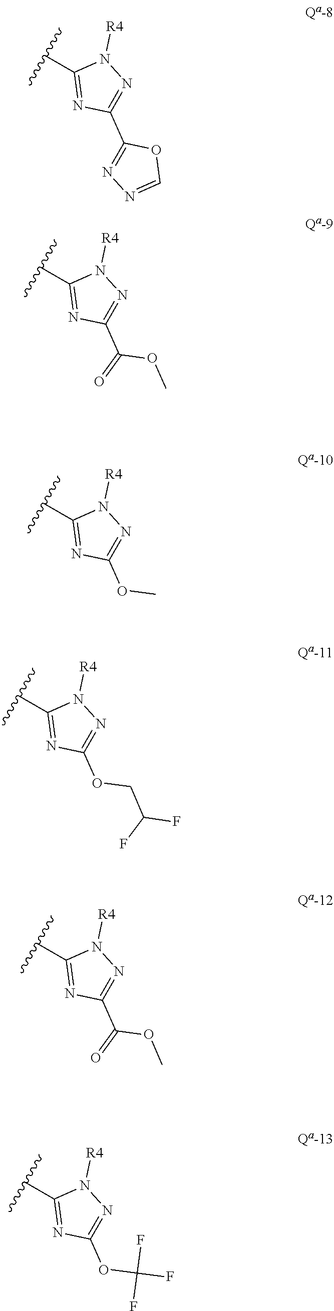

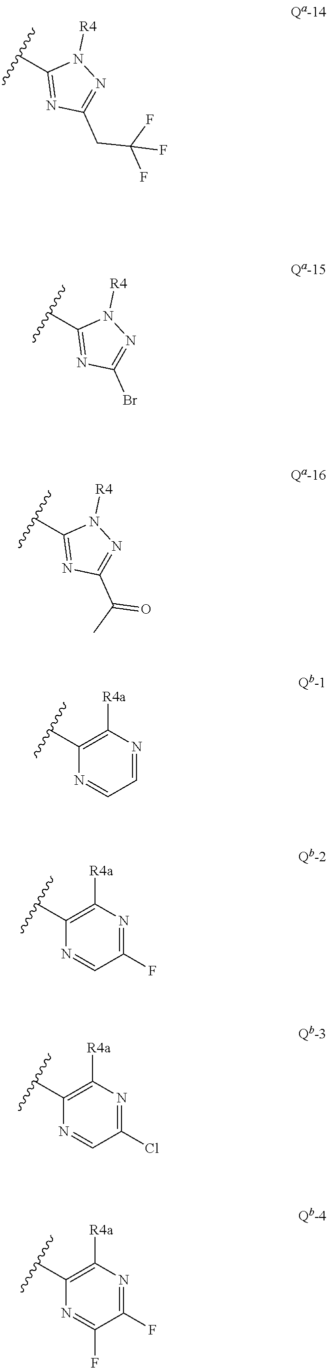

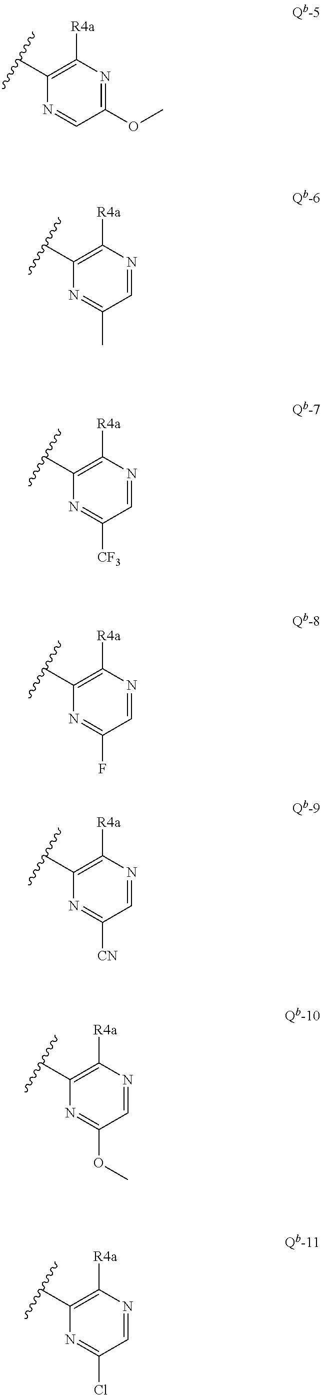

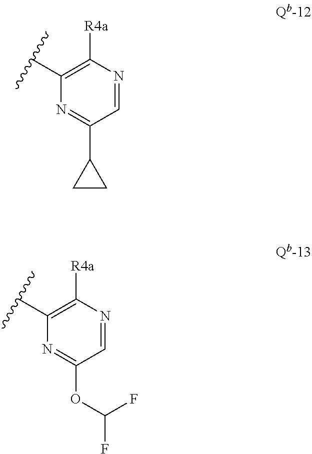

In an embodiment of each aspect of the invention, R₄ is
A. pyridine, or pyrimidine; wherein the pyridine or pyrimidine, independently of each other, is optionally substituted with one substituent selected from C₁-C₃alkyl, C₁-C₃haloalkyl, C₁-C₃alkoxy, C₃-C₄cycloalkyl, halo, hydroxyl, CN, C₁-C₄haloalkoxy, C₂-C₆haloalkenyloxy, C₂-C₆haloalkynyloxy, C₃-C₄halocycloalkoxy, C₃-CcycloalkylC₁-C₄haloalkoxy, NH₂C(O)—, NH₂C(S)—, (OH)N═C(NH₂)—, J-13 optionally substituted by 1 to 3 substituents independently selected from halogen, C₁-C₃alkyl, C₁-C₃haloalkyl, C₁-C₃alkoxy and C₁-C₃haloalkoxy, J-20 optionally substituted by 1 to 3 substituents independently selected from halogen, C₁-C₃alkyl, C₁-C₃haloalkyl, C₁-C₃alkoxy and C₁-C₃haloalkoxy and 1H-tetrazol-5-yl; or
B. pyridine or pyrimidine, wherein the pyridine or pyrimidine, independently of each other, is optionally substituted with one substituent selected from C₁-C₃alkyl, C₁-C₃haloalkyl, C₁-C₃alkoxy, C₃-C₄cycloalkyl, halo, hydroxyl, CN, C₁-C₄haloalkoxy, C₂-C₆haloalkenyloxy, C₂-C₆haloalkynyloxy, C₃-C₄halocycloalkoxy, C₃-CcycloalkylC₁-C₄haloalkoxy, NH₂C(O)—, NH₂C(S)—, (OH)N═C(NH₂)—, J-13 optionally substituted by C₁-C₃haloalkyl, J-20 optionally substituted by C₁-C₃haloalkyl and 1H-tetrazol-5-yl; or
C. pyridine, wherein the pyridine is optionally substituted with one substituent selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, C₃-C₆cycloalkylC₁-C₄haloalkoxy, NH₂C(O)—, NH₂C(S)—, (OH)N═C(NH₂)—, J-13 optionally substituted by C₁-C₃haloalkyl, J-20 optionally substituted by C₁-C₃haloalkyl and 1H-tetrazol-5-yl; or
D. pyrimidine; wherein the pyrimidine is optionally substituted with one substituent selected from C₁-C₃alkyl, C₁-C₃haloalkyl, C₁-C₃alkoxy, C₃-C₄cycloalkyl, halo, hydroxyl, CN, C₁-C₄haloalkoxy, C₂-C₆haloalkenyloxy, C₂-C₆haloalkynyloxy, C₃-C₄halocycloalkoxy, C₃-C₆cycloalkylC₁-C₄haloalkoxy, NH₂C(O)—, NH₂C(S)—, (OH)N═C(NH₂)—, J-13 optionally substituted by trifluoromethyl, J-20 optionally substituted by trifluoromethyl and 1H-tetrazol-5-yl; or
E. thiazole, pyridine, pyrimidine, pyrazine or pyridazine, wherein the thiazole, pyridine, pyrimidine, pyrazine or pyridazine is optionally substituted with one substituent selected from C₁-C₃alkyl, C₁-C₃haloalkyl, C₁-C₃alkoxy, C₃-C₄cycloalkyl, F, Cl, Br, CN and C₁-C₄haloalkoxy; or
F. thiazole, pyridine, pyrimidine, pyrazine or pyridazine, wherein the thiazole, pyridine, pyrimidine, pyrazine or pyridazine is optionally substituted with one substituent selected from C₁-C₃alkyl, C₃-C₄cycloalkyl, F, Cl, Br, CN and C₁-C₆haloalkoxy; or
G. thiazole, pyridine, pyrimidine, pyrazine or pyridazine, wherein the thiazole, pyridine, pyrimidine, pyrazine or pyridazine is optionally substituted with one substituent selected from cyclopropyl, F, Cl, Br, CN, trifluoromethoxy, difluoromethoxy, 2,2-difluoroethoxy and 2,2,2-trifluoroethoxy;
H. thiazole, pyridine, or pyrimidine, wherein the pyridine or pyrimidine is optionally substituted with one substituent selected from cyclopropyl, F, Cl, Br, CN, trifluoromethoxy, difluoromethoxy, 2,2-difluoroethoxy and 2,2,2-trifluoroethoxy; or
I. 5-cyanothiazol-2-yl, 5-cylopropylpyridine, 5-fluoropyridine, 5-chloropyridine, 5-bromopyridine, 5-difluoromethoxypyridine, 5-trifluoromethoxypyridine, 5-cyanopyridine, 5-(2,2-difluoroethoxy)-pyridine, 5-(2,2,2-trifluoroethoxy)-pyridine, pyridine, 5-cylopropylpyrimidine, 5-fluoropyrimidine, 5-chloropyrimidine, 5-bromopyrimidine, 5-difluoromethoxypyrimidine, 5-trifluoromethoxypyrimidine, 5-cyanopyrimidine, 5-(2,2-difluoroethoxy)-pyrimidine, 5-(2,2,2-trifluoroethoxy)-pyrimidine, or pyrimidine; or
J. 5-cyanothiazol-2-yl, 5-cylopropylpyridin-2-yl, 5-fluoropyridin-2-yl, 5-chloropyridin-2-yl, 5-bromopyridin-2-yl, 5-difluoromethoxypyridin-2-yl, 5-trifluoromethoxypyridin-2-yl, 5-cyanopyridin-2-yl, 5-(2,2-difluoroethoxy-pyridin-2-yl, 5-(2,2,2-trifluoroethoxy)-pyridin-2-yl, pyridin-2-yl, 5-cylopropylpyrimidin-2-yl, 5-fluoropyrimidin-2-yl, 5-chloropyrimidin-2-yl, 5-bromopyrimidin-2-yl, 5-difluoromethoxypyrimidin-2-yl, 5-trifluoromethoxypyrimidin-2-yl, 5-cyanopyrimidin-2-yl, 5-(2,2-difluoroethoxy)-pyrimidin-2-yl, 5-(2,2,2-trifluoroethoxy)-pyrimidin-2-yl, or pyrimidin-2-yl; or
K. 5-cyanothiazol-2-yl, pyrimidin-2-yl, pyridin-2-yl, 5-bromopyrimidin-2-yl, 5-bromopyridin-2-yl, 5-cyanopyrimidin-2-yl, or 5-cyanopyridin-2-yl; or
L. 5-cyanothiazol-2-yl, pyrimidin-2-yl, 5-bromopyrimidin-2-yl, 5-bromopyridin-2-yl, or 5-cyanopyridin-2-yl; or
M. 5-cyanothiazol-2-yl, pyrimidin-2-yl, or 5-cyanopyridin-2-yl; or
N. 5-cyanothiazol-2-yl; or
O. pyrimidin-2-yl; or
P. 5-cyanopyridin-2-yl.

In an embodiment of each aspect of the invention, R₄ₐ is
A. thiazole, pyridine, pyrimidine, pyrazine or pyridazine, wherein the thiazole, pyridine, pyrimidine, pyrazine or pyridazine, independent of each other, is optionally substituted with one substituent selected from C₁-C₃haloalkyl, C₃-C₄cycloalkyl, halogen, cyano and C₁-C₃haloalkoxy. or is selected from Y1 to Y4; or
B. thiazole, pyridine, pyrimidine, pyrazine or pyridazine, wherein the thiazole, pyridine, pyrimidine, pyrazine or pyridazine, independent of each other, is optionally substituted with one substituent selected from F, Cl, Br, CN, trifluoromethoxy, difluoromethoxy, 2,2-difluoroethoxy and 2,2,2-trifluoroethoxy, or is selected from Y1 to Y4; or
C. thiazole, pyridine or pyrimidine, wherein the thiazole, pyridine or pyrimidine is optionally substituted with one substituent selected from C₁-C₃haloalkyl, C₃-C₄cycloalkyl, halogen, cyano and C₁-C₃haloalkoxy, or is selected from Y1 to Y4; or
D. thiazole, pyridine or pyrimidine, wherein the thiazole, pyridine or pyrimidine is optionally substituted with one substituent selected from cyclopropyl, F, Cl, Br, CN, trifluoromethoxy, difluoromethoxy, 2,2-difluoroethoxy and 2,2,2-trifluoroethoxy, or is selected from Y1 to Y4; or
E. 5-cyanothiazol-2-yl, 5-cylopropylpyridine, 5-fluoropyridine, 5-chloropyridine, 5-bromopyridine, 5-difluoromethoxypyridine, 5-trifluoromethoxypyridine, 5-cyanopyridine, 5-(2,2-difluoroethoxy)-pyridine, 5-(2,2,2-trifluoroethoxy)-pyridine, pyridine, 5-cylopropylpyrimidine, 5-fluoropyrimidine, 5-chloropyrimidine, 5-bromopyrimidine, 5-difluoromethoxypyrimidine, 5-trifluoromethoxypyrimidine, 5-cyanopyrimidine, 5-(2,2-difluoroethoxy)-pyrimidine, 5-(2,2,2-trifluoroethoxy)-pyrimidine, pyrimidine, or 1,2,3-triazole; or
F. 5-cyanothiazol-2-yl, 5-cylopropylpyridin-2-yl, 5-fluoropyridin-2-yl, 5-chloropyridin-2-yl, 5-bromopyridin-2-yl, 5-difluoromethoxypyridin-2-yl, 5-trifluoromethoxypyridin-2-yl, 5-cyanopyridin-2-yl, 5-(2,2-difluoroethoxy)-pyridin-2-yl, 5-(2,2,2-trifluoroethoxy)-pyridin-2-yl, pyridin-2-yl, 5-cylopropylpyrimidin-2-yl, 5-fluoropyrimidin-2-yl, 5-chloropyrimidin-2-yl, 5-bromopyrimidin-2-yl, 5-difluoromethoxypyrimidin-2-yl, 5-trifluoromethoxypyrimidin-2-yl, 5-cyanopyrimidin-2-yl, 5-(2,2-difluoroethoxy)-pyrimidin-2-yl, 5-(2,2,2-trifluoroethoxy)-pyrimidin-2-yl, pyrimidin-2-yl, or 1,2,3-triazol-2-yl (or Y2); or
G. 5-cyanothiazol-2-yl, 1,2,3-triazol-2-yl (or Y2), pyrimidin-2-yl, or 5-cyanopyridin-2-yl; or
H. 5-cyanothiazol-2-yl; or
I. pyrimidin-2-yl; or
J. 5-cyanopyridin-2-yl.

In an embodiment of each aspect of the invention, when Y1 is selected as R₄ₐ, R′₄a and R′_{4c}, independently of each other, are
A. selected from hydrogen, halogen, CN, C₁-C₃alkyl, C₁-C₃haloalkyl, C₃-C₄cycloalkyl, C₁-C₃alkoxy and C₁-C₃haloalkoxy; or
B. selected from hydrogen, F, Cl, Br, CN, methyl, CF₃, cyclopropyl, methoxy and difluoromethoxy; or
C. both hydrogen.

In an embodiment of each aspect of the invention, when Y2 is selected as R₄ₐ,
A. R′_{4b} and R′_{4c}, independently of each other, are selected from hydrogen, halogen, CN, C₁-C₃alkyl, C₁-C₃haloalkyl, C₃-C₄cycloalkyl, C₁-C₃alkoxy and C₁-C₃haloalkoxy; or
B. R′_{4b} and R′_{4c}, independently of each other, are selected from hydrogen, F, Cl, Br, CN, methyl, CF₃, cyclopropyl, methoxy and difluoromethoxy; or
C. R′_{4b} and R′_{4c} are both hydrogen; or
D. R′_{4b} is hydrogen, and R′_{4c} is cyclopropyl.

In an embodiment of each aspect of the invention, when Y3 is selected as R₄ₐ, R′₄a and R′_{4b}, independently of each other, are
A. hydrogen, halogen, CN, C₁-C₃alkyl, C₁-C₃haloalkyl, C₃-C₄cycloalkyl, C₁-C₃alkoxy and C₁-C₃haloalkoxy; or
B. hydrogen, F, Cl, Br, CN, methyl, CF₃, cyclopropyl, methoxy and difluoromethoxy; or
C. both hydrogen.

In an embodiment of each aspect of the invention, when Y4 is selected as R₄ₐ,
A. R′₄ₐ, R′_{4b} and R′_{4c} are, independently of each other, selected from hydrogen, halogen, CN, C₁-C₃alkyl, C₁-C₃haloalkyl, C₃-C₄cycloalkyl, C₁-C₃alkoxy and C₁-C₃haloalkoxy; or
B. R′₄ₐ, R′_{4b} and R′_{4c} are, independently of each other, selected from hydrogen, F, Cl, Br, CN, methyl, CF₃, cyclopropyl, methoxy and difluoromethoxy; or
C. R′₄ₐ, R′_{4b} and R′_{4c} are all hydrogen; or
D. R′₄ₐ and R′_{4c} are hydrogen, and R′_{4b} is CN.

In an embodiment of each aspect of the invention, R₅ is
A. hydrogen, C₁-C₃alkyl, C₁-C₃haloalkyl, C₃-C₄cycloalkyl, C₁-C₃alkoxy, halogen, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), HC(O), C₁-C₃haloalkoxy or a 5-membered heteroaromatic ring wherein the 5-membered heteroaromatic ring can be optionally substituted with one to three substituents selected from C₁-C₃alkyl, C₁-C₃haloalkyl, C₁-C₃alkoxy, C₃-C₄cycloalkyl, halogen, CN or hydroxy; or
B. hydrogen, C₁-C₃alkyl, C₁-C₃haloalkyl, C₃-C₄cycloalkyl, C₁-C₃alkoxy, halogen, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), HC(O) or C₁-C₃haloalkoxy; or
C. hydrogen, C₁-C₃alkyl, C₁-C₃haloalkyl, C₃-C₄cycloalkyl, C₁-C₃alkoxy, halogen, C, Br, C₁-C₃alkoxy-C₁-C₃alkyl, C₁-C₃alkoxy-C₁-C₃alkoxy-C₁-C₃alkyl, (C₁-C₃alkyl)C(O), (C₁-C₃alkoxy)C(O), or C₁-C₂haloalkoxy; or
D. hydrogen, C₁-C₃alkyl, C₁-C₃alkoxy, C₃-C₄cycloalkyl, C₁-C₃haloalkoxy, halogen, C₁-C₃alkoxy-C₁-C₃alkyl, C₁-C₃alkoxy-C₁-C₃alkoxy-C₁-C₃alkyl, (C₁-C₃alkyl)C(O), HC(O), or (C₁-C₃alkoxy)C(O); or
E. hydrogen, C₁-C₂alkyl, C₁-C₂alkoxy, C₃-C₄cycloalkyl, C₁-C₂haloalkoxy, halogen, C₁-C₂alkoxy-C₁-C₂alkyl, C₁-C₂alkoxy-C₁-C₂alkoxy-C₁-C₂alkyl, (C₁-C₂alkyl)C(O), HC(O), or (C₁-C₂alkoxy)C(O); or
F. hydrogen, methyl, trifluoromethoxy, methoxy, cyclopropyl, 2,2-difluroroethoxy, 2,2,2-trifluroroethoxy, difluoromethoxy, 2,2,2-trifluroroethyl, chloro, bromo, methoxyethoxy, methylcarbonyl, or methoxycarbonyl; or
G. hydrogen, methyl, or bromo; or
H. methyl; or
I. bromo; or
J. hydrogen.

In an embodiment of each aspect of the invention, R₅ₐ is
A. hydrogen, halogen, CN, C₁-C₃alkyl, C₁-C₃haloalkyl, C₃-C₄cycloalkyl, C₁-C₃alkoxy or C₁-C₃haloalkoxy; or
B. hydrogen, halogen, CN, C₁-C₃alkyl, C₁-C₃haloalkyl, C₃-C₄cycloalkyl or C₁-C₃alkoxy; or
C. hydrogen, halogen, CN, C₁-C₃alkyl, C₁-C₃haloalkyl or C₁-C₃alkoxy; or
D. hydrogen, halogen, CN, C₁-C₃alkyl or C₁-C₃alkoxy; or
E. hydrogen or halogen; or
F. hydrogen.

In an embodiment of each aspect of the invention, R_{5b} is
A. hydrogen, halogen, CN, C₁-C₃haloalkyl, C₃-C₄cycloalkyl, C₁-C₃alkoxy, or C₁-C₃haloalkoxy; or
B. hydrogen, halogen or C₁-C₃alkoxy; or
C. hydrogen.

In an embodiment of each aspect of the invention, Re is
A. phenyl, benzyl, heteroaryl selected from J-1 to J-41, C₃-C₆cycloalkyl, or C₃-C₆cycloalkylC₁-C₃alkyl, each of which, independent of each other, is optionally substituted with one to three substituents independently selected from Rₓ; or
B. phenyl, benzyl, J-1, J-17, cyclopropyl, or cyclopropylmethyl, each of which, independent of each other, is optionally substituted with one to three substituents independently selected from Rₓ; or
C. phenyl, benzyl, J-1, J-17, cyclopropyl, or cyclopropylmethyl, each of which, independent of each other, is optionally substituted with one to three substituents independently selected from halogen, C₁-C₃alkyl, C₁-C₃haloalkyl, C₁-C₃alkoxy, C₁-C₃haloalkoxy and C₃-C₆cycloalkyl; or
D. phenyl, J-1, J-17, or cyclopropylmethyl, each of which, independent of each other, is optionally substituted with one to three substituents independently selected from halogen, C₁-C₃alkyl, C₁-C₃haloalkyl, C₁-C₃alkoxy, C₁-C₃haloalkoxy and C₃-C₆cycloalkyl; or
E. phenyl, J-1, J-17, or cyclopropylmethyl, each of which, independent of each other, is optionally substituted with one to three substituents independently selected from F, C, Br, and cyclopropyl.

In an embodiment of each aspect of the invention, R₇ is
A. hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; or
B. hydrogen, C₁-C₃alkyl, or C₃-C₆cycloalkyl; or
C. hydrogen, methyl, cyclopropyl, cyclobutyl, or cyclopentyl; or
D. hydrogen.

In an embodiment of each aspect of the invention, R₈ is
A. hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; or
B. C₁-C₃alkyl, or C₃-C₆cycloalkyl; or
C. methyl, cyclopropyl, cyclobutyl, or cyclopentyl; or
D. cyclopentyl.

In an embodiment of each aspect of the invention, Rₓ is independently selected from
A. halogen, C₁-C₃haloalkyl, C₁-C₃alkoxy, C₃-C₆cycloalkyl, C₁-C₃haloalkoxy and CN; or
B. F, Cl, Br, OCF₂H, CF₃, cyclopropyl, OCH₃ and CN; or
C. F, Cl, Br, and cyclopropyl.

In an embodiment of each aspect of the invention, R_{Y} is independently selected from
A. C₁-C₃ alkyl, C₃-C₄ alkenyl, C₃-C₄ cycloalkyl, C₂-C₄ alkynyl, or benzyl; or
B. methyl, vinyl, cyclopropyl, propargyl or benzyl.

In an embodiment of each aspect of the invention, R_{Z} is independently selected from
A. oxo, halogen, C₁-C₃haloalkyl, C₁-C₃alkoxy, C₁-C₃haloalkoxy or CN; or
B. oxo, F, Cl, Br, OCF₂H, OCH₃ or CN.

The present invention, accordingly, makes available a compound of formula I having the substituents T, X, R₁ₐ, R_{1b}, R₃ and Q as defined above in all combinations/each permutation.

Accordingly, made available, for example, is a compound of formula I with X as oxygen; T being an embodiment B (i.e. T is phenyl, pyridine, pyrimidine, pyrazine, pyridazine or a heteroaromatic ring selected from J-13, J-16, J-22, J-25, J-26, J-27, J-28, J-31, J-36, J-37, J-38, J-39, J-40, J-41, thieno[2,3-d]pyrimidinyl, [1,2,4]triazolo[1,5-a]pyrimidinyl, and indazolyl, each of which, independent of each other, is substituted with one to three substituents independently selected from R₂), where R₂ is of embodiment A (i.e. R₂, independently of the ring and the number of substituents, is selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ, OR₆, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring); R₁ₐ being embodiment D (i.e. R₁ₐ is hydrogen, C₁-C₆alkyl, C₁-C₆cyanoalkyl, C₁-C₃alkoxy-C₁-C₆alkyl, C₁-C₆haloalkyl, C₂-C₆alkenyl, C₂-C₆haloalkenyl, C₂-C₆alkynyl, C₂-C₆haloalkynyl, C₃-C₄cycloalkylC₁-C₂alkyl-, benzyloxycarbonyl, or benzyl); R_{1b} being embodiment I (i.e. R_{1b} is hydrogen, methyl, propargyl, or cyclopropyl-methyl); R₃ being embodiment B (i.e. R₃ is methyl or trifluoromethyl); and Q is embodiment F (i.e. Q is Q^{a}-1, or Q^{b}-1), where R₄ is embodiment K (i.e. R₄ is pyrimidin-2-yl, pyridin-2-yl, 5-bromopyrimidin-2-yl, 5-bromopyridin-2-yl, 5-cyanopyrimidin-2-yl, or 5-cyanopyridin-2-yl); R₄ₐ is embodiment G (i.e. R₄ₐ is 1,2,3-triazol-2-yl (or Y2), pyrimidin-2-yl, or 5-cyanopyridin-2-yl); R₆ is of embodiment E (i.e. R₆ is phenyl, J-1, J-17, or cyclopropylmethyl, each of which, independent of each other, is optionally substituted with one to three substituents independently selected from F, Cl, Br, and cyclopropyl); R₇ is of embodiment D (i.e. R₇ is hydrogen); R₈ is of embodiment D (i.e. R₈ is cyclopentyl); Rₓ is of embodiment B (i.e. Rₓ is independently selected from F, Cl, Br, OCF₂H, CF₃, cyclopropyl, OCH₃ and CN); and R_{z} is of embodiment B (i.e. R_{z} is independently selected from oxo, F, Cl, Br, OCF₂H, OCH₃ and CN).

In an embodiment, the compound of formula I is formula Iaa and Iab (with asterisk indicating a stereogenic centre), wherein T, R₁ₐ, R_{1b}, R₃ are as defined in the first aspect and Q₁ corresponds to Q as defined in the first aspect, each with the corresponding embodiments as described above. The preferred stereochemistry of compounds of formula Iaa and Iab is that depicted in formula I′a above.

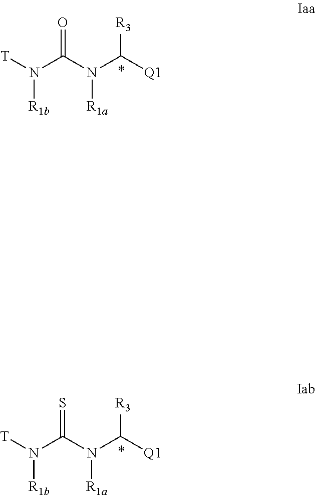

In an embodiment, Q₁ is
A. selected from Q^{aa} to Q^{ag} and Q^{ba} to Q^{bf}; or
B. selected from Q^{aa} to Q^{a}9; or
C. selected from Q^{ba} to Q^{bf}; or
D. selected from Q^{aa}, Q^{ab}, Q^{ac}, Q^{af}, Q^{ag}, Q^{ba}, Q^{bb}, Q^{bc}, Q^{bd}, Q^{be} and Q^{bf}; or
E. selected from Q^{aa}, Q^{ab}, Q^{ac}, Q^{af}, Q^{af}, Q^{ba}, Q^{bb} and Q^{bf}; or
F. selected from Q^{aa}, Q^{ab}, Q^{ac}, Q^{af}, Q^{ba}, Q^{bb} and Q^{bf}.

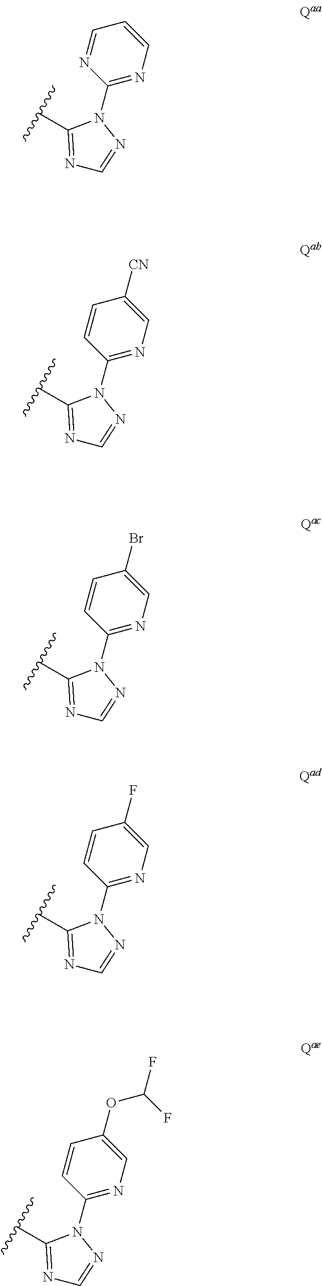 [SMILES: *c1ncnn1-c1ncccn1 ; *c1ncnn1-c1ccc(C#N)cn1 ; *c1ncnn1-c1ccc(Br)cn1 ; *c1ncnn1-c1ccc(F)cn1 ; *c1ncnn1-c1ccc(OC(F)F)cn1]

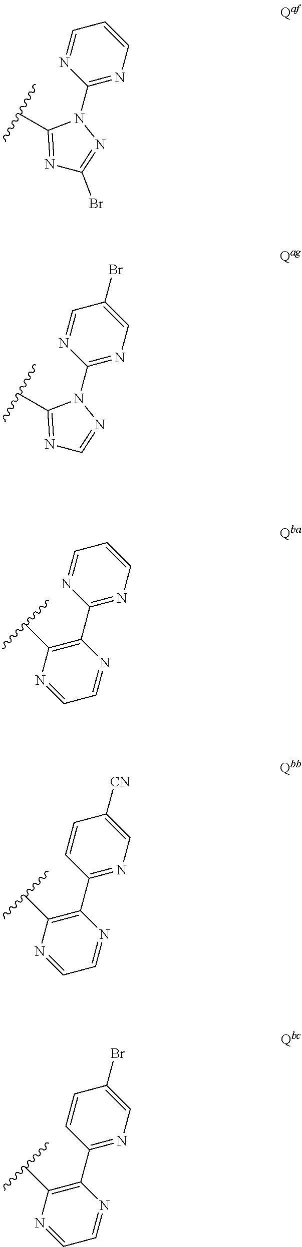 [SMILES: *c1nc(Br)nn1-c1ncccn1 ; *c1ncnn1-c1ncc(Br)cn1 ; *c1nccnc1-c1ncccn1 ; *c1nccnc1-c1ccc(C#N)cn1 ; *c1nccnc1-c1ccc(Br)cn1]

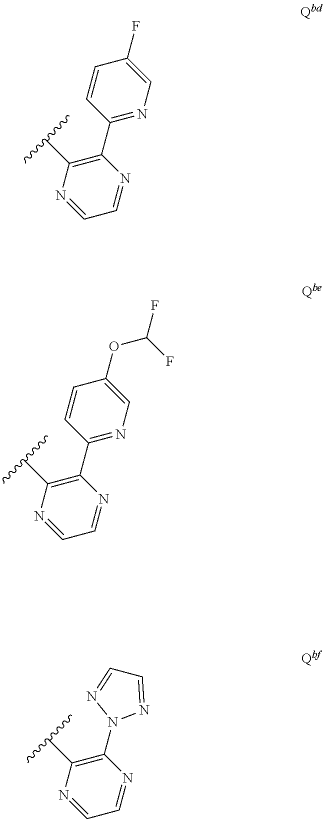 [SMILES: *c1nccnc1-c1ccc(F)cn1 ; *c1nccnc1-c1ccc(OC(F)F)cn1 ; *c1nccnc1-n1nccn1]

Similarly, the present invention makes available a compound of formulae Iaa and Iab having the substituents T, R₁ₐ, R_{1b}, R₃ and Q1 as defined in all combinations/each permutation

In an embodiment of each aspect of the invention, the compound of formula I has as X oxygen or sulfur; as T phenyl, pyridine, or a five or nine membered heteroaromatic ring, each of which, independent of each other, can be substituted with one to three substituents independently selected from R₂; where R₂, independently of the ring and of the number of substituents, is selected from halogen, C₁-C₃alkyl, C₁-C₃haloalkyl, C₁-C₃alkoxy, C₁-C₃haloalkoxy, CN, C₃-C₄cycloalkyl, 03-C₆cycloalkylcarbonyl, phenyl, heteroaryl selected from J-1 and J-41, each of C₃-C₄cycloalkyl, phenyl or heteroaryl, independent of each other, being substituted with one to three substituents R_{X}, OR₆, piperidin-2-one-1-yl, pyridin-2-one-1-yl, azetidin-1-yl optionally substituted with R_{X}, pyrrolidin-1-yl, O₃—C₆cycloalkylC₁-C₄alkyl substituted with one or two substituents R_{z}, C₃-C₆cycloalkylC₁-C₃alkoxy optionally substituted with R_{X}, C₁-C₅cyanoalkyl, C₁-C₅cyanoalkoxy, C₁-C₄alkylsulfanyl optionally substituted by one to three substituents R_{X}, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring; where RB is phenyl, benzyl, heteroaryl selected from J-1 to J-41, C₃-C₆cycloalkyl, or C₃-C₆cycloalkylC₁-C₃alkyl, each of which, independent of each other, is optionally substituted with one to three substituents independently selected from Rₓ; R₇ and R₈ independently of each other is hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; where R_{X} is independently selected from halogen, C₁-C₃haloalkyl, C₁-C₃alkoxy, C₃-C₆cycloalkyl, C₁-C₃haloalkoxy and CN; where R_{z} is independently selected from oxo, halogen, C₁-C₃haloalkyl, C₁-C₃alkoxy, C₁-C₃haloalkoxy and CN; as R₁ₐ and R_{1b}, independent of each other, hydrogen, methyl, ethyl, cyanomethyl, methoxymethyl, cyclopropyl-methyl, allyl, propargyl, benzyloxycarbonyl, or benzyl, or as R₁ₐ and R_{1b} together —CH₂—O—CH₂—; or T and R_{1b} together is nine membered bicyclic partially saturated heterocyclic ring substituted with C₁-C₃haloalkyl; as R₃ methyl or trifluoromethyl; and as Q selected from Q^{a}-1 to Q^{a}-16 and Q^{b}-1 to Q^{b}-13; where R₄ is pyridine, or pyrimidine; wherein the pyridine or pyrimidine, independently of each other, is optionally substituted with one substituent selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, C₃-C₆cycloalkylC₁-C₄haloalkoxy, NH₂C(O)—, NH₂C(S)—, (OH)N═C(NH₂)—, J-13 optionally substituted by 1 to 3 substituents independently selected from halogen, C₁-C₃alkyl, C₁-C₃haloalkyl, C₁-C₃alkoxy and C₁-C₃haloalkoxy, J-20 optionally substituted by 1 to 3 substituents independently selected from halogen, C₁-C₃alkyl, C₁-C₃haloalkyl, C₁-C₃alkoxy and C₁-C₃haloalkoxy and 1H-tetrazol-5-yl; and where R₄ₐ is pyridine, pyrimidine, pyrazine or pyridazine, wherein the pyridine, pyrimidine, pyrazine or pyridazine, independent of each other, is optionally substituted with one substituent selected from C₁-C₃haloalkyl, C₃-C₄cycloalkyl, halogen, cyano and C₁-C₃haloalkoxy. or is selected from Y1 to Y4; wherein, R′₄ₐ, R′_{4b} and R′_{4c}, independently of each other and independently of Y1 to Y4, are selected from hydrogen, halogen, CN, C₁-C₃alkyl, C₁-C₃haloalkyl, C₃-C₄cycloalkyl, C₁-C₃alkoxy and C₁-C₃haloalkoxy.

In an embodiment of each aspect of the invention, the compound of formula I has as X oxygen or sulfur; as T phenyl, pyridine, or a five or nine membered heteroaromatic ring, each of which, independent of each other, can be substituted with one to three substituents independently selected from R₂; where R₂, independently of the ring and of the number of substituents, is selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ, ORB, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring; where RB is phenyl, benzyl, heteroaryl selected from J-1 to J-41, C₃-C₆cycloalkyl, or C₃-C₆cycloalkylC₁-C₃alkyl, each of which, independent of each other, is optionally substituted with one to three substituents independently selected from Rₓ; R₇ and R₈ independently of each other is hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; where Rₓ is independently selected from halogen, C₁-C₃haloalkyl, C₁-C₃alkoxy, C₃-C₆cycloalkyl, C₁-C₃haloalkoxy and CN; where R_{z} is independently selected from oxo, halogen, C₁-C₃haloalkyl, C₁-C₃alkoxy, C₁-C₃haloalkoxy and CN; as R₁ₐ and R_{1b}, independent of each other, hydrogen, methyl, ethyl, cyanomethyl, methoxymethyl, cyclopropyl-methyl, allyl, propargyl, benzyloxycarbonyl, or benzyl, or as R₁ₐ and R_{1b} together —CH₂—O—CH₂—; or T and R_{1b} together is nine membered bicyclic partially saturated heterocyclic ring substituted with C₁-C₃haloalkyl; as R₃ methyl or trifluoromethyl; and as Q selected from Q^{aa} to Q^{ag} and Q^{ba} to Q^{bf}.

In an embodiment of each aspect of the invention, the compound of formula I has as X oxygen or sulfur; as T phenyl, pyridine, or a five or nine membered heteroaromatic ring, each of which, independent of each other, can be substituted with one to three substituents independently selected from R₂; where R₂, independently of the ring and of the number of substituents, is selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ, ORB, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkanediyl-O—, and 10 membered optionally substituted spiroheterocyclic ring; where RB is phenyl, benzyl, heteroaryl selected from J-1 to J-41, C₃-C₆cycloalkyl, or C₃-C₆cycloalkylC₁-C₃alkyl, each of which, independent of each other, is optionally substituted with one to three substituents independently selected from Rₓ; R₇ and R₈ independently of each other is hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; where Rₓ is independently selected from halogen, C₁-C₃haloalkyl, C₁-C₃alkoxy, C₃-C₆cycloalkyl, C₁-C₃haloalkoxy and CN; where R_{z} is independently selected from oxo, halogen, C₁-C₃haloalkyl, C₁-C₃alkoxy, C₁-C₃haloalkoxy and CN; as R₁ₐ and R_{1b}, independent of each other, hydrogen, methyl, ethyl, cyanomethyl, methoxymethyl, cyclopropyl-methyl, allyl, propargyl, benzyloxycarbonyl, or benzyl, or as R₁ₐ and R_{1b} together —CH₂—O—CH₂—; or T and R_{1b} together is nine membered bicyclic partially saturated heterocyclic ring substituted with C₁-C₃haloalkyl; as R₃ methyl or trifluoromethyl; and as Q selected from Q^{aa}, Q^{ab}, Q^{ba} and Q^{bb}.

In an embodiment of each aspect of the invention, the compound of formula I has as X oxygen or sulfur; as T phenyl, pyrid-2-yl (or J-1), J-2, J-3, J-4. J-5, J-6, J16, J-26, J-27, J-28, J41, thieno[2,3-d]pyrimidinyl, [1,2,4]triazolo[1,5-a]pyrimidinyl, or indazolyl each of which, independent of each other, is substituted with one to three substituents independently selected from R₂; where R₂, independently of the ring and of the number of substituents, is selected from halogen, C₁-C₃alkyl, C₁-C₃haloalkyl, C₁-C₃alkoxy, C₁-C₃haloalkoxy, CN, C₃-C₄cycloalkyl, heteroaryl selected from J-1 and J-41, each of C₃-C₄cycloalkyl, or heteroaryl, independent of each other, is substituted with one to three substituents Rₓ, ORB, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, C₃-C₆cycloalkylaminosulfonyl, —O—C₁-C₂haloalkanediyl-O—, and 10 membered optionally substituted spiroheterocyclic ring; where RB is phenyl, benzyl, heteroaryl selected from J-1 to J-41, C₃-C₆cycloalkyl, or C₃-C₆cycloalkylC₁-C₃alkyl, each of which, independent of each other, is optionally substituted with one to three substituents independently selected from Rₓ; R₇ and R₈ independently of each other is hydrogen, C₁-C₃alkyl, or C₃-C₆cycloalkyl, wherein the C₁-C₃alkyl or C₃-C₆cycloalkyl, independent of each other, is optionally substituted with one to three substituents independently selected from R_{z}; where Rₓ is independently selected from halogen, C₁-C₃haloalkyl, C₁-C₃alkoxy, C₃-C₆cycloalkyl, C₁-C₃haloalkoxy and CN; where R_{z} is independently selected from oxo, halogen, C₁-C₃haloalkyl, C₁-C₃alkoxy, C₁-C₃haloalkoxy and CN; as R₁ₐ and R_{1b}, independent of each other, hydrogen, methyl, ethyl, cyanomethyl, methoxymethyl, cyclopropyl-methyl, allyl, propargyl, benzyloxycarbonyl, or benzyl, or as R₁ₐ and R_{1b} together —CH₂—O—CH₂—; or T and R_{1b} together is nine membered bicyclic partially saturated heterocyclic ring substituted with C₁-C₃haloalkyl; as R₃ methyl or trifluoromethyl; and as Q selected from Q^{aa}, Q^{ab}, Q^{ba} and Q^{bb}.

In an embodiment of each aspect of the invention, the compound of formula I has as X oxygen or sulfur; as T phenyl, or pyridine, each of which, independent of each other, is substituted with one to three substituents independently selected from R₂, where R₂, independently of the ring and of the number of substituents, is selected from trifluoromethyl, fluorine, iodine, bromine chlorine, ORB where R is J-1 substituted by 1 to 2 substituents independently selected from chloro and trifluoromethyl, difluoromethoxy, 1,1,2,2-tettrafluoroethoxy, and —O—CF₂O—; as R₁ₐ and R_{1b}, independent of each other, hydrogen or methyl; or T and R_{1b} together is nine membered bicyclic partially saturated heterocyclic ring substituted with C₁-C₃haloalkyl; as R₃ methyl; and as Q either Q^{aa} or Q^{ab}In an embodiment of each aspect of the invention, the compound of formula I is represented by formula I′a and has as X oxygen or sulfur; as T phenyl, or pyridine, each of which, independent of each other, is substituted with one to three substituents independently selected from R₂, where R₂, independently of the ring and of the number of substituents, is selected from trifluoromethyl, fluorine, iodine, bromine chlorine, ORB where R is J-1 substituted by 1 to 2 substituents independently selected from chloro and trifluoromethyl, difluoromethoxy, 1,1,2,2-tettrafluoroethoxy, and —O—CF₂O—; as R₁ₐ and R_{1b}, independent of each other, hydrogen or methyl; or T and R_{1b} together is indolinyl substituted with C₁-C₃haloalkyl; as R₃ methyl; and as Q either Q^{aa} or Q^{ab}

In an embodiment of each aspect of the invention, the compound of formula I has as X oxygen; as T phenyl, or pyridine, each of which, independent of each other, is substituted with one to three substituents independently selected from R₂, where R₂, independently of the ring and of the number of substituents, is selected from trifluoromethyl, fluorine, iodine, bromine chlorine, difluoromethoxy, and 1,1,2,2-tettrafluoroethoxy; as R₁ₐ and R_{1b} are each hydrogen; as R₃ methyl; and as Q either Q^{aa} or Q^{ab}.

In an embodiment of each aspect of the invention, the compound of formula I is represented by formula I′a and has as X oxygen; as T phenyl, or pyridine, each of which, independent of each other, is substituted with one to three substituents independently selected from R₂, where R₂, independently of the ring and of the number of substituents, is selected from trifluoromethyl, fluorine, iodine, bromine chlorine, difluoromethoxy, and 1,1,2,2-tettrafluoroethoxy; as R₁ₐ and R_{1b} are each hydrogen; as R₃ methyl; and as Q either Q^{aa} or Q^{ab}

In a second aspect, the present invention makes available a composition comprising a compound of formula I as defined in the first aspect, one or more auxiliaries and diluent, and optionally one or more other active ingredient.

In a third aspect, the present invention makes available a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in the first aspect or a composition as defined in the second aspect.

In a fourth aspect, the present invention makes available a method for the protection of plant propagation material from the attack by insects, acarines, nematodes or molluscs, which comprises treating the propagation material or the site, where the propagation material is planted, with an effective amount of a compound of formula I as defined in the first aspect or a composition as defined in the second aspect.

In a fifth aspect, the present invention makes available a plant propagation material, such as a seed, comprising, or treated with or adhered thereto, a compound of formula I as defined in the first aspect or a composition as defined in the second aspect.

The present invention in a further aspect provides a method of controlling parasites in or on an animal in need thereof comprising administering an effective amount of a compound of the first aspect. The present invention further provides a method of controlling ectoparasites on an animal in need thereof comprising administering an effective amount of a compound of formula I as defined om the first aspect. The present invention further provides a method for preventing and/or treating diseases transmitted by ectoparasites comprising administering an effective amount of a compound of formula I as defined in the first aspect, to an animal in need thereof.

Compounds of formula I can be prepared by those skilled in the art following known methods. More specifically compounds of formulae I and I′a, and intermediates therefor can be prepared as described below in the schemes and examples. Certain stereogenic centers have been left unspecified for the clarity and are not intended to limit the teaching of the schemes in any way.

The process according to the invention for preparing compounds of formula I is carried out by methods known to those skilled in the art.

In another aspect, an example of which is shown in Schemes 1 and 2, the present invention makes available a process for preparing a compound of the formula I,

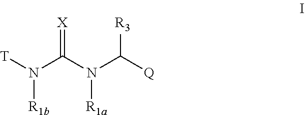

comprising reacting an amine of formula II

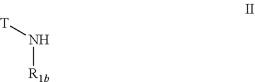

with (i) a compound of formula III when R₁ₐ is hydrogen (i.e. this corresponds to a compound of the formula I-a)

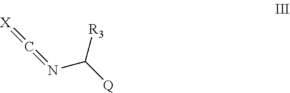

or (ii) with a compound of formula IV,

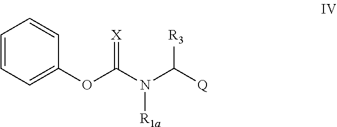

where X, R₃, R₁ₐ, R_{1b}, T and Q are as defined above for a compound of the formula I.

In another aspect, an example of which is shown in Scheme 4, the present invention makes available a process for preparing a compound of the formula I,

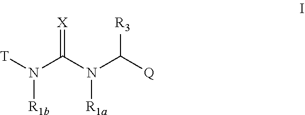

comprising reacting an amine of formula VI

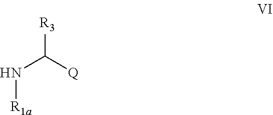

with a compound of formula VII,

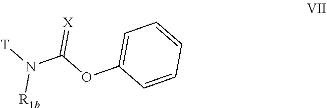

where X, R₃, R₁ₐ, R_{1b}, T and Q are as defined above for a compound of the formula I.

Compounds of formula I can be made, for example, as shown in scheme 1.

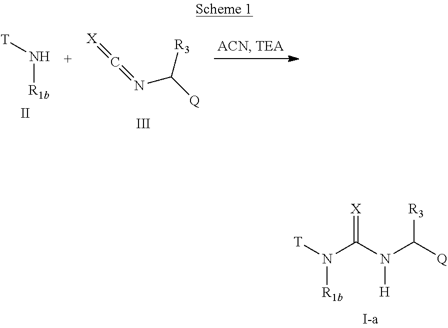

Reaction of a compound of the formula II with a compound of formula III gives a compound of the formula I-a, where X, R₃, R_{1b}, T and Q are as defined above for a compound of the formula I. The reaction can be conducted neat or in a solvent, preferably in a solvent, such as an organic solvent, for instance acetonitrile, in a temperature range of −100 to +300° C. preferably between ambient temperature and 200° C., with or without the addition of a base, such as an inorganic base, for instance potassium carbonate, or an organic base, such as, for example, triethylamine. Compounds of the formula II and III are either known, or they can be prepared by methods known to a person skilled in the art.

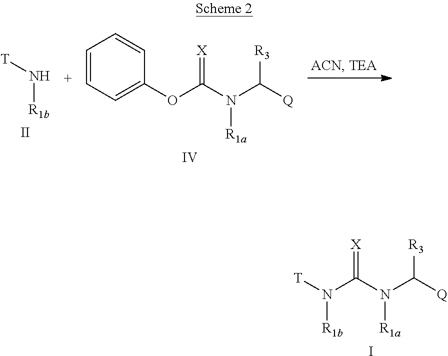

Reaction of a compound of the formula II with a compound of formula IV gives a compound of the formula I, where X, R₃, R₁ₐ, R_{1b}, T and Q are as defined above for a compound of the formula I. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Compounds of the formula II are either known, or they can be prepared by methods known to a person skilled in the art.

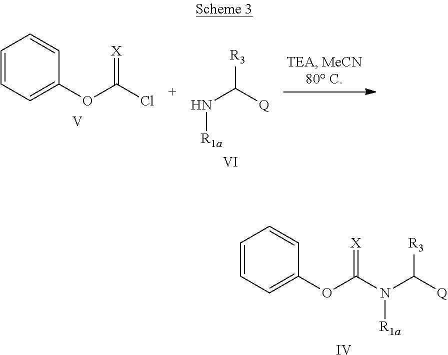

Compounds of formula IV can be made, for example, as shown in scheme 3. Treatment of a compound of the formula V, with an amine of the formula VI gives compounds of the formula IV, where X, R₃, R₁ₐ and Q are as defined above for a compound of the formula I. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine.

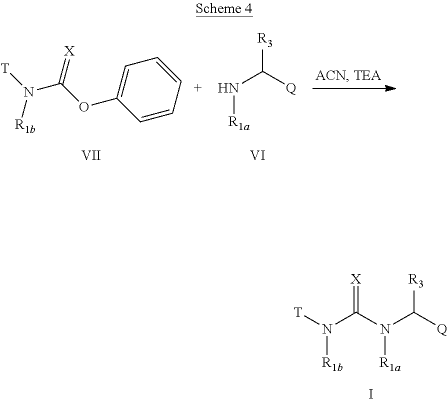

Reaction of a compound of the formula VII with a compound of formula VI gives a compound of the formula I, where X, R₃, R₁ₐ, R_{1b}, T and Q are as defined above for a compound of formula I. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Compounds of the formula II are either known, or they can be prepared by methods known to a person skilled in the art.

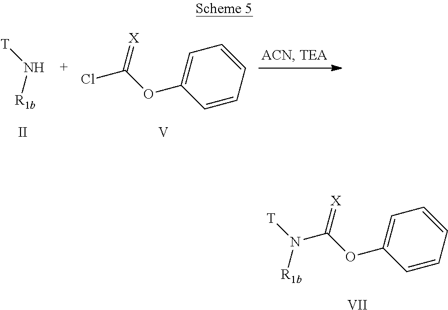

Compounds of formula VII can be made, for example, as shown in scheme 5. Treatment of a compound of the formula V, with an amine of the formula II gives compounds of the formula VII, where R_{1b}, X and T are as defined above for a compound of formula I. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine.

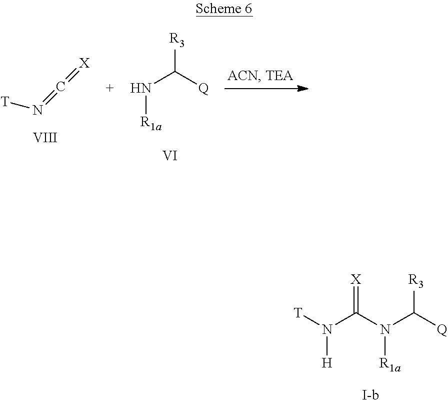

Reaction of a compound of the formula VIII with a compound of formula VI gives a compound of the formula I-b, where X, R₃, R₁ₐ, T and Q are as defined above for a compound of formula I. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Compounds of the formula VIII and VI are either known, or they can be prepared by methods known to a person skilled in the art.

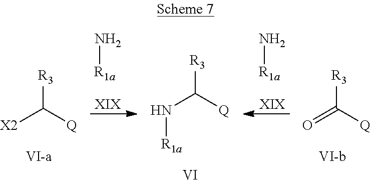

Compounds of formula VI can be made, for example, as shown in scheme 7, where R₃, R₁ₐ and Q are as defined above for a compound of formula I. Treatment of a compound of the formula VI-a, wherein X2 is a leaving group, such as a halogen or sulfonate, for instance bromide, with an amine of the formula XIX gives compounds of the formula VI. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Alternatively, treatment of a compound of the formula VI-b with an amine of the formula XIX gives compounds of the formula VI. This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C. Such methods, and the range of conditions to perform them, for the alkylation of amines and for the reductive alkylation of amines are well known to a person skilled in the art. The amines of formula XIX are either known, or they can be prepared by methods known to a person skilled in the art.

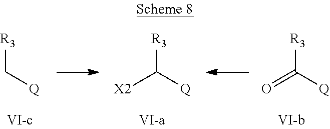

Compounds of formula VI-a can be made, for example, as shown in scheme 8, where R₃ and Q are as defined above for a compound of formula I. Treatment of a compound of the formula VI-c with a halogenating agent, such as chlorine or bromine or N-bromosuccinimide, for example, gives compound of the formula VI-a, wherein the leaving group X2 is a halogen, for instance chloride or bromide. This reaction is done with or without a solvent, preferably in a solvent, with or without an additive, such as a radical starter, such as, for example, benzoyl peroxide or azoisobutyronitrile. The reaction can be done with or without exposure to visible light, or to UV light, and it can be conducted in a temperature range of −100 to +300° C., preferably between ambient temperature and 200° C. Alternatively, a compound of the formula VI-b can be treated with a reducing agent, followed by reaction with a sulfonyl chloride, for instance methanesulfonyl chloride, to give a compound of the formula VI-a, wherein the leaving group X2 is a sulfonate, for instance a mesylate. This reaction can be done in a solvent, or without a solvent, in the presence of a base, such as an inorganic base, for instance potassium carbonate, or an organic base, such as an amine base, for instance trimethylamine, or without a base, and it can be conducted in a temperature range of −100 to +300° C., preferably between ambient temperature and 200° C. A suitable reducing agent could be, for example, hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C. Such methods for the halogenation, the reduction of carbonyl compounds and the sulfonylation of alcohols, and the range of conditions to perform them, are well known to a person skilled in the art. The compounds of formula VI-b and the compounds of formula VI-c are either known, or they can be prepared by methods known to a person skilled in the art.

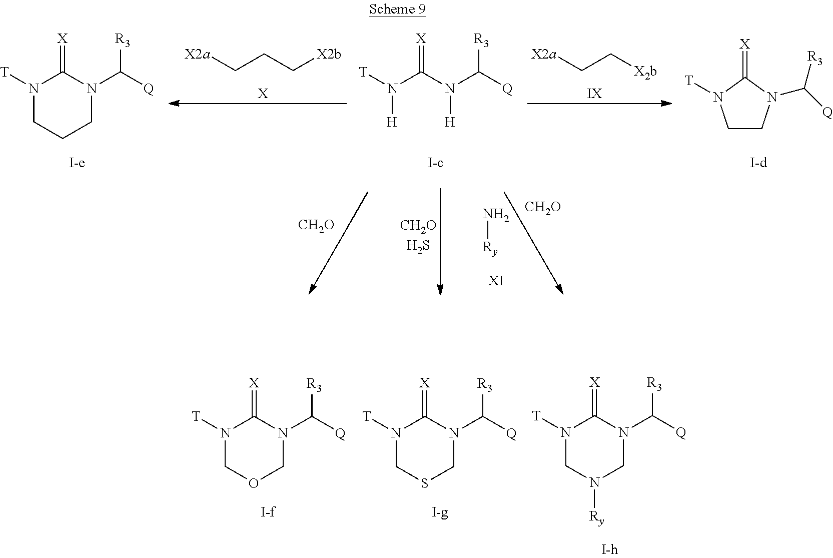

As shown in scheme 9, compounds of the formula I-d, wherein T, X, R3 and Q are as defined for formula I, can be made from compounds of formula I-c by treatment with a compound of formula IX, wherein X2a and X2b are leaving groups, such as halogen or sulfonate, for example bromide or iodide, or tosylate or mesylate. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Such transformations are known to the person skilled in the art.

As shown in scheme 9, compounds of the formula I-e, wherein T, X, R₃ and Q are as defined for formula I, can be made from compounds of formula I-c by treatment with a compound of formula X, wherein X2a and X2b are leaving groups, such as halogen or sulfonate, for example bromide or iodide, or tosylate or mesylate. The reaction can be conducted neat or in a solvent, preferably in a solvent, such as an organic solvent, for instance acetonitrile, in a temperature range of −100 to +300° C., preferably between ambient temperature and 200° C., with or without the addition of a base, such as an inorganic base, for instance potassium carbonate, or an organic base, such as, for example, triethylamine. Such transformations are known to the person skilled in the art.

As shown in scheme 9, compounds of the formula I-f, wherein T, X, R₃ and Q are as defined for formula I, can be made from compounds of formula I-c by treatment with formaldehyde. The reaction can be conducted neat or in a solvent, preferably in a solvent, such as an organic solvent, for instance 1,2-dichloroethane, in a temperature range of −100 to +300° C., preferably between ambient temperature and 200° C., with or without the addition of an acid, such as an inorganic acid, for instance hydrochloric acid, or an organic acid, such as, for example, acetic acid or trifluoroacetic acid. Such transformations are known to the person skilled in the art. For instance, such transformations are described in Synthetic Communications (1996), 26(17), 3217-3224.

As shown in scheme 9, compounds of the formula I-g, wherein T, X, R₃ and Q are as defined for formula I, can be made from compounds of formula I-c by treatment with formaldehyde and hydrogen sulfide. The reaction can be conducted neat or in a solvent, preferably in a solvent, such as an organic solvent, for instance chloroform, in a temperature range of −100 to +300° C., preferably between ambient temperature and 200° C., with or without the addition of an acid, such as an inorganic acid, for instance hydrochloric acid, or an organic acid, such as, for example, p-toluenesulfonic acid. Such transformations are known to the person skilled in the art. For instance, such transformations are described in Journal of Heterocyclic Chemistry (1972), 9(2), 231-4.

As shown in scheme 9, compounds of the formula I-h, wherein T, X, R₃ and Q are as defined for formula I, can be made from compounds of formula I-c by treatment with formaldehyde and an amine XI, wherein R_{y} has the same meaning as defined for compounds of the formula I. The amine can be used as such or in the form of a salt, such as, for instance, in the form of a hydrochloride. The reaction can be conducted neat or in a solvent, preferably in a solvent, such as an organic solvent, for instance ethanol, or in water, or in a mixture of solvents, in a temperature range of −100 to +300° C., preferably between ambient temperature and 200° C. with or without the addition of an acid, such as an inorganic acid, for instance hydrochloric acid, or an organic acid, such as, for example, p-toluenesulfonic acid. Such transformations are known to the person skilled in the art. For instance, such transformations are described in Izvestiya Vysshikh Uchebnykh Zavedenii, Khimiya i Khimicheskaya Tekhnologiya (2005), 48(3), 106-108.

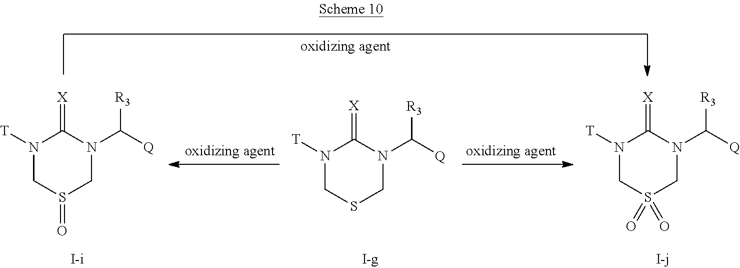

As shown in scheme 10, compounds of the formula I-i, wherein T, X, R3 and Q are as defined for formula I, can be made from compounds of formula I-g by treatment with an oxidizing agent, such as hydrogen peroxide or a peracid, for instance m-chloroperbenzoic acid. The reaction can be conducted neat or in a solvent, preferably in a solvent, such as an organic solvent, for instance dichloromethane, in a temperature range of −100 to +300° C., preferably between ambient temperature and 200° C., with or without the addition of a catalyst, such as a metal catalyst, for instance 12-tungstophosphoric acid. Such transformations are well known to the person skilled in the art.

As shown in scheme 10, compounds of the formula I-j, wherein T, X, R3 and Q are as defined for formula I, can be made from compounds of formula I-g by treatment with an oxidizing agent, such as hydrogen peroxide or a peracid, for instance m-chloroperbenzoic acid. The reaction can be conducted neat or in a solvent, preferably in a solvent, such as an organic solvent, for instance dichloromethane, in a temperature range of −100 to +300° C., preferably between ambient temperature and 200° C., with or without the addition of a catalyst, such as a metal catalyst, for instance 12-tungstophosphoric acid. Such transformations are well known to the person skilled in the art.

Alternatively, compounds of the formula I-j, wherein T, X, R3 and Q are as defined for formula I, can be made from compounds of formula I-i by treatment with an oxidizing agent, such as hydrogen peroxide or a peracid, for instance m-chloroperbenzoic acid. The reaction can be conducted neat or in a solvent, preferably in a solvent, such as an organic solvent, for instance dichloromethane, in a temperature range of −100 to +300° C., preferably between ambient temperature and 200° C., with or without the addition of a catalyst, such as a metal catalyst, for instance 12-tungstophosphoric acid. Such transformations are well known to the person skilled in the art.

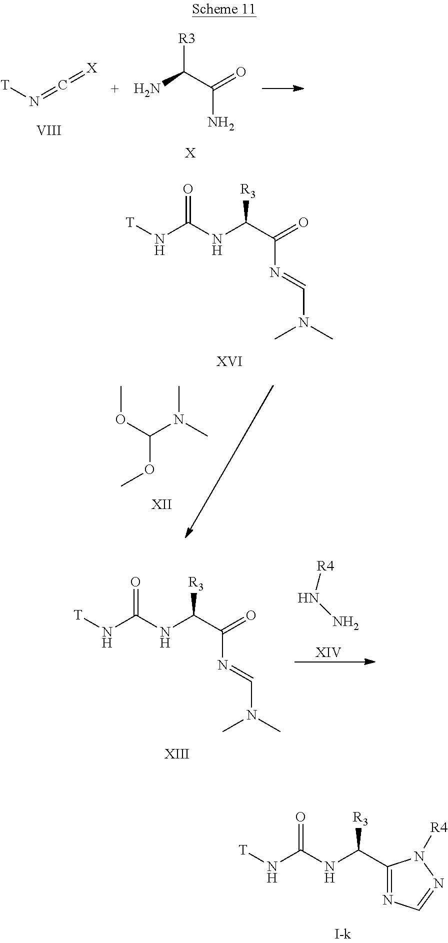

Compounds of formula I-k can be made, for example, as shown in scheme 11. Reaction of a compound of the formula X with a compound of formula VIII gives a compound of the formula XVI, wherein T and R₃ have the same meaning as given above for compounds of the formula I. The reaction can be conducted neat or in a solvent, preferably in a solvent, such as an organic solvent, for instance acetonitrile or N,N-dimethylformamide, in a temperature range of −100 to +300° C., preferably between ambient temperature and 200° C., with or without the presence of a catalyst, for instance a metal catalyst, such as a palladium complex, and with or without the addition of a base, such as an inorganic base, for instance potassium carbonate, or an organic base, such as, for example, triethylamine.

Subsequent treatment of compound XVI with the known compound XII gives a compound of the formula XIII, wherein T and R₃ have the same meaning as given above for compounds of the formula I. This reaction can be conducted neat or in a solvent, preferably in a solvent, such as an organic solvent, for instance dichloromethane, in a temperature range of −100 to +300° C., preferably between ambient temperature and 100° C., or between ambient temperature and 50° C., without a base or in the presence of a base, such as an inorganic base, for instance potassium carbonate, or an organic base, such as, for example, triethylamine.

Further reaction of compound XIII with hydrazine XIV gives the compound of formula I-k, wherein T, R₃ and R₄ have the same meaning as given above for compounds of the formula I. This reaction can be conducted neat or in a solvent, preferably in a solvent, such as an organic solvent, for instance 1,4-dioxane, or acetic acid, or a mixture of 1,4-dioxane and acetic acid, in a temperature range of −100 to +300° C., preferably between ambient temperature and 200° C., or between ambient temperature and 80° C. Within this sequence of transformations, the intermediate compounds of formula XVI and of formula XIII can be used as crude products for the subsequent step, or they can be purified, for instance by chromatography, and used in purified form for the next transformation.

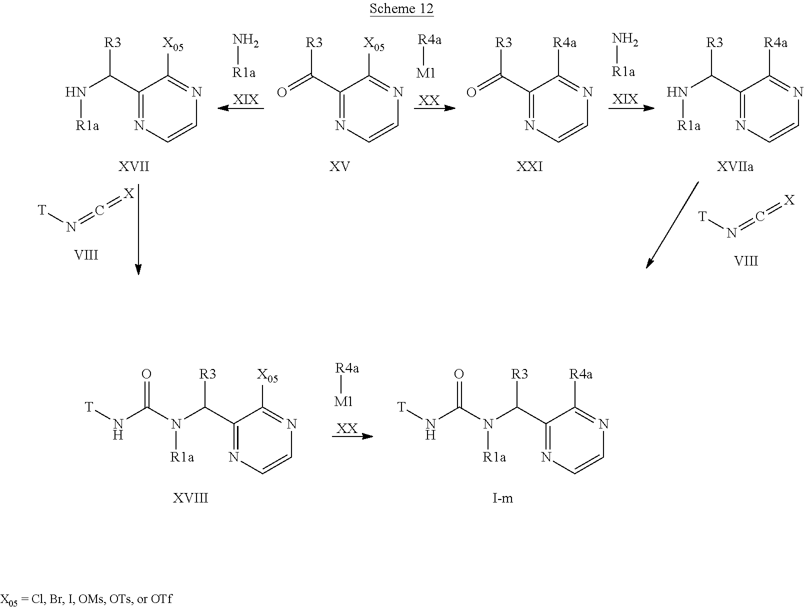

Compounds of formula I-m can be made, for example, as shown in scheme 12. Reaction of a compound of the formula XV (wherein X₀₅ is a leaving group such as chlorine, bromine, iodine, arysulfonate, alkylsulfonate or trifluoromethanesulfonate) with an amine of the formula XIX gives compounds of the formula XVII, wherein R₁ₐ and R₃ have the same meaning as given above for compounds of the formula I. This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C. Such methods, and the range of conditions to perform them, for the reductive alkylation of amines are well known to a person skilled in the art.

Subsequent reaction of the intermediate of the formula XVII with a compound of the formula VIII gives a compound of the formula XIII, wherein T, R₁ₐ and R₃ have the same meaning as given above for compounds of the formula I. This reaction can be conducted neat or in a solvent, preferably in a solvent, such as an organic solvent, for instance acetonitrile, in a temperature range of −100 to +300° C., preferably between ambient temperature and 200° C., with or without the presence of a catalyst, for instance a metal catalyst, such as a palladium complex, and with or without the addition of a base, such as an inorganic base, for instance potassium carbonate, or an organic base, such as, for example, triethylamine.

Subsequently, the intermediate of the formula XVIII is reacted with a compound of the formula XX to give the compound of formula I-m, wherein T, R₁ₐ, R₃ and R₄ₐ have the same meaning as given above for compounds of the formula I, and M₁ in R₄ₐ-M₁ is a metal, such as for instance lithium, or —MgCl, or —ZnBr, or —B(OH)₂; or R₄ₐ-M₁ represents a boronate, such as a pinacol ester of a boronic acid, or a stannane such as R₄ₐ—Sn(n-Bu)₃. Such transformations are known to a person skilled in the art as Suzuki-, Kumada-, Negishi- or Stille-coupling reactions, respectively. Such reactions are carried out in a temperature range of −100 to +300° C., preferably between ambient temperature and 200° C., in the presence of a catalyst, such as a metal catalyst, for instance a palladium catalyst, and a ligand, such as for example a phosphine ligand, or an N-heterocyclic carbene (NHC) ligand, or a phosphite ligand. The reaction can be done in the presence or absence of an additional metal catalyst, such as, for example, a copper salt, for instance CuI. The reaction is done with or without a base, which can be an inorganic base, such as potassium carbonate, or sodium hydroxide, or cesium carbonate, or an organic base, such as an amine base, for instance triethyl amine. This reaction is done with or without a solvent, preferentially in a solvent. Where the reaction mixture is heated, the reaction can be conducted under microwave irradiation or with conventional heating, such as heating the reaction vessel in an oil bath.

By an alternative route, compound XV can be reacted with a compound of the formula XX to give intermediate XXI, wherein R₃ and R₄ₐ have the same meaning as given above for compounds of the formula I. This reaction is done essentially under in the same range of conditions as described for the transformation of intermediate XVIII to the compound of formula I-m. Subsequently, the intermediate XXI is reacted with amine XIX to give intermediate XVIIa, wherein R₁ₐ, R₃ and R₄ₐ have the same meaning as given above for compounds of the formula I. This reaction is done in the presence of a reducing agent, essentially under the same conditions as described above for the transformation of compound XV to intermediate XVII.

Subsequently, the intermediate of the formula XVIIa is reacted with a compound of the formula VIII to give the compound of the formula I-m, wherein T, R₁ₐ, R₃ and R₄ₐ have the same meaning as given above for compounds of the formula I. This reaction is done essentially under the same conditions as described above for the transformation of intermediate XVII to intermediate XVIII.

Within these different multistep sequences, the intermediate compounds of formulas XVII, XVIIa, XVIII and XXI can be used as crude products for the respective subsequent step, or they can be purified, for instance by chromatography, and used in purified form for the next transformation. Compounds of the formula XV are known, or they can be prepared by methods known to a person skilled in the art.

Compounds of the formula I-n

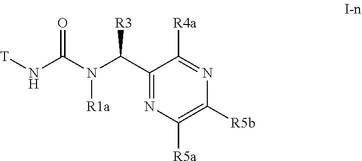

can be prepared by the reaction of an amine of the formula XVIIb

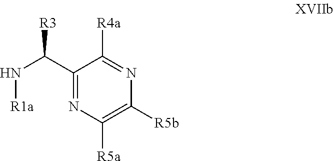

wherein R₁ₐ, R₃, R₄ₐ, R₅ₐ and R_{5b} are as described in formula I with a compound of the formula VIII

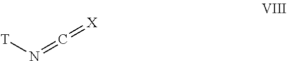

wherein T is as described in formula I.

The chemistry is described in more detail in Scheme 6.

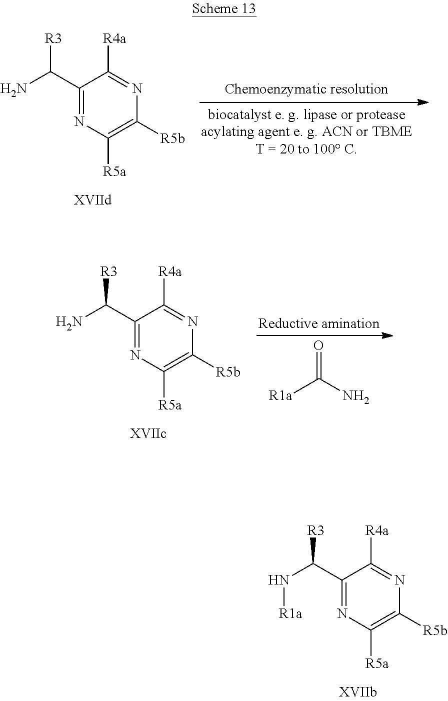

Amines of formula XVIIc may be obtained by biocatalyzed deracemization of amines of formula XVIId, wherein R₃, R₄ₐ, R₅ₐ, and R_{5b} are as defined in formula I. This may be done for instance using a lipase, e.g. Candida Antarctica lipase B or Pseudomonas fluorescens lipase, eventually in immobilized form (e.g. Novozym® 435) in presence of an acyl donor, e.g. ethyl methoxyacetate or vinyl acetate, in a suitable solvent such as acetonitrile or methyl tert-butyl ether at temperatures between 20° C. to 100° C. Such processes are described for instance in J. Org. Chem. 2007, 72, 6918-6923 or Adv. Synth. Catal. 2007, 349, 1481-1488. The expected stereochemical outcome of such enzymatic deracemization are known of those skilled in the art and are documented in the literature, for instance in J. Org. Chem. 1991, 56, 2656-2665 or J. Am. Chem. Soc. 2015, 137, 3996-4009.

Treatment of a compound of the formula XVIIc with an amine of the formula XXII gives compounds of the formula XVIIb. This reaction is done in the presence of a reducing agent, such as for example hydrogen, or a hydride, such as sodium borohydride, with or without a catalyst, such as a hydrogenation catalyst, for example palladium on carbon, with or without the presence of an acid, such as acetic acid, or a Lewis acid, such as zinc bromide, in a solvent or without a solvent, such as, for instance, methanol. The reaction can be conducted in a temperature range of −100 to +300° C., preferably between ambient temperature and 200° C. Such methods, and the range of conditions to perform them, for the alkylation of amines and for the reductive alkylation of amines are well known to a person skilled in the art.

In an alternative process, compounds of formula XVIIc can be obtained from compounds of the formula XVIId wherein R₃, R₄ₐ, R₅ₐ, and R_{5b} are as described in formula I, following the synthesis described in Scheme 14:

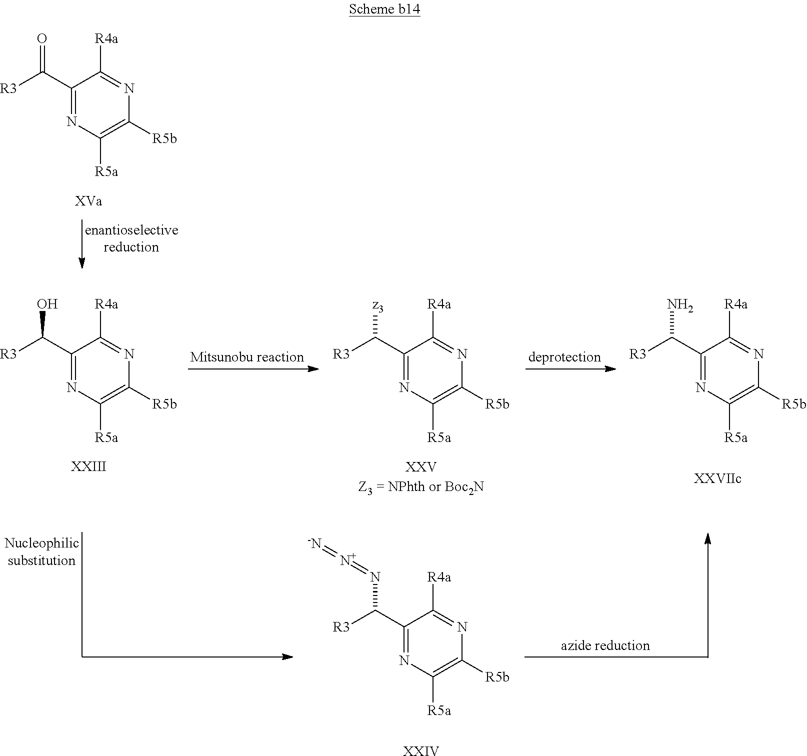

Amines of formula XVIIc may be obtained from intermediates of formula XXV, wherein R₃, R₄ₐ, R₅ₐ, and R_{5b} are as described in formula I and Z₃ is NPhth or NBoc₂. Such intermediates can be obtained from alcohols of formula XXIII by a Mitsunobu reaction, which involves treating alcohols of formula XXIII with diisopropyl azodicarboxylate in the presence of a phosphine such as triphenylphosphine or tributylphosphine and of an amine such as phthalimide or bis(tert-butoxycarbonyl)amine. Mitsunobu reactions are known by those skilled in the art to proceed with inversion of the stereocenter, as described for instance in Chem. Rev. 2009, 109, 2551-2651. Compounds of formula XXV can then be transformed into amines of formula XVIIb by treatment with hydrazine if Z₃=NPhth or with acid, for example trifluoroacetic acid, if Z₃=NBoc₂.

Alternatively, amines of formula XVIIc may be obtained by reduction of azides of formula XXIV, wherein R₃, R₄ₐ, R₅ₐ, and R_{5b} are as described in formula I, by treatment with triphenylphosphine and water (Staudinger reaction) or by hydrogenation for example using a palladium catalyst in the presence of hydrogen. Azides of formula XXIV may be obtained by treatment of alcohols of formula XXIII, wherein R₃, R₄ₐ, R₅ₐ, and R_{5b} are as described in formula I, with an azidation reagent such as diphenyl phosphoryl azide in a solvent such as toluene or THE in presence of a base such as DBU. Such processes are known by those skilled in the art to proceed with inversion of the stereocenter and are described in the literature for instance in Adv. Synth. Catal. 2018, 360, 2157-2165.

Alcohols of formula XXIII may be obtained by enantioselective reduction of ketones of formula XVa, wherein R₃, R₄ₐ, R₅ₐ, and R_{5b} are as described in formula I. Such reductions can be done using a catalyst, for instance a ruthenium or a rhodium catalyst with a chiral ligand such as RuCl[(R,R)-TsDPEN](mesitylene) or RuBF₄[(R,R)-TsDPEN](p-cymene) in the presence of a hydrogen donor system such as for example HCOOH/Et₃N or HCO₂NH₄. Such processes are described in the literature for instance in J. Org. Chem. 2017, 82, 5607.

Alternatively, compounds of formula XVIIc may also be prepared as outlined in Scheme 15.

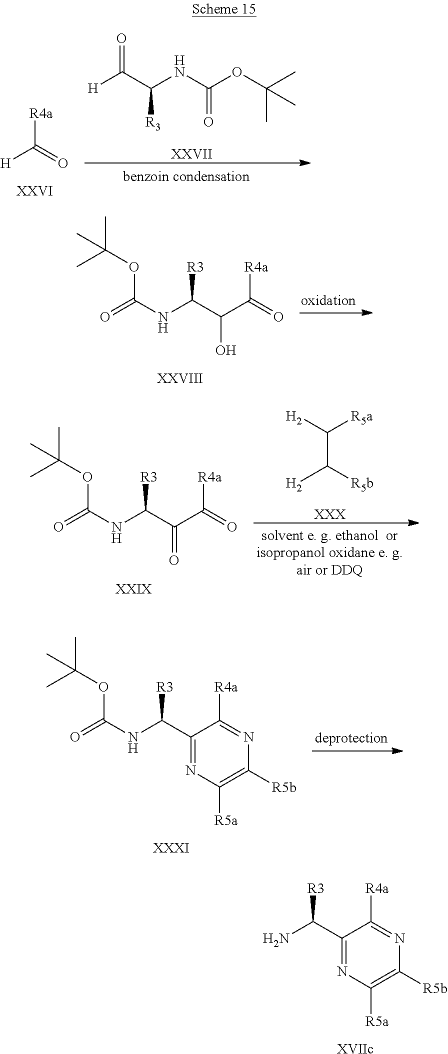

Amines of formula XVIIc can be prepared by deprotection of amines of formula XXXI, wherein R₃, R₄ₐ, R₅ₐ, and R_{5b} are as described in formula I, for instance using an acid such as trifluoroacetic acid or hydrochloric acid. Amines of formula XXXI can be obtained by condensation of diamines of formula XXX, wherein R₅ₐ, and R_{5b} are as described in formula I, on diketones of formula XXIX, wherein R₃, and R₄ₐ are as described in formula I. This condensation can take place in the presence of a suitable solvent such as ethanol or isopropanol in presence of an oxidant such as air or DDQ. Diketones of formula XXIX may be formed by oxidation of hydroxyketones of formula XXVIII wherein R₃, and R₄ₐ are as described in formula I. This oxidation can involve for instance SO₃-pyridine in presence of DMSO and a base for instance triethylamine or alternatively sodium hypochlorite in presence of a catalyst such as TEMPO/Bu₄NHSO₄. Examples of such oxidations can be found in the literature, for instance in Synlett, 2014, 25, 596 or J. Am. Chem. Soc. 1990, 112, 5290-5313. Hydroxyketones of formula XXVIII may be synthesized by cross-benzoin condensation between aldehydes of formula XXVII, wherein R₄ₐ is as described in formula I, and aldehydes of formula XXVI, wherein R₃ is as described in formula 1. Aldehydes of formula XXVI are commercially available in chiral form, like for instance Boc-L-alaninal (CAS 79069-50-4) or tert-butyl N-[(1S)-1-(cyclopropylmethyl)-2-oxo-ethyl]carbamate (CAS 881902-36-9). Cross-benzoin condensations are done in the usual way by employing an organocatalyst such as a triazolium salt or a thiazolium salt in the presence of a base such as potassium tert-butoxide or isopropyldiethylamine in a suitable solvent such as DCM or THE at a temperature between −20° C. and the boiling point of the solvent. Examples of catalysts for such transformations have been described in the literature for instance in J. Am. Chem. Soc. 2014, 136, 7539-7542 or in Org. Lett. 2016, 18, 4518-4521.

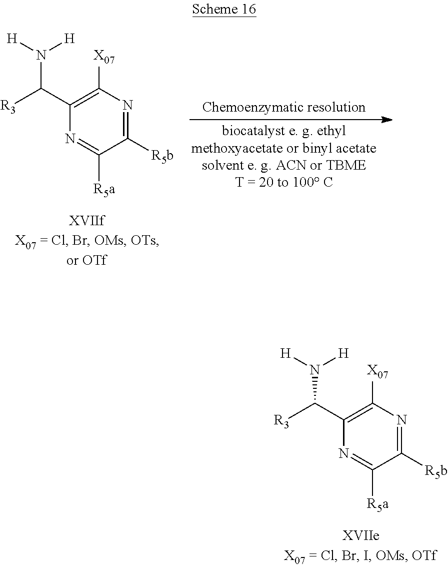

Amines of formula XVIIe may be obtained by biocatalyzed deracemization of amines of formula XVIIf, wherein R₃, R₅ₐ, and R_{5b} are as defined in formula I and X₀₇ is a leaving group such as bromine, chlorine or iodine. This may be done for instance using a lipase, e.g. Candida antarctica lipase B or Pseudomonas fluorescens lipase, eventually in immobilized form (e.g. Novozym® 435) in presence of an acyl donor, e.g. ethyl methoxyacetate or vinyl acetate, in a suitable solvent such as acetonitrile or methyl tert-butyl ether at temperatures between 20° C. to 100° C. Such processes are described for instance in J. Org. Chem. 2007, 72, 6918-6923 or Adv. Synth. Catal. 2007, 349, 1481-1488. The expected stereochemical outcome of such enzymatic deracemization are known of those skilled in the art and are documented in the literature, for instance in J. Org. Chem. 1991, 56, 2656-2665 or J. Am. Chem. Soc. 2015, 137, 3996-4009.

Alternatively, resolution of amines of formula XVIIf to give amines of formula XVIIe may be achieved using a chiral auxiliary, as described in Scheme 17.

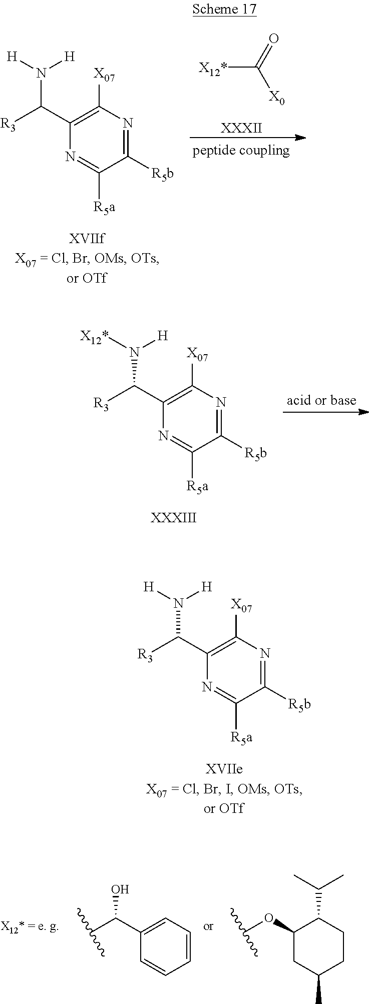

Amines of formula XVIIe can be prepared from intermediates of formula XXXIII, wherein R₃, R₅ₐ, and R_{5b} are as in compounds of the formula I, X₀₇ is a leaving group such as bromine, chlorine or iodine, and X₁₂* is a chiral auxiliary, by treatment with acids such as HCl or bases such as NaOH. Chiral auxiliaries of formula XXXII are for instance mandelic acid or (1R)-menthylchloroformate. Intermediates of formula XXXIII can be formed by coupling of a chiral auxiliary of formula XXXII, wherein Xo is a leaving group, such as chlorine, with amines of the formula XVIIf. The reaction can be conducted neat or in a solvent, preferably in a solvent, such as an organic solvent, for instance acetonitrile, in a temperature range of −100 to +300° C., preferably between ambient temperature and 200° C., with or without the presence of a catalyst, for instance a metal catalyst, such as a palladium complex, and with or without the addition of a base, such as an inorganic base, for instance potassium carbonate, or an organic base, such as, for example, triethylamine. Compounds of the formula XXXII are either known, or they can be prepared by methods known to a person skilled in the art. Examples of such deracemization processes are reported in the literature, for instance in J. Org. Chem. 2007, 72, 485-493.

Alternatively, amines of formula XVIIe can be formed as described in Scheme 18.

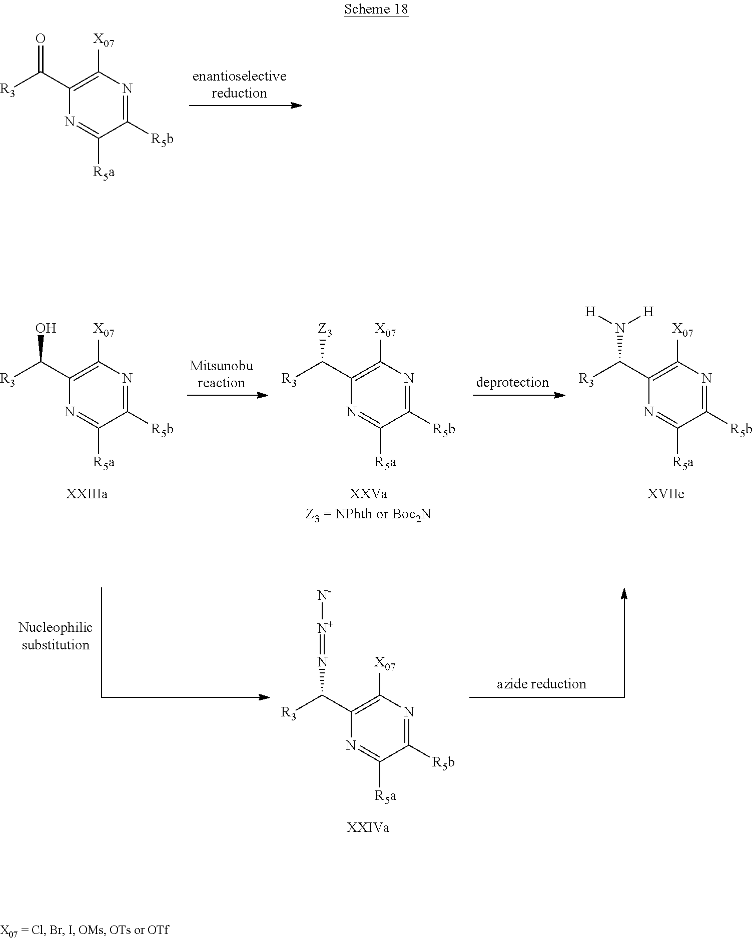

Alternatively, amines of formula XVIIe may be obtained from intermediates of formula XXVa, wherein R₃, R₅ₐ, and R_{5b} are as described in formula I, X₀₇ is a leaving group such as a halogen or sulfonate, for instance bromide, and Z₃ is NPhth or NBoc₂. Such intermediates can be obtained from alcohols of formula XXIIIa, wherein R₃, R₅ₐ, and R_{5b} are as described in formula I and X₀₇ is a leaving group, by a Mitsunobu reaction, which involves treating alcohols of formula XXIIIa with diisopropyl azodicarboxylate in the presence of a phosphine such as triphenylphosphine or tributylphosphine and of an amine such as phthalimide or bis(tert-butoxycarbonyl)amine. Mitsunobu reactions are known by those skilled in the art to proceed with inversion of the stereocenter, as described for instance in Chem. Rev. 2009, 109, 2551-2651. Amines of formula XXVa can then be transformed into amines of formula XVIIe by treatment with hydrazine if Z₃=NPhth or with an acid, such as TFA, if Z₃=NBoc₂.

Alternatively, amines of formula XVIIe may be obtained by reduction of azides of formula XXIVa, wherein R₃, R₅ₐ, and R_{5b} are as described in formula I and X₀₇ is a leaving group such as a halogen or sulfonate, for instance bromide, by treatment with triphenylphosphine and water (Staudinger reaction) or by hydrogenation for example using a palladium catalyst in the presence of hydrogen. Azides of formula XXIVa may be obtained by treatment of alcohols of formula XXIIIa with an azidation reagent such as diphenyl phosphoryl azide in a solvent such as toluene or THE in presence of a base such as DBU. Such processes are known by those skilled in the art to proceed with inversion of the stereocenter and are described in the literature for instance in Adv. Synth. Catal. 2018, 360, 2157-2165.

Alcohols of formula XXIIIa may be obtained by enantioselective reduction of ketones of formula XVb, wherein R₃, R₅ₐ, and R_{5b} are as described in formula I and X₀₇ is a leaving group such as a halogen or sulfonate, for instance bromide. Such reductions can be done using catalysts, for instance a ruthenium or a rhodium catalyst with a chiral ligand such as RuCl[(R,R)-TsDPEN](mesitylene) or RuBF₄[(R,R)-TsDPEN](p-cymene) in the presence of a hydrogen donor system such as for example HCOOH/Et₃N or HCO₂NH₄. Such processes are described in the literature for instance in J. Org. Chem. 2017, 82, 5607.

Depending on the procedure or the reaction conditions, the reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diastereomers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I (when A2 and A3 are each N) with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula I-A according to the following Tables A-1 to A-243 can be prepared according to the methods described above. The examples which follow are intended to illustrate the invention and show preferred compounds of formula I, in the form of a compound of formula I-A.

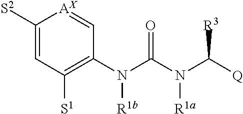

Table A-1 provides 8 compounds A-1.001 to A-1.008 of formula I-A wherein Ax is C—O—CF₂CF₂H, S¹ is Cl, S² is Cl, R^{1b} is H, R^{1a} is H, R³ is CH₃ and Q are as defined in table Z. For example, A-1.002 is

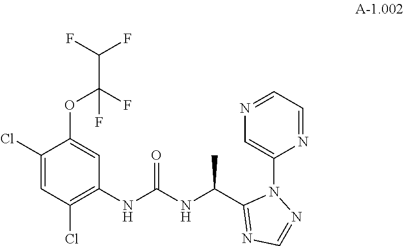 [SMILES: C[C@H](NC(=O)Nc1cc(OC(F)(F)C(F)F)c(Cl)cc1Cl)c1ncnn1-c1cnccn1]

Table A-2 provides 8 compounds A-2.001 to A-2.008 of formula I-A wherein Ax is C—O—CF₂CF₂H, S¹ is Cl, S² is Cl, R^{1b} is H, R^{1a} is CH₃, R³ is CH₃ and Q are as defined in table Z.

Table A-3 provides 8 compounds A-3.001 to A-3.008 of formula I-A wherein Ax is C—O—CF₂CF₂H, S¹ is Cl, S² is Cl, R^{1b} is H, R^{1a} is CH₂-cyclopropyl, R³ is CH₃ and Q are as defined in table Z.

Table A-4 provides 8 compounds A-4.001 to A-4.008 of formula I-A wherein Ax is C—O—CF₂CF₂H, S¹ is Cl, S² is Cl, R^{1b} is CH₃, R^{1a} is H, R³ is CH₃ and Q are as defined in table Z.

Table A-5 provides 8 compounds A-5.001 to A-5.008 of formula I-A wherein Ax is C—O—CF₂CF₂H, S¹ is Cl, S² is Cl, R^{1b} is CH₃, R^{1a} is CH₃, R³ is CH₃ and Q are as defined in table Z.

Table A-6 provides 8 compounds A-6.001 to A-6.008 of formula I-A wherein Ax is C—O—CF₂CF₂H, S¹ is Cl, S² is Cl, R^{1b} is CH₃, R^{1a} is CH₂-cyclopropyl, R³ is CH₃ and Q are as defined in table Z.

Table A-7 provides 8 compounds A-7.001 to A-7.008 of formula I-A wherein Ax is C—O—CF₂CF₂H, S¹ is Cl, S² is Cl, R^{1b} is CH₂-cyclopropyl, R^{1a} is H, R³ is CH₃ and Q are as defined in table Z.

Table A-8 provides 8 compounds A-8.001 to A-8.008 of formula I-A wherein Ax is C—O—CF₂CF₂H, S¹ is Cl, S² is Cl, R^{1b} is CH₂-cyclopropyl, R^{1a} is CH₃, R³ is CH₃ and Q are as defined in table Z.

Table A-9 provides 8 compounds A-9.001 to A-9.008 of formula I-A wherein Ax is C—O—CF₂CF₂H, S¹ is Cl, S² is Cl, R^{1b} is CH₂-cyclopropyl, R₁ₐ is CH₂-cyclopropyl, R³ is CH₃ and Q are as defined in table Z.

Table A-10 provides 8 compounds A-10.001 to A-10.008 of formula I-A wherein Ax is C—O—CF₂CF₂H, S¹ is Cl, S² is Br, R^{1b} is H, R^{1a} is H, R³ is CH₃ and Q are as defined in table Z.

Table A-11 provides 8 compounds A-11.001 to A-11.008 of formula I-A wherein Ax is C—O—CF₂CF₂H, S¹ is Cl, S² is Br, R^{1b} is H, R₁ₐ is CH₃, R³ is CH₃ and Q are as defined in table Z.

Table A-12 provides 8 compounds A-12.001 to A-12.008 of formula I-A wherein Ax is C—O—CF₂CF₂H, S¹ is Cl, S² is Br, R^{1b} is H, R^{1a} is CH₂-cyclopropyl, R³ is CHs and Q are as defined in table Z.

Table A-13 provides 8 compounds A-13.001 to A-13.008 of formula I-A wherein Ax is C—O—CF₂CF₂H, S¹ is Cl, S² is Br, R^{1b} is CH₃, R^{1a} is H, R³ is CH₃ and Q are as defined in table Z.

Table A-14 provides 8 compounds A-14.001 to A-14.008 of formula I-A wherein Ax is C—O—CF₂CF₂H, S¹ is Cl, S² is Br, R^{1b} is CH₃, R^{1a} is CH₃, R³ is CH₃ and Q are as defined in table Z.

Table A-15 provides 8 compounds A-15.001 to A-15.008 of formula I-A wherein Ax is C—O—CF₂CF₂H, S¹ is Cl, S² is Br, R^{1b} is CH₃, R^{1a} is CH₂-cyclopropyl, R³ is CH₃ and Q are as defined in table Z.

Table A-16 provides 8 compounds A-16.001 to A-16.008 of formula I-A wherein Ax is C—O—CF₂CF₂H, S¹ is Cl, S² is Br, R^{1b} is CH₂-cyclopropyl, R^{1a} is H, R³ is CH₃ and Q are as defined in table Z.

Table A-17 provides 8 compounds A-17.001 to A-17.008 of formula I-A wherein Ax is C—O—CF₂CF₂H, S¹ is Cl, S² is Br, R^{1b} is CH₂-cyclopropyl, R^{1a} is CH₃, R³ is CH₃ and Q are as defined in table Z.

Table A-18 provides 8 compounds A-18.001 to A-18.008 of formula I-A wherein Ax is C—O—CF₂CF₂H, S¹ is Cl, S² is Br, R^{1b} is CH₂-cyclopropyl, R^{1a} is CH₂-cyclopropyl, R³ is CH₃ and Q are as defined in table Z.

Table A-19 provides 8 compounds A-19.001 to A-19.008 of formula I-A wherein Ax is C—O—CF₂CF₂H, S¹ is Cl, S² is CF₃, R^{1b} is H, R^{1a} is H, R³ is CH₃ and Q are as defined in table Z.

Table A-20 provides 8 compounds A-20.001 to A-20.008 of formula I-A wherein Ax is C—O—CF₂CF₂H, S¹ is Cl, S² is CF₃, R^{1b} is H, R^{1a} is CH₃, R³ is CH₃ and Q are as defined in table Z.

Table A-21 provides 8 compounds A-21.001 to A-21.008 of formula I-A wherein Ax is C—O—CF₂CF₂H, S¹ is Cl, S² is CF₃, R^{1b} is H, R₁ₐ is CH₂-cyclopropyl, R³ is CH₃ and Q are as defined in table Z.

Table A-22 provides 8 compounds A-22.001 to A-22.008 of formula I-A wherein Ax is C—O—CF₂CF₂H, S¹ is Cl, S² is CF₃, R^{1b} is CH₃, R₁ₐ is H, R³ is CH₃ and Q are as defined in table Z.

Table A-23 provides 8 compounds A-23.001 to A-23.008 of formula I-A wherein Ax is C—O—CF₂CF₂H, S¹ is Cl, S² is CF₃, R^{1b} is CH₃, R₁ₐ is CH₃, R³ is CH₃ and Q are as defined in table Z.

Table A-24 provides 8 compounds A-24.001 to A-24.008 of formula I-A wherein Ax is C—O—CF₂CF₂H, S¹ is Cl, S² is CF₃, R^{1b} is CH₃, R₁ₐ is CH₂-cyclopropyl, R³ is CH₃ and Q are as defined in table Z.

Table A-25 provides 8 compounds A-25.001 to A-25.008 of formula I-A wherein Ax is C—O—CF₂CF₂H, S¹ is Cl, S² is CF₃, R^{1b} is CH₂-cyclopropyl, R₁ₐ is H, R³ is CH₃ and Q are as defined in table Z.

Table A-26 provides 8 compounds A-26.001 to A-26.008 of formula I-A wherein Ax is C—O—CF₂CF₂H, S¹ is Cl, S² is CF₃, R^{1b} is CH₂-cyclopropyl, R₁ₐ is CH₃, R³ is CH₃ and Q are as defined in table Z.

Table A-27 provides 8 compounds A-27.001 to A-27.008 of formula I-A wherein Ax is C—O—CF₂CF₂H, S¹ is Cl, S² is CF₃, R^{1b} is CH₂-cyclopropyl, R₁ₐ is CH₂-cyclopropyl, R³ is CH₃ and Q are as defined in table Z.

Table A-28 provides 8 compounds A-28.001 to A-28.008 of formula I-A wherein Ax is C—O—CF₂CF₂H, S¹ is Br, S² is Cl, R^{1b} is H, R^{1a} is H, R³ is CHs and Q are as defined in table Z.

Table A-29 provides 8 compounds A-29.001 to A-29.008 of formula I-A wherein Ax is C—O—CF₂CF₂H, S¹ is Br, S² is Cl, R^{1b} is H, R^{1a} is CH₃, R³ is CH₃ and Q are as defined in table Z.

Table A-30 provides 8 compounds A-30.001 to A-30.008 of formula I-A wherein Ax is C—O—CF₂CF₂H, S¹ is Br, S² is Cl, R^{1b} is H, R^{1a} is CH₂-cyclopropyl, R³ is CH₃ and Q are as defined in table Z.

Table A-31 provides 8 compounds A-31.001 to A-31.008 of formula I-A wherein Ax is C—O—CF₂CF₂H, S¹ is Br, S² is Cl, R^{1b} is CH₃, R^{1a} is H, R³ is CH₃ and Q are as defined in table Z.

Table A-32 provides 8 compounds A-32.001 to A-32.008 of formula I-A wherein Ax is C—O—CF₂CF₂H, S¹ is Br, S² is Cl, R^{1b} is CH₃, R^{1a} is CH₃, R³ is CH₃ and Q are as defined in table Z.

Table A-33 provides 8 compounds A-33.001 to A-33.008 of formula I-A wherein Ax is C—O—CF₂CF₂H, S¹ is Br, S² is Cl, R^{1b} is CH₃, R₁ₐ is CH₂-cyclopropyl, R³ is CH₃ and Q are as defined in table Z.

Table A-34 provides 8 compounds A-34.001 to A-34.008 of formula I-A wherein Ax is C—O—CF₂CF₂H, S¹ is Br, S² is Cl, R^{1b} is CH₂-cyclopropyl, R₁ₐ is H, R³ is CH₃ and Q are as defined in table Z.

Table A-35 provides 8 compounds A-35.001 to A-35.008 of formula I-A wherein Ax is C—O—CF₂CF₂H, S¹ is Br, S² is Cl, R^{1b} is CH₂-cyclopropyl, R₁ₐ is CH₃, R³ is CH₃ and Q are as defined in table Z.

Table A-36 provides 8 compounds A-36.001 to A-36.008 of formula I-A wherein Ax is C—O—CF₂CF₂H, S¹ is Br, S² is Cl, R^{1b} is CH₂-cyclopropyl, R₁ₐ is CH₂-cyclopropyl, R³ is CH₃ and Q are as defined in table Z.

Table A-37 provides 8 compounds A-37.001 to A-37.008 of formula I-A wherein Ax is C—O—CF₂CF₂H, S¹ is Br, S² is Br, R^{1b} is H, R^{1a} is H, R³ is CH₃ and Q are as defined in table Z.

Table A-38 provides 8 compounds A-38.001 to A-38.008 of formula I-A wherein Ax is C—O—CF₂CF₂H, S¹ is Br, S² is Br, R^{1b} is H, R₁ₐ is CH₃, R³ is CH₃ and Q are as defined in table Z.

Table A-39 provides 8 compounds A-39.001 to A-39.008 of formula I-A wherein Ax is C—O—CF₂CF₂H, S¹ is Br, S² is Br, R^{1b} is H, R₁ₐ is CH₂-cyclopropyl, R³ is CH₃ and Q are as defined in table Z.

Table A-40 provides 8 compounds A-40.001 to A-40.008 of formula I-A wherein Ax is C—O—CF₂CF₂H, S¹ is Br, S² is Br, R^{1b} is CH₃, R^{1a} is H, R³ is CH₃ and Q are as defined in table Z.

Table A-41 provides 8 compounds A-41.001 to A-41.008 of formula I-A wherein Ax is C—O—CF₂CF₂H, S¹ is Br, S² is Br, R^{1b} is CH₃, R₁ₐ is CH₃, R³ is CH₃ and Q are as defined in table Z.

Table A-42 provides 8 compounds A-42.001 to A-42.008 of formula I-A wherein Ax is C—O—CF₂CF₂H, S¹ is Br, S² is Br, R^{1b} is CH₃, R₁ₐ is CH₂-cyclopropyl, R³ is CH₃ and Q are as defined in table Z.

Table A-43 provides 8 compounds A-43.001 to A-43.008 of formula I-A wherein Ax is C—O—CF₂CF₂H, S¹ is Br, S² is Br, R^{1b} is CH₂-cyclopropyl, R₁ₐ is H, R³ is CH₃ and Q are as defined in table Z.

Table A-44 provides 8 compounds A-44.001 to A-44.008 of formula I-A wherein Ax is C—O—CF₂CF₂H, S¹ is Br, S² is Br, R^{1b} is CH₂-cyclopropyl, R^{1a} is CHs, R³ is CHs and Q are as defined in table Z.

Table A-45 provides 8 compounds A-45.001 to A-45.008 of formula I-A wherein Ax is C—O—CF₂CF₂H, S¹ is Br, S² is Br, R^{1b} is CH₂-cyclopropyl, R^{1a} is CH₂-cyclopropyl, R³ is CH₃ and Q are as defined in table Z.

Table A-46 provides 8 compounds A-46.001 to A-46.008 of formula I-A wherein Ax is C—O—CF₂CF₂H, S¹ is Br, S² is CF₃, R^{1b} is H, R^{1a} is H, R³ is CH₃ and Q are as defined in table Z.

Table A-47 provides 8 compounds A-47.001 to A-47.008 of formula I-A wherein Ax is C—O—CF₂CF₂H, S¹ is Br, S² is CF₃, R^{1b} is H, R^{1a} is CH₃, R³ is CH₃ and Q are as defined in table Z.

Table A-48 provides 8 compounds A-48.001 to A-48.008 of formula I-A wherein Ax is C—O—CF₂CF₂H, S¹ is Br, S² is CF₃, R^{1b} is H, R^{1a} is CH₂-cyclopropyl, R³ is CH₃ and Q are as defined in table Z.

Table A-49 provides 8 compounds A-49.001 to A-49.008 of formula I-A wherein Ax is C—O—CF₂CF₂H, S¹ is Br, S² is CF₃, R^{1b} is CH₃, R₁ₐ is H, R³ is CH₃ and Q are as defined in table Z.

Table A-50 provides 8 compounds A-50.001 to A-50.008 of formula I-A wherein Ax is C—O—CF₂CF₂H, S¹ is Br, S² is CF₃, R^{1b} is CH₃, R₁ₐ is CH₃, R³ is CH₃ and Q are as defined in table Z.

Table A-51 provides 8 compounds A-51.001 to A-51.008 of formula I-A wherein Ax is C—O—CF₂CF₂H, S¹ is Br, S² is CF₃, R^{1b} is CH₃, R₁ₐ is CH₂-cyclopropyl, R³ is CH₃ and Q are as defined in table Z.

Table A-52 provides 8 compounds A-52.001 to A-52.008 of formula I-A wherein Ax is C—O—CF₂CF₂H, S¹ is Br, S² is CF₃, R^{1b} is CH₂-cyclopropyl, R₁ₐ is H, R³ is CH₃ and Q are as defined in table Z.

Table A-53 provides 8 compounds A-53.001 to A-53.008 of formula I-A wherein Ax is C—O—CF₂CF₂H, S¹ is Br, S² is CF₃, R^{1b} is CH₂-cyclopropyl, R₁ₐ is CH₃, R³ is CH₃ and Q are as defined in table Z.

Table A-54 provides 8 compounds A-54.001 to A-54.008 of formula I-A wherein Ax is C—O—CF₂CF₂H, S¹ is Br, S² is CF₃, R^{1b} is CH₂-cyclopropyl, R₁ₐ is CH₂-cyclopropyl, R³ is CH₃ and Q are as defined in table Z.

Table A-55 provides 8 compounds A-55.001 to A-55.008 of formula I-A wherein Ax is C—O—CF₂CF₂H, S¹ is CF₃, S² is Cl, R^{1b} is H, R₁ₐ is H, R³ is CH₃ and Q are as defined in table Z.

Table A-56 provides 8 compounds A-56.001 to A-56.008 of formula I-A wherein Ax is C—O—CF₂CF₂H, S¹ is CF₃, S² is Cl, R^{1b} is H, R₁ₐ is CH₃, R³ is CH₃ and Q are as defined in table Z.

Table A-57 provides 8 compounds A-57.001 to A-57.008 of formula I-A wherein Ax is C—O—CF₂CF₂H, S¹ is CF₃, S² is Cl, R^{1b} is H, R₁ₐ is CH₂-cyclopropyl, R³ is CH₃ and Q are as defined in table Z.

Table A-58 provides 8 compounds A-58.001 to A-58.008 of formula I-A wherein Ax is C—O—CF₂CF₂H, S¹ is CF₃, S² is Cl, R^{1b} is CH₃, R₁ₐ is H, R³ is CH₃ and Q are as defined in table Z.

Table A-59 provides 8 compounds A-59.001 to A-59.008 of formula I-A wherein Ax is C—O—CF₂CF₂H, S¹ is CF₃, S² is Cl, R^{1b} is CH₃, R₁ₐ is CH₃, R³ is CH₃ and Q are as defined in table Z.

Table A-61 provides 8 compounds A-61.001 to A-61.008 of formula I-A wherein Ax is C—O—CF₂CF₂H, S¹ is CFs, S² is Cl, R^{1b} is CH₂-cyclopropyl, R^{1a} is H, R³ is CHs and Q are as defined in table Z.

Table A-62 provides 8 compounds A-62.001 to A-62.008 of formula I-A wherein Ax is C—O—CF₂CF₂H, S¹ is CF₃, S² is Cl, R^{1b} is CH₂-cyclopropyl, R^{1a} is CH₃, R³ is CH₃ and Q are as defined in table Z.

Table A-63 provides 8 compounds A-63.001 to A-63.008 of formula I-A wherein Ax is C—O—CF₂CF₂H, S¹ is CF₃, S² is Cl, R^{1b} is CH₂-cyclopropyl, R^{1a} is CH₂-cyclopropyl, R³ is CH₃ and Q are as defined in table Z.

Table A-64 provides 8 compounds A-64.001 to A-64.008 of formula I-A wherein Ax is C—O—CF₂CF₂H, S¹ is CF₃, S² is Br, R^{1b} is H, R^{1a} is H, R³ is CH₃ and Q are as defined in table Z.

Table A-65 provides 8 compounds A-65.001 to A-65.008 of formula I-A wherein Ax is C—O—CF₂CF₂H, S¹ is CF₃, S² is Br, R^{1b} is H, R^{1a} is CH₃, R³ is CH₃ and Q are as defined in table Z.

Table A-66 provides 8 compounds A-66.001 to A-66.008 of formula I-A wherein Ax is C—O—CF₂CF₂H, S¹ is CF₃, S² is Br, R^{1b} is H, R^{1a} is CH₂-cyclopropyl, R³ is CH₃ and Q are as defined in table Z.

Table A-67 provides 8 compounds A-67.001 to A-67.008 of formula I-A wherein Ax is C—O—CF₂CF₂H, S¹ is CF₃, S² is Br, R^{1b} is CH₃, R^{1a} is H, R³ is CH₃ and Q are as defined in table Z.

Table A-68 provides 8 compounds A-68.001 to A-68.008 of formula I-A wherein Ax is C—O—CF₂CF₂H, S¹ is CF₃, S² is Br, R^{1b} is CH₃, R^{1a} is CH₃, R³ is CH₃ and Q are as defined in table Z.

Table A-69 provides 8 compounds A-69.001 to A-69.008 of formula I-A wherein Ax is C—O—CF₂CF₂H, S¹ is CF₃, S² is Br, R^{1b} is CH₃, R^{1a} is CH₂-cyclopropyl, R³ is CH₃ and Q are as defined in table Z.

Table A-70 provides 8 compounds A-70.001 to A-70.008 of formula I-A wherein Ax is C—O—CF₂CF₂H, S¹ is CF₃, S² is Br, R^{1b} is CH₂-cyclopropyl, R₁ₐ is H, R³ is CH₃ and Q are as defined in table Z.

Table A-71 provides 8 compounds A-71.001 to A-71.008 of formula I-A wherein Ax is C—O—CF₂CF₂H, S¹ is CF₃, S² is Br, R^{1b} is CH₂-cyclopropyl, R₁ₐ is CH₃, R³ is CH₃ and Q are as defined in table Z.

Table A-72 provides 8 compounds A-72.001 to A-72.008 of formula I-A wherein Ax is C—O—CF₂CF₂H, S¹ is CF₃, S² is Br, R^{1b} is CH₂-cyclopropyl, R₁ₐ is CH₂-cyclopropyl, R³ is CH₃ and Q are as defined in table Z.

Table A-73 provides 8 compounds A-73.001 to A-73.008 of formula I-A wherein Ax is C—O—CF₂CF₂H, S¹ is CF₃, S² is CF₃, R^{1b} is H, R₁ₐ is H, R³ is CH₃ and Q are as defined in table Z.

Table A-74 provides 8 compounds A-74.001 to A-74.008 of formula I-A wherein Ax is C—O—CF₂CF₂H, S¹ is CF₃, S² is CF₃, R^{1b} is H, R₁ₐ is CH₃, R³ is CH₃ and Q are as defined in table Z.

Table A-75 provides 8 compounds A-75.001 to A-75.008 of formula I-A wherein Ax is C—O—CF₂CF₂H, S¹ is CF₃, S² is CF₃, R^{1b} is H, R₁ₐ is CH₂-cyclopropyl, R³ is CH₃ and Q are as defined in table Z.

Table A-76 provides 8 compounds A-76.001 to A-76.008 of formula I-A wherein Ax is C—O—CF₂CF₂H, S¹ is CFs, S² is CFs, R^{1b} is CHs, R^{1a} is H, R³ is CHs and Q are as defined in table Z.

Table A-77 provides 8 compounds A-77.001 to A-77.008 of formula I-A wherein Ax is C—O—CF₂CF₂H, S¹ is CF₃, S² is CF₃, R^{1b} is CH₃, R^{1a} is CH₃, R³ is CH₃ and Q are as defined in table Z.

Table A-78 provides 8 compounds A-78.001 to A-78.008 of formula I-A wherein Ax is C—O—CF₂CF₂H, S¹ is CF₃, S² is CF₃, R^{1b} is CH₃, R^{1a} is CH₂-cyclopropyl, R³ is CH₃ and Q are as defined in table Z.

Table A-79 provides 8 compounds A-79.001 to A-79.008 of formula I-A wherein Ax is C—O—CF₂CF₂H, S¹ is CF₃, S² is CF₃, R^{1b} is CH₂-cyclopropyl, R^{1a} is H, R³ is CH₃ and Q are as defined in table Z.

Table A-80 provides 8 compounds A-80.001 to A-80.008 of formula I-A wherein Ax is C—O—CF₂CF₂H, S¹ is CF₃, S² is CF₃, R^{1b} is CH₂-cyclopropyl, R^{1a} is CH₃, R³ is CH₃ and Q are as defined in table Z.

Table A-81 provides 8 compounds A-81.001 to A-81.008 of formula I-A wherein Ax is C—O—CF₂CF₂H, S¹ is CF₃, S² is CF₃, R^{1b} is CH₂-cyclopropyl, R^{1a} is CH₂-cyclopropyl, R³ is CH₃ and Q are as defined in table Z.

Table A-82 provides 8 compounds A-82.001 to A-82.008 of formula I-A wherein Ax is C—CF₃, S¹ is Cl, S² is Cl, R^{1b} is H, R^{1a} is H, R³ is CH₃ and Q are as defined in table Z.

Table A-83 provides 8 compounds A-83.001 to A-83.008 of formula I-A wherein Ax is C—CF₃, S¹ is Cl, S² is Cl, R^{1b} is H, R^{1a} is CH₃, R³ is CH₃ and Q are as defined in table Z.

Table A-84 provides 8 compounds A-84.001 to A-84.008 of formula I-A wherein Ax is C—CF₃, S¹ is Cl, S² is Cl, R^{1b} is H, R^{1a} is CH₂-cyclopropyl, R³ is CH₃ and Q are as defined in table Z.

Table A-85 provides 8 compounds A-85.001 to A-85.008 of formula I-A wherein Ax is C—CF₃, S¹ is Cl, S² is Cl, R^{1b} is CH₃, R₁ₐ is H, R³ is CH₃ and Q are as defined in table Z.

Table A-86 provides 8 compounds A-86.001 to A-86.008 of formula I-A wherein Ax is C—CF₃, S¹ is Cl, S² is Cl, R^{1b} is CH₃, R₁ₐ is CH₃, R³ is CH₃ and Q are as defined in table Z.

Table A-87 provides 8 compounds A-87.001 to A-87.008 of formula I-A wherein Ax is C—CF₃, S¹ is Cl, S² is Cl, R^{1b} is CH₃, R₁ₐ is CH₂-cyclopropyl, R³ is CH₃ and Q are as defined in table Z.

Table A-88 provides 8 compounds A-88.001 to A-88.008 of formula I-A wherein Ax is C—CF₃, S¹ is Cl, S² is Cl, R^{1b} is CH₂-cyclopropyl, R₁ₐ is H, R³ is CH₃ and Q are as defined in table Z.

Table A-89 provides 8 compounds A-89.001 to A-89.008 of formula I-A wherein Ax is C—CF₃, S¹ is Cl, S² is Cl, R^{1b} is CH₂-cyclopropyl, R₁ₐ is CH₃, R³ is CH₃ and Q are as defined in table Z.

Table A-90 provides 8 compounds A-90.001 to A-90.008 of formula I-A wherein Ax is C—CF₃, S¹ is Cl, S² is Cl, R^{1b} is CH₂-cyclopropyl, R₁ₐ is CH₂-cyclopropyl, R³ is CH₃ and Q are as defined in table Z.

Table A-91 provides 8 compounds A-91.001 to A-91.008 of formula I-A wherein Ax is C—CF₃, S¹ is Cl, S² is Br, R^{1b} is H, R^{1a} is H, R³ is CH₃ and Q are as defined in table Z.

Table A-92 provides 8 compounds A-92.001 to A-92.008 of formula I-A wherein Ax is C—CF₃, S¹ is Cl, S² is Br, R^{1b} is H, R^{1a} is CHs, R³ is CHs and Q are as defined in table Z.

Table A-93 provides 8 compounds A-93.001 to A-93.008 of formula I-A wherein Ax is C—CF₃, S¹ is Cl, S² is Br, R^{1b} is H, R^{1a} is CH₂-cyclopropyl, R³ is CH₃ and Q are as defined in table Z.

Table A-94 provides 8 compounds A-94.001 to A-94.008 of formula I-A wherein Ax is C—CF₃, S¹ is Cl, S² is Br, R^{1b} is CH₃, R^{1a} is H, R³ is CH₃ and Q are as defined in table Z.

Table A-95 provides 8 compounds A-95.001 to A-95.008 of formula I-A wherein Ax is C—CF₃, S¹ is Cl, S² is Br, R^{1b} is CH₃, R^{1a} is CH₃, R³ is CH₃ and Q are as defined in table Z.

Table A-96 provides 8 compounds A-96.001 to A-96.008 of formula I-A wherein Ax is C—CF₃, S¹ is Cl, S² is Br, R^{1b} is CH₃, R^{1a} is CH₂-cyclopropyl, R³ is CH₃ and Q are as defined in table Z.

Table A-97 provides 8 compounds A-97.001 to A-97.008 of formula I-A wherein Ax is C—CF₃, S¹ is Cl, S² is Br, R^{1b} is CH₂-cyclopropyl, R^{1a} is H, R³ is CH₃ and Q are as defined in table Z.

Table A-98 provides 8 compounds A-98.001 to A-98.008 of formula I-A wherein Ax is C—CF₃, S¹ is Cl, S² is Br, R^{1b} is CH₂-cyclopropyl, R^{1a} is CH₃, R³ is CH₃ and Q are as defined in table Z.

Table A-99 provides 8 compounds A-99.001 to A-99.008 of formula I-A wherein Ax is C—CF₃, S¹ is Cl, S² is Br, R^{1b} is CH₂-cyclopropyl, R^{1a} is CH₂-cyclopropyl, R³ is CH₃ and Q are as defined in table Z.

Table A-100 provides 8 compounds A-100.001 to A-100.008 of formula I-A wherein Ax is C—CF₃, S¹ is Cl, S² is CF₃, R^{1b} is H, R^{1a} is H, R³ is CH₃ and Q are as defined in table Z.

Table A-101 provides 8 compounds A-101.001 to A-101.008 of formula I-A wherein Ax is C—CF₃, S¹ is Cl, S² is CF₃, R^{1b} is H, R₁ₐ is CH₃, R³ is CH₃ and Q are as defined in table Z.

Table A-102 provides 8 compounds A-102.001 to A-102.008 of formula I-A wherein Ax is C—CF₃, S¹ is Cl, S² is CF₃, R^{1b} is H, R₁ₐ is CH₂-cyclopropyl, R³ is CH₃ and Q are as defined in table Z.

Table A-103 provides 8 compounds A-103.001 to A-103.008 of formula I-A wherein Ax is C—CF₃, S¹ is Cl, S² is CF₃, R^{1b} is CH₃, R₁ₐ is H, R³ is CH₃ and Q are as defined in table Z.

Table A-104 provides 8 compounds A-104.001 to A-104.008 of formula I-A wherein Ax is C—CF₃, S¹ is Cl, S² is CF₃, R^{1b} is CH₃, R₁ₐ is CH₃, R³ is CH₃ and Q are as defined in table Z.

Table A-105 provides 8 compounds A-105.001 to A-105.008 of formula I-A wherein Ax is C—CF₃, S¹ is Cl, S² is CF₃, R^{1b} is CH₃, R₁ₐ is CH₂-cyclopropyl, R³ is CH₃ and Q are as defined in table Z.

Table A-106 provides 8 compounds A-106.001 to A-106.008 of formula I-A wherein Ax is C—CF₃, S¹ is Cl, S² is CF₃, R^{1b} is CH₂-cyclopropyl, R₁ₐ is H, R³ is CH₃ and Q are as defined in table Z.

Table A-107 provides 8 compounds A-107.001 to A-107.008 of formula I-A wherein Ax is C—CF₃, S¹ is Cl, S² is CF₃, R^{1b} is CH₂-cyclopropyl, R₁ₐ is CH₃, R³ is CH₃ and Q are as defined in table Z.

Table A-108 provides 8 compounds A-108.001 to A-108.008 of formula I-A wherein Ax is C—CF₃, S¹ is Cl, S² is CFs, R^{1b} is CH₂-cyclopropyl, R^{1a} is CH₂-cyclopropyl, R³ is CHs and Q are as defined in table Z.

Table A-109 provides 8 compounds A-109.001 to A-109.008 of formula I-A wherein Ax is C—CF₃, S¹ is Br, S² is Cl, R^{1b} is H, R^{1a} is H, R³ is CH₃ and Q are as defined in table Z.

Table A-110 provides 8 compounds A-110.001 to A-110.008 of formula I-A wherein Ax is C—CF₃, S¹ is Br, S² is Cl, R^{1b} is H, R^{1a} is CH₃, R³ is CH₃ and Q are as defined in table Z.

Table A-111 provides 8 compounds A-111.001 to A-111.008 of formula I-A wherein Ax is C—CF₃, S¹ is Br, S² is Cl, R^{1b} is H, R^{1a} is CH₂-cyclopropyl, R³ is CH₃ and Q are as defined in table Z.

Table A-112 provides 8 compounds A-112.001 to A-112.008 of formula I-A wherein Ax is C—CF₃, S¹ is Br, S² is Cl, R^{1b} is CH₃, R^{1a} is H, R³ is CH₃ and Q are as defined in table Z.

Table A-113 provides 8 compounds A-113.001 to A-113.008 of formula I-A wherein Ax is C—CF₃, S¹ is Br, S² is Cl, R^{1b} is CH₃, R₁ₐ is CH₃, R³ is CH₃ and Q are as defined in table Z.

Table A-114 provides 8 compounds A-114.001 to A-114.008 of formula I-A wherein Ax is C—CF₃, S¹ is Br, S² is Cl, R^{1b} is CH₃, R₁ₐ is CH₂-cyclopropyl, R³ is CH₃ and Q are as defined in table Z.

Table A-115 provides 8 compounds A-115.001 to A-115.008 of formula I-A wherein Ax is C—CF₃, S¹ is Br, S² is Cl, R^{1b} is CH₂-cyclopropyl, R₁ₐ is H, R³ is CH₃ and Q are as defined in table Z.

Table A-116 provides 8 compounds A-116.001 to A-116.008 of formula I-A wherein Ax is C—CF₃, S¹ is Br, S² is Cl, R^{1b} is CH₂-cyclopropyl, R₁ₐ is CH₃, R³ is CH₃ and Q are as defined in table Z.

Table A-117 provides 8 compounds A-117.001 to A-117.008 of formula I-A wherein Ax is C—CF₃, S¹ is Br, S² is Cl, R^{1b} is CH₂-cyclopropyl, R₁ₐ is CH₂-cyclopropyl, R³ is CH₃ and Q are as defined in table Z.

Table A-118 provides 8 compounds A-118.001 to A-118.008 of formula I-A wherein Ax is C—CF₃, S¹ is Br, S² is Br, R^{1b} is H, R₁ₐ is H, R³ is CH₃ and Q are as defined in table Z.

Table A-119 provides 8 compounds A-119.001 to A-119.008 of formula I-A wherein Ax is C—CF₃, S¹ is Br, S² is Br, R^{1b} is H, R₁ₐ is CH₃, R³ is CH₃ and Q are as defined in table Z.

Table A-120 provides 8 compounds A-120.001 to A-120.008 of formula I-A wherein Ax is C—CF₃, S¹ is Br, S² is Br, R^{1b} is H, R₁ₐ is CH₂-cyclopropyl, R³ is CH₃ and Q are as defined in table Z.

Table A-121 provides 8 compounds A-121.001 to A-121.008 of formula I-A wherein Ax is C—CF₃, S¹ is Br, S² is Br, R^{1b} is CH₃, R₁ₐ is H, R³ is CH₃ and Q are as defined in table Z.

Table A-122 provides 8 compounds A-122.001 to A-122.008 of formula I-A wherein Ax is C—CF₃, S¹ is Br, S² is Br, R^{1b} is CH₃, R₁ₐ is CH₃, R³ is CH₃ and Q are as defined in table Z.

Table A-123 provides 8 compounds A-123.001 to A-123.008 of formula I-A wherein Ax is C—CF₃, S¹ is Br, S² is Br, R^{1b} is CH₃, R₁ₐ is CH₂-cyclopropyl, R³ is CH₃ and Q are as defined in table Z.

Table A-124 provides 8 compounds A-124.001 to A-124.008 of formula I-A wherein Ax is C—CF₃, S¹ is Br, S² is Br, R^{1b} is CH₂-cyclopropyl, R^{1a} is H, R³ is CHs and Q are as defined in table Z.

Table A-125 provides 8 compounds A-125.001 to A-125.008 of formula I-A wherein Ax is C—CF₃, S¹ is Br, S² is Br, R^{1b} is CH₂-cyclopropyl, R^{1a} is CH₃, R³ is CH₃ and Q are as defined in table Z.

Table A-126 provides 8 compounds A-126.001 to A-126.008 of formula I-A wherein Ax is C—CF₃, S¹ is Br, S² is Br, R^{1b} is CH₂-cyclopropyl, R^{1a} is CH₂-cyclopropyl, R³ is CH₃ and Q are as defined in table Z.

Table A-127 provides 8 compounds A-127.001 to A-127.008 of formula I-A wherein Ax is C—CF₃, S¹ is Br, S² is CF₃, R^{1b} is H, R^{1a} is H, R³ is CH₃ and Q are as defined in table Z.

Table A-128 provides 8 compounds A-128.001 to A-128.008 of formula I-A wherein Ax is C—CF₃, S¹ is Br, S² is CF₃, R^{1b} is H, R^{1a} is CH₃, R³ is CH₃ and Q are as defined in table Z.

Table A-129 provides 8 compounds A-129.001 to A-129.008 of formula I-A wherein Ax is C—CF₃, S¹ is Br, S² is CF₃, R^{1b} is H, R₁ₐ is CH₂-cyclopropyl, R³ is CH₃ and Q are as defined in table Z.

Table A-130 provides 8 compounds A-130.001 to A-130.008 of formula I-A wherein Ax is C—CF₃, S¹ is Br, S² is CF₃, R^{1b} is CH₃, R₁ₐ is H, R³ is CH₃ and Q are as defined in table Z.

Table A-131 provides 8 compounds A-131.001 to A-131.008 of formula I-A wherein Ax is C—CF₃, S¹ is Br, S² is CF₃, R^{1b} is CH₃, R₁ₐ is CH₃, R³ is CH₃ and Q are as defined in table Z.

Table A-132 provides 8 compounds A-132.001 to A-132.008 of formula I-A wherein Ax is C—CF₃, S¹ is Br, S² is CF₃, R^{1b} is CH₃, R₁ₐ is CH₂-cyclopropyl, R³ is CH₃ and Q are as defined in table Z.

Table A-133 provides 8 compounds A-133.001 to A-133.008 of formula I-A wherein Ax is C—CF₃, S¹ is Br, S² is CF₃, R^{1b} is CH₂-cyclopropyl, R₁ₐ is H, R³ is CH₃ and Q are as defined in table Z.

Table A-134 provides 8 compounds A-134.001 to A-134.008 of formula I-A wherein Ax is C—CF₃, S¹ is Br, S² is CF₃, R^{1b} is CH₂-cyclopropyl, R₁ₐ is CH₃, R³ is CH₃ and Q are as defined in table Z.

Table A-135 provides 8 compounds A-135.001 to A-135.008 of formula I-A wherein Ax is C—CF₃, S¹ is Br, S² is CF₃, R^{1b} is CH₂-cyclopropyl, R₁ₐ is CH₂-cyclopropyl, R³ is CH₃ and Q are as defined in table Z.

Table A-136 provides 8 compounds A-136.001 to A-136.008 of formula I-A wherein Ax is C—CF₃, S¹ is CF₃, S² is Cl, R^{1b} is H, R₁ₐ is H, R³ is CH₃ and Q are as defined in table Z.

Table A-137 provides 8 compounds A-137.001 to A-137.008 of formula I-A wherein Ax is C—CF₃, S¹ is CF₃, S² is Cl, R^{1b} is H, R₁ₐ is CH₃, R³ is CH₃ and Q are as defined in table Z.

Table A-138 provides 8 compounds A-138.001 to A-138.008 of formula I-A wherein Ax is C—CF₃, S¹ is CF₃, S² is Cl, R^{1b} is H, R₁ₐ is CH₂-cyclopropyl, R³ is CH₃ and Q are as defined in table Z.

Table A-139 provides 8 compounds A-139.001 to A-139.008 of formula I-A wherein Ax is C—CF₃, S¹ is CF₃, S² is Cl, R^{1b} is CH₃, R₁ₐ is H, R³ is CH₃ and Q are as defined in table Z.

Table A-140 provides 8 compounds A-140.001 to A-140.008 of formula I-A wherein Ax is C—CF₃, S¹ is CFs, S² is Cl, R^{1b} is CHs, R^{1a} is CHs, R³ is CHs and Q are as defined in table Z.

Table A-141 provides 8 compounds A-141.001 to A-141.008 of formula I-A wherein Ax is C—CF₃, S¹ is CF₃, S² is Cl, R^{1b} is CH₃, R^{1a} is CH₂-cyclopropyl, R³ is CH₃ and Q are as defined in table Z.

Table A-142 provides 8 compounds A-142.001 to A-142.008 of formula I-A wherein Ax is C—CF₃, S¹ is CF₃, S² is Cl, R^{1b} is CH₂-cyclopropyl, R^{1a} is H, R³ is CH₃ and Q are as defined in table Z.

Table A-143 provides 8 compounds A-143.001 to A-143.008 of formula I-A wherein Ax is C—CF₃, S¹ is CF₃, S² is Cl, R^{1b} is CH₂-cyclopropyl, R^{1a} is CH₃, R³ is CH₃ and Q are as defined in table Z.

Table A-144 provides 8 compounds A-144.001 to A-144.008 of formula I-A wherein Ax is C—CF₃, S¹ is CF₃, S² is Cl, R^{1b} is CH₂-cyclopropyl, R^{1a} is CH₂-cyclopropyl, R³ is CH₃ and Q are as defined in table Z.

Table A-145 provides 8 compounds A-145.001 to A-145.008 of formula I-A wherein Ax is C—CF₃, S¹ is CF₃, S² is Br, R^{1b} is H, R^{1a} is H, R³ is CH₃ and Q are as defined in table Z.

Table A-146 provides 8 compounds A-146.001 to A-146.008 of formula I-A wherein Ax is C—CF₃, S¹ is CF₃, S² is Br, R^{1b} is H, R^{1a} is CH₃, R³ is CH₃ and Q are as defined in table Z.

Table A-147 provides 8 compounds A-147.001 to A-147.008 of formula I-A wherein Ax is C—CF₃, S¹ is CF₃, S² is Br, R^{1b} is H, R^{1a} is CH₂-cyclopropyl, R³ is CH₃ and Q are as defined in table Z.

Table A-148 provides 8 compounds A-148.001 to A-148.008 of formula I-A wherein Ax is C—CF₃, S¹ is CF₃, S² is Br, R^{1b} is CH₃, R^{1a} is H, R³ is CH₃ and Q are as defined in table Z.

Table A-149 provides 8 compounds A-149.001 to A-149.008 of formula I-A wherein Ax is C—CF₃, S¹ is CF₃, S² is Br, R^{1b} is CH₃, R₁ₐ is CH₃, R³ is CH₃ and Q are as defined in table Z.

Table A-150 provides 8 compounds A-150.001 to A-150.008 of formula I-A wherein Ax is C—CF₃, S¹ is CF₃, S² is Br, R^{1b} is CH₃, R₁ₐ is CH₂-cyclopropyl, R³ is CH₃ and Q are as defined in table Z.

Table A-151 provides 8 compounds A-151.001 to A-151.008 of formula I-A wherein Ax is C—CF₃, S¹ is CF₃, S² is Br, R^{1b} is CH₂-cyclopropyl, R₁ₐ is H, R³ is CH₃ and Q are as defined in table Z.

Table A-152 provides 8 compounds A-152.001 to A-152.008 of formula I-A wherein Ax is C—CF₃, S¹ is CF₃, S² is Br, R^{1b} is CH₂-cyclopropyl, R₁ₐ is CH₃, R³ is CH₃ and Q are as defined in table Z.

Table A-153 provides 8 compounds A-153.001 to A-153.008 of formula I-A wherein Ax is C—CF₃, S¹ is CF₃, S² is Br, R^{1b} is CH₂-cyclopropyl, R₁ₐ is CH₂-cyclopropyl, R³ is CH₃ and Q are as defined in table Z.

Table A-154 provides 8 compounds A-154.001 to A-154.008 of formula I-A wherein Ax is C—CF₃, S¹ is CF₃, S² is CF₃, R^{1b} is H, R₁ₐ is H, R³ is CH₃ and Q are as defined in table Z.

Table A-155 provides 8 compounds A-155.001 to A-155.008 of formula I-A wherein Ax is C—CF₃, S¹ is CF₃, S² is CF₃, R^{1b} is H, R₁ₐ is CH₃, R³ is CH₃ and Q are as defined in table Z.

Table A-156 provides 8 compounds A-156.001 to A-156.008 of formula I-A wherein Ax is C—CF₃, S¹ is CFs, S² is CFs, R^{1b} is H, R^{1a} is CH₂-cyclopropyl, R³ is CHs and Q are as defined in table Z.

Table A-157 provides 8 compounds A-157.001 to A-157.008 of formula I-A wherein Ax is C—CF₃, S¹ is CF₃, S² is CF₃, R^{1b} is CH₃, R^{1a} is H, R³ is CH₃ and Q are as defined in table Z.

Table A-158 provides 8 compounds A-158.001 to A-158.008 of formula I-A wherein Ax is C—CF₃, S¹ is CF₃, S² is CF₃, R^{1b} is CH₃, R^{1a} is CH₃, R³ is CH₃ and Q are as defined in table Z.

Table A-159 provides 8 compounds A-159.001 to A-159.008 of formula I-A wherein Ax is C—CF₃, S¹ is CF₃, S² is CF₃, R^{1b} is CH₃, R^{1a} is CH₂-cyclopropyl, R³ is CH₃ and Q are as defined in table Z.

Table A-160 provides 8 compounds A-160.001 to A-160.008 of formula I-A wherein Ax is C—CF₃, S¹ is CF₃, S² is CF₃, R^{1b} is CH₂-cyclopropyl, R^{1a} is H, R³ is CH₃ and Q are as defined in table Z.

Table A-161 provides 8 compounds A-161.001 to A-161.008 of formula I-A wherein Ax is C—CF₃, S¹ is CF₃, S² is CF₃, R^{1b} is CH₂-cyclopropyl, R^{1a} is CH₃, R³ is CH₃ and Q are as defined in table Z.

Table A-162 provides 8 compounds A-162.001 to A-162.008 of formula I-A wherein Ax is C—CF₃, S¹ is CF₃, S² is CF₃, R^{1b} is CH₂-cyclopropyl, R^{1a} is CH₂-cyclopropyl, R³ is CH₃ and Q are as defined in table Z.

Table A-163 provides 8 compounds A-163.001 to A-163.008 of formula I-A wherein Ax is N, S¹ is Cl, S² is Cl, R^{1b} is H, R^{1a} is H, R³ is CH₃ and Q are as defined in table Z.

Table A-164 provides 8 compounds A-164.001 to A-164.008 of formula I-A wherein Ax is N, S¹ is Cl, S² is Cl, R^{1b} is H, R^{1a} is CH₃, R³ is CH₃ and Q are as defined in table Z.

Table A-165 provides 8 compounds A-165.001 to A-165.008 of formula I-A wherein Ax is N, S¹ is Cl, S² is Cl, R^{1b} is H, R₁ₐ is CH₂-cyclopropyl, R³ is CH₃ and Q are as defined in table Z.

Table A-166 provides 8 compounds A-166.001 to A-166.008 of formula I-A wherein Ax is N, S¹ is Cl, S² is Cl, R^{1b} is CH₃, R₁ₐ is H, R³ is CH₃ and Q are as defined in table Z.

Table A-167 provides 8 compounds A-167.001 to A-167.008 of formula I-A wherein Ax is N, S¹ is Cl, S² is Cl, R^{1b} is CH₃, R₁ₐ is CH₃, R³ is CH₃ and Q are as defined in table Z.

Table A-168 provides 8 compounds A-168.001 to A-168.008 of formula I-A wherein Ax is N, S¹ is Cl, S² is Cl, R^{1b} is CH₃, R₁ₐ is CH₂-cyclopropyl, R³ is CH₃ and Q are as defined in table Z.

Table A-169 provides 8 compounds A-169.001 to A-169.008 of formula I-A wherein Ax is N, S¹ is Cl, S² is Cl, R^{1b} is CH₂-cyclopropyl, R₁ₐ is H, R³ is CH₃ and Q are as defined in table Z.

Table A-170 provides 8 compounds A-170.001 to A-170.008 of formula I-A wherein Ax is N, S¹ is Cl, S² is Cl, R^{1b} is CH₂-cyclopropyl, R₁ₐ is CH₃, R³ is CH₃ and Q are as defined in table Z.

Table A-171 provides 8 compounds A-171.001 to A-171.008 of formula I-A wherein Ax is N, S¹ is Cl, S² is Cl, R^{1b} is CH₂-cyclopropyl, R₁ₐ is CH₂-cyclopropyl, R³ is CH₃ and Q are as defined in table Z.

Table A-172 provides 8 compounds A-172.001 to A-172.008 of formula I-A wherein Ax is N, S¹ is Cl, S² is Br, R^{1b} is H, R^{1a} is H, R³ is CHs and Q are as defined in table Z.

Table A-173 provides 8 compounds A-173.001 to A-173.008 of formula I-A wherein Ax is N, S¹ is Cl, S² is Br, R^{1b} is H, R^{1a} is CH₃, R³ is CH₃ and Q are as defined in table Z.

Table A-174 provides 8 compounds A-174.001 to A-174.008 of formula I-A wherein Ax is N, S¹ is Cl, S² is Br, R^{1b} is H, R^{1a} is CH₂-cyclopropyl, R³ is CH₃ and Q are as defined in table Z.

Table A-175 provides 8 compounds A-175.001 to A-175.008 of formula I-A wherein Ax is N, S¹ is Cl, S² is Br, R^{1b} is CH₃, R^{1a} is H, R³ is CH₃ and Q are as defined in table Z.

Table A-176 provides 8 compounds A-176.001 to A-176.008 of formula I-A wherein Ax is N, S¹ is Cl, S² is Br, R^{1b} is CH₃, R^{1a} is CH₃, R³ is CH₃ and Q are as defined in table Z.

Table A-177 provides 8 compounds A-177.001 to A-177.008 of formula I-A wherein Ax is N, S¹ is Cl, S² is Br, R^{1b} is CH₃, R₁ₐ is CH₂-cyclopropyl, R³ is CH₃ and Q are as defined in table Z.

Table A-178 provides 8 compounds A-178.001 to A-178.008 of formula I-A wherein Ax is N, S¹ is Cl, S² is Br, R^{1b} is CH₂-cyclopropyl, R₁ₐ is H, R³ is CH₃ and Q are as defined in table Z.

Table A-179 provides 8 compounds A-179.001 to A-179.008 of formula I-A wherein Ax is N, S¹ is Cl, S² is Br, R^{1b} is CH₂-cyclopropyl, R₁ₐ is CH₃, R³ is CH₃ and Q are as defined in table Z.

Table A-180 provides 8 compounds A-180.001 to A-180.008 of formula I-A wherein Ax is N, S¹ is Cl, S² is Br, R^{1b} is CH₂-cyclopropyl, R₁ₐ is CH₂-cyclopropyl, R³ is CH₃ and Q are as defined in table Z.

Table A-181 provides 8 compounds A-181.001 to A-181.008 of formula I-A wherein Ax is N, S¹ is Cl, S² is CF₃, R^{1b} is H, R₁ₐ is H, R³ is CH₃ and Q are as defined in table Z.

Table A-182 provides 8 compounds A-182.001 to A-182.008 of formula I-A wherein Ax is N, S¹ is Cl, S² is CF₃, R^{1b} is H, R₁ₐ is CH₃, R³ is CH₃ and Q are as defined in table Z.

Table A-183 provides 8 compounds A-183.001 to A-183.008 of formula I-A wherein Ax is N, S¹ is Cl, S² is CF₃, R^{1b} is H, R₁ₐ is CH₂-cyclopropyl, R³ is CH₃ and Q are as defined in table Z.

Table A-184 provides 8 compounds A-184.001 to A-184.008 of formula I-A wherein Ax is N, S¹ is Cl, S² is CF₃, R^{1b} is CH₃, R₁ₐ is H, R³ is CH₃ and Q are as defined in table Z.

Table A-185 provides 8 compounds A-185.001 to A-185.008 of formula I-A wherein Ax is N, S¹ is Cl, S² is CF₃, R^{1b} is CH₃, R₁ₐ is CH₃, R³ is CH₃ and Q are as defined in table Z.

Table A-186 provides 8 compounds A-186.001 to A-186.008 of formula I-A wherein Ax is N, S¹ is Cl, S² is CF₃, R^{1b} is CH₃, R₁ₐ is CH₂-cyclopropyl, R³ is CH₃ and Q are as defined in table Z.

Table A-187 provides 8 compounds A-187.001 to A-187.008 of formula I-A wherein Ax is N, S¹ is Cl, S² is CF₃, R^{1b} is CH₂-cyclopropyl, R₁ₐ is H, R³ is CH₃ and Q are as defined in table Z.

Table A-188 provides 8 compounds A-188.001 to A-188.008 of formula I-A wherein Ax is N, S¹ is Cl, S² is CFs, R^{1b} is CH₂-cyclopropyl, R^{1a} is CHs, R³ is CHs and Q are as defined in table Z.

Table A-189 provides 8 compounds A-189.001 to A-189.008 of formula I-A wherein Ax is N, S¹ is Cl, S² is CF₃, R^{1b} is CH₂-cyclopropyl, R^{1a} is CH₂-cyclopropyl, R³ is CH₃ and Q are as defined in table Z.

Table A-190 provides 8 compounds A-190.001 to A-190.008 of formula I-A wherein Ax is N, S¹ is Br, S² is Cl, R^{1b} is H, R^{1a} is H, R³ is CH₃ and Q are as defined in table Z.

Table A-191 provides 8 compounds A-191.001 to A-191.008 of formula I-A wherein Ax is N, S¹ is Br, S² is Cl, R^{1b} is H, R^{1a} is CH₃, R³ is CH₃ and Q are as defined in table Z.

Table A-192 provides 8 compounds A-192.001 to A-192.008 of formula I-A wherein Ax is N, S¹ is Br, S² is Cl, R^{1b} is H, R^{1a} is CH₂-cyclopropyl, R³ is CH₃ and Q are as defined in table Z.

Table A-193 provides 8 compounds A-193.001 to A-193.008 of formula I-A wherein Ax is N, S¹ is Br, S² is Cl, R^{1b} is CH₃, R₁ₐ is H, R³ is CH₃ and Q are as defined in table Z.

Table A-194 provides 8 compounds A-194.001 to A-194.008 of formula I-A wherein Ax is N, S¹ is Br, S² is Cl, R^{1b} is CH₃, R₁ₐ is CH₃, R³ is CH₃ and Q are as defined in table Z.

Table A-195 provides 8 compounds A-195.001 to A-195.008 of formula I-A wherein Ax is N, S¹ is Br, S² is Cl, R^{1b} is CH₃, R₁ₐ is CH₂-cyclopropyl, R³ is CH₃ and Q are as defined in table Z.

Table A-196 provides 8 compounds A-196.001 to A-196.008 of formula I-A wherein Ax is N, S¹ is Br, S² is Cl, R^{1b} is CH₂-cyclopropyl, R₁ₐ is H, R³ is CH₃ and Q are as defined in table Z.

Table A-197 provides 8 compounds A-197.001 to A-197.008 of formula I-A wherein Ax is N, S¹ is Br, S² is Cl, R^{1b} is CH₂-cyclopropyl, R₁ₐ is CH₃, R³ is CH₃ and Q are as defined in table Z.

Table A-198 provides 8 compounds A-198.001 to A-198.008 of formula I-A wherein Ax is N, S¹ is Br, S² is Cl, R^{1b} is CH₂-cyclopropyl, R₁ₐ is CH₂-cyclopropyl, R³ is CH₃ and Q are as defined in table Z.

Table A-199 provides 8 compounds A-199.001 to A-199.008 of formula I-A wherein Ax is N, S¹ is Br, S² is Br, R^{1b} is H, R^{1a} is H, R³ is CH₃ and Q are as defined in table Z.

Table A-200 provides 8 compounds A-200.001 to A-200.008 of formula I-A wherein Ax is N, S¹ is Br, S² is Br, R^{1b} is H, R^{1a} is CH₃, R³ is CH₃ and Q are as defined in table Z.

Table A-201 provides 8 compounds A-201.001 to A-201.008 of formula I-A wherein Ax is N, S¹ is Br, S² is Br, R^{1b} is H, R₁ₐ is CH₂-cyclopropyl, R³ is CH₃ and Q are as defined in table Z.

Table A-202 provides 8 compounds A-202.001 to A-202.008 of formula I-A wherein Ax is N, S¹ is Br, S² is Br, R^{1b} is CH₃, R^{1a} is H, R³ is CH₃ and Q are as defined in table Z.

Table A-203 provides 8 compounds A-203.001 to A-203.008 of formula I-A wherein Ax is N, S¹ is Br, S² is Br, R^{1b} is CH₃, R₁ₐ is CH₃, R³ is CH₃ and Q are as defined in table Z.

Table A-204 provides 8 compounds A-204.001 to A-204.008 of formula I-A wherein Ax is N, S¹ is Br, S² is Br, R^{1b} is CHs, R^{1a} is CH₂-cyclopropyl, R³ is CHs and Q are as defined in table Z.

Table A-205 provides 8 compounds A-205.001 to A-205.008 of formula I-A wherein Ax is N, S¹ is Br, S² is Br, R^{1b} is CH₂-cyclopropyl, R^{1a} is H, R³ is CH₃ and Q are as defined in table Z.

Table A-206 provides 8 compounds A-206.001 to A-206.008 of formula I-A wherein Ax is N, S¹ is Br, S² is Br, R^{1b} is CH₂-cyclopropyl, R^{1a} is CH₃, R³ is CH₃ and Q are as defined in table Z.

Table A-207 provides 8 compounds A-207.001 to A-207.008 of formula I-A wherein Ax is N, S¹ is Br, S² is Br, R^{1b} is CH₂-cyclopropyl, R^{1a} is CH₂-cyclopropyl, R³ is CH₃ and Q are as defined in table Z.

Table A-208 provides 8 compounds A-208.001 to A-208.008 of formula I-A wherein Ax is N, S¹ is Br, S² is CF₃, R^{1b} is H, R^{1a} is H, R³ is CH₃ and Q are as defined in table Z.

Table A-209 provides 8 compounds A-209.001 to A-209.008 of formula I-A wherein Ax is N, S¹ is Br, S² is CF₃, R^{1b} is H, R₁ₐ is CH₃, R³ is CH₃ and Q are as defined in table Z.

Table A-210 provides 8 compounds A-210.001 to A-210.008 of formula I-A wherein Ax is N, S¹ is Br, S² is CF₃, R^{1b} is H, R₁ₐ is CH₂-cyclopropyl, R³ is CH₃ and Q are as defined in table Z.

Table A-211 provides 8 compounds A-211.001 to A-211.008 of formula I-A wherein Ax is N, S¹ is Br, S² is CF₃, R^{1b} is CH₃, R₁ₐ is H, R³ is CH₃ and Q are as defined in table Z.

Table A-212 provides 8 compounds A-212.001 to A-212.008 of formula I-A wherein Ax is N, S¹ is Br, S² is CF₃, R^{1b} is CH₃, R₁ₐ is CH₃, R³ is CH₃ and Q are as defined in table Z.

Table A-213 provides 8 compounds A-213.001 to A-213.008 of formula I-A wherein Ax is N, S¹ is Br, S² is CF₃, R^{1b} is CH₃, R₁ₐ is CH₂-cyclopropyl, R³ is CH₃ and Q are as defined in table Z.

Table A-214 provides 8 compounds A-214.001 to A-214.008 of formula I-A wherein Ax is N, S¹ is Br, S² is CF₃, R^{1b} is CH₂-cyclopropyl, R₁ₐ is H, R³ is CH₃ and Q are as defined in table Z.

Table A-215 provides 8 compounds A-215.001 to A-215.008 of formula I-A wherein Ax is N, S¹ is Br, S² is CF₃, R^{1b} is CH₂-cyclopropyl, R₁ₐ is CH₃, R³ is CH₃ and Q are as defined in table Z.

Table A-216 provides 8 compounds A-216.001 to A-216.008 of formula I-A wherein Ax is N, S¹ is Br, S² is CF₃, R^{1b} is CH₂-cyclopropyl, R₁ₐ is CH₂-cyclopropyl, R³ is CH₃ and Q are as defined in table Z.

Table A-217 provides 8 compounds A-217.001 to A-217.008 of formula I-A wherein Ax is N, S¹ is CF₃, S² is Cl, R^{1b} is H, R₁ₐ is H, R³ is CH₃ and Q are as defined in table Z.

Table A-218 provides 8 compounds A-218.001 to A-218.008 of formula I-A wherein Ax is N, S¹ is CF₃, S² is Cl, R^{1b} is H, R₁ₐ is CH₃, R³ is CH₃ and Q are as defined in table Z.

Table A-219 provides 8 compounds A-219.001 to A-219.008 of formula I-A wherein Ax is N, S¹ is CF₃, S² is Cl, R^{1b} is H, R₁ₐ is CH₂-cyclopropyl, R³ is CH₃ and Q are as defined in table Z.

Table A-220 provides 8 compounds A-220.001 to A-220.008 of formula I-A wherein Ax is N, S¹ is CF₃, S² is Cl, R^{1b} is CHs, R^{1a} is H, R³ is CHs and Q are as defined in table Z.

Table A-221 provides 8 compounds A-221.001 to A-221.008 of formula I-A wherein Ax is N, S¹ is CF₃, S² is Cl, R^{1b} is CH₃, R^{1a} is CH₃, R³ is CH₃ and Q are as defined in table Z.

Table A-222 provides 8 compounds A-222.001 to A-222.008 of formula I-A wherein Ax is N, S¹ is CF₃, S² is Cl, R^{1b} is CH₃, R^{1a} is CH₂-cyclopropyl, R³ is CH₃ and Q are as defined in table Z.

Table A-223 provides 8 compounds A-223.001 to A-223.008 of formula I-A wherein Ax is N, S¹ is CF₃, S² is Cl, R^{1b} is CH₂-cyclopropyl, R^{1a} is H, R³ is CH₃ and Q are as defined in table Z.

Table A-224 provides 8 compounds A-224.001 to A-224.008 of formula I-A wherein Ax is N, S¹ is CF₃, S² is Cl, R^{1b} is CH₂-cyclopropyl, R^{1a} is CH₃, R³ is CH₃ and Q are as defined in table Z.

Table A-225 provides 8 compounds A-225.001 to A-225.008 of formula I-A wherein Ax is N, S¹ is CF₃, S² is Cl, R^{1b} is CH₂-cyclopropyl, R₁ₐ is CH₂-cyclopropyl, R³ is CH₃ and Q are as defined in table Z.

Table A-226 provides 8 compounds A-226.001 to A-226.008 of formula I-A wherein Ax is N, S¹ is CF₃, S² is Br, R^{1b} is H, R^{1a} is H, R³ is CH₃ and Q are as defined in table Z.

Table A-227 provides 8 compounds A-227.001 to A-227.008 of formula I-A wherein Ax is N, S¹ is CF₃, S² is Br, R^{1b} is H, R₁ₐ is CH₃, R³ is CH₃ and Q are as defined in table Z.

Table A-228 provides 8 compounds A-228.001 to A-228.008 of formula I-A wherein Ax is N, S¹ is CF₃, S² is Br, R^{1b} is H, R₁ₐ is CH₂-cyclopropyl, R³ is CH₃ and Q are as defined in table Z.

Table A-229 provides 8 compounds A-229.001 to A-229.008 of formula I-A wherein Ax is N, S¹ is CF₃, S² is Br, R^{1b} is CH₃, R₁ₐ is H, R³ is CH₃ and Q are as defined in table Z.

Table A-230 provides 8 compounds A-230.001 to A-230.008 of formula I-A wherein Ax is N, S¹ is CF₃, S² is Br, R^{1b} is CH₃, R₁ₐ is CH₃, R³ is CH₃ and Q are as defined in table Z.

Table A-231 provides 8 compounds A-231.001 to A-231.008 of formula I-A wherein Ax is N, S¹ is CF₃, S² is Br, R^{1b} is CH₃, R₁ₐ is CH₂-cyclopropyl, R³ is CH₃ and Q are as defined in table Z.

Table A-232 provides 8 compounds A-232.001 to A-232.008 of formula I-A wherein Ax is N, S¹ is CF₃, S² is Br, R^{1b} is CH₂-cyclopropyl, R₁ₐ is H, R³ is CH₃ and Q are as defined in table Z.

Table A-233 provides 8 compounds A-233.001 to A-233.008 of formula I-A wherein Ax is N, S¹ is CF₃, S² is Br, R^{1b} is CH₂-cyclopropyl, R₁ₐ is CH₃, R³ is CH₃ and Q are as defined in table Z.

Table A-234 provides 8 compounds A-234.001 to A-234.008 of formula I-A wherein Ax is N, S¹ is CF₃, S² is Br, R^{1b} is CH₂-cyclopropyl, R₁ₐ is CH₂-cyclopropyl, R³ is CH₃ and Q are as defined in table Z.

Table A-235 provides 8 compounds A-235.001 to A-235.008 of formula I-A wherein Ax is N, S¹ is CF₃, S² is CF₃, R^{1b} is H, R₁ₐ is H, R³ is CH₃ and Q are as defined in table Z.

Table A-236 provides 8 compounds A-236.001 to A-236.008 of formula I-A wherein Ax is N, S¹ is CF₃, S² is CFs, R^{1b} is H, R^{1a} is CHs, R³ is CHs and Q are as defined in table Z. Table A-237 provides 8 compounds A-237.001 to A-237.008 of formula I-A wherein Ax is N, S¹ is CF₃, S² is CF₃, R^{1b} is H, R^{1a} is CH₂-cyclopropyl, R³ is CH₃ and Q are as defined in table Z.

Table A-238 provides 8 compounds A-238.001 to A-238.008 of formula I-A wherein Ax is N, S¹ is CF₃, S² is CF₃, R^{1b} is CH₃, R^{1a} is H, R³ is CH₃ and Q are as defined in table Z.

Table A-239 provides 8 compounds A-239.001 to A-239.008 of formula I-A wherein Ax is N, S¹ is CF₃, S² is CF₃, R^{1b} is CH₃, R^{1a} is CH₃, R³ is CH₃ and Q are as defined in table Z.

Table A-240 provides 8 compounds A-240.001 to A-240.008 of formula I-A wherein Ax is N, S¹ is CF₃, S² is CF₃, R^{1b} is CH₃, R^{1a} is CH₂-cyclopropyl, R³ is CH₃ and Q are as defined in table Z.

Table A-241 provides 8 compounds A-241.001 to A-241.008 of formula I-A wherein Ax is N, S¹ is CF₃, S² is CF₃, R^{1b} is CH₂-cyclopropyl, R^{1a} is H, R³ is CH₃ and Q are as defined in table Z.

Table A-242 provides 8 compounds A-242.001 to A-242.008 of formula I-A wherein Ax is N, S¹ is CF₃, S² is CF₃, R^{1b} is CH₂-cyclopropyl, R₁ₐ is CH₃, R³ is CH₃ and Q are as defined in table Z.

Table A-243 provides 8 compounds A-243.001 to A-243.008 of formula I-A wherein Ax is N, S¹ is CF₃, S² is CF₃, R^{1b} is CH₂-cyclopropyl, R^{1a} is CH₂-cyclopropyl, R³ is CH₃ and Q are as defined in table Z.

The compounds of formula I-B according to the following Tables B-1 to B-81 can be prepared according to the methods described above. The examples which follow are intended to illustrate the invention and show preferred compounds of formula I, in the form of a compound of formula I-B.

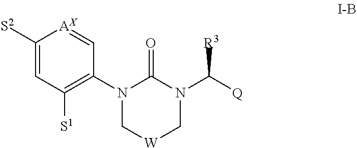

Table B-1 provides 8 compounds B-1.001 to B-1.008 of formula I-B wherein Ax is C—O—CF₂CF₂H, S¹ is Cl, S² is Cl, W is a direct bond, R³ is CH₃ and Q are as defined in table Z.

Table B-2 provides 8 compounds B-2.001 to B-2.008 of formula I-B wherein Ax is C—O—CF₂CF₂H, S¹ is Cl, S² is Cl, W is CH₂, R³ is CHs and Q are as defined in table Z. For example, B-2.002 and B-2.003 are:

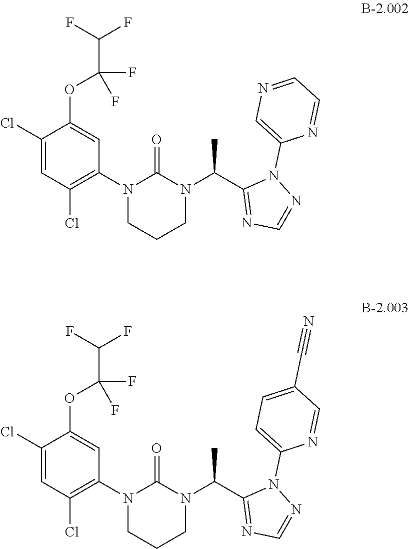 [SMILES: C[C@@H](c1ncnn1-c1cnccn1)N1CCCN(c2cc(OC(F)(F)C(F)F)c(Cl)cc2Cl)C1=O ; C[C@@H](c1ncnn1-c1ccc(C#N)cn1)N1CCCN(c2cc(OC(F)(F)C(F)F)c(Cl)cc2Cl)C1=O]

Table B-3 provides 8 compounds B-3.001 to B-3.008 of formula I-B wherein Ax is C—O—CF₂CF₂H, S¹ is Cl, S² is Cl, W is O, R³ is CH₃ and Q are as defined in table Z.

Table B-4 provides 8 compounds B-4.001 to B-4.008 of formula I-B wherein Ax is C—O—CF₂CF₂H, S¹ is Cl, S² is Br, W is a direct bond, R³ is CH₃ and Q are as defined in table Z.

Table B-5 provides 8 compounds B-5.001 to B-5.008 of formula I-B wherein Ax is C—O—CF₂CF₂H, S¹ is Cl, S² is Br, W is CH₂, R³ is CH₃ and Q are as defined in table Z.

Table B-6 provides 8 compounds B-6.001 to B-6.008 of formula I-B wherein Ax is C—O—CF₂CF₂H, S¹ is Cl, S² is Br, W is O, R³ is CH₃ and Q are as defined in table Z.

Table B-7 provides 8 compounds B-7.001 to B-7.008 of formula I-B wherein Ax is C—O—CF₂CF₂H, S¹ is Cl, S² is CF₃, W is a direct bond, R³ is CH₃ and Q are as defined in table Z.

Table B-8 provides 8 compounds B-8.001 to B-8.008 of formula I-B wherein Ax is C—O—CF₂CF₂H, S¹ is Cl, S² is CF₃, W is CH₂, R³ is CH₃ and Q are as defined in table Z.

Table B-9 provides 8 compounds B-9.001 to B-9.008 of formula I-B wherein Ax is C—O—CF₂CF₂H, S¹ is Cl, S² is CF₃, W is O, R³ is CH₃ and Q are as defined in table Z.

Table B-10 provides 8 compounds B-10.001 to B-10.008 of formula I-B wherein Ax is C—O—CF₂CF₂H, S¹ is Br, S² is Cl, W is a direct bond, R³ is CH₃ and Q are as defined in table Z.

Table B-11 provides 8 compounds B-11.001 to B-11.008 of formula I-B wherein Ax is C—O—CF₂CF₂H, S¹ is Br, S² is Cl, W is CH₂, R³ is CH₃ and Q are as defined in table Z.

Table B-12 provides 8 compounds B-12.001 to B-12.008 of formula I-B wherein Ax is C—O—CF₂CF₂H, S¹ is Br, S² is Cl, W is O, R³ is CH₃ and Q are as defined in table Z.

Table B-13 provides 8 compounds B-13.001 to B-13.008 of formula I-B wherein Ax is C—O—CF₂CF₂H, S¹ is Br, S² is Br, W is a direct bond, R³ is CH₃ and Q are as defined in table Z.

Table B-14 provides 8 compounds B-14.001 to B-14.008 of formula I-B wherein Ax is C—O—CF₂CF₂H, S¹ is Br, S² is Br, W is CH₂, R³ is CHs and Q are as defined in table Z.

Table B-15 provides 8 compounds B-15.001 to B-15.008 of formula I-B wherein Ax is C—O—CF₂CF₂H, S¹ is Br, S² is Br, W is O, R³ is CH₃ and Q are as defined in table Z.

Table B-16 provides 8 compounds B-16.001 to B-16.008 of formula I-B wherein Ax is C—O—CF₂CF₂H, S¹ is Br, S² is CF₃, W is a direct bond, R³ is CH₃ and Q are as defined in table Z.

Table B-17 provides 8 compounds B-17.001 to B-17.008 of formula I-B wherein Ax is C—O—CF₂CF₂H, S¹ is Br, S² is CF₃, W is CH₂, R³ is CH₃ and Q are as defined in table Z.

Table B-18 provides 8 compounds B-18.001 to B-18.008 of formula I-B wherein Ax is C—O—CF₂CF₂H, S¹ is Br, S² is CF₃, W is O, R³ is CH₃ and Q are as defined in table Z.

Table B-19 provides 8 compounds B-19.001 to B-19.008 of formula I-B wherein Ax is C—O—CF₂CF₂H, S¹ is CF₃, S² is Cl, W is a direct bond, R³ is CH₃ and Q are as defined in table Z.

Table B-20 provides 8 compounds B-20.001 to B-20.008 of formula I-B wherein Ax is C—O—CF₂CF₂H, S¹ is CF₃, S² is Cl, W is CH₂, R³ is CH₃ and Q are as defined in table Z.

Table B-21 provides 8 compounds B-21.001 to B-21.008 of formula I-B wherein Ax is C—O—CF₂CF₂H, S¹ is CF₃, S₂ is Cl, W is O, R³ is CH₃ and Q are as defined in table Z.

Table B-22 provides 8 compounds B-22.001 to B-22.008 of formula I-B wherein Ax is C—O—CF₂CF₂H, S¹ is CF₃, S² is Br, W is a direct bond, R³ is CH₃ and Q are as defined in table Z.

Table B-23 provides 8 compounds B-23.001 to B-23.008 of formula I-B wherein Ax is C—O—CF₂CF₂H, S¹ is CF₃, S² is Br, W is CH₂, R³ is CH₃ and Q are as defined in table Z.

Table B-24 provides 8 compounds B-24.001 to B-24.008 of formula I-B wherein Ax is C—O—CF₂CF₂H, S¹ is CF₃, S² is Br, W is O, R³ is CH₃ and Q are as defined in table Z.

Table B-25 provides 8 compounds B-25.001 to B-25.008 of formula I-B wherein Ax is C—O—CF₂CF₂H, S¹ is CF₃, S² is CF₃, W is a direct bond, R³ is CH₃ and Q are as defined in table Z.

Table B-26 provides 8 compounds B-26.001 to B-26.008 of formula I-B wherein Ax is C—O—CF₂CF₂H, S¹ is CF₃, S² is CF₃, W is CH₂, R³ is CH₃ and Q are as defined in table Z.

Table B-27 provides 8 compounds B-27.001 to B-27.008 of formula I-B wherein Ax is C—O—CF₂CF₂H, S¹ is CF₃, S² is CF₃, W is O, R³ is CH₃ and Q are as defined in table Z.

Table B-28 provides 8 compounds B-28.001 to B-28.008 of formula I-B wherein Ax is C—CF₃, S¹ is Cl, S² is Cl, W is a direct bond, R³ is CH₃ and Q are as defined in table Z.

Table B-29 provides 8 compounds B-29.001 to B-29.008 of formula I-B wherein Ax is C—CF₃, S¹ is Cl, S² is Cl, W is CH₂, R³ is CH₃ and Q are as defined in table Z.

Table B-30 provides 8 compounds B-30.001 to B-30.008 of formula I-B wherein Ax is C—CF₃, S¹ is Cl, S² is Cl, W is O, R³ is CHs and Q are as defined in table Z.

Table B-31 provides 8 compounds B-31.001 to B-31.008 of formula I-B wherein Ax is C—CF₃, S¹ is Cl, S² is Br, W is a direct bond, R³ is CH₃ and Q are as defined in table Z.

Table B-32 provides 8 compounds B-32.001 to B-32.008 of formula I-B wherein Ax is C—CF₃, S¹ is Cl, S² is Br, W is CH₂, R³ is CH₃ and Q are as defined in table Z.

Table B-33 provides 8 compounds B-33.001 to B-33.008 of formula I-B wherein Ax is C—CF₃, S¹ is Cl, S² is Br, W is O, R³ is CH₃ and Q are as defined in table Z.

Table B-34 provides 8 compounds B-34.001 to B-34.008 of formula I-B wherein Ax is C—CF₃, S¹ is Cl, S² is CF₃, W is a direct bond, R³ is CH₃ and Q are as defined in table Z.

Table B-35 provides 8 compounds B-35.001 to B-35.008 of formula I-B wherein Ax is C—CF₃, S¹ is Cl, S² is CF₃, W is CH₂, R³ is CH₃ and Q are as defined in table Z.

Table B-36 provides 8 compounds B-36.001 to B-36.008 of formula I-B wherein Ax is C—CF₃, S¹ is Cl, S² is CF₃, W is O, R³ is CH₃ and Q are as defined in table Z.

Table B-37 provides 8 compounds B-37.001 to B-37.008 of formula I-B wherein Ax is C—CF₃, S¹ is Br, S² is Cl, W is a direct bond, R³ is CH₃ and Q are as defined in table Z.

Table B-38 provides 8 compounds B-38.001 to B-38.008 of formula I-B wherein Ax is C—CF₃, S¹ is Br, S² is Cl, W is CH₂, R³ is CH₃ and Q are as defined in table Z.

Table B-39 provides 8 compounds B-39.001 to B-39.008 of formula I-B wherein Ax is C—CF₃, S¹ is Br, S² is Cl, W is O, R³ is CH₃ and Q are as defined in table Z.

Table B-40 provides 8 compounds B-40.001 to B-40.008 of formula I-B wherein Ax is C—CF₃, S¹ is Br, S² is Br, W is a direct bond, R³ is CH₃ and Q are as defined in table Z.

Table B-41 provides 8 compounds B-41.001 to B-41.008 of formula I-B wherein Ax is C—CF₃, S¹ is Br, S² is Br, W is CH₂, R³ is CH₃ and Q are as defined in table Z.

Table B-42 provides 8 compounds B-42.001 to B-42.008 of formula I-B wherein Ax is C—CF₃, S¹ is Br, S² is Br, W is O, R³ is CH₃ and Q are as defined in table Z.

Table B-43 provides 8 compounds B-43.001 to B-43.008 of formula I-B wherein Ax is C—CF₃, S¹ is Br, S² is CF₃, W is a direct bond, R³ is CH₃ and Q are as defined in table Z.

Table B-44 provides 8 compounds B-44.001 to B-44.008 of formula I-B wherein Ax is C—CF₃, S¹ is Br, S² is CF₃, W is CH₂, R³ is CH₃ and Q are as defined in table Z.

Table B-45 provides 8 compounds B-45.001 to B-45.008 of formula I-B wherein Ax is C—CF₃, S¹ is Br, S² is CF₃, W is O, R³ is CH₃ and Q are as defined in table Z.

Table B-46 provides 8 compounds B-46.001 to B-46.008 of formula I-B wherein Ax is C—CF₃, S¹ is CF₃, S² is Cl, W is a direct bond, R³ is CHs and Q are as defined in table Z.

Table B-47 provides 8 compounds B-47.001 to B-47.008 of formula I-B wherein Ax is C—CF₃, S¹ is CF₃, S² is Cl, W is CH₂, R³ is CH₃ and Q are as defined in table Z.

Table B-48 provides 8 compounds B-48.001 to B-48.008 of formula I-B wherein Ax is C—CF₃, S¹ is CF₃, S² is Cl, W is O, R³ is CH₃ and Q are as defined in table Z.

Table B-49 provides 8 compounds B-49.001 to B-49.008 of formula I-B wherein Ax is C—CF₃, S¹ is CF₃, S² is Br, W is a direct bond, R³ is CH₃ and Q are as defined in table Z.

Table B-50 provides 8 compounds B-50.001 to B-50.008 of formula I-B wherein Ax is C—CF₃, S¹ is CF₃, S² is Br, W is CH₂, R³ is CH₃ and Q are as defined in table Z.

Table B-51 provides 8 compounds B-51.001 to B-51.008 of formula I-B wherein Ax is C—CF₃, S¹ is CF₃, S² is Br, W is O, R³ is CH₃ and Q are as defined in table Z.

Table B-52 provides 8 compounds B-52.001 to B-52.008 of formula I-B wherein Ax is C—CF₃, S¹ is CF₃, S² is CF₃, W is a direct bond, R³ is CH₃ and Q are as defined in table Z.

Table B-53 provides 8 compounds B-53.001 to B-53.008 of formula I-B wherein Ax is C—CF₃, S¹ is CF₃, S² is CF₃, W is CH₂, R³ is CH₃ and Q are as defined in table Z.

Table B-54 provides 8 compounds B-54.001 to B-54.008 of formula I-B wherein Ax is C—CF₃, S¹ is CF₃, S² is CF₃, W is O, R³ is CH₃ and Q are as defined in table Z.

Table B-55 provides 8 compounds B-55.001 to B-55.008 of formula I-B wherein Ax is N, S¹ is Cl, S² is C₁, W is a direct bond, R³ is CH₃ and Q are as defined in table Z.

Table B-56 provides 8 compounds B-56.001 to B-56.008 of formula I-B wherein Ax is N, S¹ is Cl, S² is C₁, W is CH₂, R³ is CH₃ and Q are as defined in table Z.

Table B-57 provides 8 compounds B-57.001 to B-57.008 of formula I-B wherein Ax is N, S¹ is Cl, S² is C₁, W is O, R³ is CH₃ and Q are as defined in table Z.

Table B-58 provides 8 compounds B-58.001 to B-58.008 of formula I-B wherein Ax is N, S¹ is Cl, S² is Br, W is a direct bond, R³ is CH₃ and Q are as defined in table Z.

Table B-59 provides 8 compounds B-59.001 to B-59.008 of formula I-B wherein Ax is N, S¹ is Cl, S² is Br, W is CH₂, R³ is CH₃ and Q are as defined in table Z.

Table B-60 provides 8 compounds B-60.001 to B-60.008 of formula I-B wherein Ax is N, S¹ is Cl, S² is Br, W is O, R³ is CH₃ and Q are as defined in table Z.

Table B-61 provides 8 compounds B-61.001 to B-61.008 of formula I-B wherein Ax is N, S¹ is Cl, S² is CF₃, W is a direct bond, R³ is CH₃ and Q are as defined in table Z.

Table B-62 provides 8 compounds B-62.001 to B-62.008 of formula I-B wherein Ax is N, S¹ is Cl, S² is CFs, W is CH₂, R³ is CHs and Q are as defined in table Z.

Table B-63 provides 8 compounds B-63.001 to B-63.008 of formula I-B wherein Ax is N, S¹ is Cl, S² is CF₃, W is O, R³ is CH₃ and Q are as defined in table Z.

Table B-64 provides 8 compounds B-64.001 to B-64.008 of formula I-B wherein Ax is N, S¹ is Br, S² is C₁, W is a direct bond, R³ is CH₃ and Q are as defined in table Z.

Table B-65 provides 8 compounds B-65.001 to B-65.008 of formula I-B wherein Ax is N, S¹ is Br, S² is C₁, W is CH₂, R³ is CH₃ and Q are as defined in table Z.

Table B-66 provides 8 compounds B-66.001 to B-66.008 of formula I-B wherein Ax is N, S¹ is Br, S² is C₁, W is O, R³ is CH₃ and Q are as defined in table Z.

Table B-67 provides 8 compounds B-67.001 to B-67.008 of formula I-B wherein Ax is N, S¹ is Br, S² is Br, W is a direct bond, R³ is CH₃ and Q are as defined in table Z.

Table B-68 provides 8 compounds B-68.001 to B-68.008 of formula I-B wherein Ax is N, S¹ is Br, S² is Br, W is CH₂, R³ is CH₃ and Q are as defined in table Z.

Table B-69 provides 8 compounds B-69.001 to B-69.008 of formula I-B wherein Ax is N, S¹ is Br, S² is Br, W is O, R³ is CH₃ and Q are as defined in table Z.

Table B-70 provides 8 compounds B-70.001 to B-70.008 of formula I-B wherein Ax is N, S¹ is Br, S² is CF₃, W is a direct bond, R³ is CH₃ and Q are as defined in table Z.

Table B-71 provides 8 compounds B-71.001 to B-71.008 of formula I-B wherein Ax is N, S¹ is Br, S² is CF₃, W is CH₂, R³ is CH₃ and Q are as defined in table Z.

Table B-72 provides 8 compounds B-72.001 to B-72.008 of formula I-B wherein Ax is N, S¹ is Br, S² is CF₃, W is O, R³ is CH₃ and Q are as defined in table Z.

Table B-73 provides 8 compounds B-73.001 to B-73.008 of formula I-B wherein Ax is N, S¹ is CF₃, S² is C₁, W is a direct bond, R³ is CH₃ and Q are as defined in table Z.

Table B-74 provides 8 compounds B-74.001 to B-74.008 of formula I-B wherein Ax is N, S¹ is CF₃, S² is C₁, W is CH₂, R³ is CH₃ and Q are as defined in table Z.

Table B-75 provides 8 compounds B-75.001 to B-75.008 of formula I-B wherein Ax is N, S¹ is CF₃, S² is C₁, W is O, R³ is CH₃ and Q are as defined in table Z.

Table B-76 provides 8 compounds B-76.001 to B-76.008 of formula I-B wherein Ax is N, S¹ is CF₃, S² is Br, W is a direct bond, R³ is CH₃ and Q are as defined in table Z.

Table B-77 provides 8 compounds B-77.001 to B-77.008 of formula I-B wherein Ax is N, S¹ is CF₃, S² is Br, W is CH₂, R³ is CH₃ and Q are as defined in table Z.

Table B-78 provides 8 compounds B-78.001 to B-78.008 of formula I-B wherein Ax is N, S¹ is CF₃, S² is Br, W is O, R³ is CHs and Q are as defined in table Z.

Table B-79 provides 8 compounds B-79.001 to B-79.008 of formula I-B wherein AX is N, S¹ is CF₃, S² is CF₃, W is a direct bond, R³ is CH₃ and Q are as defined in table Z.

Table B-80 provides 8 compounds B-80.001 to B-80.008 of formula I-B wherein AX is N, S¹ is CF₃, S² is CF₃, W is CH₂, R³ is CH₃ and Q are as defined in table Z. Table B-81 provides 8 compounds B-81.001 to B-81.008 of formula I-B wherein AX is N, S¹ is CF₃, S² is CF₃, W is 0, R³ is CH₃ and Q are as defined in table Z.

In Tables A-i to A-243 and Tables B-i to B-8i, S¹ and S² do not represent a sulphur atom.

TABLE Z
Substituent definitions of Q:

| | |
|---|---|
| 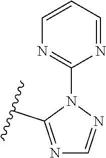 [SMILES: *c1ncnn1-c1ncccn1] | Q-1 |
| 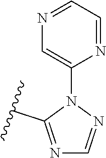 [SMILES: *c1ncnn1-c1cnccn1] | Q-2 |
| 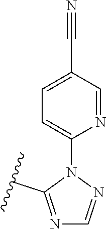 [SMILES: *c1ncnn1-c1ccc(C#N)cn1] | Q-3 |
| 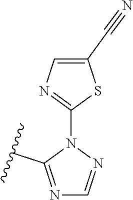 [SMILES: *c1ncnn1-c1ncc(C#N)s1] | Q-4 |
| 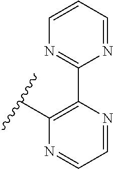 [SMILES: *c1nccnc1-c1ncccn1] | Q-5 |
| 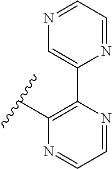 [SMILES: *c1nccnc1-c1cnccn1] | Q-6 |
| 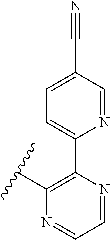 [SMILES: *c1nccnc1-c1ccc(C#N)cn1] | Q-7 |
| 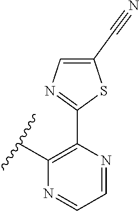 [SMILES: *c1nccnc1-c1ncc(C#N)s1] | Q-8 |

The compounds of formula I according to the following Table X can be prepared according to the methods described above. The examples which follow are intended to illustrate the invention and show preferred compounds of formula I.

TABLE X

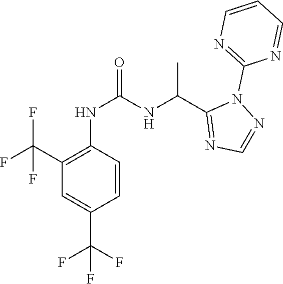 [SMILES: CC(NC(=O)Nc1ccc(C(F)(F)F)cc1C(F)(F)F)c1ncnn1-c1ncccn1]

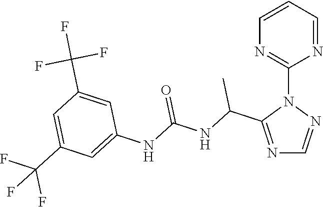 [SMILES: CC(NC(=O)Nc1cc(C(F)(F)F)cc(C(F)(F)F)c1)c1ncnn1-c1ncccn1]

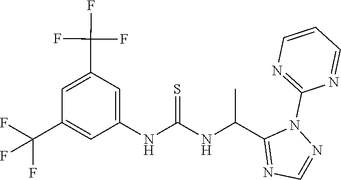 [SMILES: CC(NC(=S)Nc1cc(C(F)(F)F)cc(C(F)(F)F)c1)c1ncnn1-c1ncccn1]

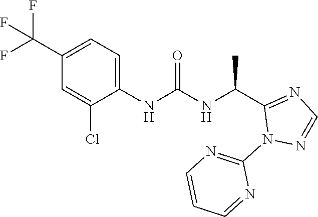 [SMILES: C[C@H](NC(=O)Nc1ccc(C(F)(F)F)cc1Cl)c1ncnn1-c1ncccn1]

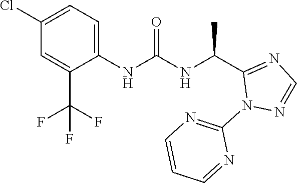 [SMILES: C[C@H](NC(=O)Nc1ccc(Cl)cc1C(F)(F)F)c1ncnn1-c1ncccn1]

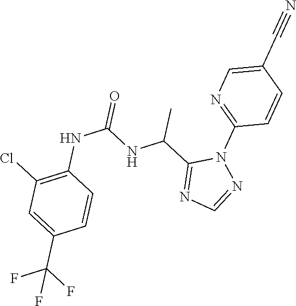 [SMILES: CC(NC(=O)Nc1ccc(C(F)(F)F)cc1Cl)c1ncnn1-c1ccc(C#N)cn1]

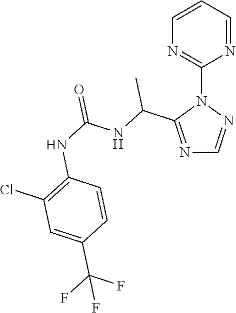 [SMILES: CC(NC(=O)Nc1ccc(C(F)(F)F)cc1Cl)c1ncnn1-c1ncccn1]

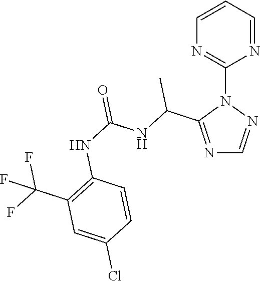 [SMILES: CC(NC(=O)Nc1ccc(Cl)cc1C(F)(F)F)c1ncnn1-c1ncccn1]

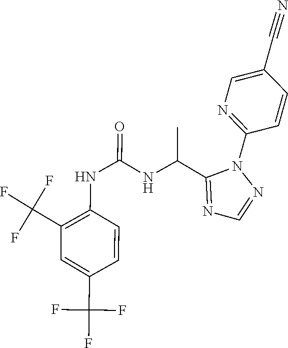 [SMILES: CC(NC(=O)Nc1ccc(C(F)(F)F)cc1C(F)(F)F)c1ncnn1-c1ccc(C#N)cn1]

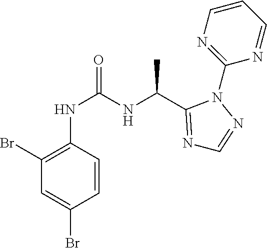 [SMILES: C[C@H](NC(=O)Nc1ccc(Br)cc1Br)c1ncnn1-c1ncccn1]

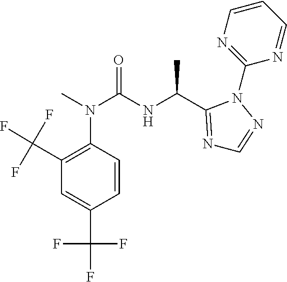 [SMILES: C[C@H](NC(=O)N(C)c1ccc(C(F)(F)F)cc1C(F)(F)F)c1ncnn1-c1ncccn1]

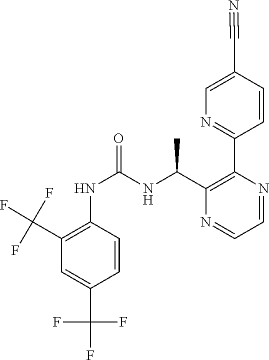 [SMILES: C[C@H](NC(=O)Nc1ccc(C(F)(F)F)cc1C(F)(F)F)c1nccnc1-c1ccc(C#N)cn1]

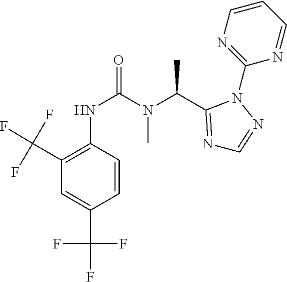 [SMILES: C[C@@H](c1ncnn1-c1ncccn1)N(C)C(=O)Nc1ccc(C(F)(F)F)cc1C(F)(F)F]

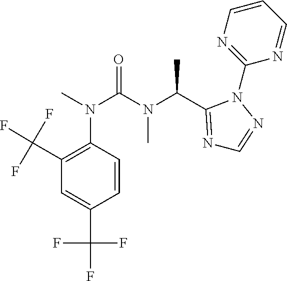 [SMILES: C[C@@H](c1ncnn1-c1ncccn1)N(C)C(=O)N(C)c1ccc(C(F)(F)F)cc1C(F)(F)F]

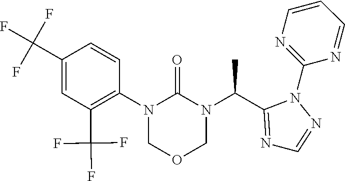 [SMILES: C[C@@H](c1ncnn1-c1ncccn1)N1COCN(c2ccc(C(F)(F)F)cc2C(F)(F)F)C1=O]

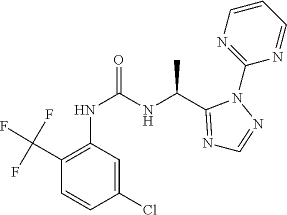 [SMILES: C[C@H](NC(=O)Nc1cc(Cl)ccc1C(F)(F)F)c1ncnn1-c1ncccn1]

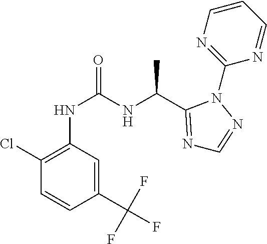 [SMILES: C[C@H](NC(=O)Nc1cc(C(F)(F)F)ccc1Cl)c1ncnn1-c1ncccn1]

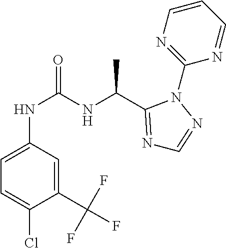 [SMILES: C[C@H](NC(=O)Nc1ccc(Cl)c(C(F)(F)F)c1)c1ncnn1-c1ncccn1]

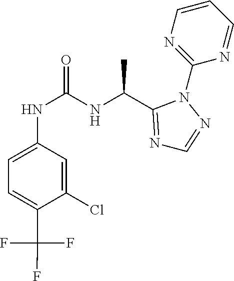 [SMILES: C[C@H](NC(=O)Nc1ccc(C(F)(F)F)c(Cl)c1)c1ncnn1-c1ncccn1]

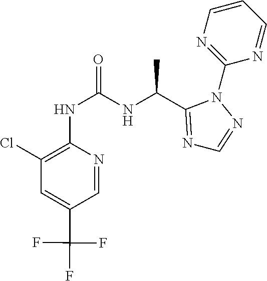 [SMILES: C[C@H](NC(=O)Nc1ncc(C(F)(F)F)cc1Cl)c1ncnn1-c1ncccn1]

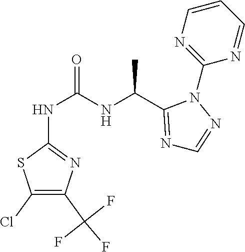 [SMILES: C[C@H](NC(=O)Nc1nc(C(F)(F)F)c(Cl)s1)c1ncnn1-c1ncccn1]

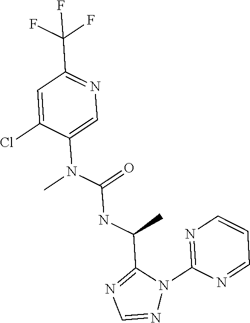 [SMILES: C[C@H](NC(=O)N(C)c1cnc(C(F)(F)F)cc1Cl)c1ncnn1-c1ncccn1]

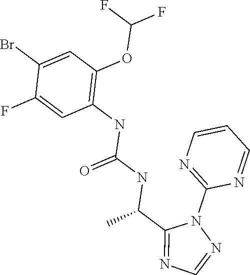 [SMILES: C[C@H](NC(=O)Nc1cc(F)c(Br)cc1OC(F)F)c1ncnn1-c1ncccn1]

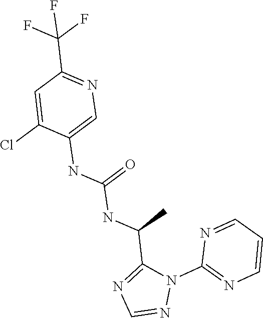 [SMILES: C[C@H](NC(=O)Nc1cnc(C(F)(F)F)cc1Cl)c1ncnn1-c1ncccn1]

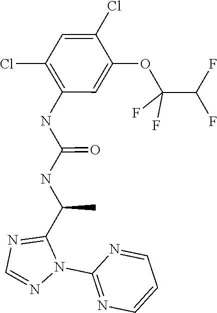 [SMILES: C[C@H](NC(=O)Nc1cc(OC(F)(F)C(F)F)c(Cl)cc1Cl)c1ncnn1-c1ncccn1]

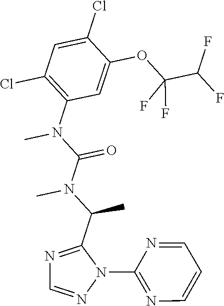 [SMILES: C[C@@H](c1ncnn1-c1ncccn1)N(C)C(=O)N(C)c1cc(OC(F)(F)C(F)F)c(Cl)cc1Cl]

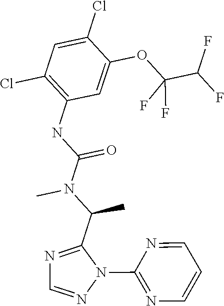 [SMILES: C[C@@H](c1ncnn1-c1ncccn1)N(C)C(=O)Nc1cc(OC(F)(F)C(F)F)c(Cl)cc1Cl]

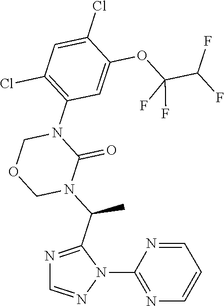 [SMILES: C[C@@H](c1ncnn1-c1ncccn1)N1COCN(c2cc(OC(F)(F)C(F)F)c(Cl)cc2Cl)C1=O]

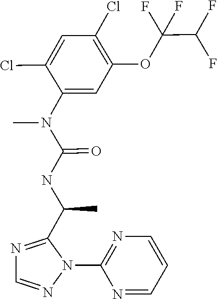 [SMILES: C[C@H](NC(=O)N(C)c1cc(OC(F)(F)C(F)F)c(Cl)cc1Cl)c1ncnn1-c1ncccn1]

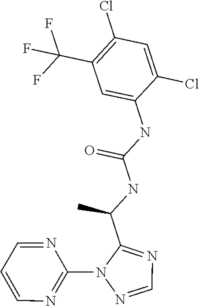 [SMILES: C[C@@H](NC(=O)Nc1cc(C(F)(F)F)c(Cl)cc1Cl)c1ncnn1-c1ncccn1]

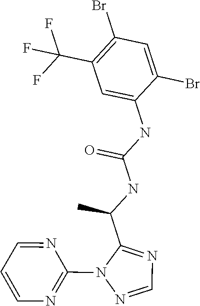 [SMILES: C[C@@H](NC(=O)Nc1cc(C(F)(F)F)c(Br)cc1Br)c1ncnn1-c1ncccn1]

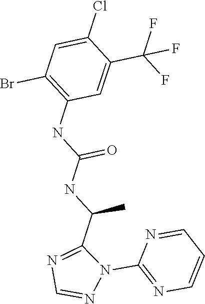 [SMILES: C[C@H](NC(=O)Nc1cc(C(F)(F)F)c(Cl)cc1Br)c1ncnn1-c1ncccn1]

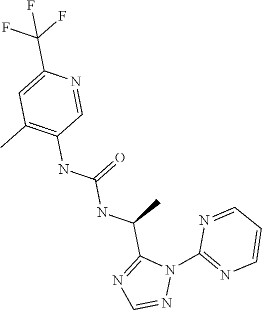 [SMILES: Cc1cc(C(F)(F)F)ncc1NC(=O)N[C@@H](C)c1ncnn1-c1ncccn1]

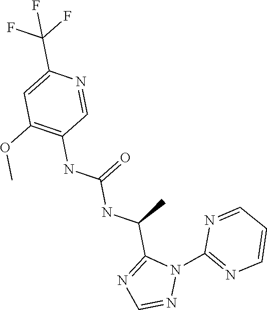 [SMILES: COc1cc(C(F)(F)F)ncc1NC(=O)N[C@@H](C)c1ncnn1-c1ncccn1]

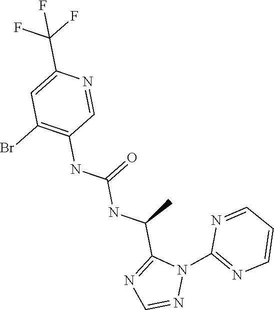 [SMILES: C[C@H](NC(=O)Nc1cnc(C(F)(F)F)cc1Br)c1ncnn1-c1ncccn1]

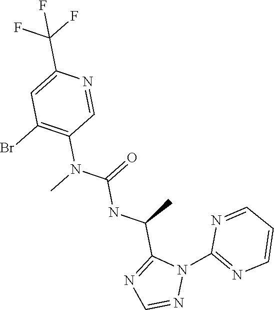 [SMILES: C[C@H](NC(=O)N(C)c1cnc(C(F)(F)F)cc1Br)c1ncnn1-c1ncccn1]

Also made available are certain intermediate compounds of formulae Ill, IV, VII, XIII, and XVI, some of which are novel.

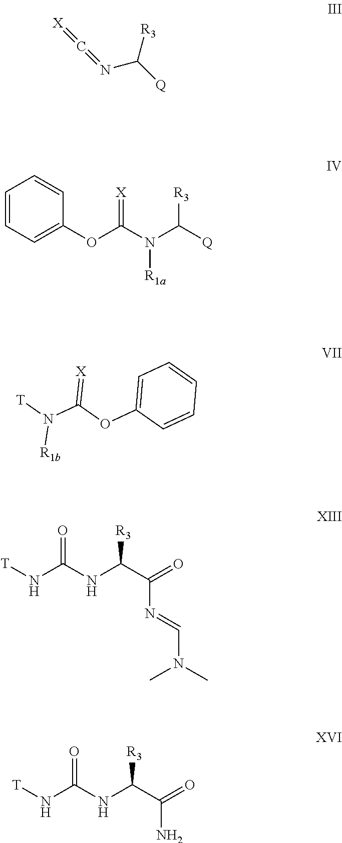

For example,
A compound of formula III, wherein Q is selected from Q^{aa} to Q^{ag} and Q^{ba} to Q^{bf}, R₃ is methyl and X is oxygen;
A compound of formula III, wherein Q is selected from Q^{aa} to Q^{ag} and Q^{ba} to Q^{bf}, R₃ is methyl and X is sulfur;
A compound of formula IV, wherein Q is selected from Q^{aa} to Q^{ag} and Q^{ba} to Q^{bf}, R₃ is methyl, R₁ₐ is hydrogen and X is oxygen;
A compound of formula IV, wherein Q is selected from Q^{aa} to Q^{ag} and Q^{ba} to Q^{bf}, R₃ is methyl, R₁ₐ is hydrogen and X is sulfur;
A compound of formula VII, wherein T is selected from 2,4-bis(trifluoromethyl)phenyl, 2,4-chloro-5-(1,1,2,2-tetrafluoroethoxy)phenyl, 2-bromo-4-chloro-5-(trifluoromethyl)phenyl, 4-bromo-2-(difluoromethoxy)-5-fluoro-phenyl, 2-bromo-4-(difluoromethoxy)phenyl, 2-iodo-4-(trifluoromethyl)phenyl, 3,5-bis(trifluoromethyl)phenyl, 2-chloro-4-(trifluoromethyl)phenyl, 4-chloro-2-(trifluoromethyl)phenyl, 2,4-dibromophenyl, 5-chloro-2-(trifluoromethyl)phenyl, 2-chloro-5-(trifluoromethyl)phenyl, 4-chloro-3-(trifluoromethyl)phenyl, 3-chloro-4-(trifluoromethyl)phenyl, 4-chloro-5-cyano-2-fluoro-phenyl, 2-methyl-4-(trifluoromethyl)phenyl, 4-chloro-6-(trifluoromethyl)-3-pyridyl, 4,6-dibromo-3-pyridyl, 4-bromo-6-(trifluoromethyl)-3-pyridyl, 4-methyl-6-(trifluoromethyl)-3-pyridyl, 4-methoxy-6-(trifluoromethyl)-3-pyridyl, 3-chloro-5-(trifluoromethyl)-2-pyridyl, 5-chloro-4-(trifluoromethyl)thiazol-2-yl, 4-methyl-6-(trifluoromethyl)-3-pyridyl, and 4-methoxy-6-(trifluoromethyl)-3-pyridyl, R_{1b} is hydrogen and X is oxygen;
A compound of formula VII, wherein T is selected from 2,4-bis(trifluoromethyl)phenyl, 2,4-chloro-5-(1,1,2,2-tetrafluoroethoxy)phenyl, 2-bromo-4-chloro-5-(trifluoromethyl)phenyl, 4-bromo-2-(difluoromethoxy)-5-fluoro-phenyl, 2-bromo-4-(difluoromethoxy)phenyl, 2-iodo-4-(trifluoromethyl)phenyl, 3,5-bis(trifluoromethyl)phenyl, 2-chloro-4-(trifluoromethyl)phenyl, 4-chloro-2-(trifluoromethyl)phenyl, 2,4-dibromophenyl, 5-chloro-2-(trifluoromethyl)phenyl, 2-chloro-5-(trifluoromethyl)phenyl, 4-chloro-3-(trifluoromethyl)phenyl, 3-chloro-4-(trifluoromethyl)phenyl, 4-chloro-5-cyano-2-fluoro-phenyl, 2-methyl-4-(trifluoromethyl)phenyl, 4-chloro-6-(trifluoromethyl)-3-pyridyl, 4,6-dibromo-3-pyridyl, 4-bromo-6-(trifluoromethyl)-3-pyridyl, 4-methyl-6-(trifluoromethyl)-3-pyridyl, 4-methoxy-6-(trifluoromethyl)-3-pyridyl, 3-chloro-5-(trifluoromethyl)-2-pyridyl, 5-chloro-4-(trifluoromethyl)thiazol-2-yl, 4-methyl-6-(trifluoromethyl)-3-pyridyl, and 4-methoxy-6-(trifluoromethyl)-3-pyridyl, R_{1b} is hydrogen and X is sulfur; and
A compound of formula XIII or of formula XVI, wherein R₃ is methyl and T is selected from 2,4-bis(trifluoromethyl)phenyl, 2,4-chloro-5-(1,1,2,2-tetrafluoroethoxy)phenyl, 2-bromo-4-chloro-5-(trifluoromethyl)phenyl, 4-bromo-2-(difluoromethoxy)-5-fluoro-phenyl, 2-bromo-4-(difluoromethoxy)phenyl, 2-iodo-4-(trifluoromethyl)phenyl, 3,5-bis(trifluoromethyl)phenyl, 2-chloro-4-(trifluoromethyl)phenyl, 4-chloro-2-(trifluoromethyl)phenyl, 2,4-dibromophenyl, 5-chloro-2-(trifluoromethyl)phenyl, 2-chloro-5-(trifluoromethyl)phenyl, 4-chloro-3-(trifluoromethyl)phenyl, 3-chloro-4-(trifluoromethyl)phenyl, 4-chloro-5-cyano-2-fluoro-phenyl, 2-methyl-4-(trifluoromethyl)phenyl, 4-chloro-6-(trifluoromethyl)-3-pyridyl, 4,6-dibromo-3-pyridyl, 4-bromo-6-(trifluoromethyl)-3-pyridyl, 4-methyl-6-(trifluoromethyl)-3-pyridyl, 4-methoxy-6-(trifluoromethyl)-3-pyridyl, 3-chloro-5-(trifluoromethyl)-2-pyridyl, 5-chloro-4-(trifluoromethyl)thiazol-2-yl, 4-methyl-6-(trifluoromethyl)-3-pyridyl, and 4-methoxy-6-(trifluoromethyl)-3-pyridyl.

In further aspect, the present invention accordingly makes available compounds of formulae III, IV, VII, XIII, and XVI, wherein in each case, as applicable, X is sulfur or oxygen, R₁ₐ, R_{1b}, R₃, Q and T are as defined for formula I in the first aspect. Furthermore, the corresponding embodiments illustrated for formula I also apply to the compounds of formulae III, IV, VII, XIII, and XVI.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the above-mentioned animal pests are:
from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,
Haematopinus spp., Linognathus spp., Pediculus spp., Pemphigus spp. and Phylloxera spp.; from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemlineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.; from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Aleurodes spp., Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euschistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Spp., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate from the order Lepidoptera, for example, Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Elasmopalpus lignosellus, Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,
Damalinea spp. and Trichodectes spp.;
from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,
Liposcelis spp.;
from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp; from the order Thysanura, for example, Lepisma saccharina.

In a further aspect, the invention may also relate to a method of controlling damage to plant and parts thereof by plant parasitic nematodes (endoparasitic, semiendoparasitic and ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The compounds of formula I are particularly suitable for control of
a pest of the order Hemiptera, for example, one or more of the species Bemisia tabaci, Aphis craccivora, Myzus persicae, Rhopalosiphum Padi, Nilaparvata lugens, and Euschistus heros (preferably in vegetables, soybeans, and sugarcane);
a pest of the order Lepidoptera, for example, one or more of the species Spodoptera littoralis, Spodoptera frugiperda, Plutella xylostella, Cnaphalocrocis medinalis, Cydia pomonella, Chrysodeixis includes, Chilo suppressalis, Elasmopalpus lignosellus, Pseudoplusia includens, and Tuta absoluta (preferably in vegetables and corn);
a pest of the order Thysanoptera, such as the family Thripidae, for example, one or more of Thrips tabaci and Frankliniella occidentalis (preferably in vegetables); and
soil pests (such as of the order Coleoptera), for example, the species Diabrotica balteata, Agriotes spp. and Leptinotarsa decemlineata (preferably in vegetables and corn).

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:
1. Bt11 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de I'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de I'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein CryF for achieving resistance to certain Lepidoptera insects and of the PAT protein for achieving tolerance to the herbicide glufosinate ammonium.
7. NK603 x MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603 x MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum for Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and storerooms and the protection of raw materials, such as wood, textiles, floor coverings or buildings, and also in the hygiene sector, especially the protection of humans, domestic animals and productive livestock against pests of the mentioned type.

The present invention provides a compound of the first aspect for use in therapy. The present invention provides a compound of the first aspect, for use in controlling parasites in or on an animal.

The present invention further provides a compound of the first aspect, for use in controlling ectoparasites on an animal. The present invention further provides a compound of the first aspect, for use in preventing and/or treating diseases transmitted by ectoparasites.

The present invention provides the use of a compound of the first aspect, for the manufacture of a medicament for controlling parasites in or on an animal. The present invention further provides the use of a compound of the first aspect, for the manufacture of a medicament for controlling ectoparasites on an animal. The present invention further provides the use of a compound of the first aspect, for the manufacture of a medicament for preventing and/or treating diseases transmitted by ectoparasites.

The present invention provides the use of a compound of the first aspect, in controlling parasites in or on an animal. The present invention further provides the use of a compound of the first aspect, in controlling ectoparasites on an animal.

The term “controlling” when used in context of parasites in or on an animal refers to reducing the number of pests or parasites, eliminating pests or parasites and/or preventing further pest or parasite infestation.

The term “treating” when used in context of parasites in or on an animal refers to restraining, slowing, stopping or reversing the progression or severity of an existing symptom or disease.

The term “preventing” when used in context of parasites in or on an animal refers to the avoidance of a symptom or disease developing in the animal.

The term “animal” when used in context of parasites in or on an animal may refer to a mammal and a non-mammal, such as a bird or fish. In the case of a mammal, it may be a human or non-human mammal. Non-human mammals include, but are not limited to, livestock animals and companion animals. Livestock animals include, but are not limited to, cattle, camellids, pigs, sheep, goats and horses. Companion animals include, but are not limited to, dogs, cats and rabbits.

A “parasite” is a pest which lives in or on the host animal and benefits by deriving nutrients at the host animal's expense. An “endoparasite” is a parasite which lives in the host animal. An “ectoparasite” is a parasite which lives on the host animal. Ectoparasites include, but are not limited to, acari, insects and crustaceans (e.g. sea lice). The Acari (or Acarina) sub-class comprises ticks and mites. Ticks include, but are not limited to, members of the following genera: Rhipicaphalus, for example, Rhipicaphalus (Boophilus) microplus and Rhipicephalus sanguineus; Amblyomrna; Dermacentor; Haemaphysalis; Hyalomma; Ixodes; Rhipicentor; Margaropus; Argas; Otobius; and Ornithodoros. Mites include, but are not limited to, members of the following genera: Chorioptes, for example Chorioptes bovis; Psoroptes, for example Psoroptes ovis; Cheyletiella; Dermanyssus; for example Dermanyssus gallinae; Ortnithonyssus; Demodex, for example Demodex canis; Sarcoptes, for example Sarcoptes scabiei; and Psorergates. Insects include, but are not limited to, members of the orders: Siphonaptera, Diptera, Phthiraptera, Lepidoptera, Coleoptera and Homoptera. Members of the Siphonaptera order include, but are not limited to, Ctenocephalides felis and Ctenocephatides canis. Members of the Diptera order include, but are not limited to, Musca spp.; bot fly, for example Gasterophilus intestinalis and Oestrus ovis; biting flies; horse flies, for example Haematopota spp. and Tabunus spp.; haematobia, for example haematobia irritans; Stomoxys; Lucilia; midges; and mosquitoes. Members of the Phthiraptera class include, but are not limited to, blood sucking lice and chewing lice, for example Bovicola Ovis and Bovicola Bovis.

The term “effective amount” when used in context of parasites in or on an animal refers to the amount or dose of the compound of the invention, or a salt thereof, which, upon single or multiple dose administration to the animal, provides the desired effect in or on the animal. The effective amount can be readily determined by the attending diagnostician, as one skilled in the art, by the use of known techniques and by observing results obtained under analogous circumstances. In determining the effective amount a number of factors are considered by the attending diagnostician, including, but not limited to: the species of mammal; its size, age, and general health; the parasite to be controlled and the degree of infestation; the specific disease or disorder involved; the degree of or involvement or the severity of the disease or disorder; the response of the individual; the particular compound administered; the mode of administration; the bioavailability characteristics of the preparation administered; the dose regimen selected; the use of concomitant medication; and other relevant circumstances.

The compounds of the invention may be administered to the animal by any route which has the desired effect including, but not limited to topically, orally, parenterally and subcutaneously. Topical administration is preferred. Formulations suitable for topical administration include, for example, solutions, emulsions and suspensions and may take the form of a pour-on, spot-on, spray-on, spray race or dip. In the alternative, the compounds of the invention may be administered by means of an ear tag or collar.

Salt forms of the compounds of the invention include both pharmaceutically acceptable salts and veterinary acceptable salts, which can be different to agrochemically acceptable salts.

Pharmaceutically and veterinary acceptable salts and common methodology for preparing them are well known in the art. See, for example, Gould, P. L., “Salt selection for basic drugs”, International Journal of Pharmaceutics, 33: 201-217 (1986); Bastin, R. J., et al. “Salt Selection and Optimization Procedures for Pharmaceutical New Chemical Entities”, Organic Process Research and Development, 4: 427-435 (2000); and Berge, S. M., et al., “Pharmaceutical Salts”, Journal of Pharmaceutical Sciences, 66: 1-19, (1977). One skilled in the art of synthesis will appreciate that the compounds of the invention are readily converted to and may be isolated as a salt, such as a hydrochloride salt, using techniques and conditions well known to one of ordinary skill in the art. In addition, one skilled in the art of synthesis will appreciate that the compounds of the invention are readily converted to and may be isolated as the corresponding free base from the corresponding salt.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE 1
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE 2
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern hophornbeam, |
| | | Dogwood, Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs, ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds of formulae I, and I′a, or salts thereof, are especially suitable for controlling one or more pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae. In a preferred embodiment of each aspect, a compound TX (where the abbreviation “TX” means “one compound selected from the compounds defined in Tables A-1 to A-243, Tables B-1 to B-81, Table X and Table P, preferably in Table X and Table P”) controls one or more of pests selected from the family: Noctuidae, Plutellidae, Chrysomelidae, Thripidae, Pentatomidae, Tortricidae, Delphacidae, Aphididae, Noctuidae, Crambidae, Meloidogynidae, and Heteroderidae.

The compounds of formulae I, and I′a, or salts thereof, are especially suitable for controlling one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp. In a preferred embodiment of each aspect, a compound TX (where the abbreviation “TX” means “one compound selected from the compounds defined in Table X and Table P”) controls one or more of pests selected from the genus: Spodoptera spp, Plutella spp, Frankliniella spp, Thrips spp, Euschistus spp, Cydia spp, Nilaparvata spp, Myzus spp, Aphis spp, Diabrotica spp, Rhopalosiphum spp, Pseudoplusia spp and Chilo spp.

The compounds of formulae I, and I′a, or salts thereof, are especially suitable for controlling one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum padi, and Chilo suppressalis.

In a preferred embodiment of each aspect, a compound TX (where the abbreviation “TX” means “one compound selected from the compounds defined in Tables A-1 to A-243, Tables B-1 to B-81, Table X and Table P, preferably from Table X and Table P”) controls one or more of Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis, such as Spodoptera littoralis+TX, Plutella xylostella+TX; Frankliniella occidentalis+TX, Thrips tabaci+TX, Euschistus heros+TX, Cydia pomonella+TX, Nilaparvata lugens+TX, Myzus persicae+TX, Chrysodeixis includens+TX, Aphis craccivora+TX, Diabrotica balteata+TX, Rhopalosiphum Padi+TX, and Chilo suppressalis+TX.

In an embodiment, of each aspect, one compound from Tables A-1 to A-243, Tables B-1 to B-81, Table X and Table P, preferably from Table X and Table P, is suitable for controlling Spodoptera littoralis, Plutella xylostella, Frankliniella occidentalis, Thrips tabaci, Euschistus heros, Cydia pomonella, Nilaparvata lugens, Myzus persicae, Chrysodeixis includens, Aphis craccivora, Diabrotica balteata, Rhopalosiphum Padia, and Chilo Suppressalis in cotton, vegetable, maize, cereal, rice and soya crops.

In an embodiment, one compound from Tables A-1 to A-243, Tables B-1 to B-81, Table X and Table P, preferably from Table X and Table P is suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile (against non-target organisms above and below ground (such as fish, birds and bees), improved physico-chemical properties, or increased biodegradability). In particular, it has been surprisingly found that certain compounds of formula I may show an advantageous safety profile with respect to non-target arthropods, in particular pollinators such as honey bees, solitary bees, and bumble bees. Most particularly, Apis mellifera.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%
   The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium | — | 6% | 10% |
| diisobutylnaphthalenesulfonate | | | |
| phenol polyethylene glycol | — | 2% | — |
| ether (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

Emulsifiable concentrate

| | |
|---|---|
| active ingredients | 10% |
| octylphenol polyethylene glycol ether (4-5 mol of ethylene oxide) | 3% |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Flowable concentrate for seed treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

The following examples further illustrate, but do not limit, the invention. Those skilled in the art will promptly recognize appropriate variations from the procedures both as to reactants and as to reaction conditions and techniques.

Throughout this description, temperatures are given in degrees Celsius (° C.). The following abbreviations are used: s=singlet; br s=broad singlet; d=doublet; br d=broad doublet; dd=double doublet; dt=double triplet; t=triplet, tt=triple triplet, q=quartet, quin=quintuplet, sept=septet; m=multiplet.

LC/MS apparatus and methods are:

### Method 1:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 41 V, Extractor: 2.00 V, Source Temperature: 150° C., Desolvation Temperature: 500° C., Cone Gas Flow: 50 l/h, Desolvation Gas Flow: 1000 l/h, Mass range: 110 to 800 Da) and an Acquity UPLC from Waters: Binary pump, heated column compartment, diode-array detector and ELSD detector. Column: Waters UPLC HSS T3, 1.8 μm, 30×2.1 mm, Temp: 40° C., PDA Wavelength range (nm): 200 to 400, Solvent Gradient: A=water+5% Acetonitrile+0.1% HCOOH, B=Acetonitrile+0.05% HCOOH, gradient: 10-100% B in 1.3 min; Flow (ml/min) 0.6.

### Method 2:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

### Method 3:

Spectra were recorded on a ACQUITY Mass Spectrometer from Waters Corporations (SQD or SQDII Single quadrupole mass spectrometer) equipped with an electrospray source (Polarity: positive or negative ions, Capillary: 3.0 kV, Cone: 30V, Extractor: 3.00 V, Source Temperature: 150° C., Desolvation Temperature: 400° C., Cone Gas Flow: 60 L/hr, Desolvation Gas Flow: 700 L/hr, Mass range: 140 to 800 Da) and an ACQUITY UPLC from Waters Corporations with solvent degasser, binary pump, heated column compartment and diode-array detector. Column: Waters UPLC HSS T3, 1.8 μm, 30 x 2.1 mm, Temp: 60° C., DAD Wavelength range (nm): 210 to 400, Solvent Gradient: A=Water/Methanol 9:1+0.1% formic acid, B=Acetonitrile+0.1% formic acid, gradient: 0-100% B in 2.5 min; Flow (ml/min) 0.75.

### Method 4:

Spectra were recorded on a 6410 Triple Quadruple Mass Spectrometer from Agilent Technologies; (Agilent 1200 Series HPLC) equipped with an electrospray source (Polarity: Positive and Negative Polarity Switch, Scan Type: MS2 Scan, Capillary (kV): 4.00, Fragmentor (V): 100.00, Gas Temperature (° C.): 350, Gas Flow (L/min): 11, Nebulizer Gas (psi): 45, Mass range: 110 to 1000 Da, DAD Wavelength range: 210 to 400 nm). Column: KINETEX EVO C18, Column length: 50 mm, Internal diameter of column: 4.6 mm, Particle Size: 2.6 p, Column oven temperature: 40° C., Optimized Chromatographic Parameter: Gradient conditions: Solvent A: Water with 0.1% formic acid:Acetonitrile: 95:5 v/v; Solvent B: Acetonitrile with 0.1% formic acid; gradient: 10-100% B in 2.5 min; Flow (ml/min) 1.8.

### Method 5:

Spectra were recorded on a Mass Spectrometer from Waters Corporation (QDA Single quadrupole mass spectrometer)), equipped with an electrospray source (Polarity: positive and negative ions), Detector Gain 1, Temperature Sample: 500° C., Cone Voltage: 10V, ESI Capillary Positive Voltage 0.8-Negative Voltage 0.8, Sampling Frequency 5 Hz, Mass range: 100 to 850 Da Source Temperature: 120° C., Desolvation Temperature: 600° C., Cone Gas Flow: 150 l/h, Desolvation Gas Flow: 1000 l/h, and an UPC2 from Waters Corporation: Binary solvent manager, heated column compartment, Sample manager, Isocratic solvent manager, convergence manager, diode-array detector, DAD Wavelength range (nm): 200 to 500, backpressure 1800 psi. Column: Daicel SFC CHIRALPAK® IC, 3 μm, 0.3 cm×10 cm, Temp: 40° C., Runtime: 4.8 min; Flow (ml/min) 2.0; Solvents: A=CO₂, B=Methanol+0.1% NH₄OH (30% in H2O); Gradient: 15% B isocratic for 4.8 min, then 100% A.

### Method 6:

Spectra were recorded on a Mass Spectrometer from Waters Corporation (QDA Single quadrupole mass spectrometer)), equipped with an electrospray source (Polarity: positive and negative ions), Detector Gain 1, Probe Temperature: 350° C., Cone Voltage: 10V, ESI Capillary Positive Voltage 0.8-Negative Voltage 0.8, Sampling Frequency 5 Hz, Mass range: 100 to 850 Da Source Temperature: 120° C., Desolvation Temperature: 600° C., Cone Gas Flow: 150 l/h, Desolvation Gas Flow: 1000 l/h, and an UPC2 from Waters Corporation: Binary solvent manager, heated column compartment, Sample manager, Isocratic solvent manager, convergence manager, diode-array detector, DAD Wavelength range (nm): 200 to 500, backpressure 1800 psi. Column: Waters Torus™ 1-AA column, 1.7 μm, 3 mm×100 mm, Temp: 40° C., Runtime: 4.8 min; Flow (ml/min) 2.0; Solvents: A=CO₂, B=Methanol; Gradient: 5% B isocratic for 4.8 min.

### Example P1: Preparation of 1-[2,4-bis(trifluoromethyl)phenyl]-3-[1-(2-pyrimidin-2-yl-1,2,4-triazol-3-yl)ethyl]urea (P1)

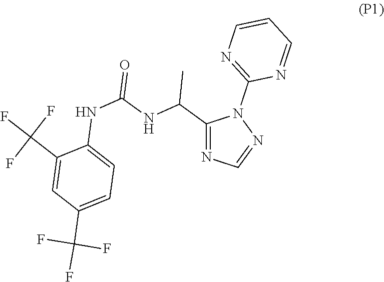 [SMILES: CC(NC(=O)Nc1ccc(C(F)(F)F)cc1C(F)(F)F)c1ncnn1-c1ncccn1]

### Step A: Preparation of 2-(2,2,2-trifluoroethylamino)-5-(trifluoromethyl)benzonitrile (1-1)

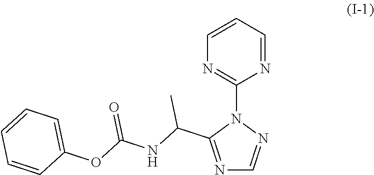 [SMILES: CC(NC(=O)Oc1ccccc1)c1ncnn1-c1ncccn1]

To a 2-neck RBF, equipped with a nitrogen inlet and condenser, charged 1-(2-pyrimidin-2-yl-1,2,4-triazol-3-yl)ethylammonium; 2,2,2-trifluoroacetate (1.5 g, 4.93 mmol) in acetonitrile (20 mL/g) then added Triethylamine (12.32 mmol, 1.26 g) at room temperature. Further phenyl chloroformate (7.39 mmol, 1.21 g) was added slowly. The reaction was heated and stirred at 80° C. for 2 hours under nitrogen atmosphere. Reaction mixture was cooled to room temperature, quenched with water, extracted with ethyl acetate twice. The combined organic layer was washed with brine, dried over anhydrous sodium sulfate and concentrated under reduced pressure to get brown gummy mass (1.96 g, crude). The crude was purified by silica gel column chromatography using 50% ethyl acetate in cyclohexane and concentrated under reduced pressure to give phenyl N-[1-(2-pyrimidin-2-yl-1,2,4-triazol-3-yl)ethyl]carbamate (1-1, 1.15 g) as white solid.

LC-MS (method 1): retention time 0.93 min, m/z 311.50 [M+H]⁺

¹H NMR (400 MHz, chloroform-d) δ ppm 8.84-8.92 (m, 2H) 7.99-8.12 (m, 1H) 7.30-7.41 (m, 3H) 7.10 (d, J=7.83 Hz, 1H) 6.96-7.21 (m, 2H) 6.33 (br d, J=8.56 Hz, 1H) 6.09-6.19 (m, 1H) 1.63-1.72 (m, 3H)

### Step B: Preparation of 1-[2,4-bis(trifluoromethyl)phenyl]-3-[1-(2-pyrimidin-2-yl-1,2,4-triazol-3-yl)ethyl]-urea (P1)

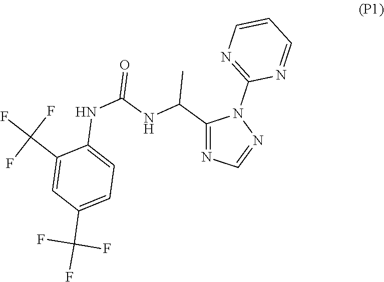 [SMILES: CC(NC(=O)Nc1ccc(C(F)(F)F)cc1C(F)(F)F)c1ncnn1-c1ncccn1]

In a clean and dry 25 ml RBF, charged phenyl N-[1-(2-pyrimidin-2-yl-1,2,4-triazol-3-yl)ethyl]carbamate (0.2 g, 0.644 mmol) and 2,4-bis(trifluoromethyl)aniline (3.223 mmol, 0.761 g) in triethylamine (20 mL/g, 28.7 mmol) at room temperature. Then reaction mass was heated and stirred at 120° C. for 2 h. The reaction was cooled to room temperature, solid precipitate out, diluted with pentane and filtered through Buchner funnel to crude which was purified by using C₁₈ column and acetonitrile and water as eluent. The desired compound was eluted at 25% MeCN:H₂O, which was lyophilized to give 1-[2,4-bis(trifluoromethyl)phenyl]-3-[1-(2-pyrimidin-2-yl-1,2,4-triazol-3-yl)ethyl]urea (180 mg) as white solid.

LC-MS (method 1): retention time 1.03 min, m/z 446.4 [M+H]⁺

¹H NMR (400 MHz, DMSO-d6) δ ppm 9.03 (d, 2H) 8.19-8.28 (m, 3H) 8.03-8.19 (m, 1H) 7.85-7.92 (m, 2H) 7.67 (t, 1H) 5.81 (t, 1H) 1.54 (d, 3H)

M.P.: 189-191° C.

Alternatively, 1-[2,4-bis(trifluoromethyl)phenyl]-3-[1-(2-pyrimidin-2-yl-1,2,4-triazol-3-yl)ethyl]-urea (P1) can be prepared according to the following procedure:

To a solution of triphosgene (0.026 g, 0.09 mmol) in ethyl acetate (2 mL) at 0° C. was added 2,4-bis(trifluoromethyl)aniline (0.05 g, 0.22 mmol) and triethyl amine (0.06 g, 0.55 mmol). The resulting suspension was stirred for 45 min at 0° C. and then a solution of 1-(2-pyrimidin-2-yl-1,2,4-triazol-3-yl)ethylammonium; 2,2,2-trifluoroacetate (0.07 g, 0.22 mmol) and triethyl amine (0.05 g, 0.45 mmol) in ethyl acetate (1 mL) was added. The resulting mixture was gradually warmed to room temperature and stirred for 18 h at this temperature. The reaction mixture was the partitioned between water and ethyl acetate. The organic layer was dried over Na₂SO₄, filtrated and concentrated under reduced pressure. The residue was purified as described in example P1, step B, to afford 1-[2,4-bis(trifluoromethyl)phenyl]-3-[1-(2-pyrimidin-2-yl-1,2,4-triazol-3-yl)ethyl]urea as white solid.

### Example P2: 1-[3,5-bis(trifluoromethyl)phenyl]-3-[1-(2-pyrimidin-2-yl-1,2,4-triazol-3-yl)ethyl]urea (P2)

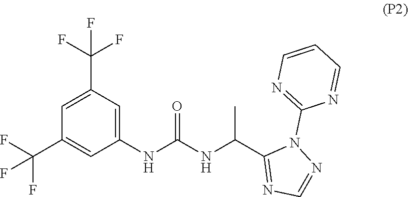 [SMILES: CC(NC(=O)Nc1cc(C(F)(F)F)cc(C(F)(F)F)c1)c1ncnn1-c1ncccn1]

1-(2-pyrimidin-2-yl-1,2,4-triazol-3-yl)ethanamine (200 mg, 1.05 mmol) was dissolved in tetrahydrofuran (3.5 mL/mmol) under Nitrogen at RT, then 1-isocyanato-3,5-bis(trifluoromethyl)benzene (1 equiv., 1.05 mmol) was added slowly followed by N,N-diethylethanamine (1 equiv., 1.0515 mmol) and stirred at RT for 16 h. The reaction mixture was diluted with water/brine and EtOAc. The aqueous layer was extracted twice with EtOAc. The combined organic layers were washed twice with water. The organic layer was dried, filtered and evaporated, to get 1-[3,5-bis(trifluoromethyl)phenyl]-3-[1-(2-pyrimidin-2-yl-1,2,4-triazol-3-yl) ethyl]urea (250 mg, 53%) as off white solid.

LC-MS (method 1): retention time 0.99 min, m/z 446.15 [M+H]⁺

¹H NMR (400 MHz, DMSO-d6) δ ppm 9.33 (s, 1H) 9.02 (d, 2H) 8.20 (s, 1H) 7.98 (s, 2H) 7.67 (t, 1H) 7.56 (s, 1H) 7.22 (d, 1H) 5.82 (s, 1H) 1.52 (d, 3H)

M.P.: 124-127° C.

### Example P3: 1-[3,5-bis(trifluoromethyl)phenyl]-3-[1-(2-pyrimidin-2-yl-1,2,4-triazol-3-yl)ethyl]thiourea (P3)

1-(2-pyrimidin-2-yl-1,2,4-triazol-3-yl)ethanamine (100 mg, 0.525 mmol) was dissolved in tetrahydrofuran (3.5 mL/mmol) under argon at RT, then 1-isothiocyanato-3,5-bis(trifluoromethyl)benzene (1 equiv., 0.525 mmol) was added slowly, followed by N,N-diethylethanamine (1 equiv., 0.525 mmol) and stirred at RT for 15 h. The reaction mixture was diluted with water/brine. The aqueous layer was extracted twice with EtOAc. The combined organic layers were washed twice with water. The organic layer was dried, filtered and evaporated to get 1-[3,5-bis(trifluoromethyl)phenyl]-3-[1-(2-pyrimidin-2-yl-1,2,4-triazol-3-yl)ethyl]thiourea (177 mg, 72%) as off white solid.

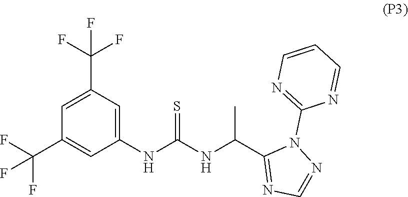 [SMILES: CC(NC(=S)Nc1cc(C(F)(F)F)cc(C(F)(F)F)c1)c1ncnn1-c1ncccn1]

LC-MS (method 1): retention time 1.02 min, m/z 460.16 [M−H]⁺

¹H NMR (400 MHz, DMSO-d6) δ ppm 10.15 (br s, 1H) 9.03 (d, 2H) 8.84-8.98 (m, 1H) 8.21 (d, 3H) 7.74 (s, 1H) 7.66 (t, 1H) 6.33 (br d, 1H) 1.62 (d, 3H)

### Example P103: 1-[4-chloro-6-(trifluoromethyl)-3-pyridyl]-3-[(1S)-1-(2-pyrimidin-2-yl-1,2,4-triazol-3-yl)ethyl]urea (P103)

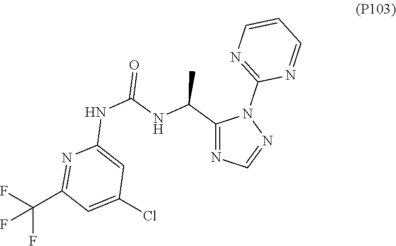 [SMILES: C[C@H](NC(=O)Nc1cc(Cl)cc(C(F)(F)F)n1)c1ncnn1-c1ncccn1]

Under argon atmosphere, to a solution of triphosgene (0.151 g, 0.509 mmol, 0.5 equiv.) in acetonitrile (5 mL) cooled at 0° C. was added 4-chloro-6-(trifluoromethyl)pyridin-3-amine (0.20 g, 1.017 mmol, 1.0 equiv.) in acetonitrile (5 mL) and triethylamine (0.358 mL, 2.54 mmol, 2.5 equiv.). The reaction was stirred at 0° C. for 45 minutes. Then, a solution of [(1S)-1-(2-pyrimidin-2-yl-1,2,4-triazol-3-yl)ethyl]ammonium; 2,2,2-trifluoroacetate (0.309 g, 1.017 mmol, 1.0 equiv.) and triethylamine (0.358 mL, 2.54 mmol, 2.5 equiv.) in acetonitrile (5 mL) was added to the reaction mixture at 0° C. and the solution was stirred at room temperature overnight. The reaction was quenched with water and the aqueous layer was extracted with ethyl acetate. The combined organic layers were washed with water, dried over sodium sulfate, filtered and concentrated under reduced pressure. The crude was precipitated in diisopropylether, the resulting off-white solid was filtered and washed three times with diisopropylether to afford the desired compound, 1-[4-chloro-6-(trifluoromethyl)-3-pyridyl]-3-[(1S)-1-(2-pyrimidin-2-yl-1,2,4-triazol-3-yl)ethyl]urea, as an off white solid.

LC-MS (method 2): retention time 0.82 min, m/z 413 [M+H⁺].

¹H NMR (400 MHz, chloroform-d) δ ppm 9.54 (s, 1H) 8.96 (d, J=4.72 Hz, 2H) 8.18 (s, 1H) 8.08 (brd, J=9.08 Hz, 1H) 7.42-7.49 (m, 3H) 6.30-6.38 (m, 1H) 1.15 (d, J=6.18 Hz, 3H).

¹⁹F NMR (377 MHz, chloroform-d) δ ppm −67.51 (s, 3F).

M.P.: 191-193° C.

### Example P47: Preparation of methyl (2S)-2-[[2,4-dichloro-5-(1,1,2,2-tetrafluoroethoxy)phenyl]carbamoylamino]propanoate (P134)

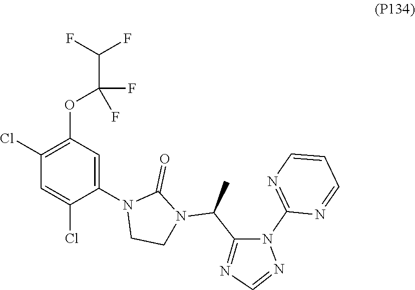 [SMILES: C[C@@H](c1ncnn1-c1ncccn1)N1CCN(c2cc(OC(F)(F)C(F)F)c(Cl)cc2Cl)C1=O]

### Step A: Preparation of 1,5-dichloro-2-nitro-4-(1,1,2,2-tetrafluoroethoxy)benzene (1-2)

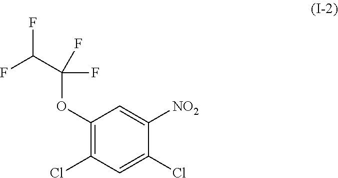 [SMILES: O=[N+]([O-])c1cc(OC(F)(F)C(F)F)c(Cl)cc1Cl]

Under argon atmosphere. to a solution of 2,4-dichloro-5-nitrophenol (5.0 g, 24 mmol, 1.0 equiv.) in N,N-dimethylformamide (50 mL) was added portion wise potassium carbonate (11.0 g, 82 mmol, 3.5 equiv.). The reaction mixture was heated to 70° C. and stirred overnight at this temperature. The solution was quenched with water and the aqueous layer was extracted twice with ethyl acetate. The combined organic layers were washed with water, dried over sodium sulfate, filtered and concentrated under reduced pressure. The crude was purified by flash chromatography (ethyl acetate in cyclohexane) to afford the desired compound, 1,5-dichloro-2-nitro-4-(1,1,2,2-tetrafluoroethoxy)benzene as an orange oil.

LC-MS (method 2): retention time 1.22 min.

¹H NMR (400 MHz, Chloroform-d) δ ppm 7.98 (s, 1H) 7.75 (s, 1H) 5.88-6.19 (m, 1H)

¹⁹F NMR (377 MHz, Chloroform-d) δ ppm −88.09 (s, 1F) −136.49 (s, 1F)

### Step B: Preparation of 2,4-dichloro-5-(1,1,2,2-tetrafluoroethoxy)aniline (1-3)

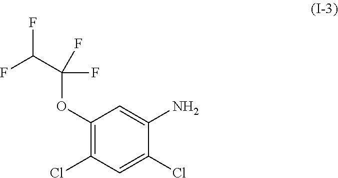 [SMILES: Nc1cc(OC(F)(F)C(F)F)c(Cl)cc1Cl]

Under argon atmosphere, to a solution of 1,5-dichloro-2-nitro-4-(1,1,2,2-tetrafluoroethoxy)benzene (1.53 g, 4.97 mmol, 1.0 equiv.) in methanol (20 mL) was added palladium (0.265 g, 0.249 mmol, 0.05 equiv.). The reaction mixture was let under hydrogen atmosphere (3bar) for 2 hours at room temperature. Then, the mixture was diluted with methanol, filtered under argon atmosphere through a pad of Hyflo. The filtrate was concentrated under reduced pressure and the crude was purified by flash chromatography (ethyl acetate in cyclohexane) to afford the desired compound, 2,4-dichloro-5-(1,1,2,2-tetrafluoroethoxy)aniline, as a yellow oil.

LC-MS (method 2): retention time 1.06 min, m/z 278 [M+H⁺].

¹H NMR (400 MHz, Chloroform-d) δ ppm 7.36 (s, 1H) 6.77-6.80 (m, 1H) 5.81-6.12 (m, 1H) 4.18 (br d, J=8.72 Hz, 2H).

¹⁹F NMR (377 MHz, chloroform-d) δ ppm −88.33 (s, 2F) −136.53 (s, 2F).

### Step C: Preparation of methyl (2S)-2-[[2,4-dichloro-5-(1,1,2,2-tetrafluoroethoxy)phenyl]carbamoylamino]propanoate (1-4)

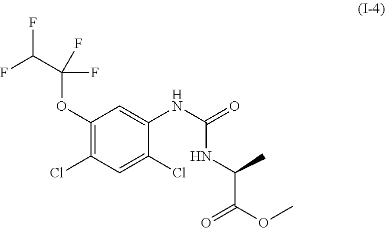 [SMILES: COC(=O)[C@H](C)NC(=O)Nc1cc(OC(F)(F)C(F)F)c(Cl)cc1Cl]

Under argon atmosphere, to a solution of triphosgene (0.133 g, 0.45 mmol, 0.5 equiv.) in acetonitrile (4 mL) cooled at 0° C. was added 2,4-dichloro-5-(1,1,2,2-tetrafluoroethoxy)aniline (0.25 g, 0.899 mmol, 1.0 equiv.) in acetonitrile (2 mL) and triethylamine (0.313 mL, 2.25 mmol, 2.5 equiv.). The reaction was stirred at 0° C. for 45 minutes. Then, a solution [(1S)-2-methoxy-1-methyl-2-oxo-ethyl]ammonium; chloride (0.126 g, 0.90 mmol, 1.0 equiv.) and triethylamine (0.314 mL, 2.25 mmol, 2.5 equiv.) in acetonitrile (3 mL) was added to the reaction mixture at 0° C. and the solution was stirred at room temperature overnight. The reaction was quenched with water and the aqueous layer was extracted with ethyl acetate. The combined organic layers were washed with water, dried over sodium sulfate, filtered and concentrated under reduced pressure. The crude was purified by flash chromatography (ethyl acetate in cyclohexane) to afford the desired compound, methyl (2S)-2-[[2,4-dichloro-5-(1,1,2,2-tetrafluoroethoxy)phenyl] carbamoylamino]propanoate, as a white solid.

LC-MS (method 2): retention time 1.04 min, m/z 407 [M+H⁺].

¹H NMR (400 MHz, Chloroform-d) δ ppm 8.38 (s, 1H) 7.42 (s, 1H) 6.96 (s, 1H) 5.82-6.11 (m, 1H) 5.74 (br d, J=7.63 Hz, 1H) 4.61 (quin, J=7.27 Hz, 1H) 3.83 (s, 3H) 1.49 (d, J=7.27 Hz, 3H).

¹⁹F NMR (377 MHz, Chloroform-d) δ ppm −88.32 (s, 2F) −136.56 (s, 2F).

### Step D: Preparation of methyl (2S)-2-[3-[2,4-dichloro-5-(1,1,2,2-tetrafluoroethoxy)phenyl]-2-oxo-imidazolidin-1-yl]propanoate (1-5)

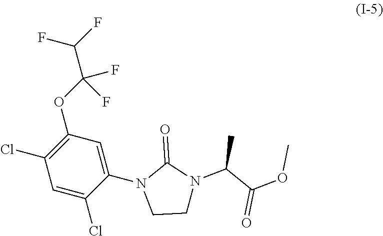 [SMILES: COC(=O)[C@H](C)N1CCN(c2cc(OC(F)(F)C(F)F)c(Cl)cc2Cl)C1=O]

To a solution of methyl (2S)-2-[[2,4-dichloro-5-(1,1,2,2-tetrafluoroethoxy)phenyl]carbamoylamino]propanoate (0.100 g, 0.246 mmol, 1.0 equiv.) and 1,2-dibromoethane (0.064 mL, 0.737 mmol, 3.0 equiv.) in tetrahydrofuran (1.23 mL) was slowly added sodium hydride (60 mass %, 0.025 g, 0.614 mmol, 2.5 equiv.) at 0° C. The reaction was stirred at 0° C. for 20 minutes, then it was allowed to warm to room temperature. The reaction was quenched with water and the aqueous layer was extracted twice with ethyl acetate. The combined organic layers were washed with water, dried over sodium sulfate, filtered and concentrated under reduced pressure. The crude was purified by flash chromatography (ethyl acetate/ethanol (3:1) in cyclohexane) to afford the desired compound, methyl (2S)-2-[3-[2,4-dichloro-5-(1,1,2,2-tetrafluoroethoxy)phenyl]-2-oxo-imidazolidin-1-yl]propanoate, as a beige solid.

LC-MS (method 2): retention time 1.05 min, m/z 433 [M+H⁺].

### Step E: Preparation of (2S)-2-[3-[2,4-dichloro-5-(1,1,2,2-tetrafluoroethoxy)phenyl]-2-oxo-imidazolidin-1-yl]propanamide (1-6)

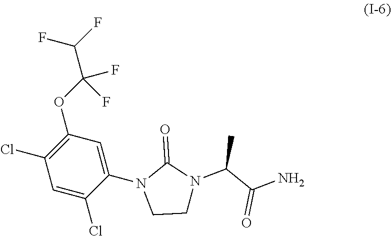 [SMILES: C[C@@H](C(N)=O)N1CCN(c2cc(OC(F)(F)C(F)F)c(Cl)cc2Cl)C1=O]

A solution of methyl (2S)-2-[3-[2,4-dichloro-5-(1,1,2,2-tetrafluoroethoxy)phenyl]-2-oxo-imidazolidin-1-yl]propanoate (0.102 g, 0.235 mmol, 1.0 equiv.) in ammonia (7M in methanol, 0.47 mL, 3.3 mmol, 14 equiv.) was heated in microwave at 50° C. overnight. The reaction was concentrated under reduced pressure to afford the desired compound, (2S)-2-[3-[2,4-dichloro-5-(1,1,2,2-tetrafluoroethoxy)phenyl]-2-oxo-imidazolidin-1-yl]propenamide, as an off-white solid.

LC-MS (method 2): retention time 0.91 min. m/z 418 [M+H⁺].

### Example P47: Preparation of methyl (2S)-2-[[2,4-dichloro-5-(1,1,2,2-tetrafluoroethoxy)phenyl]carbamoylamino]propanoate (P134)

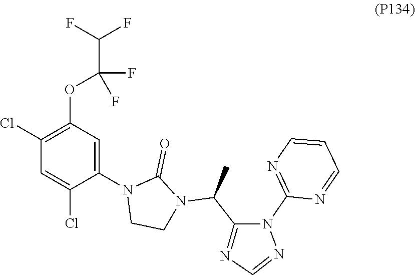 [SMILES: C[C@@H](c1ncnn1-c1ncccn1)N1CCN(c2cc(OC(F)(F)C(F)F)c(Cl)cc2Cl)C1=O]

Under argon atmosphere, to a solution of (2S)-2-[3-[2,4-dichloro-5-(1,1,2,2-tetrafluoroethoxy)phenyl]-2-oxo-imidazolidin-1-yl]propenamide (preparation described in example 3 step B) (0.103 g, 0.246 mmol, 1.0 equiv.) in dichloromethane (2.46 mL) was added N,N-dimethylformamide dimethyl acetal (0.066 mL, 0.493 mmol, 2.0 equiv.). The reaction mixture was stirred at 50° C. for 30 minutes, then concentrated under reduced pressure. The residue was dissolved in dioxane (1 mL) and pyrimidin-2-ylhydrazine (0.054 g, 0.495 mmol, 2.0 equiv.) and acetic acid (0.618 mL, 10.8 mmol, 43.6 equiv.) were added to the solution. The reaction mixture was stirred at 80° C. for 1 hour. The reaction mixture was quenched with sodium bicarbonate. The aqueous layer was extracted twice with ethyl acetate, the combined organic layers were washed twice with water, dried over sodium sulfate, filtered and concentrated under reduced pressure. The crude was purified by flash chromatography (ethyl acetate in cyclohexane) to afford the desired compound, 1-[2,4-dichloro-5-(1,1,2,2-tetrafluoroethoxy)phenyl]-3-[(1S)-1-(2-pyrimidin-2-yl-1,2,4-triazol-3-yl)ethyl]imidazolidin-2-one, as a white solid.

LC-MS (method 1): retention time 0.98 min, m/z 520 [M+H⁺].

¹H NMR (400 MHz, Chloroform-d) δ ppm 8.48-8.58 (m, 2H) 7.89-7.98 (m, 1H) 7.55-7.61 (m, 1H) 6.84-6.92 (m, 1H) 5.83-6.18 (m, 2H) 3.46-3.55 (m, 1H) 1.89-1.99 (m, 3H) 1.54-1.57 (m, 3H) 1.20-1.27 (m, 1H)

M.P.: 207-209° C.

TABLE P
Examples of compounds of formula I:

| | | | Rt | [M + H] | | MP |
|---|---|---|---|---|---|---|
| Entry | IUPAC name | STRUCTURE | (min) | (measured) | Method | ° C. |
| P1 | 1-[2,4-bis(trifluoromethyl)phenyl]-3- [1-(2-pyrimidin-2-yl-1,2,4-triazol-3- yl)ethyl]urea | 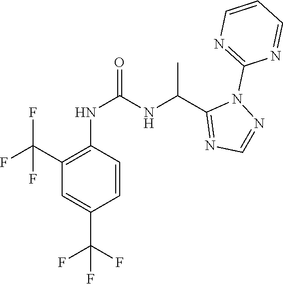 [SMILES: CC(NC(=O)Nc1ccc(C(F)(F)F)cc1C(F)(F)F)c1ncnn1-c1ncccn1] | 1.03 | 446.4 | 1 | 189 - 191 |
| P2 | 1-[3,5-bis(trifluoromethyl)phenyl]-3- [1-(2-pyrimidin-2-yl-1,2,4-triazol-3- yl)ethyl]urea | 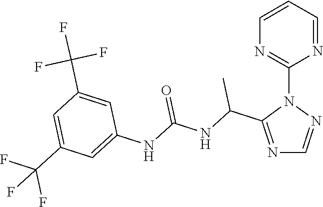 [SMILES: CC(NC(=O)Nc1cc(C(F)(F)F)cc(C(F)(F)F)c1)c1ncnn1-c1ncccn1] | 0.99 | 446.15 | 1 | 124 - 127 |
| P3 | 1-[3,5-bis(trifluoromethyl)phenyl]-3- [1-(2-pyrimidin-2-yl-1,2,4-triazol-3- yl)ethyl]thiourea | 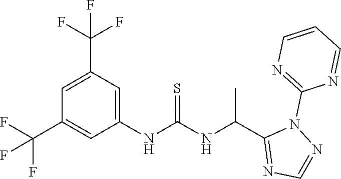 [SMILES: CC(NC(=S)Nc1cc(C(F)(F)F)cc(C(F)(F)F)c1)c1ncnn1-c1ncccn1] | 1.02 | 460.16 | 1 | 213 - 215 |
| P4 | 1-[2-chloro-4- (trifluoromethyl)phenyl]-3-[1-[2-(5- cyano-2-pyridyl)-1,2,4-triazol-3- yl]ethyl]urea | 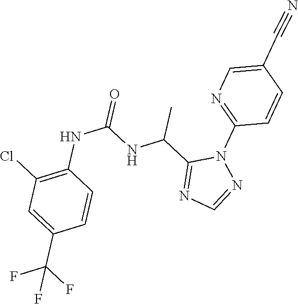 [SMILES: CC(NC(=O)Nc1ccc(C(F)(F)F)cc1Cl)c1ncnn1-c1ccc(C#N)cn1] | 1.02 | 436.0 | 2 | 204 - 206 |
| P5 | 1-[2-chloro-4- (trifluoromethyl)phenyl]-3-[1-(2- pyrimidin-2-yl-1,2,4-triazol-3- yl)ethyl]urea | 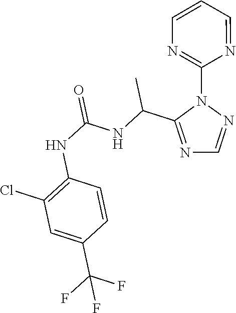 [SMILES: CC(NC(=O)Nc1ccc(C(F)(F)F)cc1Cl)c1ncnn1-c1ncccn1] | 0.91 | 412.0 | 2 | 196 - 197 |
| P6 | 1-[4-chloro-2- (trifluoromethyl)phenyl]-3-[1-(2- pyrimidin-2-yl-1,2,4-triazol-3- yl)ethyl]urea | 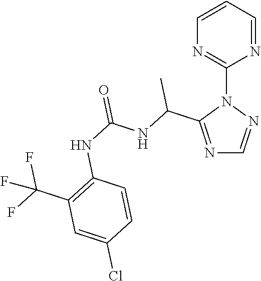 [SMILES: CC(NC(=O)Nc1ccc(Cl)cc1C(F)(F)F)c1ncnn1-c1ncccn1] | 0.87 | 412.0 | 2 | 206 - 208 |
| P7 | 1-[4-chloro-3- (trifluoromethyl)phenyl]-3-[(1S)-1-[3- (5-cyano-2-pyridyl)pyrazin-2- yl]ethyl]urea | 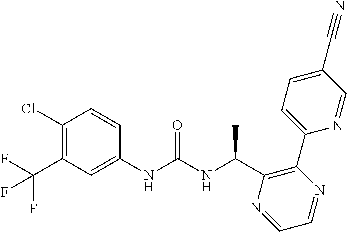 [SMILES: C[C@H](NC(=O)Nc1ccc(Cl)c(C(F)(F)F)c1)c1nccnc1-c1ccc(C#N)cn1] | 1.55 | 447.05 | 1 | 179 - 181 |
| P8 | 1-(3,4-dibromophenyl)-3-[(1S)-1-(3- pyrimidin-2-ylpyrazin-2-yl)ethyl]urea | 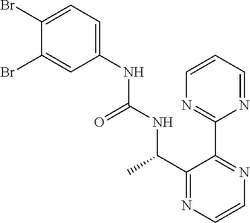 [SMILES: C[C@H](NC(=O)Nc1ccc(Br)c(Br)c1)c1nccnc1-c1ncccn1] | 0.99 | 477.2 | 1 | 220 - 222 |
| P9 | 1-[3-chloro-4- (trifluoromethyl)phenyl]-3-[(1S)-1-[3- (5-cyano-2-pyridyl)pyrazin-2- yl]ethyl]urea | 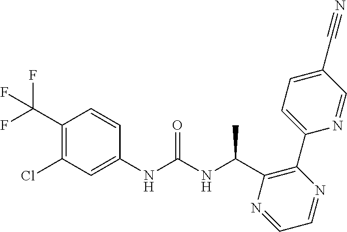 [SMILES: C[C@H](NC(=O)Nc1ccc(C(F)(F)F)c(Cl)c1)c1nccnc1-c1ccc(C#N)cn1] | 1.08 | 447.3 | 1 | 182 - 184 |
| P10 | 1-[3-chloro-4- (trifluoromethyl)phenyl]-3-[(1S)-1- (3-pyrimidin-2-ylpyrazin-2- yl)ethyl]urea | 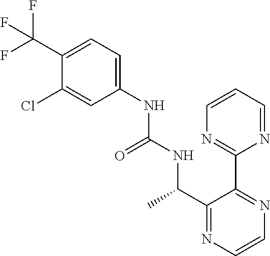 [SMILES: C[C@H](NC(=O)Nc1ccc(C(F)(F)F)c(Cl)c1)c1nccnc1-c1ncccn1] | 1 | 423.3 | 1 | 130 - 132 |
| P11 | 1-[4-chloro-3- (trifluoromethyl)phenyl]-3-[(1S)-1- (3-pyrimidin-2-ylpyrazin-2- yl)ethyl]urea | 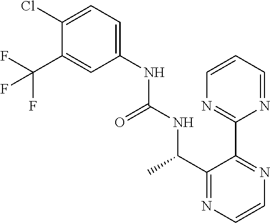 [SMILES: C[C@H](NC(=O)Nc1ccc(Cl)c(C(F)(F)F)c1)c1nccnc1-c1ncccn1] | 1 | 423.4 | 1 | 109 - 111 |
| P12 | 1-[5-chloro-2- (trifluoromethyl)phenyl]-3-[(1S)-1- (2-pyrimidin-2-yl-1,2,4-triazol-3- yl)ethyl]urea | 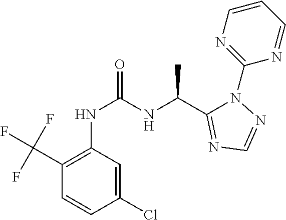 [SMILES: C[C@H](NC(=O)Nc1cc(Cl)ccc1C(F)(F)F)c1ncnn1-c1ncccn1] | 1.3 | 412.1 | 4 | 234 - 236 |
| P13 | 1-[2,4-bis(trifluoromethyl)phenyl]-3- [(1S)-1-[2-(5-cyano-2-pyridyl)-1,2,4- triazol-3-yl]ethyl]urea | 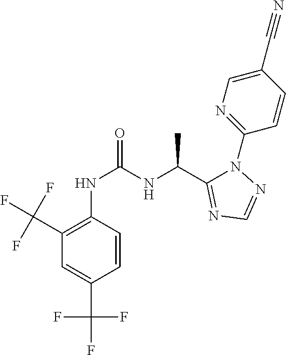 [SMILES: C[C@H](NC(=O)Nc1ccc(C(F)(F)F)cc1C(F)(F)F)c1ncnn1-c1ccc(C#N)cn1] | 1.45 | 470.2 | 4 | 235 - 237 |
| P14 | 1-[2-chloro-5- (trifluoromethyl)phenyl]-3-[(1S)-1- (2-pyrimidin-2-yl-1,2,4-triazol-3- yl)ethyl]urea | 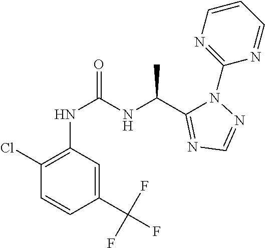 [SMILES: C[C@H](NC(=O)Nc1cc(C(F)(F)F)ccc1Cl)c1ncnn1-c1ncccn1] | 1.35 | 412.1 | 4 | 182 - 184 |
| P15 | 1-[4-chloro-3- (trifluoromethyl)phenyl]-3-[(1S)-1- (2-pyrimidin-2-yl-1,2,4-triazol-3- yl)ethyl]thiourea | 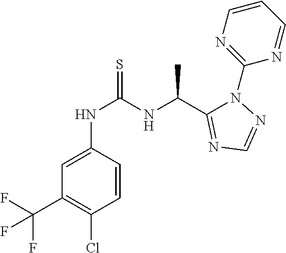 [SMILES: C[C@H](NC(=S)Nc1ccc(Cl)c(C(F)(F)F)c1)c1ncnn1-c1ncccn1] | 1.38 | 428.1 | 4 | 107 - 109 |
| P16 | 1-[2-(3-chloro-2-pyridyl)-5- (trifluoromethyl)pyrazol-3-yl]-3- [(1S)-1-(2-pyrimidin-2-yl-1,2,4- triazol-3-yl)ethyl]urea | 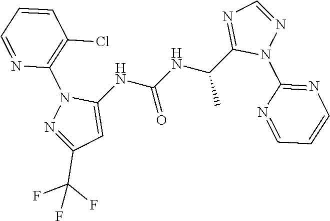 [SMILES: C[C@H](NC(=O)Nc1cc(C(F)(F)F)nn1-c1ncccc1Cl)c1ncnn1-c1ncccn1] | 1.19 | 479.0 | 3 | |
| P17 | 1-[2-(5-chloro-2-pyridyl)pyrazol-3- yl]-3-[(1S)-1-(2-pyrimidin-2-yl-1,2,4- triazol-3-yl)ethyl]urea | 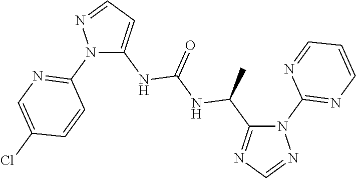 [SMILES: C[C@H](NC(=O)Nc1ccnn1-c1ccc(Cl)cn1)c1ncnn1-c1ncccn1] | 1.15 | 411.0 | 3 | |
| P18 | 1-[2-chloro-6-(trifluoromethyl)-3- pyridyl]-3-[(1S)-1-(2-pyrimidin-2-yl- 1,2,4-triazol-3-yl)ethyl]urea | 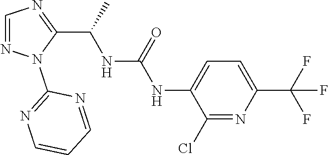 [SMILES: C[C@H](NC(=O)Nc1ccc(C(F)(F)F)nc1Cl)c1ncnn1-c1ncccn1] | 1.23 | 413.0 | 3 | |
| P19 | 1-[(1S)-1-(2-pyrimidin-2-yl-1,2,4- triazol-3-yl)ethyl]-3-[3-[4- (trifluoromethyl)pyrazol-1- yl]phenyl]urea | 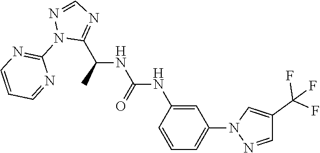 [SMILES: C[C@H](NC(=O)Nc1cccc(-n2cc(C(F)(F)F)cn2)c1)c1ncnn1-c1ncccn1] | 1.3 | 444.0 | 3 | |
| P20 | 1-(4-chlorophenyl)-3-[(1S)-1-(2- pyrimidin-2-yl-1,2,4-triazol-3- yl)ethyl]urea | 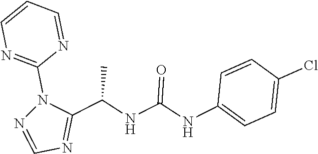 [SMILES: C[C@H](NC(=O)Nc1ccc(Cl)cc1)c1ncnn1-c1ncccn1] | 1.07 | 344.0 | 3 | |
| P21 | 1-(2,5-dichloro-3-pyridyl)-3-[(1S)-1- (2-pyrimidin-2-yl-1,2,4-triazol-3- yl)ethyl]urea | 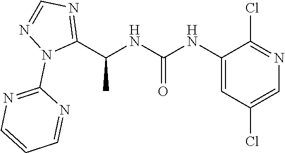 [SMILES: C[C@H](NC(=O)Nc1cc(Cl)cnc1Cl)c1ncnn1-c1ncccn1] | 1.08 | 379.0 | 3 | |
| P22 | 1-[3-fluoro-5- (trifluoromethyl)phenyl]-3-[(1S)-1- (2-pyrimidin-2-yl-1,2,4-triazol-3- yl)ethyl]urea | 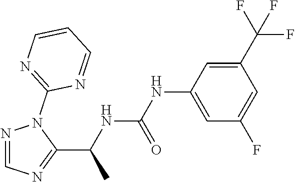 [SMILES: C[C@H](NC(=O)Nc1cc(F)cc(C(F)(F)F)c1)c1ncnn1-c1ncccn1] | 1.3 | 396.0 | 3 | |
| P23 | 1-[4-[(3,5-dichloro-2- pyridyl)oxy]phenyl]-3-[(1S)-1-(2- pyrimidin-2-yl-1,2,4-triazol-3- yl)ethyl]urea | 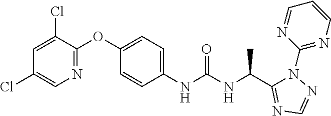 [SMILES: C[C@H](NC(=O)Nc1ccc(Oc2ncc(Cl)cc2Cl)cc1)c1ncnn1-c1ncccn1] | 1.41 | 471.0 | 3 | |
| P24 | 1-(3,4-dicyanophenyl)-3-[(1S)-1-(2- pyrimidin-2-yl-1,2,4-triazol-3- yl)ethyl]urea | 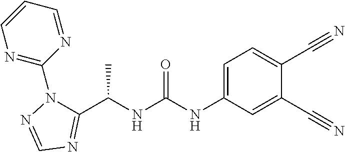 [SMILES: C[C@H](NC(=O)Nc1ccc(C#N)c(C#N)c1)c1ncnn1-c1ncccn1] | 0.97 | 360.0 | 3 | |
| P25 | 1-[3-chloro-5-(trifluoromethyl)-2- pyridyl]-3-[(1S)-1-(2-pyrimidin-2-yl- 1,2,4-triazol-3-yl)ethyl]urea | 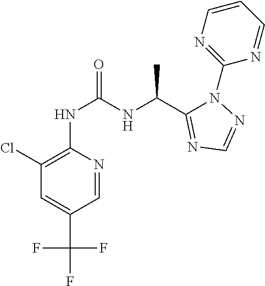 [SMILES: C[C@H](NC(=O)Nc1ncc(C(F)(F)F)cc1Cl)c1ncnn1-c1ncccn1] | 1.28 | 413.0 | 3 | |
| P26 | 1-[3,5-dichloro-4-[[3-chloro-5- (trifluoromethyl)-2- pyridyl]oxy]phenyl]-3-[(1S)-1-(2- pyrimidin-2-yl-1,2,4-triazol-3- yl)ethyl]urea | 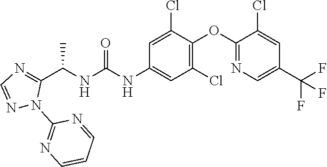 [SMILES: C[C@H](NC(=O)Nc1cc(Cl)c(Oc2ncc(C(F)(F)F)cc2Cl)c(Cl)c1)c1ncnn1-c1ncccn1] | 1.75 | 573.0 | 3 | |
| P27 | 1-[(1S)-1-(2-pyrimidin-2-yl-1,2,4- triazol-3-yl)ethyl]-3-[2- (trifluoromethyl)phenyl]urea | 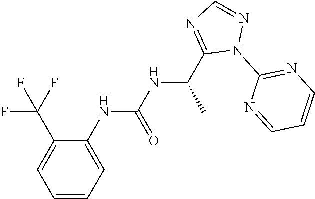 [SMILES: C[C@H](NC(=O)Nc1ccccc1C(F)(F)F)c1ncnn1-c1ncccn1] | 1.06 | 378.0 | 3 | |
| P28 | 1-[3-chloro-2-fluoro-5- (trifluoromethyl)phenyl]-3-[(1S)-1- (2-pyrimidin-2-yl-1,2,4-triazol-3- yl)ethyl]urea | 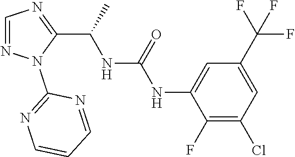 [SMILES: C[C@H](NC(=O)Nc1cc(C(F)(F)F)cc(Cl)c1F)c1ncnn1-c1ncccn1] | 1.45 | 430.0 | 3 | |
| P29 | 1-[5-chloro-2-(3-chloro-2- pyridyl)pyrazol-3-yl]-3-[(1S)-1-(2- pyrimidin-2-yl-1,2,4-triazol-3- yl)ethyl]urea | 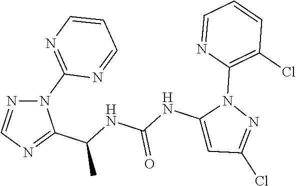 [SMILES: C[C@H](NC(=O)Nc1cc(Cl)nn1-c1ncccc1Cl)c1ncnn1-c1ncccn1] | 1.01 | 445.0 | 3 | |
| P30 | 1-(2-bromo-4-cyano-phenyl)-3- [(1S)-1-(2-pyrimidin-2-yl-1,2,4- triazol-3-yl)ethyl]urea | 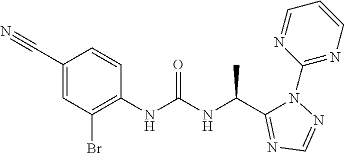 [SMILES: C[C@H](NC(=O)Nc1ccc(C#N)cc1Br)c1ncnn1-c1ncccn1] | 1.06 | 413.0 | 3 | |
| P31 | 1-[(1S)-1-(2-pyrimidin-2-yl-1,2,4- triazol-3-yl)ethyl]-3-[6- (trifluoromethylsulfanyl)pyrimidin-4- yl]urea | 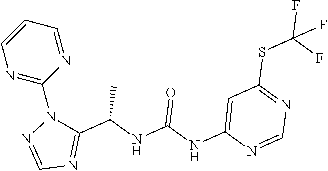 [SMILES: C[C@H](NC(=O)Nc1cc(SC(F)(F)F)ncn1)c1ncnn1-c1ncccn1] | 1.16 | 412.0 | 3 | |
| P32 | 1-[(1S)-1-(2-pyrimidin-2-yl-1,2,4- triazol-3-yl)ethyl]-3-[6- (trifluoromethyl)pyrimidin-4-yl]urea | 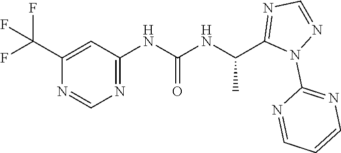 [SMILES: C[C@H](NC(=O)Nc1cc(C(F)(F)F)ncn1)c1ncnn1-c1ncccn1] | 1 | 380.0 | 3 | |
| P33 | 1-(4,6-dicyclopropylpyrimidin-2-yl)- 3-[(1S)-1-(2-pyrimidin-2-yl-1,2,4- triazol-3-yl)ethyl]urea | 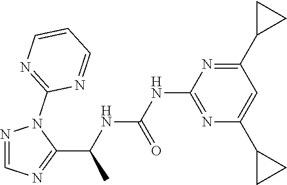 [SMILES: C[C@H](NC(=O)Nc1nc(C2CC2)cc(C2CC2)n1)c1ncnn1-c1ncccn1] | 1.31 | 392.0 | 3 | |
| P34 | 1-[4-[(5-cyclopropyl-1,3,4- thiadiazol-2-yl)oxy]-2-methyl- phenyl]-3-[(1S)-1-(2-pyrimidin-2-yl- 1,2,4-triazol-3-yl)ethyl]urea | 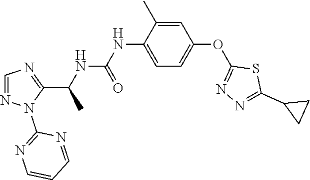 [SMILES: Cc1cc(Oc2nnc(C3CC3)s2)ccc1NC(=O)N[C@@H](C)c1ncnn1-c1ncccn1] | 1.11 | 464.0 | 3 | |
| P35 | 1-[4-chloro-3-(1,1,2,2- tetrafluoroethoxy)phenyl]-3-[(1S)-1- (2-pyrimidin-2-yl-1,2,4-triazol-3- yl)ethyl]urea | 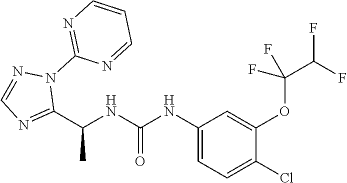 [SMILES: C[C@H](NC(=O)Nc1ccc(Cl)c(OC(F)(F)C(F)F)c1)c1ncnn1-c1ncccn1] | 1.36 | 460.0 | 3 | |
| P36 | 1-[(1S)-1-(2-pyrimidin-2-yl-1,2,4- triazol-3-yl)ethyl]-3-[4- (trifluoromethyl)pyrimidin-2-yl]urea | 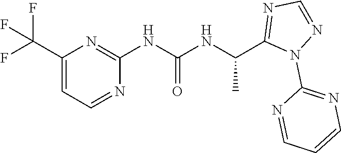 [SMILES: C[C@H](NC(=O)Nc1nccc(C(F)(F)F)n1)c1ncnn1-c1ncccn1] | 1.03 | 380.0 | 3 | |
| P37 | 1-(6-chloro-5-methyl-3-pyridyl)-3- [(1S)-1-(2-pyrimidin-2-yl-1,2,4- triazol-3-yl)ethyl]urea | 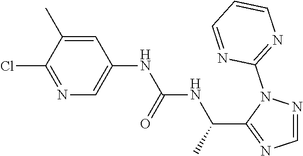 [SMILES: Cc1cc(NC(=O)N[C@@H](C)c2ncnn2-c2ncccn2)cnc1Cl] | 0.95 | 359.0 | 3 | |
| P38 | 1-[(1S)-1-(2-pyrimidin-2-yl-1,2,4- triazol-3-yl)ethyl]-3-[4- (trifluoromethyl)thieno[2,3- d]pyrimidin-2-yl]urea | 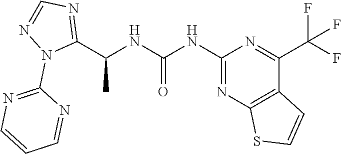 [SMILES: C[C@H](NC(=O)Nc1nc(C(F)(F)F)c2ccsc2n1)c1ncnn1-c1ncccn1] | 1.26 | 436.0 | 3 | |
| P39 | 1-[3-(difluoromethoxy)phenyl]-3- [(1S)-1-(2-pyrimidin-2-yl-1,2,4- triazol-3-yl)ethyl]urea | 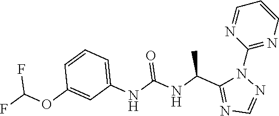 [SMILES: C[C@H](NC(=O)Nc1cccc(OC(F)F)c1)c1ncnn1-c1ncccn1] | 1.07 | 376.0 | 3 | |
| P40 | 1-(3-bromo-4-isopropyl-phenyl)-3- [(1S)-1-(2-pyrimidin-2-yl-1,2,4- triazol-3-yl)ethyl]urea | 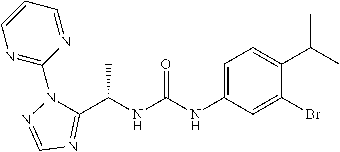 [SMILES: CC(C)c1ccc(NC(=O)N[C@@H](C)c2ncnn2-c2ncccn2)cc1Br] | 1.45 | 430.0 | 3 | |
| P41 | 1-[3-(methanesulfonamido)-4- methyl-phenyl]-3-[(1S)-1-(2- pyrimidin-2-yl-1,2,4-triazol-3- yl)ethyl]urea | 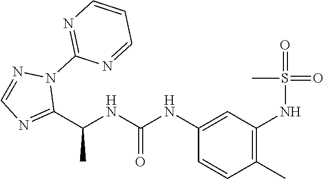 [SMILES: Cc1ccc(NC(=O)N[C@@H](C)c2ncnn2-c2ncccn2)cc1NS(C)(=O)=O] | 0.8 | 417.0 | 3 | |
| P42 | 1-(4-chloro-3-sulfamoyl-phenyl)-3- [(1S)-1-(2-pyrimidin-2-yl-1,2,4- triazol-3-yl)ethyl]urea | 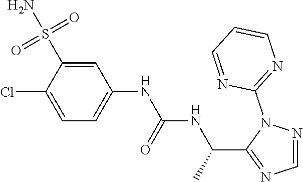 [SMILES: C[C@H](NC(=O)Nc1ccc(Cl)c(S(N)(=O)=O)c1)c1ncnn1-c1ncccn1] | 0.77 | 423.0 | 3 | |
| P43 | 1-[(1S)-1-(2-pyrimidin-2-yl-1,2,4- triazol-3-yl)ethyl]-3-(2,4,5- trichlorophenyl)urea | 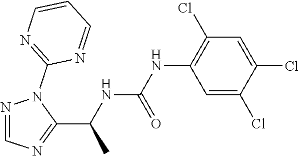 [SMILES: C[C@H](NC(=O)Nc1cc(Cl)c(Cl)cc1Cl)c1ncnn1-c1ncccn1] | 1.42 | 412.0 | 3 | |
| P44 | 1-(3,4-dibromophenyl)-3-[(1S)-1-(2- pyrimidin-2-yl-1,2,4-triazol-3- yl)ethyl]urea | 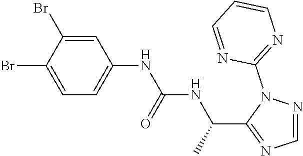 [SMILES: C[C@H](NC(=O)Nc1ccc(Br)c(Br)c1)c1ncnn1-c1ncccn1] | 1.3 | 466.0 | 3 | |
| P45 | 1-[2-(1,1-difluoroethyl)-4-fluoro- phenyl]-3-[(1S)-1-(2-pyrimidin-2-yl- 1,2,4-triazol-3-yl)ethyl]urea | 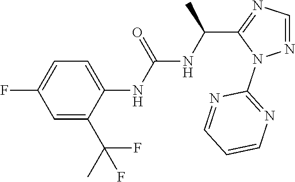 [SMILES: C[C@H](NC(=O)Nc1ccc(F)cc1C(C)(F)F)c1ncnn1-c1ncccn1] | 1.09 | 392.0 | 3 | |
| P46 | 1-[2,4-dichloro-5-(1,1,2,2- tetrafluoroethoxy)phenyl]-3-[(1S)-1- (2-pyrimidin-2-yl-1,2,4-triazol-3- yl)ethyl]urea | 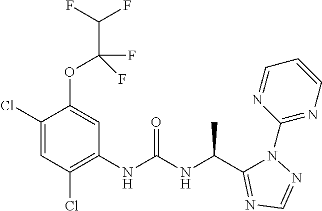 [SMILES: C[C@H](NC(=O)Nc1cc(OC(F)(F)C(F)F)c(Cl)cc1Cl)c1ncnn1-c1ncccn1] | 1.52 | 494.0 | 3 | 201 - 205 |
| P47 | 1-[2-(2-pyridyloxy)phenyl]-3-[(1S)-1- (2-pyrimidin-2-yl-1,2,4-triazol-3- yl)ethyl]urea | 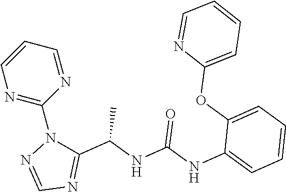 [SMILES: C[C@H](NC(=O)Nc1ccccc1Oc1ccccn1)c1ncnn1-c1ncccn1] | 0.73 | 403.0 | 3 | |
| P48 | 1-[2-bromo-4-chloro-5- (trifluoromethyl)phenyl]-3-[(1S)-1- (2-pyrimidin-2-yl-1,2,4-triazol-3- yl)ethyl]urea | 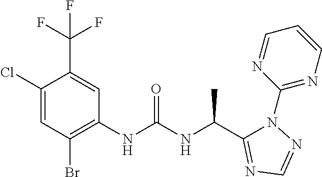 [SMILES: C[C@H](NC(=O)Nc1cc(C(F)(F)F)c(Cl)cc1Br)c1ncnn1-c1ncccn1] | 1.53 | 490.08 | 3 | 214 - 216 |
| P49 | 1-[2-chloro-4-(8-methoxy-1-oxa-2,8- diazaspiro[4.5]dec-2-en-3- yl)phenyl]-3-[(1S)-1-(2-pyrimidin-2- yl-1,2,4-triazol-3-yl)ethyl]urea | 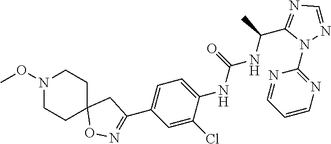 [SMILES: CON1CCC2(CC1)CC(c1ccc(NC(=O)N[C@@H](C)c3ncnn3-c3ncccn3)c(Cl)c1)=NO2] | 1.17 | 512.0 | 3 | |
| P50 | N-[(1S)-1-(2-pyrimidin-2-yl-1,2,4- triazol-3-yl)ethyl]-6- (trifluoromethyl)indoline-1- carboxamide | 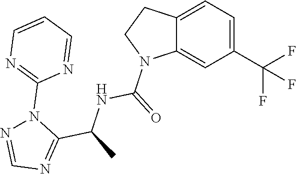 [SMILES: C[C@H](NC(=O)N1CCc2ccc(C(F)(F)F)cc21)c1ncnn1-c1ncccn1] | 1.32 | 404.0 | 3 | |
| P51 | 1-[4-chloro-2-(4- chlorophenoxy)phenyl]-3-[(1S)-1-(2- pyrimidin-2-yl-1,2,4-triazol-3- yl)ethyl]urea | 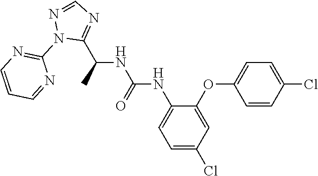 [SMILES: C[C@H](NC(=O)Nc1ccc(Cl)cc1Oc1ccc(Cl)cc1)c1ncnn1-c1ncccn1] | 1.63 | 470.0 | 3 | |
| P52 | 1-[3-[(3,5-dichloro-2- pyridyl)oxy]phenyl]-3-[(1S)-1-(2- pyrimidin-2-yl-1,2,4-triazol-3- yl)ethyl]urea | 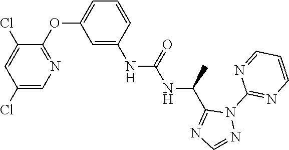 [SMILES: C[C@H](NC(=O)Nc1cccc(Oc2ncc(Cl)cc2Cl)c1)c1ncnn1-c1ncccn1] | 1.43 | 471.0 | 3 | |
| P53 | 1-[1-(difluoromethyl)-5-methyl- pyrazol-4-yl]-3-[(1S)-1-(2-pyrimidin- 2-yl-1,2,4-triazol-3-yl)ethyl]urea | 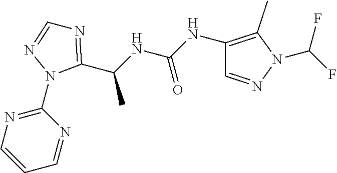 [SMILES: Cc1c(NC(=O)N[C@@H](C)c2ncnn2-c2ncccn2)cnn1C(F)F] | 0.74 | 364.0 | 3 | |
| P54 | 1-(4-chloro-3-cyano-phenyl)-3- [(1S)-1-(2-pyrimidin-2-yl-1,2,4- triazol-3-yl)ethyl]urea | 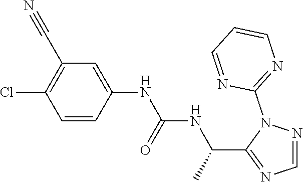 [SMILES: C[C@H](NC(=O)Nc1ccc(Cl)c(C#N)c1)c1ncnn1-c1ncccn1] | 1.08 | 369.0 | 3 | |
| P55 | 1-(4,6-dibromo-3-pyridyl)-3-[(1S)-1- (2-pyrimidin-2-yl-1,2,4-triazol-3- yl)ethyl]urea | 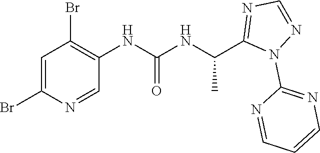 [SMILES: C[C@H](NC(=O)Nc1cnc(Br)cc1Br)c1ncnn1-c1ncccn1] | 1.04 | 467.0 | 3 | |
| P56 | 1-(3-bromo-6-methyl-2-pyridyl)-3- [(1S)-1-(2-pyrimidin-2-yl-1,2,4- triazol-3-yl)ethyl]urea | 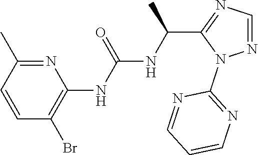 [SMILES: Cc1ccc(Br)c(NC(=O)N[C@@H](C)c2ncnn2-c2ncccn2)n1] | 1.22 | 403.0 | 3 | |
| P57 | N-[(1S)-1-(2-pyrimidin-2-yl-1,2,4- triazol-3-yl)ethyl]-5- (trifluoromethyl)indoline-1- carboxamide | 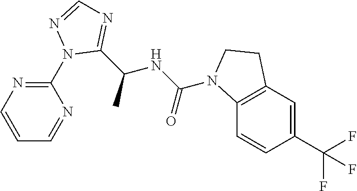 [SMILES: C[C@H](NC(=O)N1CCc2cc(C(F)(F)F)ccc21)c1ncnn1-c1ncccn1] | 1.33 | 404.0 | 3 | |
| P58 | 1-[4-bromo-2- (difluoromethoxy)phenyl]-3-[(1S)-1- (2-pyrimidin-2-yl-1,2,4-triazol-3- yl)ethyl]urea | 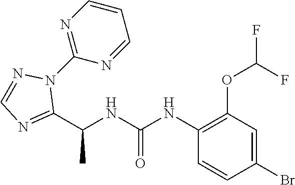 [SMILES: C[C@H](NC(=O)Nc1ccc(Br)cc1OC(F)F)c1ncnn1-c1ncccn1] | 1.29 | 454.0 | 3 | |
| P59 | 1-[(1S)-1-(2-pyrimidin-2-yl-1,2,4- triazol-3-yl)ethyl]-3- ([1,2,4]triazolo[1,5-a]pyrimidin-7- yl)urea | 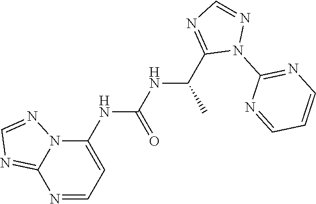 [SMILES: C[C@H](NC(=O)Nc1ccnc2ncnn12)c1ncnn1-c1ncccn1] | 0.68 | 352.0 | 3 | |
| P60 | 1-[3-(cyclopropylsulfamoyl)-4- methyl-phenyl]-3-[(1S)-1-(2- pyrimidin-2-yl-1,2,4-triazol-3- yl)ethyl]urea | 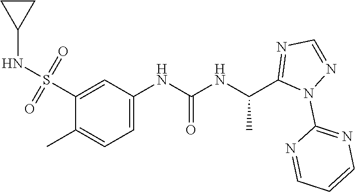 [SMILES: Cc1ccc(NC(=O)N[C@@H](C)c2ncnn2-c2ncccn2)cc1S(=O)(=O)NC1CC1] | 0.97 | 443.0 | 3 | |
| P61 | 1-(3,5-difluoro-4-methylsulfonyl- phenyl)-3-[(1S)-1-(2-pyrimidin-2-yl- 1,2,4-triazol-3-yl)ethyl]urea | 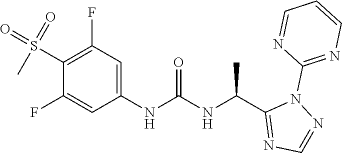 [SMILES: C[C@H](NC(=O)Nc1cc(F)c(S(C)(=O)=O)c(F)c1)c1ncnn1-c1ncccn1] | 0.86 | 424.0 | 3 | |
| P62 | 1-(2-cyano-4-fluoro-phenyl)-3-[(1S)- 1-(2-pyrimidin-2-yl-1,2,4-triazol-3- yl)ethyl]urea | 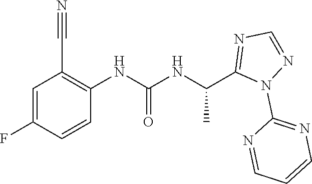 [SMILES: C[C@H](NC(=O)Nc1ccc(F)cc1C#N)c1ncnn1-c1ncccn1] | 0.97 | 353.0 | 3 | |
| P63 | 1-(4-chloro-3-methylsulfonyl- phenyl)-3-[(1S)-1-(2-pyrimidin-2-yl- 1,2,4-triazol-3-yl)ethyl]urea | 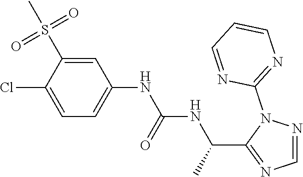 [SMILES: C[C@H](NC(=O)Nc1ccc(Cl)c(S(C)(=O)=O)c1)c1ncnn1-c1ncccn1] | 0.9 | 422.0 | 3 | |
| P64 | 1-[2-(cyclopropylmethoxy)-4-fluoro- phenyl]-3-[(1S)-1-(2-pyrimidin-2-yl- 1,2,4-triazol-3-yl)ethyl]urea | 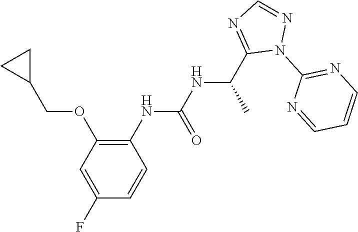 [SMILES: C[C@H](NC(=O)Nc1ccc(F)cc1OCC1CC1)c1ncnn1-c1ncccn1] | 1.27 | 398.0 | 3 | |
| P65 | 1-(5-cyclopropylisoxazol-4-yl)-3- [(1S)-1-(2-pyrimidin-2-yl-1,2,4- triazol-3-yl)ethyl]urea | 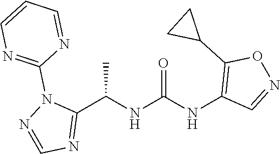 [SMILES: C[C@H](NC(=O)Nc1cnoc1C1CC1)c1ncnn1-c1ncccn1] | 0.77 | 341.0 | 3 | |
| P66 | N-cyclopentyl-4-[[(1S)-1-(2- pyrimidin-2-yl-1,2,4-triazol-3- yl)ethyl]carbamoylamino]benzamide | 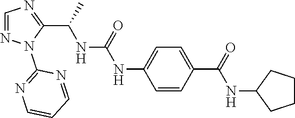 [SMILES: C[C@H](NC(=O)Nc1ccc(C(=O)NC2CCCC2)cc1)c1ncnn1-c1ncccn1] | 1.02 | 421.0 | 3 | |
| P67 | 1-[(1S)-1-(2-pyrimidin-2-yl-1,2,4- triazol-3-yl)ethyl]-3-[3- (trifluoromethyl)phenyl]urea | 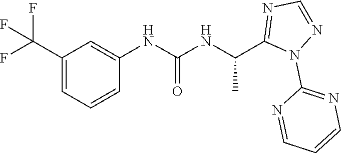 [SMILES: C[C@H](NC(=O)Nc1cccc(C(F)(F)F)c1)c1ncnn1-c1ncccn1] | 1.2 | 378.0 | 3 | |
| P68 | 1-(2,6-dichloro-4-pyridyl)-3-[(1S)-1- (2-pyrimidin-2-yl-1,2,4-triazol-3- yl)ethyl]urea | 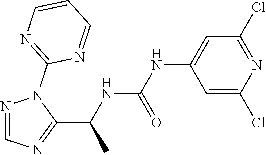 [SMILES: C[C@H](NC(=O)Nc1cc(Cl)nc(Cl)c1)c1ncnn1-c1ncccn1] | 1.08 | 379.0 | 3 | |
| P69 | 1-[(1S)-1-(2-pyrimidin-2-yl-1,2,4- triazol-3-yl)ethyl]-3-(3,4,5- trichlorophenyl)urea | 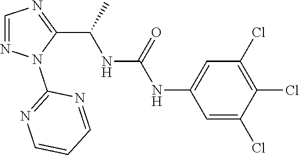 [SMILES: C[C@H](NC(=O)Nc1cc(Cl)c(Cl)c(Cl)c1)c1ncnn1-c1ncccn1] | 1.44 | 412.0 | 3 | |
| P70 | 1-[2-bromo-5- (trifluoromethyl)phenyl]-3-[(1S)-1- (2-pyrimidin-2-yl-1,2,4-triazol-3- yl)ethyl]urea | 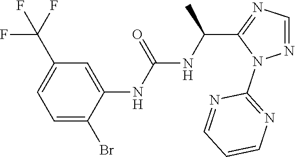 [SMILES: C[C@H](NC(=O)Nc1cc(C(F)(F)F)ccc1Br)c1ncnn1-c1ncccn1] | 1.36 | 456.0 | 3 | |
| P71 | 1-(2-chloro-6-methyl-phenyl)-3- [(1S)-1-(2-pyrimidin-2-yl-1,2,4- triazol-3-yl)ethyl]urea | 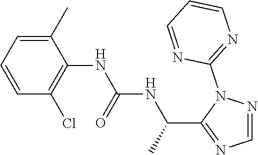 [SMILES: Cc1cccc(Cl)c1NC(=O)N[C@@H](C)c1ncnn1-c1ncccn1] | 0.93 | 358.0 | 3 | |
| P72 | 1-[2-iodo-4-(trifluoromethyl)phenyl]- 3-[(1S)-1-(2-pyrimidin-2-yl-1,2,4- triazol-3-yl)ethyl]urea | 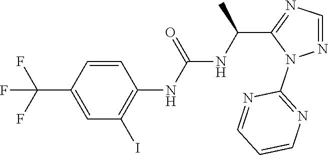 [SMILES: C[C@H](NC(=O)Nc1ccc(C(F)(F)F)cc1I)c1ncnn1-c1ncccn1] | 1.39 | 504.0 | 3 | |
| P73 | 7-bromo-N-[(1S)-1-(2-pyrimidin-2- yl-1,2,4-triazol-3-yl)ethyl]indoline-1- carboxamide | 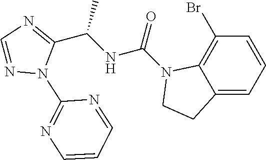 [SMILES: C[C@H](NC(=O)N1CCc2cccc(Br)c21)c1ncnn1-c1ncccn1] | 1.06 | 414.0 | 3 | |
| P74 | 1-(5-bromo-6-methyl-3-pyridyl)-3- [(1S)-1-(2-pyrimidin-2-yl-1,2,4- triazol-3-yl)ethyl]urea | 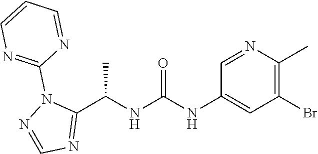 [SMILES: Cc1ncc(NC(=O)N[C@@H](C)c2ncnn2-c2ncccn2)cc1Br] | 0.91 | 403.0 | 3 | |
| P75 | 1-(4-chloro-3-methyl-phenyl)-3- [(1S)-1-(2-pyrimidin-2-yl-1,2,4- triazol-3-yl)ethyl]urea | 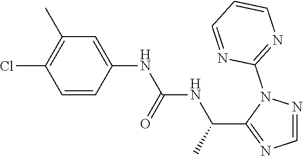 [SMILES: Cc1cc(NC(=O)N[C@@H](C)c2ncnn2-c2ncccn2)ccc1Cl] | 1.19 | 358.0 | 3 | |
| P76 | 1-(4-bromo-2-iodo-phenyl)-3-[(1S)- 1-(2-pyrimidin-2-yl-1,2,4-triazol-3- yl)ethyl]urea | 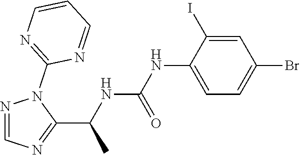 [SMILES: C[C@H](NC(=O)Nc1ccc(Br)cc1I)c1ncnn1-c1ncccn1] | 1.29 | 514.0 | 3 | |
| P77 | 1-[2-bromo-4- (trifluoromethoxy)phenyl]-3-[(1S)-1- (2-pyrimidin-2-yl-1,2,4-triazol-3- yl)ethyl]urea | 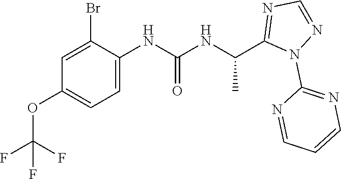 [SMILES: C[C@H](NC(=O)Nc1ccc(OC(F)(F)F)cc1Br)c1ncnn1-c1ncccn1] | 1.38 | 472.0 | 3 | |
| P78 | 1-(3-chloro-4-cyano-phenyl)-3- [(1S)-1-(2-pyrimidin-2-yl-1,2,4- triazol-3-yl)ethyl]urea | 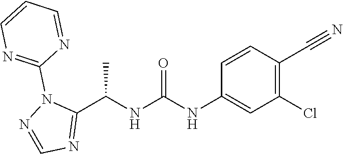 [SMILES: C[C@H](NC(=O)Nc1ccc(C#N)c(Cl)c1)c1ncnn1-c1ncccn1] | 1.07 | 369.0 | 3 | |
| P79 | 1-(6-bromo-2,2-difluoro-1,3- benzodioxol-5-yl)-3-[(1S)-1-(2- pyrimidin-2-yl-1,2,4-triazol-3- yl)ethyl]urea | 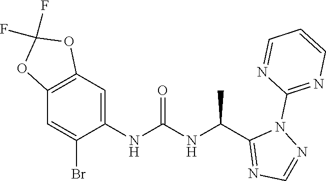 [SMILES: C[C@H](NC(=O)Nc1cc2c(cc1Br)OC(F)(F)O2)c1ncnn1-c1ncccn1] | 1.34 | 468.13 | 3 | |
| P80 | 1-(2,4-dichloropyrimidin-5-yl)-3- [(1S)-1-(2-pyrimidin-2-yl-1,2,4- triazol-3-yl)ethyl]urea | 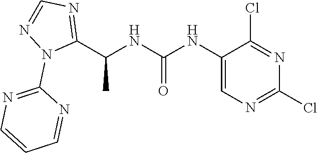 [SMILES: C[C@H](NC(=O)Nc1cnc(Cl)nc1Cl)c1ncnn1-c1ncccn1] | 0.96 | 380.0 | 3 | |
| P81 | 1-(2-bromo-4-isopropyl-phenyl)-3- [(1S)-1-(2-pyrimidin-2-yl-1,2,4- triazol-3-yl)ethyl]urea | 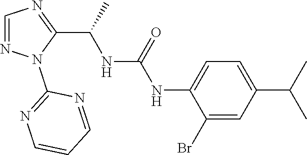 [SMILES: CC(C)c1ccc(NC(=O)N[C@@H](C)c2ncnn2-c2ncccn2)c(Br)c1] | 1.39 | 430.0 | 3 | |
| P82 | 1-(3,5-dichloro-2-pyridyl)-3-[(1S)-1- (2-pyrimidin-2-yl-1,2,4-triazol-3- yl)ethyl]urea | 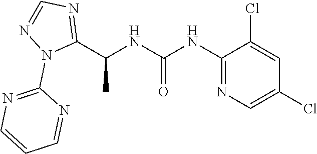 [SMILES: C[C@H](NC(=O)Nc1ncc(Cl)cc1Cl)c1ncnn1-c1ncccn1] | 1.16 | 379.0 | 3 | |
| P83 | 1-[6-chloro-4-(trifluoromethyl)-2- pyridyl]-3-[(1S)-1-(2-pyrimidin-2-yl- 1,2,4-triazol-3-yl)ethyl]urea | 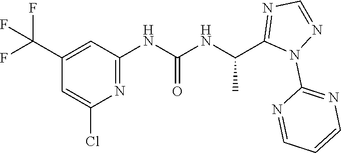 [SMILES: C[C@H](NC(=O)Nc1cc(C(F)(F)F)cc(Cl)n1)c1ncnn1-c1ncccn1] | 1.36 | 413.0 | 3 | |
| P84 | 1-(2-cyanophenyl)-3-[(1S)-1-(2- pyrimidin-2-yl-1,2,4-triazol-3- yl)ethyl]urea | 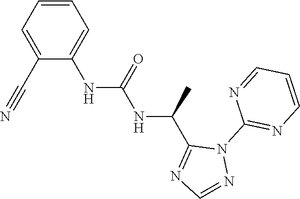 [SMILES: C[C@H](NC(=O)Nc1ccccc1C#N)c1ncnn1-c1ncccn1] | 0.86 | 335.0 | 3 | |
| P85 | 1-(5-bromo-1-methyl-indazol-3-yl)- 3-[(1S)-1-(2-pyrimidin-2-yl-1,2,4- triazol-3-yl)ethyl]urea | 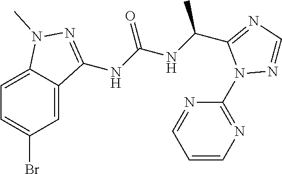 [SMILES: C[C@H](NC(=O)Nc1nn(C)c2ccc(Br)cc12)c1ncnn1-c1ncccn1] | 1.17 | 442.0 | 3 | |
| P86 | 1-(2-bromo-3-fluoro-phenyl)-3- [(1S)-1-(2-pyrimidin-2-yl-1,2,4- triazol-3-yl)ethyl]urea | 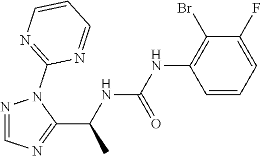 [SMILES: C[C@H](NC(=O)Nc1cccc(F)c1Br)c1ncnn1-c1ncccn1] | 1.1 | 406.0 | 3 | |
| P87 | 1-(2-chloro-4,5-difluoro-phenyl)-3- [(1S)-1-(2-pyrimidin-2-yl-1,2,4- triazol-3-yl)ethyl]urea | 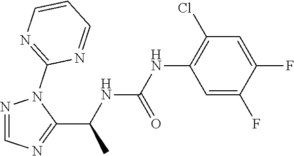 [SMILES: C[C@H](NC(=O)Nc1cc(F)c(F)cc1Cl)c1ncnn1-c1ncccn1] | 1.18 | 380.0 | 3 | |
| P88 | 1-(5-cyano-6-methyl-2-pyridyl)-3- [(1S)-1-(2-pyrimidin-2-yl-1,2,4- triazol-3-yl)ethyl]urea | 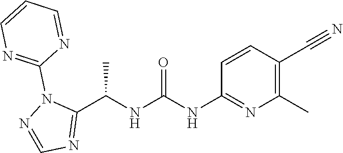 [SMILES: Cc1nc(NC(=O)N[C@@H](C)c2ncnn2-c2ncccn2)ccc1C#N] | 0.98 | 350.0 | 3 | |
| P89 | 1-(4-cyano-3,5-difluoro-phenyl)-3- [(1S)-1-(2-pyrimidin-2-yl-1,2,4- triazol-3-yl)ethyl]urea | 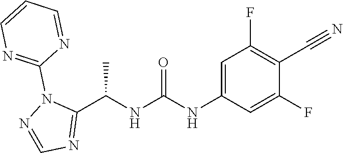 [SMILES: C[C@H](NC(=O)Nc1cc(F)c(C#N)c(F)c1)c1ncnn1-c1ncccn1] | 1.11 | 371.0 | 3 | |
| P90 | 1-(2-fluoro-4-methylsulfonyl- phenyl)-3-[(1S)-1-(2-pyrimidin-2-yl- 1,2,4-triazol-3-yl)ethyl]urea | 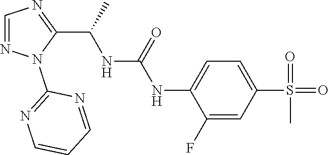 [SMILES: C[C@H](NC(=O)Nc1ccc(S(C)(=O)=O)cc1F)c1ncnn1-c1ncccn1] | 0.81 | 406.0 | 3 | |
| P91 | 1-[4,6-bis(trifluoromethyl)-2-pyridyl]- 3-[(1S)-1-(2-pyrimidin-2-yl-1,2,4- triazol-3-yl)ethyl]urea | 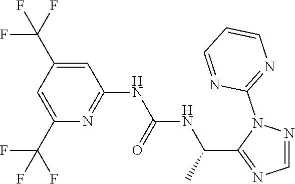 [SMILES: C[C@H](NC(=O)Nc1cc(C(F)(F)F)cc(C(F)(F)F)n1)c1ncnn1-c1ncccn1] | 1.45 | 447.0 | 3 | |
| P92 | 1-(3,4-dichloro-2-cyano-phenyl)-3- [(1S)-1-(2-pyrimidin-2-yl-1,2,4- triazol-3-yl)ethyl]urea | 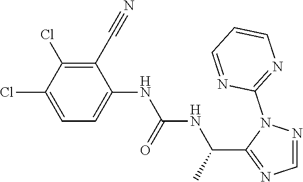 [SMILES: C[C@H](NC(=O)Nc1ccc(Cl)c(Cl)c1C#N)c1ncnn1-c1ncccn1] | 1.25 | 403.0 | 3 | |
| P93 | 1-(2-cyclopropylphenyl)-3-[(1S)-1- (2-pyrimidin-2-yl-1,2,4-triazol-3- yl)ethyl]urea | 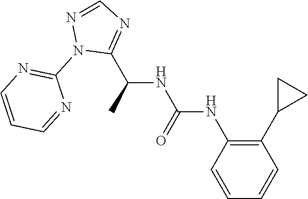 [SMILES: C[C@H](NC(=O)Nc1ccccc1C1CC1)c1ncnn1-c1ncccn1] | 1.08 | 350.0 | 3 | |
| P94 | 1-(2,6-dichlorophenyl)-3-[(1S)-1-(2- pyrimidin-2-yl-1,2,4-triazol-3- yl)ethyl]urea | 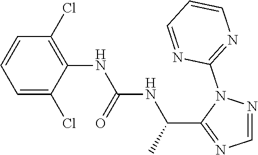 [SMILES: C[C@H](NC(=O)Nc1c(Cl)cccc1Cl)c1ncnn1-c1ncccn1] | 0.91 | 378.0 | 3 | |
| P95 | 1-[(1S)-1-(2-pyrimidin-2-yl-1,2,4- triazol-3-yl)ethyl]-3-(2,3,4- trichlorophenyl)urea | 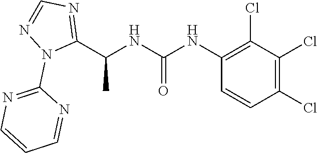 [SMILES: C[C@H](NC(=O)Nc1ccc(Cl)c(Cl)c1Cl)c1ncnn1-c1ncccn1] | 1.38 | 412.0 | 3 | |
| P96 | 1-[(1S)-1-(2-pyrimidin-2-yl-1,2,4- triazol-3-yl)ethyl]-3-[4- (trifluoromethylsulfonyl)phenyl]urea | 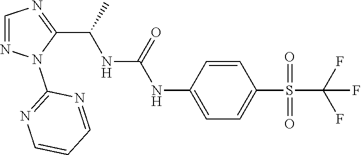 [SMILES: C[C@H](NC(=O)Nc1ccc(S(=O)(=O)C(F)(F)F)cc1)c1ncnn1-c1ncccn1] | 1.25 | 442.0 | 3 | |
| P97 | 1-(4-cyanophenyl)-3-[(1S)-1-(2- pyrimidin-2-yl-1,2,4-triazol-3- yl)ethyl]urea | 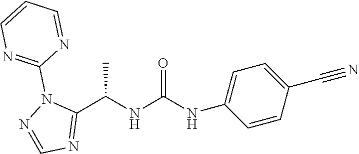 [SMILES: C[C@H](NC(=O)Nc1ccc(C#N)cc1)c1ncnn1-c1ncccn1] | 0.89 | 335.0 | 3 | |
| P98 | 1-(4-bromo-2,6-dichloro-phenyl)-3- [(1S)-1-(2-pyrimidin-2-yl-1,2,4- triazol-3-yl)ethyl]urea | 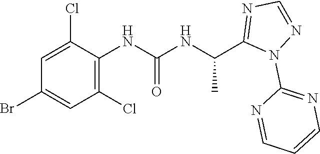 [SMILES: C[C@H](NC(=O)Nc1c(Cl)cc(Br)cc1Cl)c1ncnn1-c1ncccn1] | 1.15 | 456.0 | 3 | |
| P99 | 1-(4-fluoro-2-methylsulfonyl- phenyl)-3-[(1S)-1-(2-pyrimidin-2-yl- 1,2,4-triazol-3-yl)ethyl]urea | 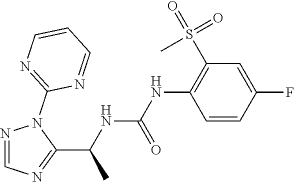 [SMILES: C[C@H](NC(=O)Nc1ccc(F)cc1S(C)(=O)=O)c1ncnn1-c1ncccn1] | 0.9 | 406.0 | 3 | |
| P100 | 1-(4-bromo-3-chloro-phenyl)-3- [(1S)-1-(2-pyrimidin-2-yl-1,2,4- triazol-3-yl)ethyl]urea | 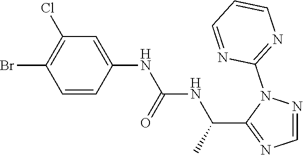 [SMILES: C[C@H](NC(=O)Nc1ccc(Br)c(Cl)c1)c1ncnn1-c1ncccn1] | 1.28 | 422.0 | 3 | |
| P101 | 1-(3-chlorophenyl)-3-[(1S)-1-(2- pyrimidin-2-yl-1,2,4-triazol-3- yl)ethyl]urea | 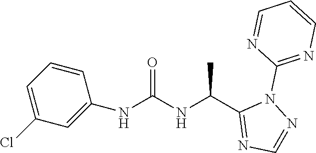 [SMILES: C[C@H](NC(=O)Nc1cccc(Cl)c1)c1ncnn1-c1ncccn1] | 1.09 | 344.0 | 3 | |
| P102 | 5-bromo-N-[(1S)-1-(2-pyrimidin-2- yl-1,2,4-triazol-3-yl)ethyl]indoline-1- carboxamide | 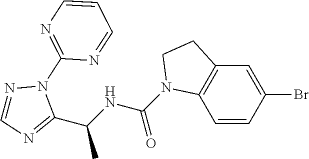 [SMILES: C[C@H](NC(=O)N1CCc2cc(Br)ccc21)c1ncnn1-c1ncccn1] | 1.26 | 414.0 | 3 | |
| P103 | 1-[4-chloro-6-(trifluoromethyl)-3- pyridyl]-3-[(1S)-1-(2-pyrimidin-2-yl- 1,2,4-triazol-3-yl)ethyl]urea | 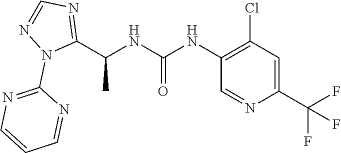 [SMILES: C[C@H](NC(=O)Nc1cnc(C(F)(F)F)cc1Cl)c1ncnn1-c1ncccn1] | 1.15 | 413.0 | 3 | 191 - 193 |
| P104 | 1-(3-chloro-4-methyl-phenyl)-3- [(1S)-1-(2-pyrimidin-2-yl-1,2,4- triazol-3-yl)ethyl]urea | 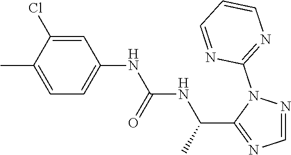 [SMILES: Cc1ccc(NC(=O)N[C@@H](C)c2ncnn2-c2ncccn2)cc1Cl] | 1.19 | 358.0 | 3 | |
| P105 | 1-(2,6-dibromo-3-pyridyl)-3-[(1S)-1- (2-pyrimidin-2-yl-1,2,4-triazol-3- yl)ethyl]urea | 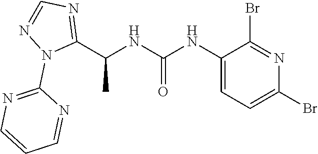 [SMILES: C[C@H](NC(=O)Nc1ccc(Br)nc1Br)c1ncnn1-c1ncccn1] | 1.11 | 467.0 | 3 | |
| P106 | 1-(4-bromo-2-methyl-phenyl)-3- [(1S)-1-(2-pyrimidin-2-yl-1,2,4- triazol-3-yl)ethyl]urea | 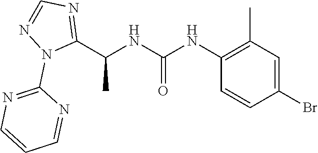 [SMILES: Cc1cc(Br)ccc1NC(=O)N[C@@H](C)c1ncnn1-c1ncccn1] | 1.16 | 402.0 | 3 | |
| P107 | 1-[2-(difluoromethoxy)phenyl]-3- [(1S)-1-(2-pyrimidin-2-yl-1,2,4- triazol-3-yl)ethyl]urea | 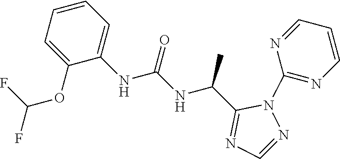 [SMILES: C[C@H](NC(=O)Nc1ccccc1OC(F)F)c1ncnn1-c1ncccn1] | 1.05 | 376.0 | 3 | |
| P108 | 1-[1-methyl-5- (trifluoromethyl)pyrazol-3-yl]-3- [(1S)-1-(2-pyrimidin-2-yl-1,2,4- triazol-3-yl)ethyl]urea | 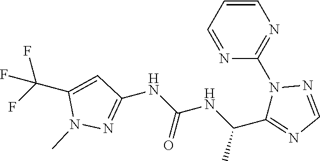 [SMILES: C[C@H](NC(=O)Nc1cc(C(F)(F)F)n(C)n1)c1ncnn1-c1ncccn1] | 1.02 | 382.0 | 3 | |
| P109 | 1-(5-cyclopropyl-2-methyl-pyrazol- 3-yl)-3-[(1S)-1-(2-pyrimidin-2-yl- 1,2,4-triazol-3-yl)ethyl]urea | 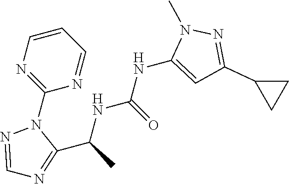 [SMILES: C[C@H](NC(=O)Nc1cc(C2CC2)nn1C)c1ncnn1-c1ncccn1] | 0.77 | 354.0 | 3 | |
| P110 | 1-(2-bromophenyl)-3-[(1S)-1-(2- pyrimidin-2-yl-1,2,4-triazol-3- yl)ethyl]urea | 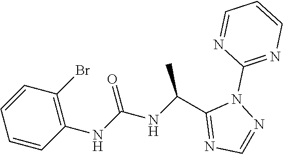 [SMILES: C[C@H](NC(=O)Nc1ccccc1Br)c1ncnn1-c1ncccn1] | 1.03 | 388.0 | 3 | |
| P111 | 1-(2-bromo-4,6-dichloro-phenyl)-3- [(1S)-1-(2-pyrimidin-2-yl-1,2,4- triazol-3-yl)ethyl]urea | 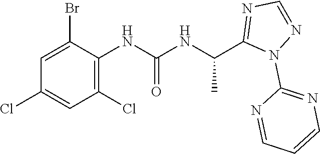 [SMILES: C[C@H](NC(=O)Nc1c(Cl)cc(Cl)cc1Br)c1ncnn1-c1ncccn1] | 1.13 | 456.0 | 3 | |
| P112 | 1-(2,3-dichlorophenyl)-3-[(1S)-1-(2- pyrimidin-2-yl-1,2,4-triazol-3- yl)ethyl]urea | 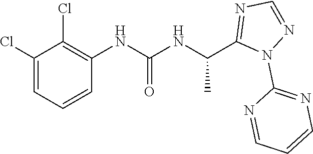 [SMILES: C[C@H](NC(=O)Nc1cccc(Cl)c1Cl)c1ncnn1-c1ncccn1] | 1.2 | 378.0 | 3 | |
| P113 | 1-(2,3-dichloro-4-pyridyl)-3-[(1S)-1- (2-pyrimidin-2-yl-1,2,4-triazol-3- yl)ethyl]urea | 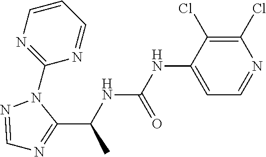 [SMILES: C[C@H](NC(=O)Nc1ccnc(Cl)c1Cl)c1ncnn1-c1ncccn1] | 1.01 | 379.0 | 3 | |
| P114 | 1-(2,4-dichlorophenyl)-1-methyl-3- [(1S)-1-(2-pyrimidin-2-yl-1,2,4- triazol-3-yl)ethyl]urea | 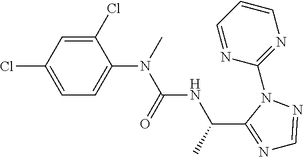 [SMILES: C[C@H](NC(=O)N(C)c1ccc(Cl)cc1Cl)c1ncnn1-c1ncccn1] | 1.19 | 392.0 | 3 | |
| P115 | 1-(2-bromo-4-methyl-phenyl)-3- [(1S)-1-(2-pyrimidin-2-yl-1,2,4- triazol-3-yl)ethyl]urea | 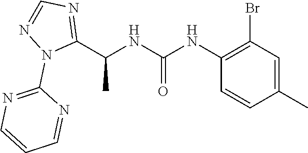 [SMILES: Cc1ccc(NC(=O)N[C@@H](C)c2ncnn2-c2ncccn2)c(Br)c1] | 0.71 | 378.0 | 3 | |
| P116 | 1-[4-fluoro-2- (trifluoromethyl)phenyl]-3-[(1S)-1- (2-pyrimidin-2-yl-1,2,4-triazol-3- yl)ethyl]urea | 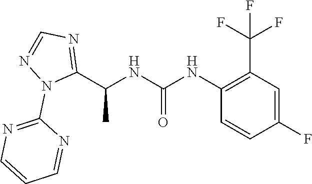 [SMILES: C[C@H](NC(=O)Nc1ccc(F)cc1C(F)(F)F)c1ncnn1-c1ncccn1] | 1.15 | 402.0 | 3 | |
| P117 | 1-(2,5-dibromophenyl)-3-[(1S)-1-(2- pyrimidin-2-yl-1,2,4-triazol-3- yl)ethyl]urea | 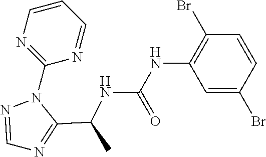 [SMILES: C[C@H](NC(=O)Nc1cc(Br)ccc1Br)c1ncnn1-c1ncccn1] | 1.09 | 396.0 | 3 | |
| P118 | 1-(4,5-dichloro-2-fluoro-phenyl)-3- [(1S)-1-(2-pyrimidin-2-yl-1,2,4- triazol-3-yl)ethyl]urea | 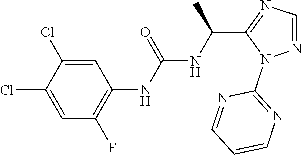 [SMILES: C[C@H](NC(=O)Nc1cc(Cl)c(Cl)cc1F)c1ncnn1-c1ncccn1] | 1.29 | 396.25 | 3 | |
| P119 | 1-[(1S)-1-(2-pyrimidin-2-yl-1,2,4- triazol-3-yl)ethyl]-3-(2,2,3,3- tetrafluoro-1,4-benzodioxin-6- yl)urea | 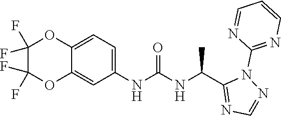 [SMILES: C[C@H](NC(=O)Nc1ccc2c(c1)OC(F)(F)C(F)(F)O2)c1ncnn1-c1ncccn1] | 1.4 | 440.0 | 3 | |
| P120 | 1-[(1S)-1-(2-pyrimidin-2-yl-1,2,4- triazol-3-yl)ethyl]-3-(2-thiazol-2- ylphenyl)urea | 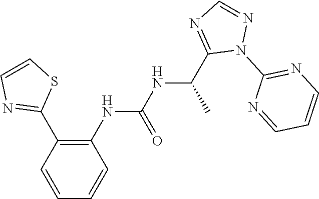 [SMILES: C[C@H](NC(=O)Nc1ccccc1-c1nccs1)c1ncnn1-c1ncccn1] | 1.17 | 393.0 | 3 | |
| P121 | 1-(3,5-dichlorophenyl)-3-[(1S)-1-(2- pyrimidin-2-yl-1,2,4-triazol-3- yl)ethyl]urea | 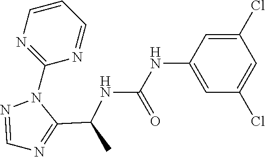 [SMILES: C[C@H](NC(=O)Nc1cc(Cl)cc(Cl)c1)c1ncnn1-c1ncccn1] | 1.32 | 378.0 | 3 | |
| P122 | 1-[(1S)-1-(2-pyrimidin-2-yl-1,2,4- triazol-3-yl)ethyl]-3-[4- (trifluoromethyl)phenyl]urea | 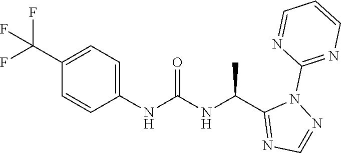 [SMILES: C[C@H](NC(=O)Nc1ccc(C(F)(F)F)cc1)c1ncnn1-c1ncccn1] | 1.21 | 378.0 | 3 | |
| P123 | 1-(3-bromo-2,6-dimethyl-phenyl)-3- [(1S)-1-(2-pyrimidin-2-yl-1,2,4- triazol-3-yl)ethyl]urea | 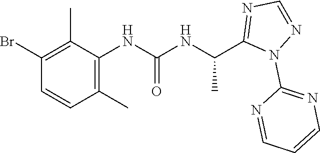 [SMILES: Cc1ccc(Br)c(C)c1NC(=O)N[C@@H](C)c1ncnn1-c1ncccn1] | 1.13 | 416.0 | 3 | |
| P124 | 1-(3-cyanophenyl)-3-[(1S)-1-(2- pyrimidin-2-yl-1,2,4-triazol-3- yl)ethyl]urea | 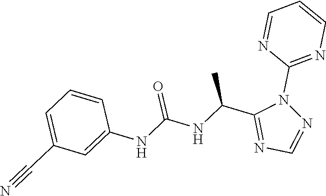 [SMILES: C[C@H](NC(=O)Nc1cccc(C#N)c1)c1ncnn1-c1ncccn1] | 0.9 | 335.0 | 3 | |
| P125 | 1-(2-bromo-6-fluoro-phenyl)-3- [(1S)-1-(2-pyrimidin-2-yl-1,2,4- triazol-3-yl)ethyl]urea | 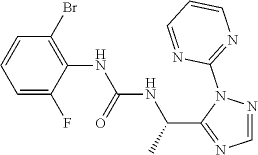 [SMILES: C[C@H](NC(=O)Nc1c(F)cccc1Br)c1ncnn1-c1ncccn1] | 0.88 | 406.0 | 3 | |
| P126 | 1-(5-bromo-6-chloro-2-pyridyl)-3- [(1S)-1-(2-pyrimidin-2-yl-1,2,4- triazol-3-yl)ethyl]urea | 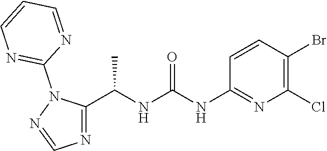 [SMILES: C[C@H](NC(=O)Nc1ccc(Br)c(Cl)n1)c1ncnn1-c1ncccn1] | 1.24 | 423.0 | 3 | |
| P127 | 1-[2-chloro-5-(trifluoromethyl)-3- pyridyl]-3-[(1S)-1-(2-pyrimidin-2-yl- 1,2,4-triazol-3-yl)ethyl]urea | 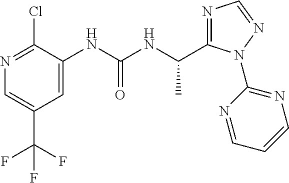 [SMILES: C[C@H](NC(=O)Nc1cc(C(F)(F)F)cnc1Cl)c1ncnn1-c1ncccn1] | 1.21 | 413.0 | 3 | |
| P128 | 1-[3-chloro-4- (trifluoromethyl)phenyl]-3-[(1S)-1- (2-pyrimidin-2-yl-1,2,4-triazol-3- yl)ethyl]urea | 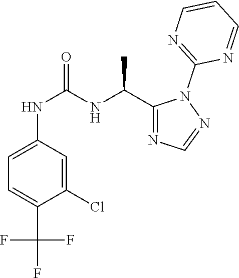 [SMILES: C[C@H](NC(=O)Nc1ccc(C(F)(F)F)c(Cl)c1)c1ncnn1-c1ncccn1] | 1.36 | 412.0 | 3 | 103 - 105 |
| P129 | 1-[2-chloro-4- (trifluoromethyl)phenyl]-3-[(1S)-1- (2-pyrimidin-2-yl-1,2,4-triazol-3- yl)ethyl]thiourea | 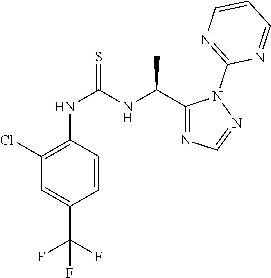 [SMILES: C[C@H](NC(=S)Nc1ccc(C(F)(F)F)cc1Cl)c1ncnn1-c1ncccn1] | 1.03 | 426.4 | 1 | 168 - 170 |
| P130 | 1-(2,4-dibromophenyl)-3-[(1S)-1-(2- pyrimidin-2-yl-1,2,4-triazol-3- yl)ethyl]urea | 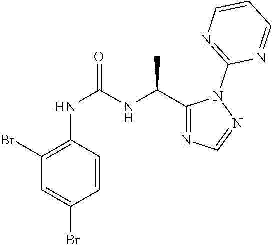 [SMILES: C[C@H](NC(=O)Nc1ccc(Br)cc1Br)c1ncnn1-c1ncccn1] | 1.29 | 465.0 | 3 | 220 - 222 |
| P131 | 1-[4-chloro-3- (trifluoromethyl)phenyl]-3-[(1S)-1- (2-pyrimidin-2-yl-1,2,4-triazol-3- yl)ethyl]urea | 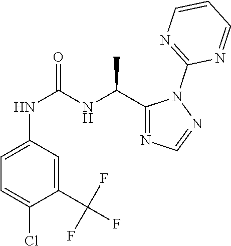 [SMILES: C[C@H](NC(=O)Nc1ccc(Cl)c(C(F)(F)F)c1)c1ncnn1-c1ncccn1] | 1.34 | 412.0 | 3 | 106 - 108 |
| P132 | 1-[2,4-dichloro-5-(1,1,2,2- tetrafluoroethoxy)phenyl]-1,3- dimethyl-3-[(1S)-1-(2-pyrimidin-2-yl- 1,2,4-triazol-3-yl)ethyl]urea | 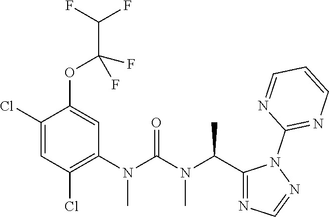 [SMILES: C[C@@H](c1ncnn1-c1ncccn1)N(C)C(=O)N(C)c1cc(OC(F)(F)C(F)F)c(Cl)cc1Cl] | 1.01 | 522.2 | 2 | 115 - 119 |
| P133 | 3-[2,4-dichloro-5-(1,1,2,2- tetrafluoroethoxy)phenyl]-1-methyl- 1-[(1S)-1-(2-pyrimidin-2-yl-1,2,4- triazol-3-yl)ethyl]urea | 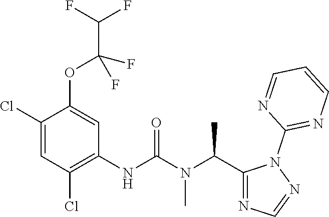 [SMILES: C[C@@H](c1ncnn1-c1ncccn1)N(C)C(=O)Nc1cc(OC(F)(F)C(F)F)c(Cl)cc1Cl] | 1.01 | 508.0 | 2 | 89 - 91 |
| P134 | 1-[2,4-dichloro-5-(1,1,2,2- tetrafluoroethoxy)phenyl]-3-[(1S)-1- (2-pyrimidin-2-yl-1,2,4-triazol-3- yl)ethyl]imidazolidin-2-one | 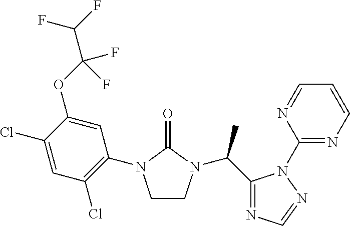 [SMILES: C[C@@H](c1ncnn1-c1ncccn1)N1CCN(c2cc(OC(F)(F)C(F)F)c(Cl)cc2Cl)C1=O] | 0.98 | 520.1 | 2 | 207 - 209 |
| P135 | 1-[2,4-dichloro-5-(1,1,2,2- tetrafluoroethoxy)phenyl]-3-[(1S)-1- (3-pyrimidin-2-ylpyrazin-2- yl)ethyl]urea | 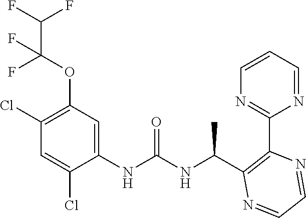 [SMILES: C[C@H](NC(=O)Nc1cc(OC(F)(F)C(F)F)c(Cl)cc1Cl)c1nccnc1-c1ncccn1] | 2.5 | | 5 | |
| P136 | 1-[2,4-dichloro-5-(1,1,2,2- tetrafluoroethoxy)phenyl]-3-[(1R)-1- (3-pyrimidin-2-ylpyrazin-2- yl)ethyl]urea | 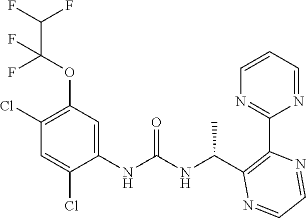 [SMILES: C[C@@H](NC(=O)Nc1cc(OC(F)(F)C(F)F)c(Cl)cc1Cl)c1nccnc1-c1ncccn1] | 1.36 | | 5 | |
| P137 | 3-[2,4-dichloro-5-(1,1,2,2- tetrafluoroethoxy)phenyl]-5-[(1S)-1- (2-pyrimidin-2-yl-1,2,4-triazol-3- yl)ethyl]-1,3,5-oxadiazinan-4-one | 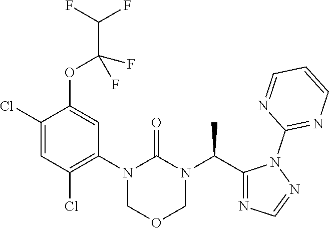 [SMILES: C[C@@H](c1ncnn1-c1ncccn1)N1COCN(c2cc(OC(F)(F)C(F)F)c(Cl)cc2Cl)C1=O] | 0.96 | 536.2 | 2 | 114 - 116 |
| P138 | 1-(2,4-dichloro-5-pyrimidin-2-yloxy- phenyl)-3-[(1S)-1-(2-pyrimidin-2-yl- 1,2,4-triazol-3-yl)ethyl]urea | 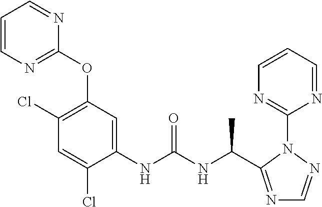 [SMILES: C[C@H](NC(=O)Nc1cc(Oc2ncccn2)c(Cl)cc1Cl)c1ncnn1-c1ncccn1] | 0.81 | 472.1 | 2 | 113 - 115 |
| P139 | 1-[2,4-dichloro-5- (trifluoromethyl)phenyl]-3-[(1S)-1-[2- [5-[(2R,6S)-2,6-dimethylmorpholine- 4-carbonyl]-2-pyridyl]-1,2,4-triazol- 3-yl]ethyl]urea | 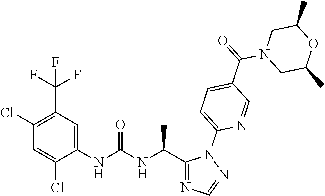 [SMILES: C[C@@H]1CN(C(=O)c2ccc(-n3ncnc3[C@H](C)NC(=O)Nc3cc(C(F)(F)F)c(Cl)cc3Cl)nc2)C[C@H](C)O1] | 1.17 | 588.1 | 4 | 138 - 140 |
| P140 | 1-[(1S)-1-[2-(5-cyanothiazol-2-yl)- 1,2,4-triazol-3-yl]ethyl]-3-[2,4- dichloro-5- (trifluoromethyl)phenyl]urea | 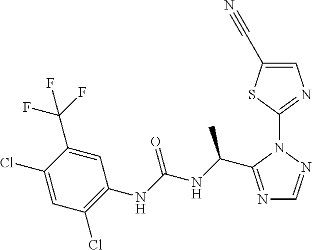 [SMILES: C[C@H](NC(=O)Nc1cc(C(F)(F)F)c(Cl)cc1Cl)c1ncnn1-c1ncc(C#N)s1] | 1.25 | 476.0 | 4 | 201 - 204 |
| P141 | 1-[2,4-dichloro-5-[(2-chloro-4- pyridyl)methoxy]phenyl]-3-[(1S)-1- (2-pyrimidin-2-yl-1,2,4-triazol-3- yl)ethyl]urea | 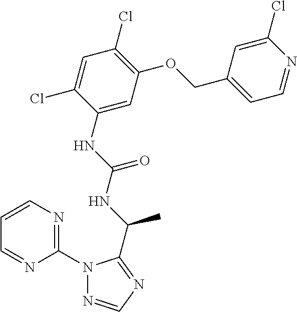 [SMILES: C[C@H](NC(=O)Nc1cc(OCc2ccnc(Cl)c2)c(Cl)cc1Cl)c1ncnn1-c1ncccn1] | 1.62 | 519.27 | 3 | |
| P142 | 1-[2,4-dichloro-5-[(5-chloro-2- pyridyl)methoxy]phenyl]-3-[(1S)-1- (2-pyrimidin-2-yl-1,2,4-triazol-3- yl)ethyl]urea | 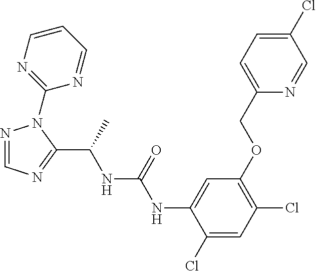 [SMILES: C[C@H](NC(=O)Nc1cc(OCc2ccc(Cl)cn2)c(Cl)cc1Cl)c1ncnn1-c1ncccn1] | 1.67 | 519.3 | 3 | |
| P143 | 1-[2,4-dichloro-5-[(2-chloro-3- pyridyl)methoxy]phenyl]-3-[(1S)-1- (2-pyrimidin-2-yl-1,2,4-triazol-3- yl)ethyl]urea | 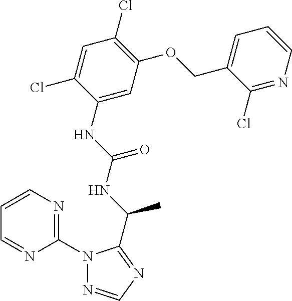 [SMILES: C[C@H](NC(=O)Nc1cc(OCc2cccnc2Cl)c(Cl)cc1Cl)c1ncnn1-c1ncccn1] | 1.58 | 519.31 | 3 | |
| P144 | 1-[2,4-dichloro-5- (cyclobutylmethoxy)phenyl]-3-[(1S)- 1-(2-pyrimidin-2-yl-1,2,4-triazol-3- yl)ethyl]urea | 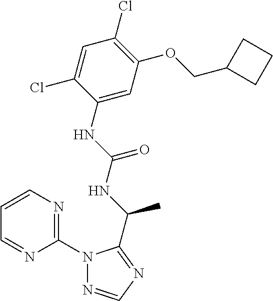 [SMILES: C[C@H](NC(=O)Nc1cc(OCC2CCC2)c(Cl)cc1Cl)c1ncnn1-c1ncccn1] | 1.82 | 462.3 | 3 | |
| P145 | 1-[5-[(6-bromo-2-pyridyl)methoxy]- 2,4-dichloro-phenyl]-3-[(1S)-1-(2- pyrimidin-2-yl-1,2,4-triazol-3- yl)ethyl]urea | 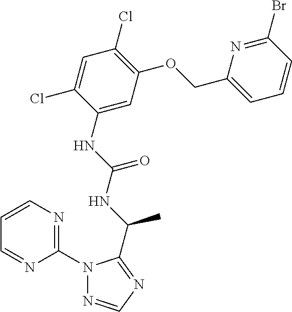 [SMILES: C[C@H](NC(=O)Nc1cc(OCc2cccc(Br)n2)c(Cl)cc1Cl)c1ncnn1-c1ncccn1] | 1.68 | 563.26 | 3 | |
| P146 | 1-(2,4-dichloro-5- tetrahydrothiopyran-4-yloxy- phenyl)-3-[(1S)-1-(2-pyrimidin-2-yl- 1,2,4-triazol-3-yl)ethyl]urea | 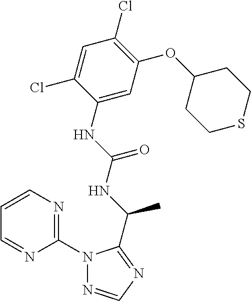 [SMILES: C[C@H](NC(=O)Nc1cc(OC2CCSCC2)c(Cl)cc1Cl)c1ncnn1-c1ncccn1] | 1.7 | 494.3 | 3 | |
| P147 | 1-[2,4-dichloro-5-(tetrahydrofuran-3- ylmethoxy)phenyl]-3-[(1S)-1-(2- pyrimidin-2-yl-1,2,4-triazol-3- yl)ethyl]urea | 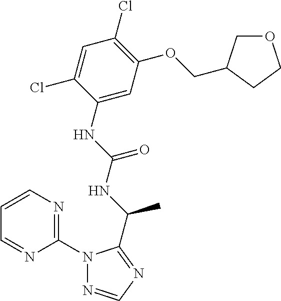 [SMILES: C[C@H](NC(=O)Nc1cc(OCC2CCOC2)c(Cl)cc1Cl)c1ncnn1-c1ncccn1] | 1.45 | 478.32 | 3 | |
| P148 | 1-[5-[(2-bromo-4-pyridyl)methoxy]- 2,4-dichloro-phenyl]-3-[(1S)-1-(2- pyrimidin-2-yl-1,2,4-triazol-3- yl)ethyl]urea | 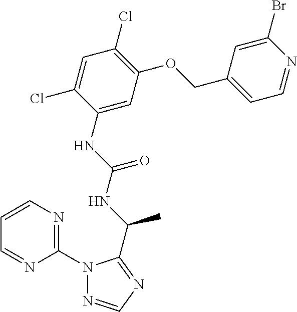 [SMILES: C[C@H](NC(=O)Nc1cc(OCc2ccnc(Br)c2)c(Cl)cc1Cl)c1ncnn1-c1ncccn1] | 1.65 | 563.25 | 3 | |
| P149 | 1-[2,4-dichloro-5-[(6-fluoro-2- pyridyl)methoxy]phenyl]-3-[(1S)-1- (2-pyrimidin-2-yl-1,2,4-triazol-3- yl)ethyl]urea | 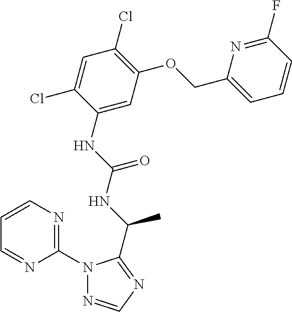 [SMILES: C[C@H](NC(=O)Nc1cc(OCc2cccc(F)n2)c(Cl)cc1Cl)c1ncnn1-c1ncccn1] | 1.57 | 503.3 | 3 | |
| P150 | 1-[5-[(2-bromothiazol-5-yl)methoxy]- 2,4-dichloro-phenyl]-3-[(1S)-1-(2- pyrimidin-2-yl-1,2,4-triazol-3- yl)ethyl]urea | 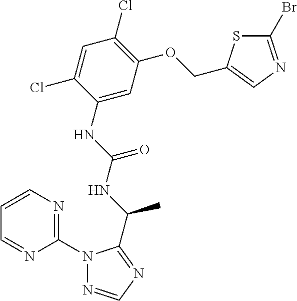 [SMILES: C[C@H](NC(=O)Nc1cc(OCc2cnc(Br)s2)c(Cl)cc1Cl)c1ncnn1-c1ncccn1] | 1.68 | 569.21 | 3 | |
| P151 | 1-[5-[3-(benzenesulfonyl)propoxy]- 2,4-dichloro-phenyl]-3-[(1S)-1-(2- pyrimidin-2-yl-1,2,4-triazol-3- yl)ethyl]urea | 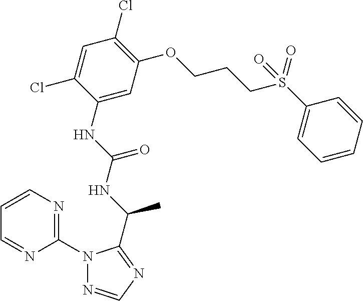 [SMILES: C[C@H](NC(=O)Nc1cc(OCCCS(=O)(=O)c2ccccc2)c(Cl)cc1Cl)c1ncnn1-c1ncccn1] | 1.54 | 576.35 | 3 | |
| P152 | 1-[2,4-dichloro-5-(2-pyrazol-1- ylethoxy)phenyl]-3-[(1S)-1-(2- pyrimidin-2-yl-1,2,4-triazol-3- yl)ethyl]urea | 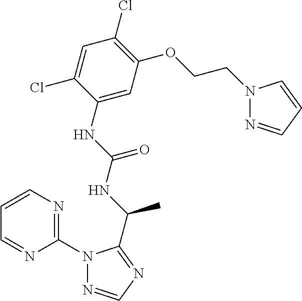 [SMILES: C[C@H](NC(=O)Nc1cc(OCCn2cccn2)c(Cl)cc1Cl)c1ncnn1-c1ncccn1] | 1.39 | 488.31 | 3 | |
| P153 | 1-[2,4-dichloro-5-(2-pyrrol-1- ylethoxy)phenyl]-3-[(1S)-1-(2- pyrimidin-2-yl-1,2,4-triazol-3- yl)ethyl]urea | 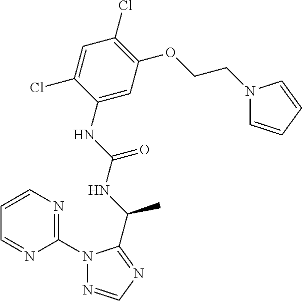 [SMILES: C[C@H](NC(=O)Nc1cc(OCCn2cccc2)c(Cl)cc1Cl)c1ncnn1-c1ncccn1] | 1.62 | 487.31 | 3 | |
| P154 | 1-[5-(1,2-benzoxazol-3-ylmethoxy)- 2,4-dichloro-phenyl]-3-[(1S)-1-(2- pyrimidin-2-yl-1,2,4-triazol-3- yl)ethyl]urea | 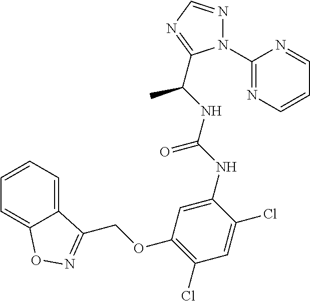 [SMILES: C[C@H](NC(=O)Nc1cc(OCc2noc3ccccc23)c(Cl)cc1Cl)c1ncnn1-c1ncccn1] | 1.68 | 525.32 | 3 | |
| P155 | 1-[2,4-dichloro-5-[2-(2- methoxyethoxy)ethoxy]phenyl]-3- [(1S)-1-(2-pyrimidin-2-yl-1,2,4- triazol-3-yl)ethyl]urea | 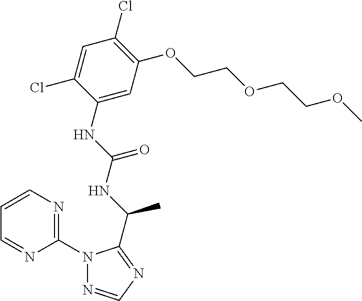 [SMILES: COCCOCCOc1cc(NC(=O)N[C@@H](C)c2ncnn2-c2ncccn2)c(Cl)cc1Cl] | 1.39 | 496.32 | 3 | |
| P156 | 1-[2,4-dichloro-5-[(3- cyanophenyl)methoxy]phenyl]-3- [(1S)-1-(2-pyrimidin-2-yl-1,2,4- triazol-3-yl)ethyl]urea | 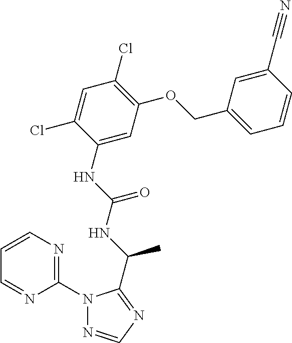 [SMILES: C[C@H](NC(=O)Nc1cc(OCc2cccc(C#N)c2)c(Cl)cc1Cl)c1ncnn1-c1ncccn1] | 1.64 | 509.32 | 3 | |
| P157 | 1-[2,4-dichloro-5-(4- pyridylmethoxy)phenyl]-3-[(1S)-1- (2-pyrimidin-2-yl-1,2,4-triazol-3- yl)ethyl]urea | 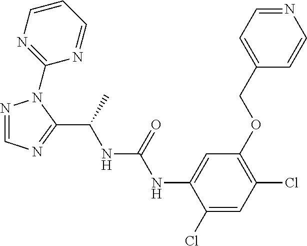 [SMILES: C[C@H](NC(=O)Nc1cc(OCc2ccncc2)c(Cl)cc1Cl)c1ncnn1-c1ncccn1] | 1.05 | 485.29 | 3 | |
| P158 | 1-[2,4-dichloro-5-[2-(1- methylpyrazol-4-yl)ethoxy]phenyl]- 3-[(1S)-1-(2-pyrimidin-2-yl-1,2,4- triazol-3-yl)ethyl]urea | 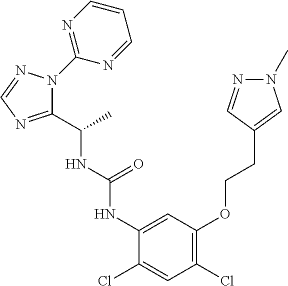 [SMILES: C[C@H](NC(=O)Nc1cc(OCCc2cnn(C)c2)c(Cl)cc1Cl)c1ncnn1-c1ncccn1] | 1.39 | 502.33 | 3 | |
| P159 | 1-[2,4-dichloro-5-[(3-cyano-4-fluoro- phenyl)methoxy]phenyl]-3-[(1S)-1- (2-pyrimidin-2-yl-1,2,4-triazol-3- yl)ethyl]urea | 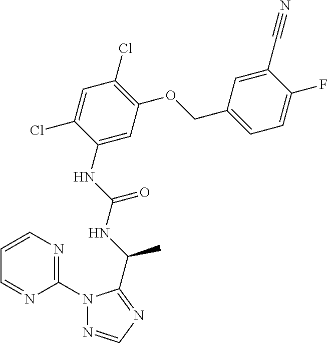 [SMILES: C[C@H](NC(=O)Nc1cc(OCc2ccc(F)c(C#N)c2)c(Cl)cc1Cl)c1ncnn1-c1ncccn1] | 1.7 | 527.3 | 3 | |
| P160 | 1-(5-allyloxy-2,4-dichloro-phenyl)-3- [(1S)-1-(2-pyrimidin-2-yl-1,2,4- triazol-3-yl)ethyl]urea | 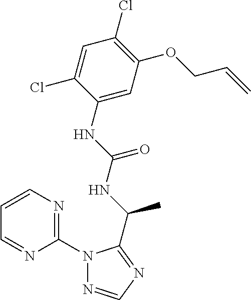 [SMILES: C=CCOc1cc(NC(=O)N[C@@H](C)c2ncnn2-c2ncccn2)c(Cl)cc1Cl] | 1.54 | 434.25 | 3 | |
| P161 | 1-[2,4-dichloro-5- (cyanomethoxy)phenyl]-3-[(1S)-1- (2-pyrimidin-2-yl-1,2,4-triazol-3- yl)ethyl]urea | 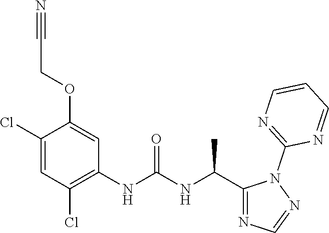 [SMILES: C[C@H](NC(=O)Nc1cc(OCC#N)c(Cl)cc1Cl)c1ncnn1-c1ncccn1] | 1.32 | 433.24 | 3 | |
| P162 | 1-[2,4-dichloro-5-[(4- fluorophenyl)methoxy]phenyl]-3- [(1S)-1-(2-pyrimidin-2-yl-1,2,4- triazol-3-yl)ethyl]urea | 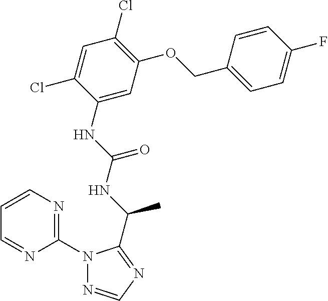 [SMILES: C[C@H](NC(=O)Nc1cc(OCc2ccc(F)cc2)c(Cl)cc1Cl)c1ncnn1-c1ncccn1] | 1.75 | 502.3 | 3 | |
| P163 | 1-(5-benzyloxy-2,4-dichloro- phenyl)-3-[(1S)-1-(2-pyrimidin-2-yl- 1,2,4-triazol-3-yl)ethyl]urea | 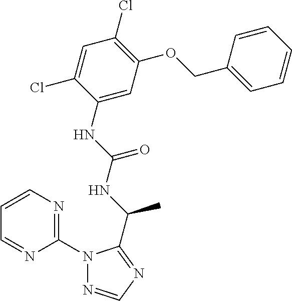 [SMILES: C[C@H](NC(=O)Nc1cc(OCc2ccccc2)c(Cl)cc1Cl)c1ncnn1-c1ncccn1] | 1.73 | 484.3 | 3 | |
| P164 | tert-butyl 3-[[2,4-dichloro-5-[[(1S)-1- (2-pyrimidin-2-yl-1,2,4-triazol-3- yl)ethyl]carbamoylamino]phenoxy] methyl]azetidine-1-carboxylate | 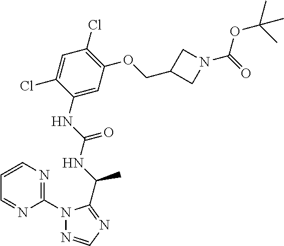 [SMILES: C[C@H](NC(=O)Nc1cc(OCC2CN(C(=O)OC(C)(C)C)C2)c(Cl)cc1Cl)c1ncnn1-c1ncccn1] | 1.73 | 563.39 | 3 | |
| P165 | 1-[2,4-dichloro-5-[(4-cyano-3-fluoro- phenyl)methoxy]phenyl]-3-[(1S)-1- (2-pyrimidin-2-yl-1,2,4-triazol-3- yl)ethyl]urea | 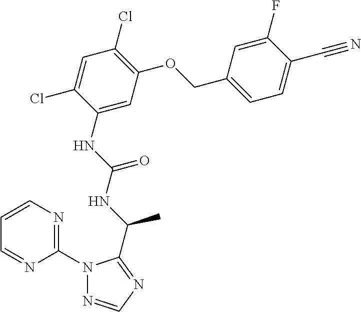 [SMILES: C[C@H](NC(=O)Nc1cc(OCc2ccc(C#N)c(F)c2)c(Cl)cc1Cl)c1ncnn1-c1ncccn1] | 1.71 | 527.3 | 3 | |
| P166 | 1-[2,4-dichloro-5-[(4-cyano-2-fluoro- phenyl)methoxy]phenyl]-3-[(1S)-1- (2-pyrimidin-2-yl-1,2,4-triazol-3- yl)ethyl]urea | 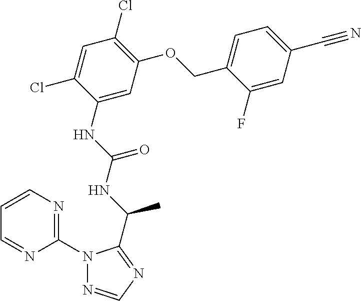 [SMILES: C[C@H](NC(=O)Nc1cc(OCc2ccc(C#N)cc2F)c(Cl)cc1Cl)c1ncnn1-c1ncccn1] | 1.67 | 527.3 | 3 | |
| P167 | 1-[2,4-dichloro-5-[(4- cyanophenyl)methoxy]phenyl]-3- [(1S)-1-(2-pyrimidin-2-yl-1,2,4- triazol-3-yl)ethyl]urea | 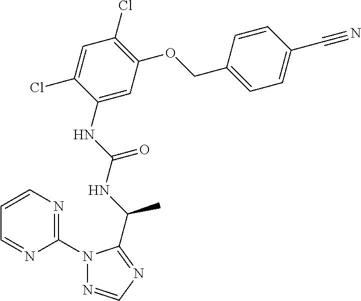 [SMILES: C[C@H](NC(=O)Nc1cc(OCc2ccc(C#N)cc2)c(Cl)cc1Cl)c1ncnn1-c1ncccn1] | 1.65 | 509.32 | 3 | |
| P168 | 1-[2,4-dichloro-5-[(3-fluoro-4- methoxy-phenyl)methoxy]phenyl]-3- [(1S)-1-(2-pyrimidin-2-yl-1,2,4- triazol-3-yl)ethyl]urea | 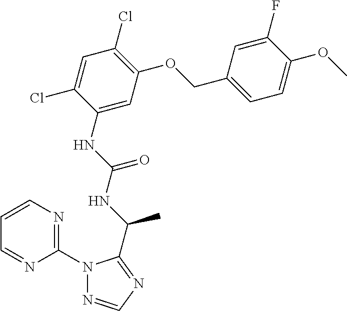 [SMILES: COc1ccc(COc2cc(NC(=O)N[C@@H](C)c3ncnn3-c3ncccn3)c(Cl)cc2Cl)cc1F] | 1.72 | 532.33 | 3 | |
| P169 | 1-[2,4-dichloro-5-[(2-fluoro-4- methylsulfonyl- phenyl)methoxy]phenyl]-3-[(1S)-1- (2-pyrimidin-2-yl-1,2,4-triazol-3- yl)ethyl]urea | 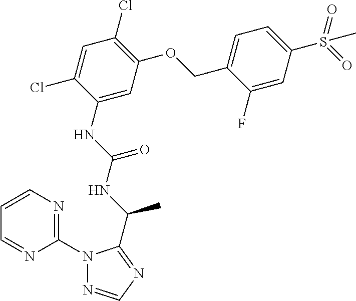 [SMILES: C[C@H](NC(=O)Nc1cc(OCc2ccc(S(C)(=O)=O)cc2F)c(Cl)cc1Cl)c1ncnn1-c1ncccn1] | 1.5 | 580.31 | 3 | |
| P170 | 2-[5-[(1S)-1-[[2,4-dichloro-5- (trifluoromethyl)phenyl]carbamoyl- amino]ethyl]-1,2,4-triazol-1-yl]-N, N- dimethyl-thiazole-5-carboxamide | 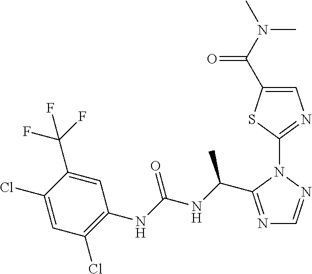 [SMILES: C[C@H](NC(=O)Nc1cc(C(F)(F)F)c(Cl)cc1Cl)c1ncnn1-c1ncc(C(=O)N(C)C)s1] | 1.17 | 524.0 | 4 | 158 - 160 |
| P171 | N-[1-[2-(5-cyano-2-pyridyl)-1,2,4- triazol-3-yl]ethyl]-5-(trifluoromethyl)- 2,3-dihydropyrrolo[2,3-c]pyridine-1- carboxamide | 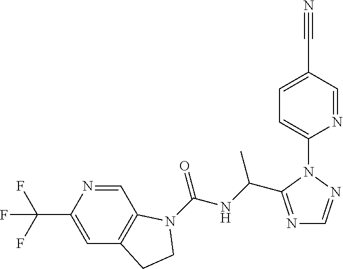 [SMILES: CC(NC(=O)N1CCc2cc(C(F)(F)F)ncc21)c1ncnn1-c1ccc(C#N)cn1] | 0.89 | 429.0 | 2 | |
| P172 | N-[(1S)-1-(2-pyrimidin-2-yl-1,2,4- triazol-3-yl)ethyl]-5-(trifluoromethyl)- 2,3-dihydropyrrolo[2,3-c]pyridine-1- carboxamide | 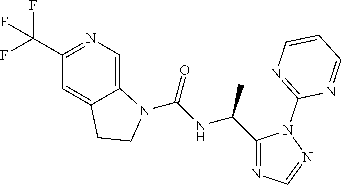 [SMILES: C[C@H](NC(=O)N1CCc2cc(C(F)(F)F)ncc21)c1ncnn1-c1ncccn1] | 0.78 | 405.0 | 2 | 132 - 135 |
| P173 | 1-[(1S)-1-[2-(5-cyano-2-pyridyl)- 1,2,4-triazol-3-yl]ethyl]-3-[2,4- dichloro-5- (trifluoromethyl)phenyl]urea | 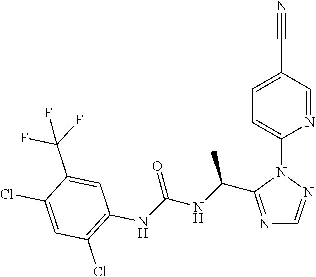 [SMILES: C[C@H](NC(=O)Nc1cc(C(F)(F)F)c(Cl)cc1Cl)c1ncnn1-c1ccc(C#N)cn1] | 1.18 | 470.1 | 4 | 196 - 198 |
| P174 | 6-[5-[(1S)-1-[[2,4-dichloro-5- (trifluoromethyl)phenyl]carbamoyl- amino]ethyl]-1,2,4-triazol-1- yl]pyridine-3-carboxylic acid | 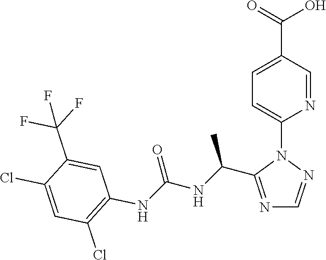 [SMILES: C[C@H](NC(=O)Nc1cc(C(F)(F)F)c(Cl)cc1Cl)c1ncnn1-c1ccc(C(=O)O)cn1] | 1.1 | 491.1 | 4 | |
| P175 | 1-[2,4-dichloro-5- (trifluoromethyl)phenyl]-3-[(1S)-1-[2- [5-(morpholine-4-carbonyl)-2- pyridyl]-1,2,4-triazol-3-yl]ethyl]urea | 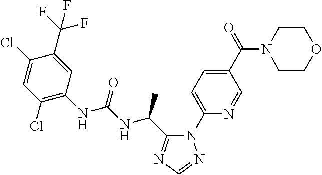 [SMILES: C[C@H](NC(=O)Nc1cc(C(F)(F)F)c(Cl)cc1Cl)c1ncnn1-c1ccc(C(=O)N2CCOCC2)cn1] | 1.11 | 560.0 | 4 | 130 - 132 |
| P176 | 1-[2,4-dichloro-5-(1,1,2,2- tetrafluoroethoxy)phenyl]-1-methyl- 3-[(1S)-1-(2-pyrimidin-2-yl-1,2,4- triazol-3-yl)ethyl]urea | 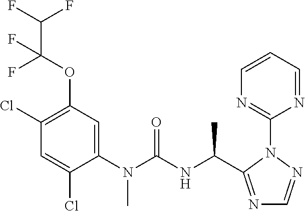 [SMILES: C[C@H](NC(=O)N(C)c1cc(OC(F)(F)C(F)F)c(Cl)cc1Cl)c1ncnn1-c1ncccn1] | 0.95 | 508.1 | 2 | 104 - 105 |
| P177 | 1-[2,4-dichloro-5-(1,1,2,2- tetrafluoroethoxy)phenyl]-3-[1-(2- pyrimidin-2-yl-1,2,4-triazol-3- yl)ethyl]urea | 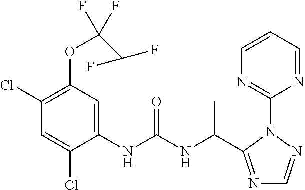 [SMILES: CC(NC(=O)Nc1cc(OC(F)(F)C(F)F)c(Cl)cc1Cl)c1ncnn1-c1ncccn1] | 0.99 | 494.0 | 2 | 231 - 233 |
| P178 | 1-[2,4-dichloro-5- (trifluoromethyl)phenyl]-3-[(1S)-1- (2-pyrimidin-2-yl-1,2,4-triazol-3- yl)ethyl]urea | 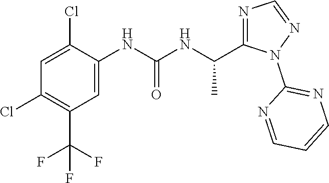 [SMILES: C[C@H](NC(=O)Nc1cc(C(F)(F)F)c(Cl)cc1Cl)c1ncnn1-c1ncccn1] | 1.52 | 446.15 | 3 | |
| P179 | 1-(2,5-dichloro-4-cyano-phenyl)-3- [(1S)-1-(2-pyrimidin-2-yl-1,2,4- triazol-3-yl)ethyl]urea | 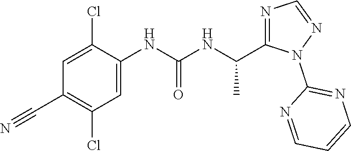 [SMILES: C[C@H](NC(=O)Nc1cc(Cl)c(C#N)cc1Cl)c1ncnn1-c1ncccn1] | 1.25 | 403.15 | 3 | |
| P180 | 1-[2,4-dichloro-5-(3,3- dimethylbutoxy)phenyl]-3-[(1S)-1- (2-pyrimidin-2-yl-1,2,4-triazol-3- yl)ethyl]urea | 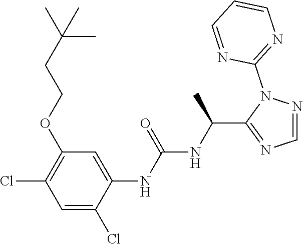 [SMILES: C[C@H](NC(=O)Nc1cc(OCCC(C)(C)C)c(Cl)cc1Cl)c1ncnn1-c1ncccn1] | 1.9 | 478.26 | 3 | |
| P181 | [2-fluoro-4-methyl-5-[[(1S)-1-(2- pyrimidin-2-yl-1,2,4-triazol-3- yl)ethyl]carbamoylamino]phenyl] methanesulfonate | 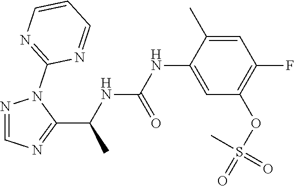 [SMILES: Cc1cc(F)c(OS(C)(=O)=O)cc1NC(=O)N[C@@H](C)c1ncnn1-c1ncccn1] | 1 | 436.22 | 3 | |
| P182 | 1-(4-chloro-5-cyano-2-fluoro- phenyl)-3-[(1S)-1-(2-pyrimidin-2-yl- 1,2,4-triazol-3-yl)ethyl]urea | 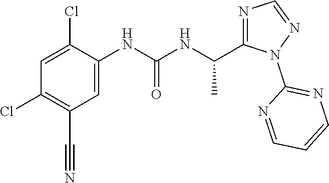 [SMILES: C[C@H](NC(=O)Nc1cc(C#N)c(Cl)cc1Cl)c1ncnn1-c1ncccn1] | 1.13 | 387.19 | 3 | |
| P183 | 1-[2,5-dichloro-4- (isobutylsulfamoyl)phenyl]-3-[(1S)- 1-(2-pyrimidin-2-yl-1,2,4-triazol-3- yl)ethyl]urea | 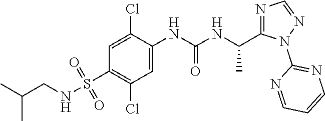 [SMILES: CC(C)CNS(=O)(=O)c1cc(Cl)c(NC(=O)N[C@@H](C)c2ncnn2-c2ncccn2)cc1Cl] | 1.49 | 513.22 | 3 | |
| P184 | 1-[4-chloro-2-fluoro-5-[[2-methyl-5- (trifluoromethyl)-1,2,4-triazol-3- yl]oxy]phenyl]-3-[(1S)-1-(2- pyrimidin-2-yl-1,2,4-triazol-3- yl)ethyl]urea | 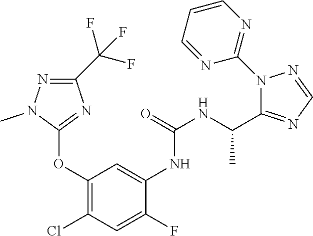 [SMILES: C[C@H](NC(=O)Nc1cc(Oc2nc(C(F)(F)F)nn2C)c(Cl)cc1F)c1ncnn1-c1ncccn1] | 1.43 | 527.24 | 3 | |
| P185 | 1-[2,5-dichloro-4-(1,1- difluoroethoxy)phenyl]-3-[(1S)-1-(2- pyrimidin-2-yl-1,2,4-triazol-3- yl)ethyl]urea | 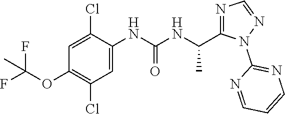 [SMILES: C[C@H](NC(=O)Nc1cc(Cl)c(OC(C)(F)F)cc1Cl)c1ncnn1-c1ncccn1] | 1.46 | 458.16 | 3 | |
| P186 | 1-(5-chloro-2,4-dimethyl-phenyl)-3- [(1S)-1-(2-pyrimidin-2-yl-1,2,4- triazol-3-yl)ethyl]urea | 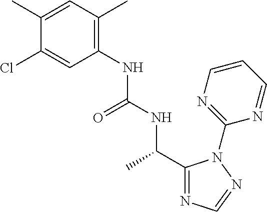 [SMILES: Cc1cc(C)c(NC(=O)N[C@@H](C)c2ncnn2-c2ncccn2)cc1Cl] | 1.26 | 372.22 | 3 | |
| P187 | 2,4-dichloro-N-isopropyl-5-[[(1S)-1- (2-pyrimidin-2-yl-1,2,4-triazol-3- yl)ethyl]carbamoylamino]benzamide | 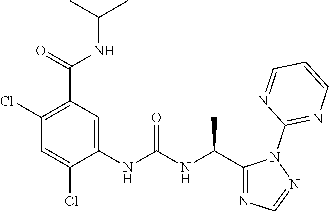 [SMILES: CC(C)NC(=O)c1cc(NC(=O)N[C@@H](C)c2ncnn2-c2ncccn2)c(Cl)cc1Cl] | 1.11 | 463.22 | 3 | |
| P188 | [5-chloro-4-methyl-2-[[(1S)-1-(2- pyrimidin-2-yl-1,2,4-triazol-3- yl)ethyl]carbamoylamino]phenyl] methanesulfonate | 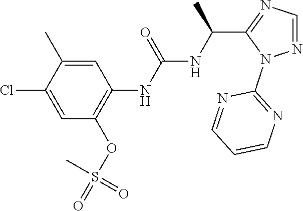 [SMILES: Cc1cc(NC(=O)N[C@@H](C)c2ncnn2-c2ncccn2)c(OS(C)(=O)=O)cc1Cl] | 1.24 | 452.18 | 3 | |
| P189 | 1-[5-(1,1-dioxo-1,2-thiazolidin-2-yl)- 2,4-dimethyl-phenyl]-3-[(1S)-1-(2- pyrimidin-2-yl-1,2,4-triazol-3- yl)ethyl]urea | 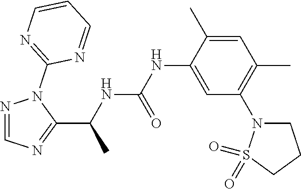 [SMILES: Cc1cc(C)c(N2CCCS2(=O)=O)cc1NC(=O)N[C@@H](C)c1ncnn1-c1ncccn1] | 0.96 | 457.27 | 3 | |
| P190 | 1-[2,5-dichloro-4-(1,1,2- trifluoroethoxy)phenyl]-3-[(1S)-1-(2- pyrimidin-2-yl-1,2,4-triazol-3- yl)ethyl]urea | 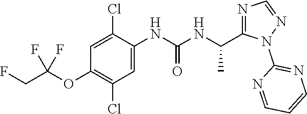 [SMILES: C[C@H](NC(=O)Nc1cc(Cl)c(OC(F)(F)CF)cc1Cl)c1ncnn1-c1ncccn1] | 1.41 | 476.16 | 3 | |
| P191 | 1-(4,5-dichloro-2-methyl-phenyl)-3- [(1S)-1-(2-pyrimidin-2-yl-1,2,4- triazol-3-yl)ethyl]urea | 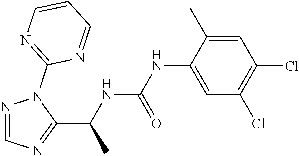 [SMILES: Cc1cc(Cl)c(Cl)cc1NC(=O)N[C@@H](C)c1ncnn1-c1ncccn1] | 1.33 | 392.18 | 3 | |
| P192 | 1-(5-chloro-4-methoxy-2- methylsulfonyl-phenyl)-3-[(1S)-1-(2- pyrimidin-2-yl-1,2,4-triazol-3- yl)ethyl]urea | 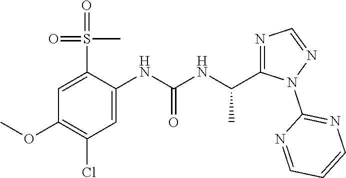 [SMILES: COc1cc(S(C)(=O)=O)c(NC(=O)N[C@@H](C)c2ncnn2-c2ncccn2)cc1Cl] | 1.08 | 452.18 | 3 | |
| P193 | methyl 4-chloro-2-fluoro-5-[[(1S)-1- (2-pyrimidin-2-yl-1,2,4-triazol-3- yl)ethyl]carbamoylamino]benzoate | 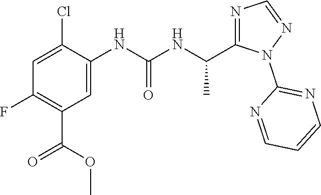 [SMILES: COC(=O)c1cc(NC(=O)N[C@@H](C)c2ncnn2-c2ncccn2)c(Cl)cc1F] | 1.11 | 420.18 | 3 | |
| P194 | 1-(2,5-dichloro-4-ethoxy-phenyl)-3- [(1S)-1-(2-pyrimidin-2-yl-1,2,4- triazol-3-yl)ethyl]urea | 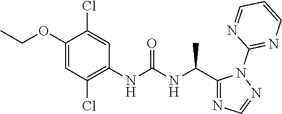 [SMILES: CCOc1cc(Cl)c(NC(=O)N[C@@H](C)c2ncnn2-c2ncccn2)cc1Cl] | 1.31 | 422.17 | 3 | |
| P195 | 1-[2,4-dichloro-5-(1,3- dioxoisoindolin-2-yl)phenyl]-3-[(1S)- 1-(2-pyrimidin-2-yl-1,2,4-triazol-3- yl)ethyl]urea | 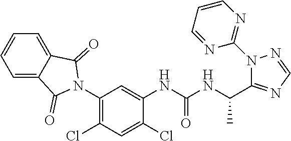 [SMILES: C[C@H](NC(=O)Nc1cc(N2C(=O)c3ccccc3C2=O)c(Cl)cc1Cl)c1ncnn1-c1ncccn1] | 1.39 | 523.21 | 3 | |
| P196 | 1-[2,4-dimethyl-5-(3-methyl-2-oxo- imidazolidin-1-yl)phenyl]-3-[(1S)-1- (2-pyrimidin-2-yl-1,2,4-triazol-3- yl)ethyl]urea | 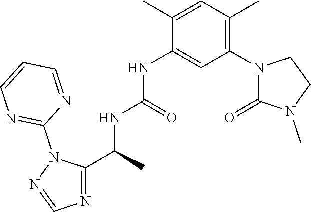 [SMILES: Cc1cc(C)c(N2CCN(C)C2=O)cc1NC(=O)N[C@@H](C)c1ncnn1-c1ncccn1] | 0.94 | 436.3 | 3 | |
| P197 | 1-[4-(2-bromo-1,1,2-trifluoro- ethoxy)-2,5-dichloro-phenyl]-3- [(1S)-1-(2-pyrimidin-2-yl-1,2,4- triazol-3-yl)ethyl]urea | 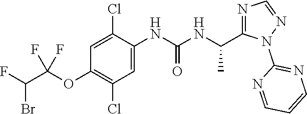 [SMILES: C[C@H](NC(=O)Nc1cc(Cl)c(OC(F)(F)C(F)Br)cc1Cl)c1ncnn1-c1ncccn1] | 1.57 | 554.1 | 3 | |
| P198 | 1-[2,5-dichloro-4- (methylsulfamoyl)phenyl]-3-[(1S)-1- (2-pyrimidin-2-yl-1,2,4-triazol-3- yl)ethyl]urea | 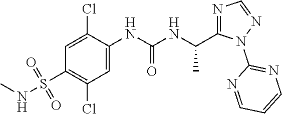 [SMILES: CNS(=O)(=O)c1cc(Cl)c(NC(=O)N[C@@H](C)c2ncnn2-c2ncccn2)cc1Cl] | 1.17 | 471.14 | 3 | |
| P199 | methyl 4,5-dichloro-2-[[(1S)-1-(2- pyrimidin-2-yl-1,2,4-triazol-3- yl)ethyl]carbamoylamino]benzoate | 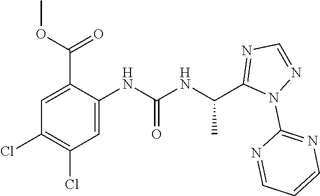 [SMILES: COC(=O)c1cc(Cl)c(Cl)cc1NC(=O)N[C@@H](C)c1ncnn1-c1ncccn1] | 1.5 | 436.15 | 3 | |
| P200 | 1-(2,4-dibromo-5-methoxy-phenyl)- 3-[(1S)-1-(2-pyrimidin-2-yl-1,2,4- triazol-3-yl)ethyl]urea | 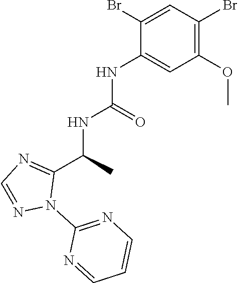 [SMILES: COc1cc(NC(=O)N[C@@H](C)c2ncnn2-c2ncccn2)c(Br)cc1Br] | 1.35 | 496.08 | 3 | |
| P201 | 1-(6-fluoro-3-methyl-2-oxo-1,3- benzothiazol-5-yl)-3-[(1S)-1-(2- pyrimidin-2-yl-1,2,4-triazol-3- yl)ethyl]urea | 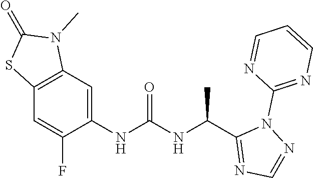 [SMILES: C[C@H](NC(=O)Nc1cc2c(cc1F)sc(=O)n2C)c1ncnn1-c1ncccn1] | 0.99 | 415.19 | 3 | |
| P202 | 1-[4-chloro-5-(1-cyano-1-methyl- ethyl)-2-fluoro-phenyl]-3-[(1S)-1-(2- pyrimidin-2-yl-1,2,4-triazol-3- yl)ethyl]urea | 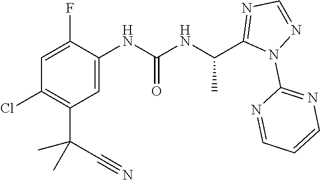 [SMILES: C[C@H](NC(=O)Nc1cc(C(C)(C)C#N)c(Cl)cc1F)c1ncnn1-c1ncccn1] | 1.24 | 429.22 | 3 | |
| P203 | 1-(4-bromo-2-fluoro-5-prop-2- ynoxy-phenyl)-3-[(1S)-1-(2- pyrimidin-2-yl-1,2,4-triazol-3- yl)ethyl]urea | 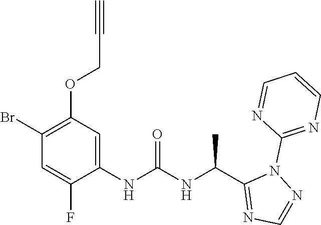 [SMILES: C#CCOc1cc(NC(=O)N[C@@H](C)c2ncnn2-c2ncccn2)c(F)cc1Br] | 1.27 | 460.14 | 3 | |
| P204 | 1-(2,5-dichloro-4-methoxy-phenyl)- 3-[(1S)-1-(2-pyrimidin-2-yl-1,2,4- triazol-3-yl)ethyl]urea | 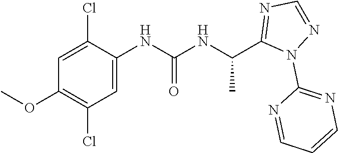 [SMILES: COc1cc(Cl)c(NC(=O)N[C@@H](C)c2ncnn2-c2ncccn2)cc1Cl] | 1.16 | 408.16 | 3 | |
| P205 | 1-[2,4-dibromo-5- (trifluoromethyl)phenyl]-3-[(1S)-1- (2-pyrimidin-2-yl-1,2,4-triazol-3- yl)ethyl]urea | 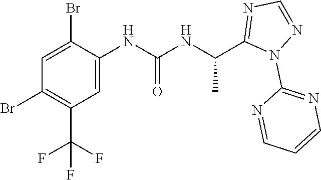 [SMILES: C[C@H](NC(=O)Nc1cc(C(F)(F)F)c(Br)cc1Br)c1ncnn1-c1ncccn1] | 1.56 | 534.06 | 3 | |
| P206 | 1-(2,5-dichloro-4-methylsulfonyl- phenyl)-3-[(1S)-1-(2-pyrimidin-2-yl- 1,2,4-triazol-3-yl)ethyl]urea | 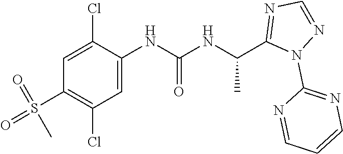 [SMILES: C[C@H](NC(=O)Nc1cc(Cl)c(S(C)(=O)=O)cc1Cl)c1ncnn1-c1ncccn1] | 1.05 | 456.12 | 3 | |
| P207 | 1-(6-chloro-2-methyl-1,3-dioxo- isoindolin-5-yl)-3-[(1S)-1-(2- pyrimidin-2-yl-1,2,4-triazol-3- yl)ethyl]urea | 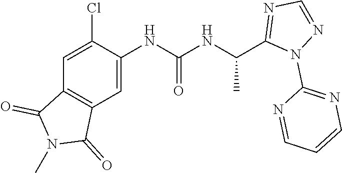 [SMILES: C[C@H](NC(=O)Nc1cc2c(cc1Cl)C(=O)N(C)C2=O)c1ncnn1-c1ncccn1] | 1.08 | 427.17 | 3 | |
| P208 | 1-(6-methyl-3-oxo-4H-1,4- benzoxazin-7-yl)-3-[(1S)-1-(2- pyrimidin-2-yl-1,2,4-triazol-3- yl)ethyl]urea | 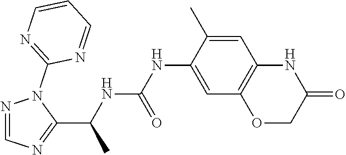 [SMILES: Cc1cc2c(cc1NC(=O)N[C@@H](C)c1ncnn1-c1ncccn1)OCC(=O)N2] | 0.74 | 395.25 | 3 | |
| P209 | 1-[2,4-dichloro-5-[[5- (trifluoromethyl)-2- pyridyl]oxy]phenyl]-3-[(1S)-1-(2- pyrimidin-2-yl-1,2,4-triazol-3- yl)ethyl]urea | 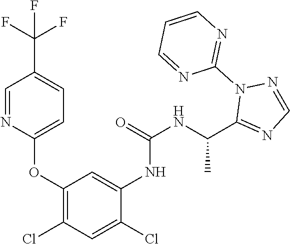 [SMILES: C[C@H](NC(=O)Nc1cc(Oc2ccc(C(F)(F)F)cn2)c(Cl)cc1Cl)c1ncnn1-c1ncccn1] | 1.66 | 539.2 | 3 | |
| P210 | methyl 2-chloro-5-[[(1S)-1-(2- pyrimidin-2-yl-1,2,4-triazol-3- yl)ethyl]carbamoylamino]-4- (trifluoromethyl)benzoate | 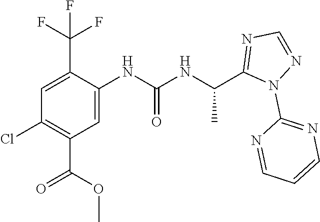 [SMILES: COC(=O)c1cc(NC(=O)N[C@@H](C)c2ncnn2-c2ncccn2)c(C(F)(F)F)cc1Cl] | 1.32 | 470.18 | 3 | |
| P211 | 1-[2,5-dichloro-4-(1,1,2,3,3,3- hexafluoropropoxy)phenyl]-3-[(1S)- 1-(2-pyrimidin-2-yl-1,2,4-triazol-3- yl)ethyl]urea | 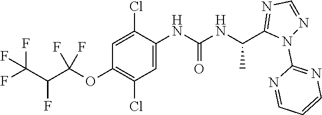 [SMILES: C[C@H](NC(=O)Nc1cc(Cl)c(OC(F)(F)C(F)C(F)(F)F)cc1Cl)c1ncnn1-c1ncccn1] | 1.63 | 544.18 | 3 | |
| P212 | 1-[2,4-dimethyl-5-(2-oxopyrrolidin-1- yl)phenyl]-3-[(1S)-1-(2-pyrimidin-2- yl-1,2,4-triazol-3-yl)ethyl]urea | 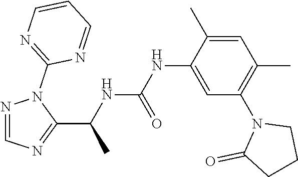 [SMILES: Cc1cc(C)c(N2CCCC2=O)cc1NC(=O)N[C@@H](C)c1ncnn1-c1ncccn1] | 0.92 | 421.29 | 3 | |
| P213 | 1-[2-chloro-5-(4-chloro-5-cyano-1- methyl-pyrazol-3-yl)-4-fluoro- phenyl]-3-[(1S)-1-(2-pyrimidin-2-yl- 1,2,4-triazol-3-yl)ethyl]urea | 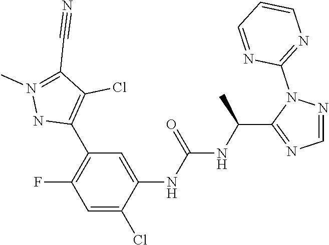 | 1.42 | 501.2 | 3 | |
| P214 | 1-[4-chloro-2-fluoro-5- (trifluoromethoxy)phenyl]-3-[(1S)-1- (2-pyrimidin-2-yl-1,2,4-triazol-3- yl)ethyl]urea | 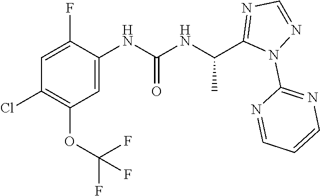 [SMILES: C[C@H](NC(=O)Nc1cc(OC(F)(F)F)c(Cl)cc1F)c1ncnn1-c1ncccn1] | 1.48 | 446.15 | 3 | |
| P215 | 1-[4-chloro-2-fluoro-5-(1,1,2,3,3,3- hexafluoropropoxy)phenyl]-3-[(1S)- 1-(2-pyrimidin-2-yl-1,2,4-triazol-3- yl)ethyl]urea | 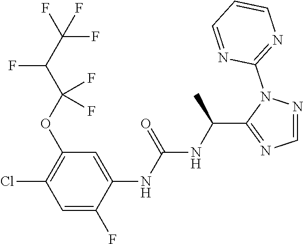 [SMILES: C[C@H](NC(=O)Nc1cc(OC(F)(F)C(F)C(F)(F)F)c(Cl)cc1F)c1ncnn1-c1ncccn1] | 1.59 | 528.2 | 3 | |
| P216 | 1-[4-chloro-5-[(Z)-1,2- dichlorovinyloxy]-2-fluoro-phenyl]-3- [(1S)-1-(2-pyrimidin-2-yl-1,2,4- triazol-3-yl)ethyl]urea | 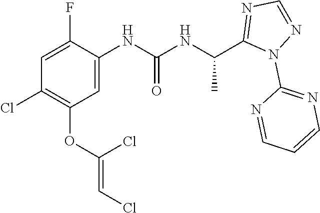 [SMILES: C[C@H](NC(=O)Nc1cc(O/C(Cl)=C/Cl)c(Cl)cc1F)c1ncnn1-c1ncccn1] | 1.54 | 472.12 | 3 | |
| P217 | isopropyl 2-chloro-4-fluoro-5-[[(1S)- 1-(2-pyrimidin-2-yl-1,2,4-triazol-3- yl)ethyl]carbamoylamino]benzoate | 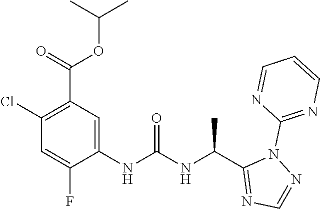 [SMILES: CC(C)OC(=O)c1cc(NC(=O)N[C@@H](C)c2ncnn2-c2ncccn2)c(F)cc1Cl] | 1.38 | 448.21 | 3 | |
| P218 | 1-[4-chloro-5-(dimethylsulfamoyl)-2- fluoro-phenyl]-3-[(1S)-1-(2- pyrimidin-2-yl-1,2,4-triazol-3- yl)ethyl]urea | 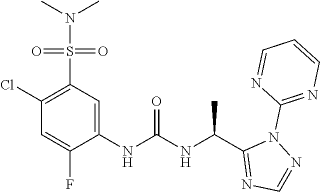 [SMILES: C[C@H](NC(=O)Nc1cc(S(=O)(=O)N(C)C)c(Cl)cc1F)c1ncnn1-c1ncccn1] | 1.12 | 469.19 | 3 | |
| P219 | 1-[4-chloro-5- [chloro(difluoro)methoxy]-2-fluoro- phenyl]-3-[(1S)-1-(2-pyrimidin-2-yl- 1,2,4-triazol-3-yl)ethyl]urea | 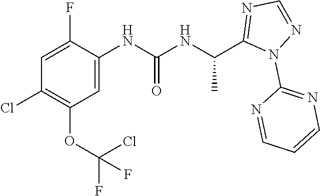 [SMILES: C[C@H](NC(=O)Nc1cc(OC(F)(F)Cl)c(Cl)cc1F)c1ncnn1-c1ncccn1] | 1.53 | 462.12 | 3 | |
| P220 | 1-(4-bromo-2-fluoro-5-methoxy- phenyl)-3-[(1S)-1-(2-pyrimidin-2-yl- 1,2,4-triazol-3-yl)ethyl]urea | 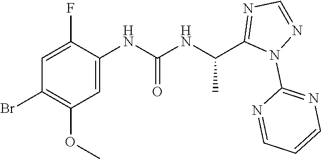 [SMILES: COc1cc(NC(=O)N[C@@H](C)c2ncnn2-c2ncccn2)c(F)cc1Br] | 1.22 | 436.12 | 3 | |
| P221 | 1-(4-bromo-2-fluoro-5-isopropoxy- phenyl)-3-[(1S)-1-(2-pyrimidin-2-yl- 1,2,4-triazol-3-yl)ethyl]urea | 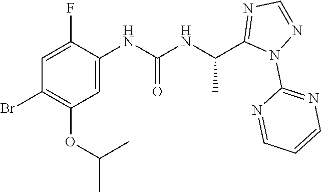 [SMILES: CC(C)Oc1cc(NC(=O)N[C@@H](C)c2ncnn2-c2ncccn2)c(F)cc1Br] | 1.46 | 464.17 | 3 | |
| P222 | 1-(4-chloro-2-fluoro-5-pyrrolidin-1- ylsulfonyl-phenyl)-3-[(1S)-1-(2- pyrimidin-2-yl-1,2,4-triazol-3- yl)ethyl]urea | 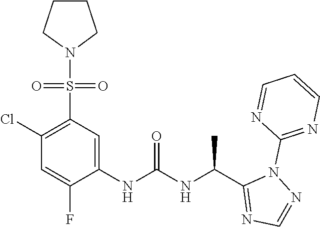 [SMILES: C[C@H](NC(=O)Nc1cc(S(=O)(=O)N2CCCC2)c(Cl)cc1F)c1ncnn1-c1ncccn1] | 1.22 | 495.21 | 3 | |
| P223 | 1-[2,4-dichloro-5-(1,1,2,3,3,3- hexafluoropropoxy)phenyl]-3-[(1S)- 1-(2-pyrimidin-2-yl-1,2,4-triazol-3- yl)ethyl]urea | 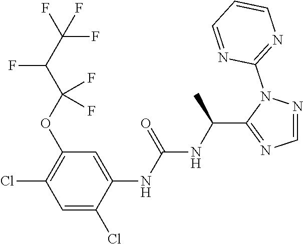 [SMILES: C[C@H](NC(=O)Nc1cc(OC(F)(F)C(F)C(F)(F)F)c(Cl)cc1Cl)c1ncnn1-c1ncccn1] | 1.68 | 544.16 | 3 | |
| P224 | 1-[2,4-dimethyl-5- (trifluoromethylsulfonylamino)phenyl]- 3-[(1S)-1-(2-pyrimidin-2-yl-1,2,4- triazol-3-yl)ethyl]urea | 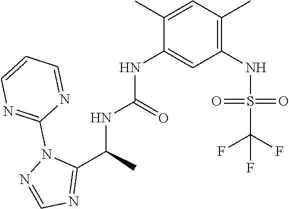 [SMILES: Cc1cc(C)c(NS(=O)(=O)C(F)(F)F)cc1NC(=O)N[C@@H](C)c1ncnn1-c1ncccn1] | 1.21 | 485.23 | 3 | |
| P225 | 1-[2,4-dimethyl-5- [methyl(methylsulfonyl)amino]phenyl]- 3-[(1S)-1-(2-pyrimidin-2-yl-1,2,4- triazol-3-yl)ethyl]urea | 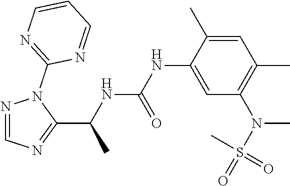 [SMILES: Cc1cc(C)c(N(C)S(C)(=O)=O)cc1NC(=O)N[C@@H](C)c1ncnn1-c1ncccn1] | 0.99 | 445.25 | 3 | |
| P226 | 1-[2,4-dimethyl-5-(2- oxoimidazolidin-1-yl)phenyl]-3- [(1S)-1-(2-pyrimidin-2-yl-1,2,4- triazol-3-yl)ethyl]urea | 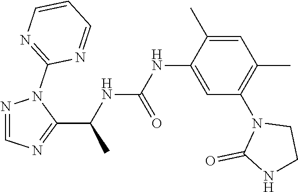 [SMILES: Cc1cc(C)c(N2CCNC2=O)cc1NC(=O)N[C@@H](C)c1ncnn1-c1ncccn1] | 0.84 | 422.27 | 3 | |
| P227 | 1-[2,4-dimethyl-5-(2-oxooxazolidin- 3-yl)phenyl]-3-[(1S)-1-(2-pyrimidin- 2-yl-1,2,4-triazol-3-yl)ethyl]urea | 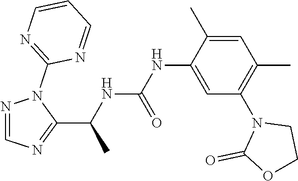 [SMILES: Cc1cc(C)c(N2CCOC2=O)cc1NC(=O)N[C@@H](C)c1ncnn1-c1ncccn1] | 0.87 | 423.24 | 3 | |
| P228 | methyl 2-bromo-4-fluoro-5-[[(1S)-1- (2-pyrimidin-2-yl-1,2,4-triazol-3- yl)ethyl]carbamoylamino]benzoate | 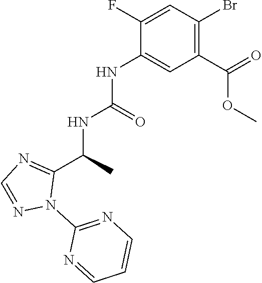 [SMILES: COC(=O)c1cc(NC(=O)N[C@@H](C)c2ncnn2-c2ncccn2)c(F)cc1Br] | 1.18 | 464.13 | 3 | |
| P229 | 1-(5-acetyl-2,4-dichloro-phenyl)-3- [(1S)-1-(2-pyrimidin-2-yl-1,2,4- triazol-3-yl)ethyl]urea | 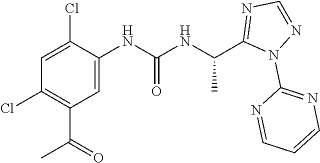 [SMILES: CC(=O)c1cc(NC(=O)N[C@@H](C)c2ncnn2-c2ncccn2)c(Cl)cc1Cl] | 1.2 | 420.14 | 3 | |
| P230 | N-[2,4-dimethyl-5-[[(1S)-1-(2- pyrimidin-2-yl-1,2,4-triazol-3- yl)ethyl]carbamoylamino]phenyl]-N- methyl-acetamide | 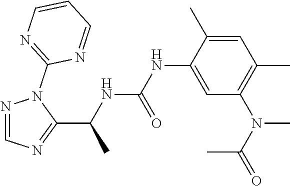 [SMILES: CC(=O)N(C)c1cc(NC(=O)N[C@@H](C)c2ncnn2-c2ncccn2)c(C)cc1C] | 0.92 | 409.28 | 3 | |
| P231 | 1-(4-bromo-2-fluoro-5-propoxy- phenyl)-3-[(1S)-1-(2-pyrimidin-2-yl- 1,2,4-triazol-3-yl)ethyl]urea | 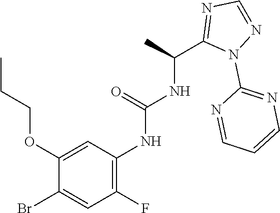 [SMILES: CCCOc1cc(NC(=O)N[C@@H](C)c2ncnn2-c2ncccn2)c(F)cc1Br] | 1.5 | 464.17 | 3 | |
| P232 | 1-[4-bromo-5-[(E)-3-chloroallyloxy]- 2-fluoro-phenyl]-3-[(1S)-1-(2- pyrimidin-2-yl-1,2,4-triazol-3- yl)ethyl]urea | 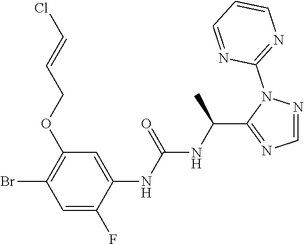 [SMILES: C[C@H](NC(=O)Nc1cc(OC/C=C/Cl)c(Br)cc1F)c1ncnn1-c1ncccn1] | 1.52 | 496.13 | 3 | |
| P233 | 1-(4-cyano-2-fluoro-5-methoxy- phenyl)-3-[(1S)-1-(2-pyrimidin-2-yl- 1,2,4-triazol-3-yl)ethyl]urea | 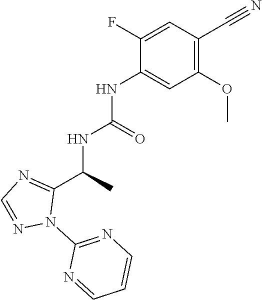 [SMILES: COc1cc(NC(=O)N[C@@H](C)c2ncnn2-c2ncccn2)c(F)cc1C#N] | 1.04 | 383.21 | 3 | |
| P234 | 5-chloro-6-methoxy-N-[(1S)-1-(2- pyrimidin-2-yl-1,2,4-triazol-3- yl)ethyl]indoline-1-carboxamide | 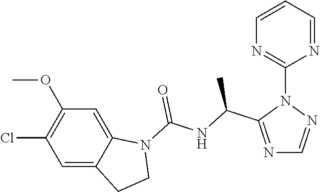 [SMILES: COc1cc2c(cc1Cl)CCN2C(=O)N[C@@H](C)c1ncnn1-c1ncccn1] | 0.85 | 400.2 | 2 | |
| P235 | 1-[4-chloro-6-(trifluoromethyl)-3- pyridyl]-1-methyl-3-[(1S)-1-(2- pyrimidin-2-yl-1,2,4-triazol-3- yl)ethyl]urea | 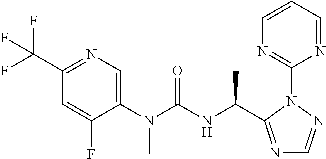 [SMILES: C[C@H](NC(=O)N(C)c1cnc(C(F)(F)F)cc1F)c1ncnn1-c1ncccn1] | 0.8 | 427.0 | 2 | 88 - 90 |
| P236 | 1-(2,4-dichloro-5-isopropoxy- phenyl)-3-[(1S)-1-(2-pyrimidin-2-yl- 1,2,4-triazol-3-yl)ethyl]urea | 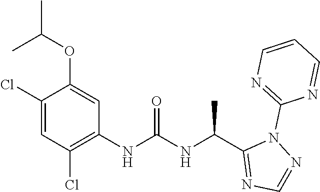 [SMILES: CC(C)Oc1cc(NC(=O)N[C@@H](C)c2ncnn2-c2ncccn2)c(Cl)cc1Cl] | 1.53 | 436.18 | 3 | |
| P237 | 1-(2,4-dichloro-5-hydroxy-phenyl)- 3-[(1S)-1-(2-pyrimidin-2-yl-1,2,4- triazol-3-yl)ethyl]urea | 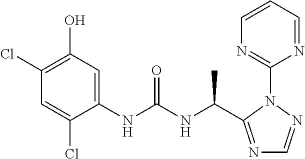 [SMILES: C[C@H](NC(=O)Nc1cc(O)c(Cl)cc1Cl)c1ncnn1-c1ncccn1] | 0.77 | 394.0 | 2 | 187 - 190 |
| P238 | 1-[2,4-dichloro-5-(1,1,2,2- tetrafluoroethoxy)phenyl]-3-[1-(3- pyrimidin-2-ylpyrazin-2-yl)ethyl]urea | 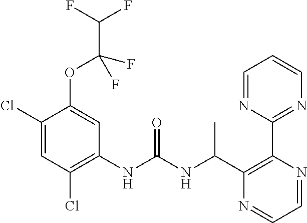 [SMILES: CC(NC(=O)Nc1cc(OC(F)(F)C(F)F)c(Cl)cc1Cl)c1nccnc1-c1ncccn1] | 1.49 | 504.99 | 3 | |
| P239 | 1-[4-chloro-3- (trifluoromethyl)phenyl]-3-[1-(3- pyrimidin-2-ylpyrazin-2-yl)ethyl]urea | 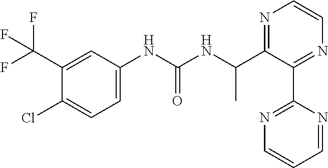 [SMILES: CC(NC(=O)Nc1ccc(Cl)c(C(F)(F)F)c1)c1nccnc1-c1ncccn1] | 1.31 | 423.03 | 3 | |
| P240 | 1-[2-bromo-4-chloro-5- (trifluoromethyl)phenyl]-3-[1-(3- pyrimidin-2-ylpyrazin-2-yl)ethyl]urea | 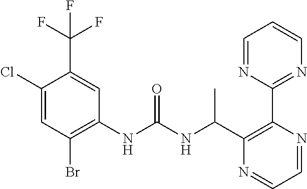 [SMILES: CC(NC(=O)Nc1cc(C(F)(F)F)c(Cl)cc1Br)c1nccnc1-c1ncccn1] | 1.49 | 500.94 | 3 | |
| P241 | 1-[4-bromo-2- (difluoromethoxy)phenyl]-3-[1-(3- pyrimidin-2-ylpyrazin-2-yl)ethyl]urea | 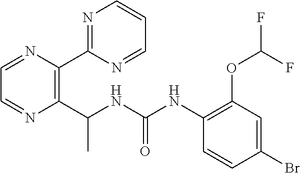 [SMILES: CC(NC(=O)Nc1ccc(Br)cc1OC(F)F)c1nccnc1-c1ncccn1] | 1.25 | 464.99 | 3 | |
| P242 | 1-(4,6-dibromo-3-pyridyl)-3-[1-(3- pyrimidin-2-ylpyrazin-2-yl)ethyl]urea | 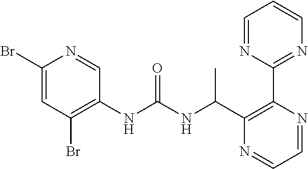 [SMILES: CC(NC(=O)Nc1cnc(Br)cc1Br)c1nccnc1-c1ncccn1] | 1.01 | 477.89 | 3 | |
| P243 | 1-[2-iodo-4-(trifluoromethyl)phenyl]- 3-[1-(3-pyrimidin-2-ylpyrazin-2- yl)ethyl]urea | 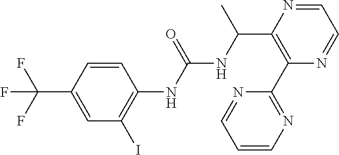 [SMILES: CC(NC(=O)Nc1ccc(C(F)(F)F)cc1I)c1nccnc1-c1ncccn1] | 1.36 | 514.98 | 3 | |
| P244 | 1-[4-chloro-6-(trifluoromethyl)-3- pyridyl]-3-[1-(3-pyrimidin-2- ylpyrazin-2-yl)ethyl]urea | 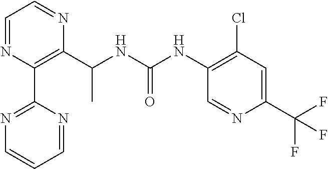 [SMILES: CC(NC(=O)Nc1cnc(C(F)(F)F)cc1Cl)c1nccnc1-c1ncccn1] | 1.12 | 424.02 | 3 | |
| P245 | 1-[(1S)-1-[3-(5-cyano-2- pyridyl)pyrazin-2-yl]ethyl]-3-[2,4- dichloro-5-(1,1,2,2- tetrafluoroethoxy)phenyl]urea | 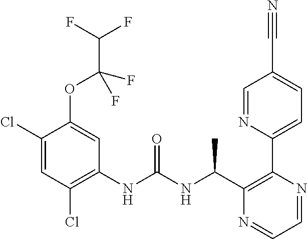 [SMILES: C[C@H](NC(=O)Nc1cc(OC(F)(F)C(F)F)c(Cl)cc1Cl)c1nccnc1-c1ccc(C#N)cn1] | 1.71 | 529.01 | 3 | |
| P246 | 1-[2-bromo-4-chloro-5- (trifluoromethyl)phenyl]-3-[(1S)-1-[3- (5-cyano-2-pyridyl)pyrazin-2- yl]ethyl]urea | 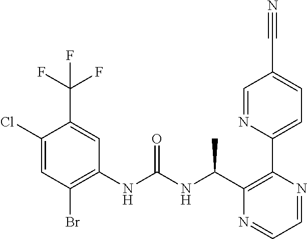 [SMILES: C[C@H](NC(=O)Nc1cc(C(F)(F)F)c(Cl)cc1Br)c1nccnc1-c1ccc(C#N)cn1] | 1.74 | 524.96 | 3 | |
| P247 | 1-[4-bromo-2- (difluoromethoxy)phenyl]-3-[(1S)-1- [3-(5-cyano-2-pyridyl)pyrazin-2- yl]ethyl]urea | 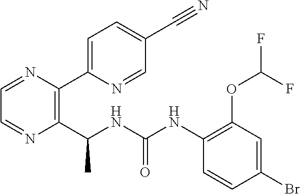 [SMILES: C[C@H](NC(=O)Nc1ccc(Br)cc1OC(F)F)c1nccnc1-c1ccc(C#N)cn1] | 1.51 | 488.98 | 3 | |
| P248 | 1-[(1S)-1-[3-(5-cyano-2- pyridyl)pyrazin-2-yl]ethyl]-3-(4,6- dibromo-3-pyridyl)urea | 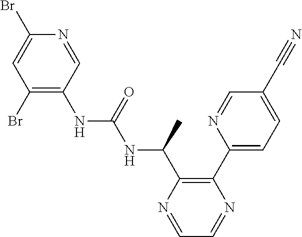 [SMILES: C[C@H](NC(=O)Nc1cnc(Br)cc1Br)c1nccnc1-c1ccc(C#N)cn1] | 1.29 | 502.28 | 3 | |
| P249 | 1-[(1S)-1-[3-(5-cyano-2- pyridyl)pyrazin-2-yl]ethyl]-3-[2-iodo- 4-(trifluoromethyl)phenyl]urea | 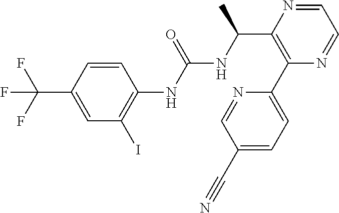 [SMILES: C[C@H](NC(=O)Nc1ccc(C(F)(F)F)cc1I)c1nccnc1-c1ccc(C#N)cn1] | 1.62 | 538.96 | 3 | |
| P250 | 1-[4-chloro-6-(trifluoromethyl)-3- pyridyl]-3-[(1S)-1-[3-(5-cyano-2- pyridyl)pyrazin-2-yl]ethyl]urea | 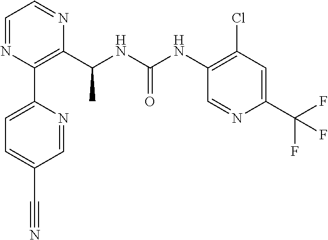 [SMILES: C[C@H](NC(=O)Nc1cnc(C(F)(F)F)cc1Cl)c1nccnc1-c1ccc(C#N)cn1] | 1.39 | 448.02 | 3 | |
| P251 | 1-[(1S)-1-[2-(5-cyano-2-pyridyl)- 1,2,4-triazol-3-yl]ethyl]-3-[2,4- dichloro-5-(1,1,2,2- tetrafluoroethoxy)phenyl]urea | 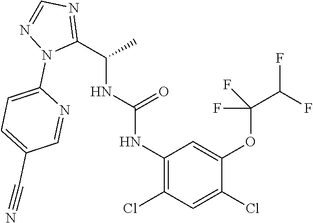 [SMILES: C[C@H](NC(=O)Nc1cc(OC(F)(F)C(F)F)c(Cl)cc1Cl)c1ncnn1-c1ccc(C#N)cn1] | 1.64 | 518.0 | 3 | |
| P252 | 1-[4-chloro-3- (trifluoromethyl)phenyl]-3-[(1S)-1-[2- (5-cyano-2-pyridyl)-1,2,4-triazol-3- yl]ethyl]urea | 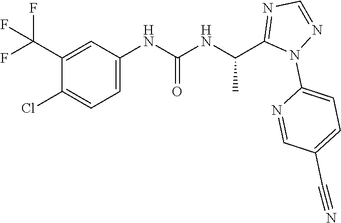 [SMILES: C[C@H](NC(=O)Nc1ccc(Cl)c(C(F)(F)F)c1)c1ncnn1-c1ccc(C#N)cn1] | 1.49 | 436.02 | 3 | |
| P253 | 1-[2-bromo-4-chloro-5- (trifluoromethyl)phenyl]-3-[(1S)-1-[2- (5-cyano-2-pyridyl)-1,2,4-triazol-3- yl]ethyl]urea | 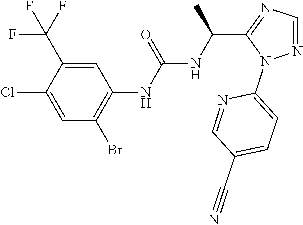 [SMILES: C[C@H](NC(=O)Nc1cc(C(F)(F)F)c(Cl)cc1Br)c1ncnn1-c1ccc(C#N)cn1] | 1.66 | 513.92 | 3 | |
| P254 | 1-[4-bromo-2- (difluoromethoxy)phenyl]-3-[(1S)-1- [2-(5-cyano-2-pyridyl)-1,2,4-triazol- 3-yl]ethyl]urea | 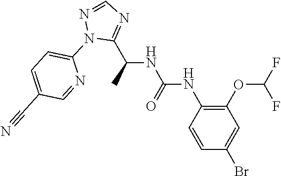 [SMILES: C[C@H](NC(=O)Nc1ccc(Br)cc1OC(F)F)c1ncnn1-c1ccc(C#N)cn1] | 1.44 | 477.98 | 3 | |
| P255 | 1-[(1S)-1-[2-(5-cyano-2-pyridyl)- 1,2,4-triazol-3-yl]ethyl]-3-(4,6- dibromo-3-pyridyl)urea | 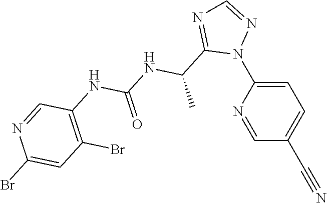 [SMILES: C[C@H](NC(=O)Nc1cnc(Br)cc1Br)c1ncnn1-c1ccc(C#N)cn1] | 1.22 | 490.89 | 3 | |
| P256 | 1-[(1S)-1-[2-(5-cyano-2-pyridyl)- 1,2,4-triazol-3-yl]ethyl]-3-[2-iodo-4- (trifluoromethyl)phenyl]urea | 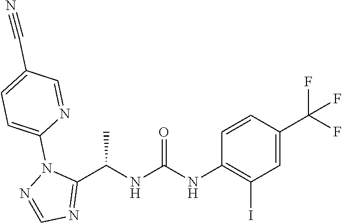 [SMILES: C[C@H](NC(=O)Nc1ccc(C(F)(F)F)cc1I)c1ncnn1-c1ccc(C#N)cn1] | 1.54 | 527.96 | 3 | |
| P257 | 1-[4-chloro-6-(trifluoromethyl)-3- pyridyl]-3-[(1S)-1-[2-(5-cyano-2- pyridyl)-1,2,4-triazol-3-yl]ethyl]urea | 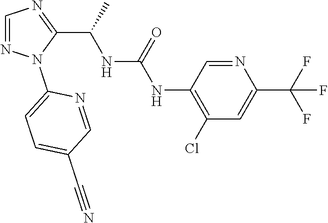 [SMILES: C[C@H](NC(=O)Nc1cnc(C(F)(F)F)cc1Cl)c1ncnn1-c1ccc(C#N)cn1] | 1.32 | 437.02 | 3 | |
| P258 | 1-[2,4-dichloro-5-(1,1,2,2- tetrafluoroethoxy)phenyl]-3-[1-[3- (triazol-2-yl)pyrazin-2-yl]ethyl]urea | 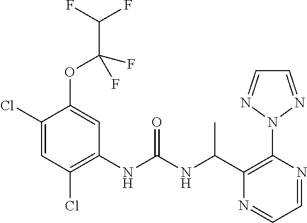 [SMILES: CC(NC(=O)Nc1cc(OC(F)(F)C(F)F)c(Cl)cc1Cl)c1nccnc1-n1nccn1] | 1.6 | 494.0 | 3 | |
| P259 | 1-[4-chloro-3- (trifluoromethyl)phenyl]-3-[1-[3- (triazol-2-yl)pyrazin-2-yl]ethyl]urea | 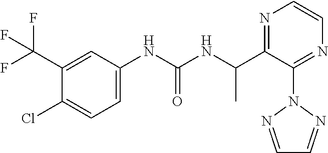 [SMILES: CC(NC(=O)Nc1ccc(Cl)c(C(F)(F)F)c1)c1nccnc1-n1nccn1] | 1.42 | 412.03 | 3 | |
| P260 | 1-[2-bromo-4-chloro-5- (trifluoromethyl)phenyl]-3-[1-[3- (triazol-2-yl)pyrazin-2-yl]ethyl]urea | 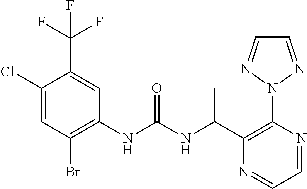 [SMILES: CC(NC(=O)Nc1cc(C(F)(F)F)c(Cl)cc1Br)c1nccnc1-n1nccn1] | 1.61 | 489.97 | 3 | |
| P261 | 1-[4-bromo-2- (difluoromethoxy)phenyl]-3-[1-[3- (triazol-2-yl)pyrazin-2-yl]ethyl]urea | 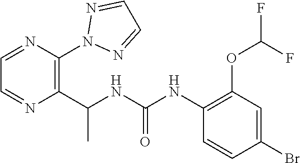 [SMILES: CC(NC(=O)Nc1ccc(Br)cc1OC(F)F)c1nccnc1-n1nccn1] | 1.38 | 453.97 | 3 | |
| P262 | 1-(4,6-dibromo-3-pyridyl)-3-[1-[3- (triazol-2-yl)pyrazin-2-yl]ethyl]urea | 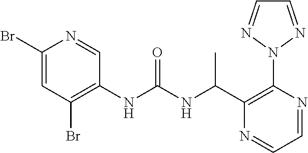 [SMILES: CC(NC(=O)Nc1cnc(Br)cc1Br)c1nccnc1-n1nccn1] | 1.13 | 466.89 | 3 | |
| P263 | 1-[2-iodo-4-(trifluoromethyl)phenyl]- 3-[1-[3-(triazol-2-yl)pyrazin-2- yl]ethyl]urea | 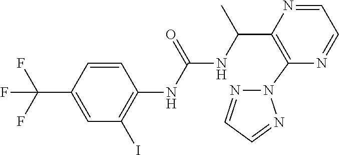 [SMILES: CC(NC(=O)Nc1ccc(C(F)(F)F)cc1I)c1nccnc1-n1nccn1] | 1.48 | 503.96 | 3 | |
| P264 | 1-[4-chloro-6-(trifluoromethyl)-3- pyridyl]-3-[1-[3-(triazol-2-yl)pyrazin- 2-yl]ethyl]urea | 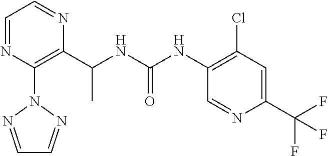 [SMILES: CC(NC(=O)Nc1cnc(C(F)(F)F)cc1Cl)c1nccnc1-n1nccn1] | 1.24 | 413.01 | 3 | |
| P265 | 1-(2,4-dichloro-5-methoxy-phenyl)- 3-[(1S)-1-(2-pyrimidin-2-yl-1,2,4- triazol-3-yl)ethyl]urea | 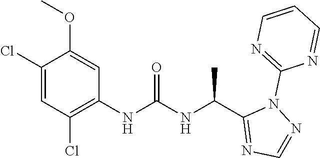 [SMILES: COc1cc(NC(=O)N[C@@H](C)c2ncnn2-c2ncccn2)c(Cl)cc1Cl] | 1.25 | 408.26 | 3 | 235 - 238 |
| P266 | 1-[3-chloro-5- (trifluoromethyl)phenyl]-3-[(1S)-1- (2-pyrimidin-2-yl-1,2,4-triazol-3- yl)ethyl]thiourea | 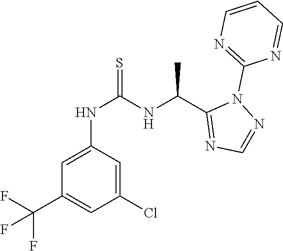 [SMILES: C[C@H](NC(=S)Nc1cc(Cl)cc(C(F)(F)F)c1)c1ncnn1-c1ncccn1] | 1.4 | 428.1 | 4 | 106 - 108 |
| P267 | 1-[3-chloro-5-(trifluoromethyl)-2- pyridyl]-1,3-dimethyl-3-[(1S)-1-(2- pyrimidin-2-yl-1,2,4-triazol-3- yl)ethyl]urea | 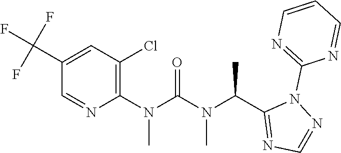 [SMILES: C[C@@H](c1ncnn1-c1ncccn1)N(C)C(=O)N(C)c1ncc(C(F)(F)F)cc1Cl] | 1.33 | 442.1 | 4 | |
| P268 | 1-(4,5-dichloro-2-iodo-phenyl)-3- [(1S)-1-(2-pyrimidin-2-yl-1,2,4- triazol-3-yl)ethyl]urea | 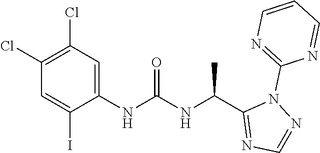 [SMILES: C[C@H](NC(=O)Nc1cc(Cl)c(Cl)cc1I)c1ncnn1-c1ncccn1] | 0.97 | 502.9 | 2 | 236 - 249 |
| P269 | 1-[2,4-dichloro-5-(1,1,2,2- tetrafluoroethoxy)phenyl]-3-[(1S)-1- [2-(1-methyl-6-oxo-pyridazin-3-yl)- 1,2,4-triazol-3-yl]ethyl]urea | 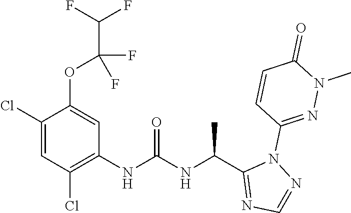 [SMILES: C[C@H](NC(=O)Nc1cc(OC(F)(F)C(F)F)c(Cl)cc1Cl)c1ncnn1-c1ccc(=O)n(C)n1] | 1.04 | 524.0 | 1 | 102 - 106 |
| P270 | 1-[4-chloro-6-(trifluoromethyl)-3- pyridyl]-1,3-dimethyl-3-[(1S)-1-(2- pyrimidin-2-yl-1,2,4-triazol-3- yl)ethyl]urea | 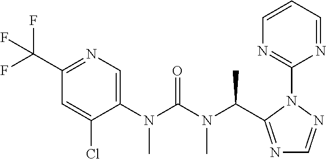 [SMILES: C[C@@H](c1ncnn1-c1ncccn1)N(C)C(=O)N(C)c1cnc(C(F)(F)F)cc1Cl] | 1.5 | 441.3 | 6 | |
| P271 | 1-(6-chloro-4-iodo-3-pyridyl)-3- [(1S)-1-(2-pyrimidin-2-yl-1,2,4- triazol-3-yl)ethyl]urea | 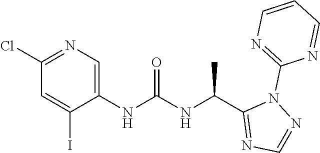 [SMILES: C[C@H](NC(=O)Nc1cnc(Cl)cc1I)c1ncnn1-c1ncccn1] | 0.75 | 471.0 | 2 | 194 - 215 |
| P272 | 1-[2,4-dichloro-5- (difluoromethoxy)phenyl]-3-[(1S)-1- (2-pyrimidin-2-yl-1,2,4-triazol-3- yl)ethyl]urea | 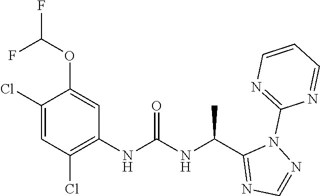 [SMILES: C[C@H](NC(=O)Nc1cc(OC(F)F)c(Cl)cc1Cl)c1ncnn1-c1ncccn1] | 0.92 | 444.0 | 2 | 201 - 203 |
| P273 | 1-[4-bromo-6-(trifluoromethyl)-3- pyridyl]-1-methyl-3-[(1S)-1-(2- pyrimidin-2-yl-1,2,4-triazol-3- yl)ethyl]urea | 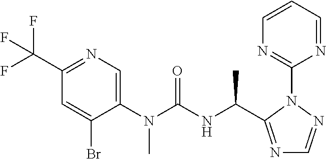 [SMILES: C[C@H](NC(=O)N(C)c1cnc(C(F)(F)F)cc1Br)c1ncnn1-c1ncccn1] | 0.8 | 471.1 | 2 | 136 - 138 |
| P274 | 1-[2,4-dichloro-5-[(5-cyano-2- pyridyl)oxy]phenyl]-3-[(1S)-1-(2- pyrimidin-2-yl-1,2,4-triazol-3- yl)ethyl]urea | 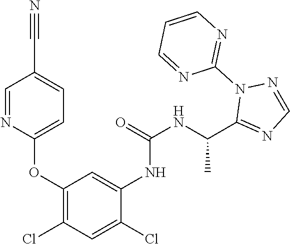 [SMILES: C[C@H](NC(=O)Nc1cc(Oc2ccc(C#N)cn2)c(Cl)cc1Cl)c1ncnn1-c1ncccn1] | 1.02 | 466.2 | 1 | 142 - 144 |
| P275 | 1-[(1S)-1-[3-(5-cyano-2- pyridyl)pyrazin-2-yl]ethyl]-3-[4,6- dichloro-2-iodo-3-(1,1,2,2- tetrafluoroethoxy)phenyl]urea | 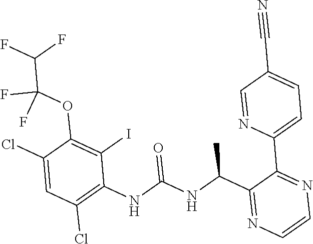 [SMILES: C[C@H](NC(=O)Nc1c(Cl)cc(Cl)c(OC(F)(F)C(F)F)c1I)c1nccnc1-c1ccc(C#N)cn1] | 1.13 | 655.0 | 1 | 230 - 232 |
| P276 | 1-[2,4-dichloro-5-(1,1,2,2- tetrafluoroethoxy)phenyl]-3-[(1S)-1- (5-methyl-2-pyrimidin-2-yl-1,2,4- triazol-3-yl)ethyl]urea | 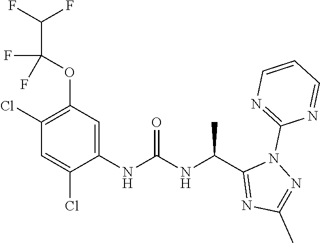 [SMILES: Cc1nc([C@H](C)NC(=O)Nc2cc(OC(F)(F)C(F)F)c(Cl)cc2Cl)n(-c2ncccn2)n1] | 1.11 | 508.1 | 1 | 124 - 126 |
| P277 | 1-[4-chloro-6-(trifluoromethyl)-3- pyridyl]-3-[(1S)-1-(2-pyrazin-2-yl- 1,2,4-triazol-3-yl)ethyl]urea | 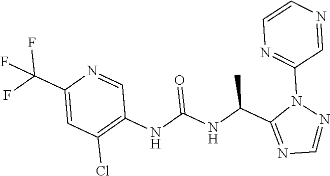 [SMILES: C[C@H](NC(=O)Nc1cnc(C(F)(F)F)cc1Cl)c1ncnn1-c1cnccn1] | 1.03 | 413.2 | 1 | 213 - 215 |
| P278 | 1-[2,4-dichloro-5-(1,1,2,2- tetrafluoroethoxy)phenyl]-3-[(1S)-1- (2-pyrazin-2-yl-1,2,4-triazol-3- yl)ethyl]urea | 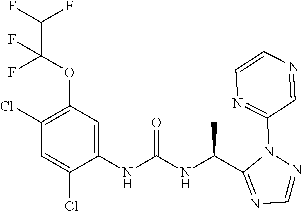 [SMILES: C[C@H](NC(=O)Nc1cc(OC(F)(F)C(F)F)c(Cl)cc1Cl)c1ncnn1-c1cnccn1] | 1.15 | 494.0 | 1 | 130 - 132 |
| P279 | 1-[2-bromo-4-chloro-5- (trifluoromethyl)phenyl]-3-[(1S)-1- (2-pyrazin-2-yl-1,2,4-triazol-3- yl)ethyl]urea | 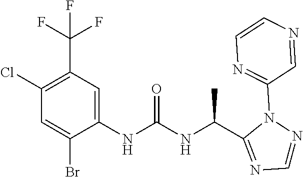 [SMILES: C[C@H](NC(=O)Nc1cc(C(F)(F)F)c(Cl)cc1Br)c1ncnn1-c1cnccn1] | 1.16 | 490.0 | 1 | 204 - 206 |
| P280 | 1-[4-bromo-6-(trifluoromethyl)-3- pyridyl]-3-[(1S)-1-(2-pyrimidin-2-yl- 1,2,4-triazol-3-yl)ethyl]urea | 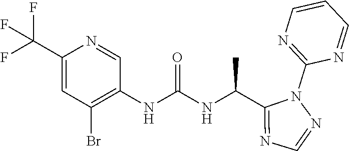 [SMILES: C[C@H](NC(=O)Nc1cnc(C(F)(F)F)cc1Br)c1ncnn1-c1ncccn1] | 0.83 | 457.0 | 2 | 170 - 172 |
| P281 | 5-chloro-N-[(1S)-1-(2-pyrimidin-2-yl- 1,2,4-triazol-3-yl)ethyl]-6- (trifluoromethyl)indoline-1- carboxamide | 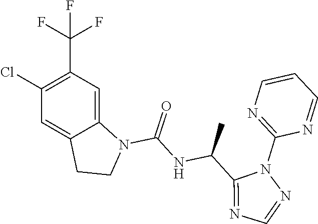 [SMILES: C[C@H](NC(=O)N1CCc2cc(Cl)c(C(F)(F)F)cc21)c1ncnn1-c1ncccn1] | 0.88 | 438.5 | 2 | |
| P282 | 1-(2,5-dimethyl-4-sulfamoyl- phenyl)-3-[(1S)-1-(2-pyrimidin-2-yl- 1,2,4-triazol-3-yl)ethyl]urea | 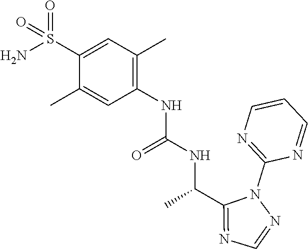 [SMILES: Cc1cc(S(N)(=O)=O)c(C)cc1NC(=O)N[C@@H](C)c1ncnn1-c1ncccn1] | 0.73 | 417.28 | 3 | |
| P283 | methyl 2-[4-chloro-2-methyl-5- [[(1S)-1-(2-pyrimidin-2-yl-1,2,4- triazol-3- yl)ethyl]carbamoylamino]phenoxy]- acetate | 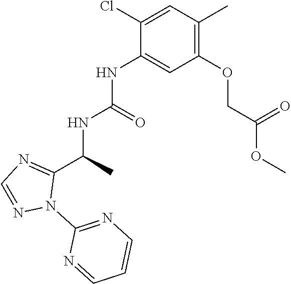 [SMILES: COC(=O)COc1cc(NC(=O)N[C@@H](C)c2ncnn2-c2ncccn2)c(Cl)cc1C] | 1.21 | 446.28 | 3 | |
| P284 | 1-(5-chloro-2-methyl-1,3- benzothiazol-6-yl)-3-[(1S)-1-(2- pyrimidin-2-yl-1,2,4-triazol-3- yl)ethyl]urea | 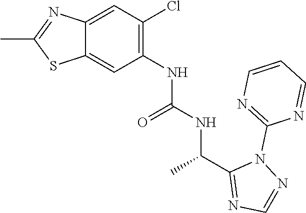 [SMILES: Cc1nc2cc(Cl)c(NC(=O)N[C@@H](C)c3ncnn3-c3ncccn3)cc2s1] | 1.04 | 415.22 | 3 | |
| P285 | 1-(5-chloro-2-fluoro-4-methyl- phenyl)-3-[(1S)-1-(2-pyrimidin-2-yl- 1,2,4-triazol-3-yl)ethyl]urea | 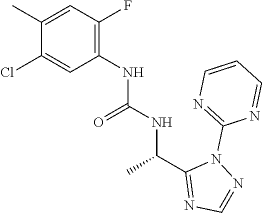 [SMILES: Cc1cc(F)c(NC(=O)N[C@@H](C)c2ncnn2-c2ncccn2)cc1Cl] | 1.23 | 376.23 | 3 | |
| P286 | 1-(7-fluoro-2-oxo-3,4-dihydro-1H- quinolin-6-yl)-3-[(1S)-1-(2-pyrimidin- 2-yl-1,2,4-triazol-3-yl)ethyl]urea | 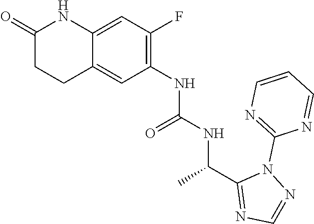 [SMILES: C[C@H](NC(=O)Nc1cc2c(cc1F)NC(=O)CC2)c1ncnn1-c1ncccn1] | 0.71 | 397.3 | 3 | |
| P287 | 1-(6-fluoro-2-oxo-3,4-dihydro-1H- quinolin-7-yl)-3-[(1S)-1-(2-pyrimidin- 2-yl-1,2,4-triazol-3-yl)ethyl]urea | 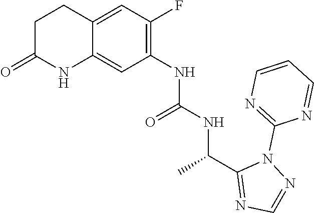 [SMILES: C[C@H](NC(=O)Nc1cc2c(cc1F)CCC(=O)N2)c1ncnn1-c1ncccn1] | 0.74 | 397.3 | 3 | |
| P288 | 1-(4-chloro-2-iodo-5-methoxy- phenyl)-3-[(1S)-1-(2-pyrimidin-2-yl- 1,2,4-triazol-3-yl)ethyl]urea | 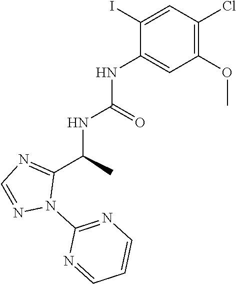 [SMILES: COc1cc(NC(=O)N[C@@H](C)c2ncnn2-c2ncccn2)c(I)cc1Cl] | 1.28 | 500.18 | 3 | |
| P289 | 1-(5-fluoro-2-oxo-3H-1,3- benzoxazol-6-yl)-3-[(1S)-1-(2- pyrimidin-2-yl-1,2,4-triazol-3- yl)ethyl]urea | 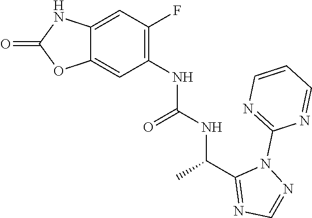 [SMILES: C[C@H](NC(=O)Nc1cc2oc(=O)[nH]c2cc1F)c1ncnn1-c1ncccn1] | 0.71 | 385.26 | 3 | |
| P290 | 1-(4-bromo-2-chloro-5-methyl- phenyl)-3-[(1S)-1-(2-pyrimidin-2-yl- 1,2,4-triazol-3-yl)ethyl]urea | 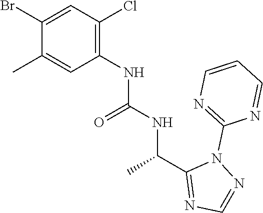 [SMILES: Cc1cc(NC(=O)N[C@@H](C)c2ncnn2-c2ncccn2)c(Cl)cc1Br] | 1.38 | 436.17 | 3 | |
| P291 | 1-(4-bromo-2,5-dimethyl-phenyl)-3- [(1S)-1-(2-pyrimidin-2-yl-1,2,4- triazol-3-yl)ethyl]urea | 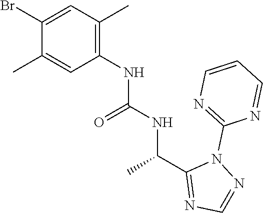 [SMILES: Cc1cc(NC(=O)N[C@@H](C)c2ncnn2-c2ncccn2)c(C)cc1Br] | 1.26 | 416.22 | 3 | |
| P292 | 1-(4-chloro-5-fluoro-2-methoxy- phenyl)-3-[(1S)-1-(2-pyrimidin-2-yl- 1,2,4-triazol-3-yl)ethyl]urea | 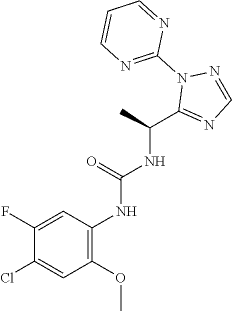 [SMILES: COc1cc(Cl)c(F)cc1NC(=O)N[C@@H](C)c1ncnn1-c1ncccn1] | 1.23 | 392.25 | 3 | |
| P293 | methyl 5-chloro-2-methoxy-4-[[(1S)- 1-(2-pyrimidin-2-yl-1,2,4-triazol-3- yl)ethyl]carbamoylamino]benzoate | 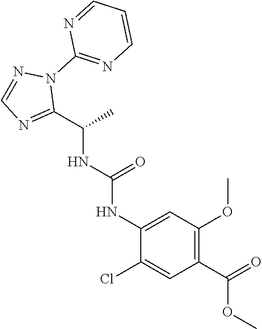 [SMILES: COC(=O)c1cc(Cl)c(NC(=O)N[C@@H](C)c2ncnn2-c2ncccn2)cc1OC] | 1.08 | 432.26 | 3 | |
| P294 | 1-(2-bromo-4-chloro-5-methoxy- phenyl)-3-[(1S)-1-(2-pyrimidin-2-yl- 1,2,4-triazol-3-yl)ethyl]urea | 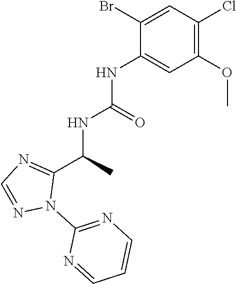 [SMILES: COc1cc(NC(=O)N[C@@H](C)c2ncnn2-c2ncccn2)c(Br)cc1Cl] | 1.29 | 452.18 | 3 | |
| P295 | 1-(4-fluoro-2,5-dimethyl-phenyl)-3- [(1S)-1-(2-pyrimidin-2-yl-1,2,4- triazol-3-yl)ethyl]urea | 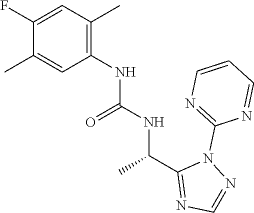 [SMILES: Cc1cc(NC(=O)N[C@@H](C)c2ncnn2-c2ncccn2)c(C)cc1F] | 1.06 | 356.29 | 3 | |
| P296 | 1-(5-cyano-4-fluoro-2-methyl- phenyl)-3-[(1S)-1-(2-pyrimidin-2-yl- 1,2,4-triazol-3-yl)ethyl]urea | 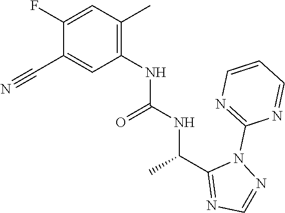 [SMILES: Cc1cc(F)c(C#N)cc1NC(=O)N[C@@H](C)c1ncnn1-c1ncccn1] | 0.93 | 367.27 | 3 | |
| P297 | 1-[4-fluoro-2-methyl-5- (trifluoromethyl)phenyl]-3-[(1S)-1- (2-pyrimidin-2-yl-1,2,4-triazol-3- yl)ethyl]urea | 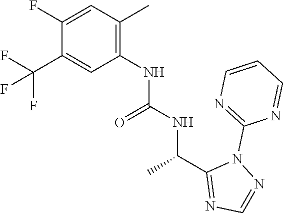 [SMILES: Cc1cc(F)c(C(F)(F)F)cc1NC(=O)N[C@@H](C)c1ncnn1-c1ncccn1] | 1.25 | 410.25 | 3 | |
| P298 | 1-(5-methoxy-2-methyl-1,3- benzothiazol-6-yl)-3-[(1S)-1-(2- pyrimidin-2-yl-1,2,4-triazol-3- yl)ethyl]urea | 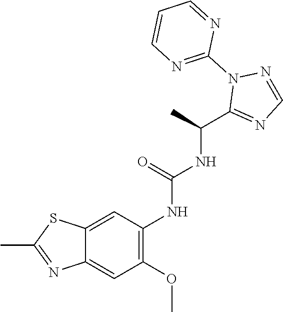 [SMILES: COc1cc2nc(C)sc2cc1NC(=O)N[C@@H](C)c1ncnn1-c1ncccn1] | 0.99 | 411.26 | 3 | |
| P299 | 1-(4-bromo-5-chloro-2-methyl- phenyl)-3-[(1S)-1-(2-pyrimidin-2-yl- 1,2,4-triazol-3-yl)ethyl]urea | 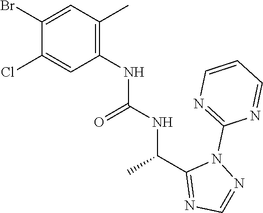 [SMILES: Cc1cc(Br)c(Cl)cc1NC(=O)N[C@@H](C)c1ncnn1-c1ncccn1] | 1.32 | 436.15 | 3 | |
| P300 | 1-(5-bromo-4-chloro-2-fluoro- phenyl)-3-[(1S)-1-(2-pyrimidin-2-yl- 1,2,4-triazol-3-yl)ethyl]urea | 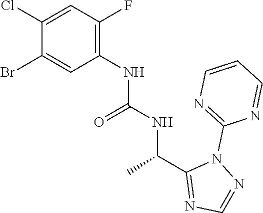 [SMILES: C[C@H](NC(=O)Nc1cc(Br)c(Cl)cc1F)c1ncnn1-c1ncccn1] | 1.33 | 440.14 | 3 | |
| P301 | 1-(6-butyl-4-methyl-3-pyridyl)-3- [(1S)-1-(2-pyrimidin-2-yl-1,2,4- triazol-3-yl)ethyl]urea | 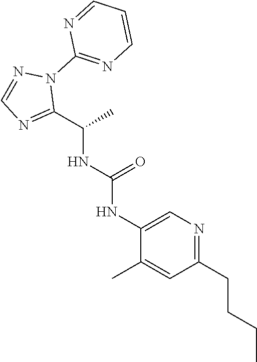 [SMILES: CCCCc1cc(C)c(NC(=O)N[C@@H](C)c2ncnn2-c2ncccn2)cn1] | 0.67 | 381.31 | 3 | |
| P302 | 1-[4-(difluoromethoxy)-2,5-dimethyl- phenyl]-3-[(1S)-1-(2-pyrimidin-2-yl- 1,2,4-triazol-3-yl)ethyl]urea | 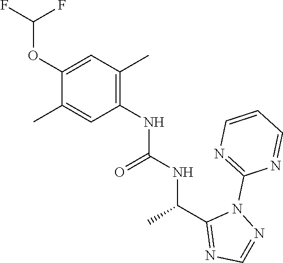 [SMILES: Cc1cc(OC(F)F)c(C)cc1NC(=O)N[C@@H](C)c1ncnn1-c1ncccn1] | 1.15 | 404.29 | 3 | |
| P303 | 1-(6-bromoindan-5-yl)-3-[(1S)-1-(2- pyrimidin-2-yl-1,2,4-triazol-3- yl)ethyl]urea | 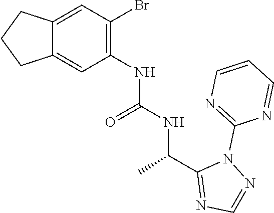 [SMILES: C[C@H](NC(=O)Nc1cc2c(cc1Br)CCC2)c1ncnn1-c1ncccn1] | 1.29 | 428.2 | 3 | |
| P304 | 1-(2-chloro-4,5-dimethoxy-phenyl)- 3-[(1S)-1-(2-pyrimidin-2-yl-1,2,4- triazol-3-yl)ethyl]urea | 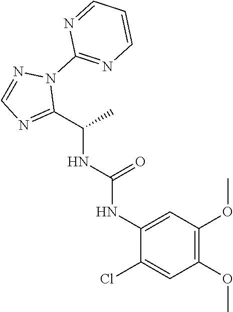 [SMILES: COc1cc(Cl)c(NC(=O)N[C@@H](C)c2ncnn2-c2ncccn2)cc1OC] | 0.94 | 404.24 | 3 | |
| P305 | 1-(5-bromo-4-chloro-2-methyl- phenyl)-3-[(1S)-1-(2-pyrimidin-2-yl- 1,2,4-triazol-3-yl)ethyl]urea | 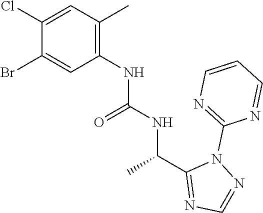 [SMILES: Cc1cc(Cl)c(Br)cc1NC(=O)N[C@@H](C)c1ncnn1-c1ncccn1] | 1.31 | 436.15 | 3 | |
| P306 | 1-(6-bromo-2,3-dihydro-1,4- benzodioxin-7-yl)-3-[(1S)-1-(2- pyrimidin-2-yl-1,2,4-triazol-3- yl)ethyl]urea | 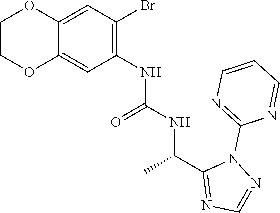 [SMILES: C[C@H](NC(=O)Nc1cc2c(cc1Br)OCCO2)c1ncnn1-c1ncccn1] | 1 | 446.2 | 3 | |
| P307 | 1-[4,5-dimethoxy-2- (trifluoromethyl)phenyl]-3-[(1S)-1- (2-pyrimidin-2-yl-1,2,4-triazol-3- yl)ethyl]urea | 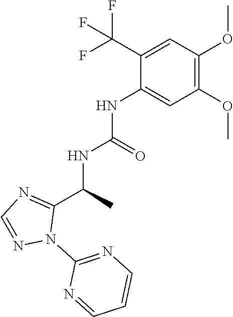 [SMILES: COc1cc(NC(=O)N[C@@H](C)c2ncnn2-c2ncccn2)c(C(F)(F)F)cc1OC] | 1.01 | 438.28 | 3 | |
| P308 | 1-(5-bromo-2,4-dimethyl-phenyl)-3- [(1S)-1-(2-pyrimidin-2-yl-1,2,4- triazol-3-yl)ethyl]urea | 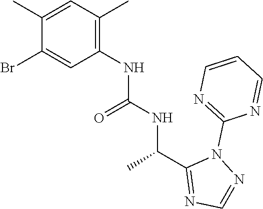 [SMILES: Cc1cc(C)c(NC(=O)N[C@@H](C)c2ncnn2-c2ncccn2)cc1Br] | 1.25 | 416.21 | 3 | |
| P309 | 1-(2-chloro-4-cyclopropyl-5-methyl- phenyl)-3-[(1S)-1-(2-pyrimidin-2-yl- 1,2,4-triazol-3-yl)ethyl]urea | 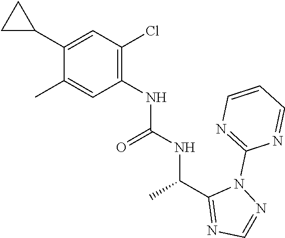 [SMILES: Cc1cc(NC(=O)N[C@@H](C)c2ncnn2-c2ncccn2)c(Cl)cc1C1CC1] | 1.38 | 398.29 | 3 | |
| P310 | 1-(2-bromo-4-iodo-5-methyl- phenyl)-3-[(1S)-1-(2-pyrimidin-2-yl- 1,2,4-triazol-3-yl)ethyl]urea | 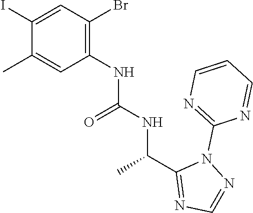 [SMILES: Cc1cc(NC(=O)N[C@@H](C)c2ncnn2-c2ncccn2)c(Br)cc1I] | 1.36 | 528.17 | 3 | |
| P311 | 1-[2,4-dichloro-5-(1,1,2,2- tetrafluoroethoxy)phenyl]-3-[(1S)-1- (2-pyrimidin-2-yl-1,2,4-triazol-3- yl)ethyl]hexahydropyrimidin-2-one | 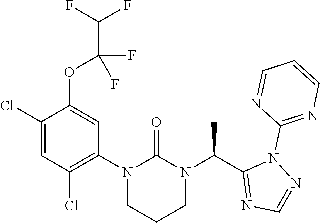 [SMILES: C[C@@H](c1ncnn1-c1ncccn1)N1CCCN(c2cc(OC(F)(F)C(F)F)c(Cl)cc2Cl)C1=O] | | | 3 | |
| P312 | 1-(4-chloro-2,5-dimethyl-phenyl)-3- [(1S)-1-(2-pyrimidin-2-yl-1,2,4- triazol-3-yl)ethyl]urea | 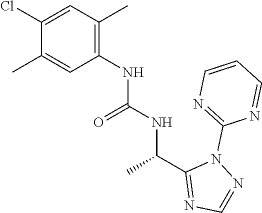 [SMILES: Cc1cc(NC(=O)N[C@@H](C)c2ncnn2-c2ncccn2)c(C)cc1Cl] | 1.21 | 372.31 | 3 | |
| P313 | 1-[(1S)-1-(2-pyrimidin-2-yl-1,2,4- triazol-3-yl)ethyl]-3-(2,4,5- trifluorophenyl)urea | 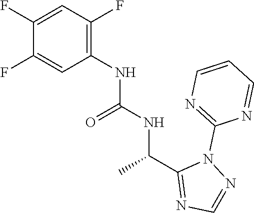 [SMILES: C[C@H](NC(=O)Nc1cc(F)c(F)cc1F)c1ncnn1-c1ncccn1] | 1.04 | 364.28 | 3 | |
| P314 | 1-(4-bromo-2-chloro-5-fluoro- phenyl)-3-[(1S)-1-(2-pyrimidin-2-yl- 1,2,4-triazol-3-yl)ethyl]urea | 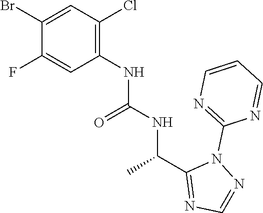 [SMILES: C[C@H](NC(=O)Nc1cc(F)c(Br)cc1Cl)c1ncnn1-c1ncccn1] | 1.35 | 440.21 | 3 | |
| P315 | methyl 2,4-dimethyl-5-[[(1S)-1-(2- pyrimidin-2-yl-1,2,4-triazol-3- yl)ethyl]carbamoylamino]benzoate | 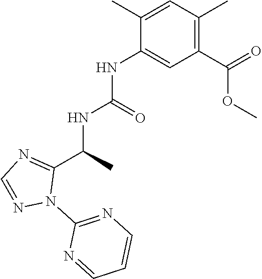 [SMILES: COC(=O)c1cc(NC(=O)N[C@@H](C)c2ncnn2-c2ncccn2)c(C)cc1C] | 1.05 | 396.37 | 3 | |
| P316 | 1-[2-bromo-4-fluoro-5- (trifluoromethoxy)phenyl]-3-[(1S)-1- (2-pyrimidin-2-yl-1,2,4-triazol-3- yl)ethyl]urea | 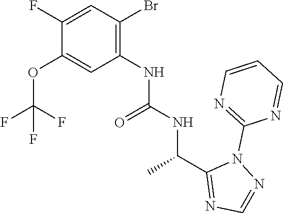 [SMILES: C[C@H](NC(=O)Nc1cc(OC(F)(F)F)c(F)cc1Br)c1ncnn1-c1ncccn1] | 1.42 | 490.26 | 3 | |
| P317 | 1-(7-chloro-3,4-dihydro-2H-1,5- benzodioxepin-8-yl)-3-[(1S)-1-(2- pyrimidin-2-yl-1,2,4-triazol-3- yl)ethyl]urea | 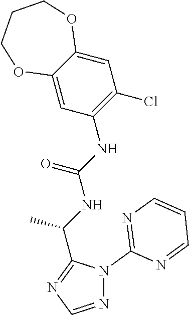 [SMILES: C[C@H](NC(=O)Nc1cc2c(cc1Cl)OCCCO2)c1ncnn1-c1ncccn1] | 1.05 | 416.32 | 3 | |
| P318 | 1-(6-chloro-4-methyl-3-pyridyl)-3- [(1S)-1-(2-pyrimidin-2-yl-1,2,4- triazol-3-yl)ethyl]urea | 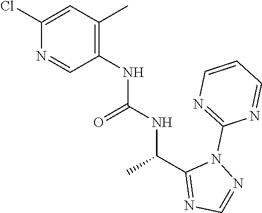 [SMILES: Cc1cc(Cl)ncc1NC(=O)N[C@@H](C)c1ncnn1-c1ncccn1] | 0.82 | 359.3 | 3 | |
| P319 | 1-(2-acetyl-4-bromo-5-fluoro- phenyl)-3-[(1S)-1-(2-pyrimidin-2-yl- 1,2,4-triazol-3-yl)ethyl]urea | 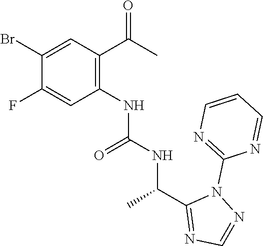 [SMILES: CC(=O)c1cc(Br)c(F)cc1NC(=O)N[C@@H](C)c1ncnn1-c1ncccn1] | 1.75 | 447.14 | 3 | |
| P320 | methyl 4-fluoro-2-methoxy-5-[[(1S)- 1-(2-pyrimidin-2-yl-1,2,4-triazol-3- yl)ethyl]carbamoylamino]benzoate | 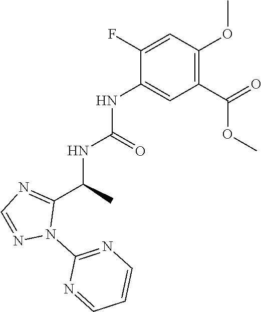 [SMILES: COC(=O)c1cc(NC(=O)N[C@@H](C)c2ncnn2-c2ncccn2)c(F)cc1OC] | 1.13 | 416.35 | 3 | |
| P321 | 1-(5-bromo-2-chloro-4-methyl- phenyl)-3-[(1S)-1-(2-pyrimidin-2-yl- 1,2,4-triazol-3-yl)ethyl]urea | 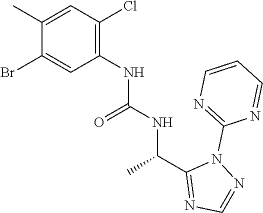 [SMILES: Cc1cc(Cl)c(NC(=O)N[C@@H](C)c2ncnn2-c2ncccn2)cc1Br] | 1.36 | 436.23 | 3 | |
| P322 | 1-(6-bromo-2-oxo-3,4-dihydro-1H- quinolin-7-yl)-3-[(1S)-1-(2-pyrimidin- 2-yl-1,2,4-triazol-3-yl)ethyl]urea | 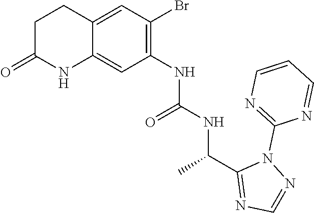 [SMILES: C[C@H](NC(=O)Nc1cc2c(cc1Br)CCC(=O)N2)c1ncnn1-c1ncccn1] | 0.89 | 457.28 | 3 | |
| P323 | 1-(2,5-dimethyl-4-nitro-phenyl)-3- [(1S)-1-(2-pyrimidin-2-yl-1,2,4- triazol-3-yl)ethyl]urea | 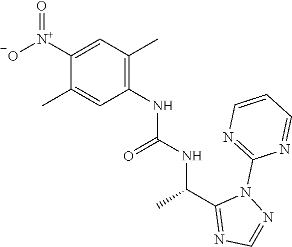 [SMILES: Cc1cc([N+](=O)[O-])c(C)cc1NC(=O)N[C@@H](C)c1ncnn1-c1ncccn1] | 1.12 | 383.32 | 3 | |
| P324 | 1-(4-chloro-2-fluoro-5-methoxy- phenyl)-3-[(1S)-1-(2-pyrimidin-2-yl- 1,2,4-triazol-3-yl)ethyl]urea | 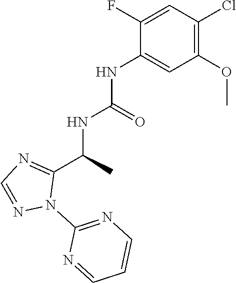 [SMILES: COc1cc(NC(=O)N[C@@H](C)c2ncnn2-c2ncccn2)c(F)cc1Cl] | 1.13 | 392.29 | 3 | |
| P325 | 1-(4-bromo-2-fluoro-5-methyl- phenyl)-3-[(1S)-1-(2-pyrimidin-2-yl- 1,2,4-triazol-3-yl)ethyl]urea | 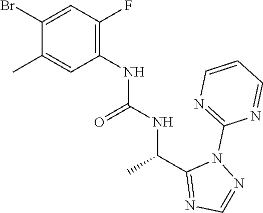 [SMILES: Cc1cc(NC(=O)N[C@@H](C)c2ncnn2-c2ncccn2)c(F)cc1Br] | 1.27 | 420.25 | 3 | |
| P326 | 1-(2-chloro-4,5-dimethyl-phenyl)-3- [(1S)-1-(2-pyrimidin-2-yl-1,2,4- triazol-3-yl)ethyl]urea | 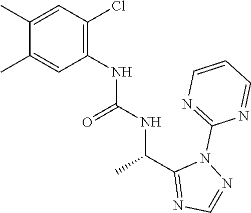 [SMILES: Cc1cc(Cl)c(NC(=O)N[C@@H](C)c2ncnn2-c2ncccn2)cc1C] | 1.21 | 372.31 | 3 | |
| P327 | 1-(2,5-difluoro-4-methoxy-phenyl)- 3-[(1S)-1-(2-pyrimidin-2-yl-1,2,4- triazol-3-yl)ethyl]urea | 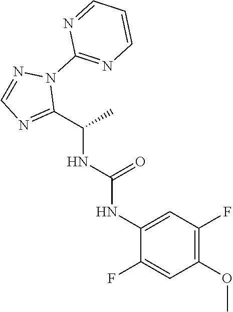 [SMILES: COc1cc(F)c(NC(=O)N[C@@H](C)c2ncnn2-c2ncccn2)cc1F] | 0.94 | 376.31 | 3 | |
| P328 | 1-[4-methyl-6-(trifluoromethyl)-3- pyridyl]-3-[(1S)-1-(2-pyrimidin-2-yl- 1,2,4-triazol-3-yl)ethyl]urea | 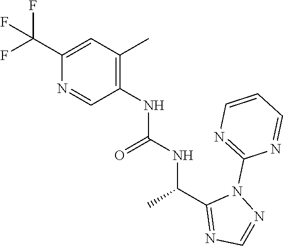 [SMILES: Cc1cc(C(F)(F)F)ncc1NC(=O)N[C@@H](C)c1ncnn1-c1ncccn1] | 0.99 | 393.34 | 3 | |
| P329 | methyl 2,4-difluoro-5-[[(1S)-1-(2- pyrimidin-2-yl-1,2,4-triazol-3- yl)ethyl]carbamoylamino]benzoate | 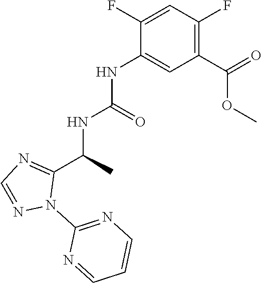 [SMILES: COC(=O)c1cc(NC(=O)N[C@@H](C)c2ncnn2-c2ncccn2)c(F)cc1F] | 0.97 | 404.33 | 3 | |
| P330 | 1-(2-bromo-5-chloro-4-methyl- phenyl)-3-[(1S)-1-(2-pyrimidin-2-yl- 1,2,4-triazol-3-yl)ethyl]urea | 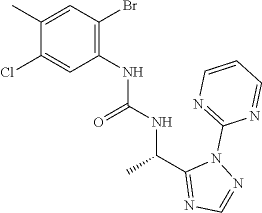 [SMILES: Cc1cc(Br)c(NC(=O)N[C@@H](C)c2ncnn2-c2ncccn2)cc1Cl] | 1.34 | 436.24 | 3 | |
| P331 | 1-(5-bromo-4-fluoro-2-methyl- phenyl)-3-[(1S)-1-(2-pyrimidin-2-yl- 1,2,4-triazol-3-yl)ethyl]urea | 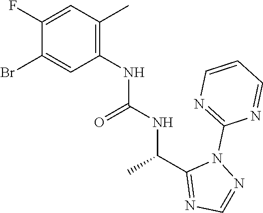 [SMILES: Cc1cc(F)c(Br)cc1NC(=O)N[C@@H](C)c1ncnn1-c1ncccn1] | 1.14 | 420.25 | 3 | |
| P332 | 1-[2,5-difluoro-4- (trifluoromethyl)phenyl]-3-[(1S)-1- (2-pyrimidin-2-yl-1,2,4-triazol-3- yl)ethyl]urea | 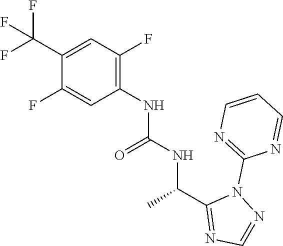 [SMILES: C[C@H](NC(=O)Nc1cc(F)c(C(F)(F)F)cc1F)c1ncnn1-c1ncccn1] | 1.34 | 414.3 | 3 | |
| P333 | 1-(5-bromo-2-methoxy-4-methyl- phenyl)-3-[(1S)-1-(2-pyrimidin-2-yl- 1,2,4-triazol-3-yl)ethyl]urea | 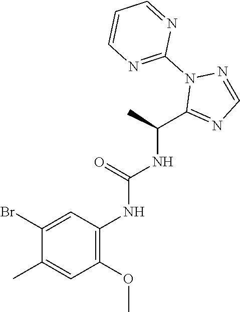 [SMILES: COc1cc(C)c(Br)cc1NC(=O)N[C@@H](C)c1ncnn1-c1ncccn1] | 1.28 | 432.28 | 3 | |
| P334 | 1-(2,4-difluoro-5-methoxy-phenyl)- 3-[(1S)-1-(2-pyrimidin-2-yl-1,2,4- triazol-3-yl)ethyl]urea | 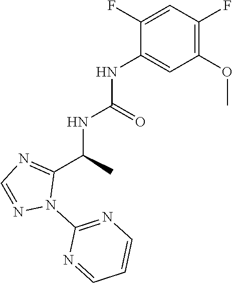 [SMILES: COc1cc(NC(=O)N[C@@H](C)c2ncnn2-c2ncccn2)c(F)cc1F] | 0.97 | 376.31 | 3 | |
| P335 | 1-(6-fluoro-3H-benzotriazol-5-yl)-3- [(1S)-1-(2-pyrimidin-2-yl-1,2,4- triazol-3-yl)ethyl]urea | 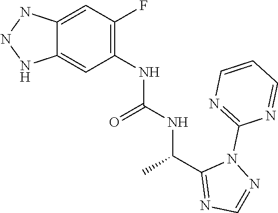 [SMILES: C[C@H](NC(=O)Nc1cc2c(cc1F)NNN2)c1ncnn1-c1ncccn1] | 0.7 | 369.31 | 3 | |
| P336 | ethyl 4-fluoro-2-methyl-5-[[(1S)-1- (2-pyrimidin-2-yl-1,2,4-triazol-3- yl)ethyl]carbamoylamino]benzoate | 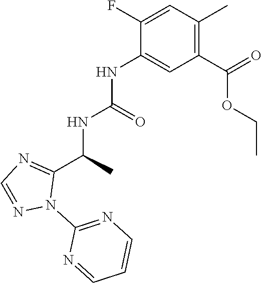 [SMILES: CCOC(=O)c1cc(NC(=O)N[C@@H](C)c2ncnn2-c2ncccn2)c(F)cc1C] | 1.19 | 414.36 | 3 | |
| P337 | 1-(6-chloroindan-5-yl)-3-[(1S)-1-(2- pyrimidin-2-yl-1,2,4-triazol-3- yl)ethyl]urea | 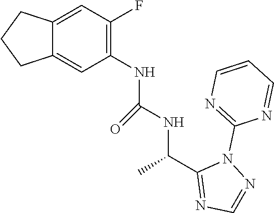 [SMILES: C[C@H](NC(=O)Nc1cc2c(cc1F)CCC2)c1ncnn1-c1ncccn1] | 1.27 | 384.33 | 3 | |
| P338 | 1-(2,5-dichloro-4-methyl-phenyl)-3- [(1S)-1-(2-pyrimidin-2-yl-1,2,4- triazol-3-yl)ethyl]urea | 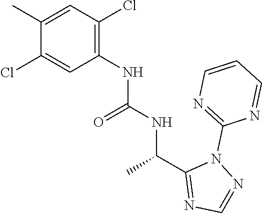 [SMILES: Cc1cc(Cl)c(NC(=O)N[C@@H](C)c2ncnn2-c2ncccn2)cc1Cl] | 1.33 | 392.28 | 3 | |
| P339 | 1-(4-bromo-2,5-difluoro-phenyl)-3- [(1S)-1-(2-pyrimidin-2-yl-1,2,4- triazol-3-yl)ethyl]urea | 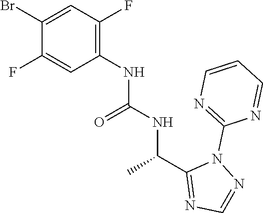 [SMILES: C[C@H](NC(=O)Nc1cc(F)c(Br)cc1F)c1ncnn1-c1ncccn1] | 1.23 | 424.23 | 3 | |
| P340 | 1-(2-fluoro-4,5-dimethyl-phenyl)-3- [(1S)-1-(2-pyrimidin-2-yl-1,2,4- triazol-3-yl)ethyl]urea | 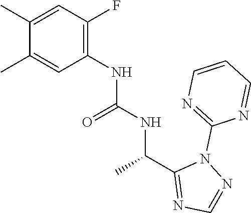 [SMILES: Cc1cc(F)c(NC(=O)N[C@@H](C)c2ncnn2-c2ncccn2)cc1C] | 1.11 | 356.32 | 3 | |
| P341 | 1-(5-bromo-4-fluoro-2-methoxy- phenyl)-3-[(1S)-1-(2-pyrimidin-2-yl- 1,2,4-triazol-3-yl)ethyl]urea | 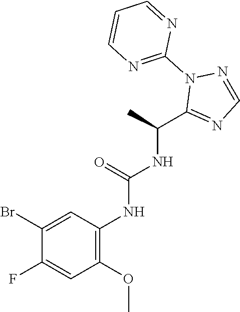 [SMILES: COc1cc(F)c(Br)cc1NC(=O)N[C@@H](C)c1ncnn1-c1ncccn1] | 1.06 | 436.25 | 3 | |
| P342 | 1-(6-bromo-4-methyl-3-pyridyl)-3- [(1S)-1-(2-pyrimidin-2-yl-1,2,4- triazol-3-yl)ethyl]urea | 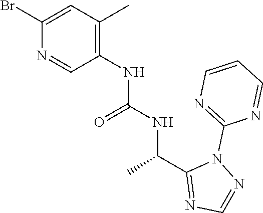 [SMILES: Cc1cc(Br)ncc1NC(=O)N[C@@H](C)c1ncnn1-c1ncccn1] | 0.88 | 403.27 | 3 | |
| P343 | 1-(5-bromo-2,4-difluoro-phenyl)-3- [(1S)-1-(2-pyrimidin-2-yl-1,2,4- triazol-3-yl)ethyl]urea | 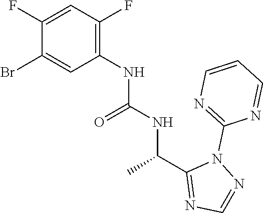 [SMILES: C[C@H](NC(=O)Nc1cc(Br)c(F)cc1F)c1ncnn1-c1ncccn1] | 1.16 | 424.23 | 3 | |
| P344 | 1-(6-methyl-1,3-benzodioxol-5-yl)-3- [(1S)-1-(2-pyrimidin-2-yl-1,2,4- triazol-3-yl)ethyl]urea | 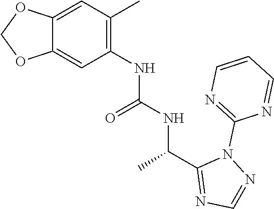 [SMILES: Cc1cc2c(cc1NC(=O)N[C@@H](C)c1ncnn1-c1ncccn1)OCO2] | 0.85 | 368.32 | 3 | |
| P345 | 1-(2-bromo-4-fluoro-5-nitro-phenyl)- 3-[(1S)-1-(2-pyrimidin-2-yl-1,2,4- triazol-3-yl)ethyl]urea | 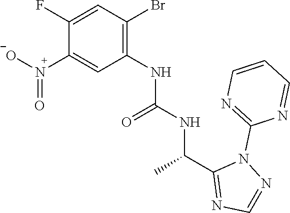 [SMILES: C[C@H](NC(=O)Nc1cc([N+](=O)[O-])c(F)cc1Br)c1ncnn1-c1ncccn1] | 1.13 | 451.23 | 3 | |
| P346 | 1-(2,5-difluoro-4-methyl-phenyl)-3- [(1S)-1-(2-pyrimidin-2-yl-1,2,4- triazol-3-yl)ethyl]urea | 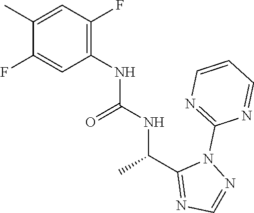 [SMILES: Cc1cc(F)c(NC(=O)N[C@@H](C)c2ncnn2-c2ncccn2)cc1F] | 1.11 | 360.3 | 3 | |
| P347 | 1-(2-bromo-5-fluoro-4-methoxy- phenyl)-3-[(1S)-1-(2-pyrimidin-2-yl- 1,2,4-triazol-3-yl)ethyl]urea | 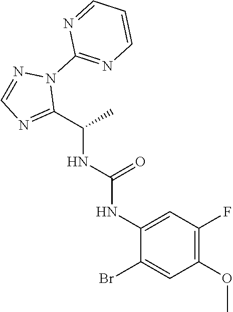 [SMILES: COc1cc(Br)c(NC(=O)N[C@@H](C)c2ncnn2-c2ncccn2)cc1F] | 1.05 | 436.25 | 3 | |
| P348 | 1-[4-methoxy-6-(trifluoromethyl)-3- pyridyl]-3-[(1S)-1-(2-pyrimidin-2-yl- 1,2,4-triazol-3-yl)ethyl]urea | 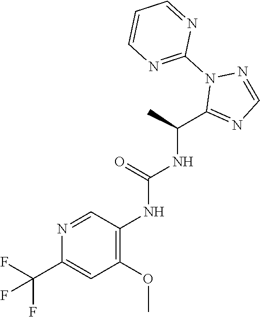 [SMILES: COc1cc(C(F)(F)F)ncc1NC(=O)N[C@@H](C)c1ncnn1-c1ncccn1] | 0.98 | 409.33 | 3 | |
| P349 | 1-(6-fluoro-3-oxo-4H-1,4- benzoxazin-7-yl)-3-[(1S)-1-(2- pyrimidin-2-yl-1,2,4-triazol-3- yl)ethyl]urea | 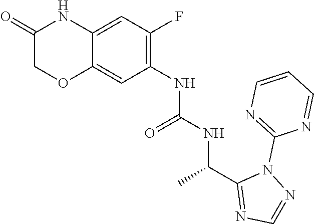 [SMILES: C[C@H](NC(=O)Nc1cc2c(cc1F)NC(=O)CO2)c1ncnn1-c1ncccn1] | 0.71 | 399.33 | 3 | |
| P350 | 1-[5-bromo-2-fluoro-4- (trifluoromethyl)phenyl]-3-[(1S)-1- (2-pyrimidin-2-yl-1,2,4-triazol-3- yl)ethyl]urea | 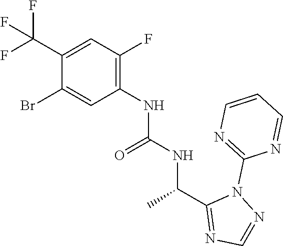 [SMILES: C[C@H](NC(=O)Nc1cc(Br)c(C(F)(F)F)cc1F)c1ncnn1-c1ncccn1] | 1.43 | 474.25 | 3 | |
| P351 | 1-(4-bromo-2-chloro-5-methoxy- phenyl)-3-[(1S)-1-(2-pyrimidin-2-yl- 1,2,4-triazol-3-yl)ethyl]urea | 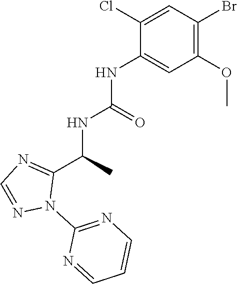 [SMILES: COc1cc(NC(=O)N[C@@H](C)c2ncnn2-c2ncccn2)c(Cl)cc1Br] | 1.29 | 452.23 | 3 | |
| P352 | 1-(4,5-dichloro-2-methoxy-phenyl)- 3-[(1S)-1-(2-pyrimidin-2-yl-1,2,4- triazol-3-yl)ethyl]urea | 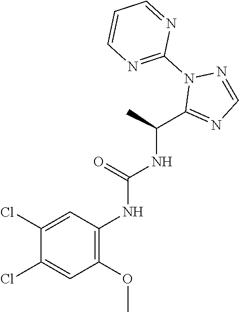 [SMILES: COc1cc(Cl)c(Cl)cc1NC(=O)N[C@@H](C)c1ncnn1-c1ncccn1] | 1.3 | 408.27 | 3 | |
| P353 | 4-fluoro-N,2-dimethyl-5-[[(1S)-1-(2- pyrimidin-2-yl-1,2,4-triazol-3- yl)ethyl]carbamoylamino]benzamide | 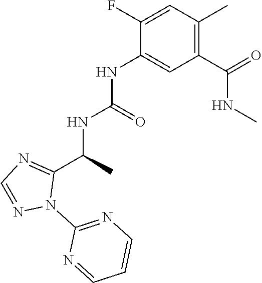 [SMILES: CNC(=O)c1cc(NC(=O)N[C@@H](C)c2ncnn2-c2ncccn2)c(F)cc1C] | 0.74 | 399.36 | 3 | |
| P354 | 1-(2,4-difluoro-5-methyl-phenyl)-3- [(1S)-1-(2-pyrimidin-2-yl-1,2,4- triazol-3-yl)ethyl]urea | 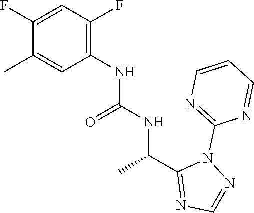 [SMILES: Cc1cc(NC(=O)N[C@@H](C)c2ncnn2-c2ncccn2)c(F)cc1F] | 1.05 | 360.3 | 3 | |
| P355 | 1-(4-chloro-2-cyclopropyl-5- methoxy-phenyl)-3-[(1S)-1-(2- pyrimidin-2-yl-1,2,4-triazol-3- yl)ethyl]urea | 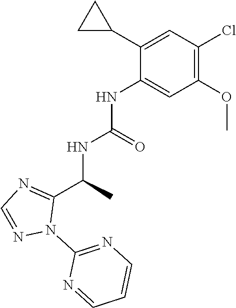 [SMILES: COc1cc(NC(=O)N[C@@H](C)c2ncnn2-c2ncccn2)c(C2CC2)cc1Cl] | 1.28 | 414.33 | 3 | |
| P356 | 1-(4-chloro-2,5-dimethoxy-phenyl)- 3-[(1S)-1-(2-pyrimidin-2-yl-1,2,4- triazol-3-yl)ethyl]urea | 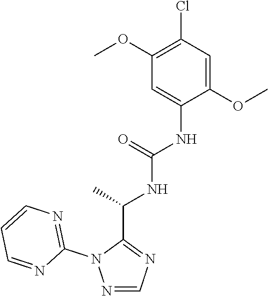 [SMILES: COc1cc(NC(=O)N[C@@H](C)c2ncnn2-c2ncccn2)c(OC)cc1Cl] | 1.13 | 404.32 | 3 | |
| P357 | 1-(4-chloro-2-methyl-5-nitro- phenyl)-3-[(1S)-1-(2-pyrimidin-2-yl- 1,2,4-triazol-3-yl)ethyl]urea | 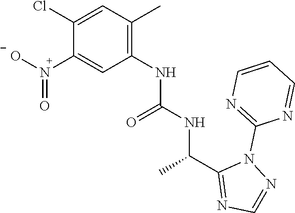 [SMILES: Cc1cc(Cl)c([N+](=O)[O-])cc1NC(=O)N[C@@H](C)c1ncnn1-c1ncccn1] | 1.17 | 403.31 | 3 | |
| P358 | 1-(4-ethyl-2-methoxy-5-methyl- phenyl)-3-[(1S)-1-(2-pyrimidin-2-yl- 1,2,4-triazol-3-yl)ethyl]urea | 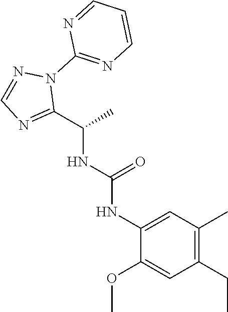 [SMILES: CCc1cc(OC)c(NC(=O)N[C@@H](C)c2ncnn2-c2ncccn2)cc1C] | 1.25 | 382.37 | 3 | |
| P359 | 1-[4-methoxy-2-methyl-5- (trifluoromethyl)phenyl]-3-[(1S)-1- (2-pyrimidin-2-yl-1,2,4-triazol-3- yl)ethyl]urea | 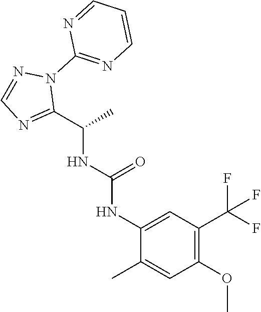 [SMILES: COc1cc(C)c(NC(=O)N[C@@H](C)c2ncnn2-c2ncccn2)cc1C(F)(F)F] | 1.14 | 422.35 | 3 | |
| P360 | 1-[4-chloro-2-methylsulfanyl-5- (trifluoromethyl)phenyl]-3-[(1S)-1- (2-pyrimidin-2-yl-1,2,4-triazol-3- yl)ethyl]urea | 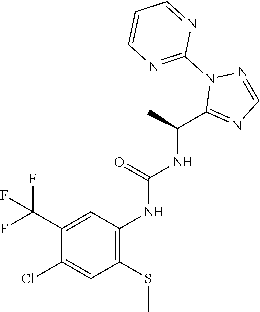 [SMILES: CSc1cc(Cl)c(C(F)(F)F)cc1NC(=O)N[C@@H](C)c1ncnn1-c1ncccn1] | 1.43 | 458.3 | 3 | |
| P361 | 1-(4,5-difluoro-2-iodo-phenyl)-3- [(1S)-1-(2-pyrimidin-2-yl-1,2,4- triazol-3-yl)ethyl]urea | 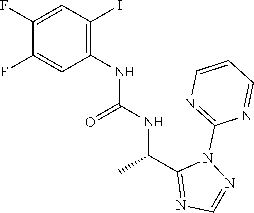 [SMILES: C[C@H](NC(=O)Nc1cc(F)c(F)cc1I)c1ncnn1-c1ncccn1] | 1.16 | 472.24 | 3 | |
| P362 | 1-(5-bromo-2,4-dimethoxy-phenyl)- 3-[(1S)-1-(2-pyrimidin-2-yl-1,2,4- triazol-3-yl)ethyl]urea | 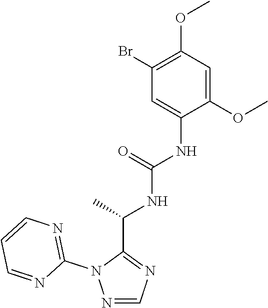 [SMILES: COc1cc(OC)c(NC(=O)N[C@@H](C)c2ncnn2-c2ncccn2)cc1Br] | 1.08 | 448.28 | 3 | |
| P363 | 1-(4-methoxy-2,5-dimethyl-phenyl)- 3-[(1S)-1-(2-pyrimidin-2-yl-1,2,4- triazol-3-yl)ethyl]urea | 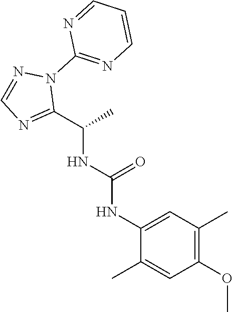 [SMILES: COc1cc(C)c(NC(=O)N[C@@H](C)c2ncnn2-c2ncccn2)cc1C] | 1 | 368.36 | 3 | |
| P364 | methyl 2-fluoro-5-methyl-4-[[(1S)-1- (2-pyrimidin-2-yl-1,2,4-triazol-3- yl)ethyl]carbamoylamino]benzoate | 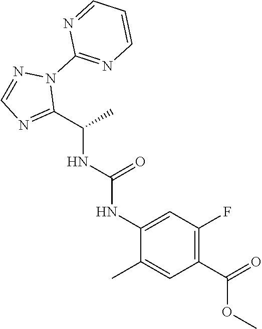 [SMILES: COC(=O)c1cc(C)c(NC(=O)N[C@@H](C)c2ncnn2-c2ncccn2)cc1F] | 1.05 | 400.34 | 3 | |
| P365 | 1-[4-(difluoromethoxy)-2,5-difluoro- phenyl]-3-[(1S)-1-(2-pyrimidin-2-yl- 1,2,4-triazol-3-yl)ethyl]urea | 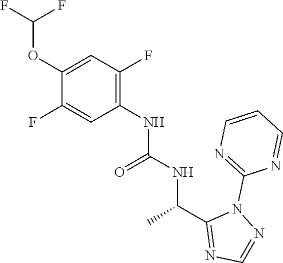 [SMILES: C[C@H](NC(=O)Nc1cc(F)c(OC(F)F)cc1F)c1ncnn1-c1ncccn1] | 1.14 | 412.31 | 3 | |
| P366 | 1-(5-chloro-2-fluoro-4-methoxy- phenyl)-3-[(1S)-1-(2-pyrimidin-2-yl- 1,2,4-triazol-3-yl)ethyl]urea | 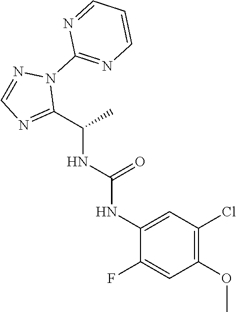 [SMILES: COc1cc(F)c(NC(=O)N[C@@H](C)c2ncnn2-c2ncccn2)cc1Cl] | 1.03 | 392.29 | 3 | |
| P367 | 1-(4-bromo-5-fluoro-2-methyl- phenyl)-3-[(1S)-1-(2-pyrimidin-2-yl- 1,2,4-triazol-3-yl)ethyl]urea | 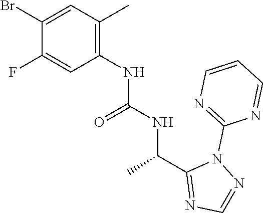 [SMILES: Cc1cc(Br)c(F)cc1NC(=O)N[C@@H](C)c1ncnn1-c1ncccn1] | 1.23 | 420.25 | 3 | |
| P368 | 1-(4-bromo-5-chloro-2-methoxy- phenyl)-3-[(1S)-1-(2-pyrimidin-2-yl- 1,2,4-triazol-3-yl)ethyl]urea | 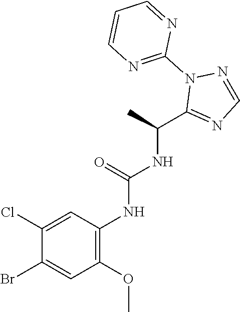 [SMILES: COc1cc(Br)c(Cl)cc1NC(=O)N[C@@H](C)c1ncnn1-c1ncccn1] | 1.34 | 452.23 | 3 | |
| P369 | methyl 2,2-difluoro-6-[[(1S)-1-(2- pyrimidin-2-yl-1,2,4-triazol-3- yl)ethyl]carbamoylamino]-1,3- benzodioxole-5-carboxylate | 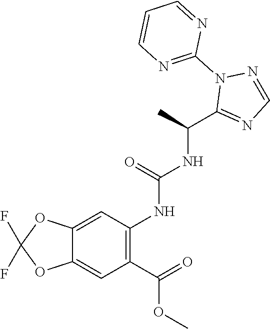 [SMILES: COC(=O)c1cc2c(cc1NC(=O)N[C@@H](C)c1ncnn1-c1ncccn1)OC(F)(F)O2] | 1.4 | 448.33 | 3 | |
| P370 | 1-(6-chloro-1,3-benzodioxol-5-yl)-3- [(1S)-1-(2-pyrimidin-2-yl-1,2,4- triazol-3-yl)ethyl]urea | 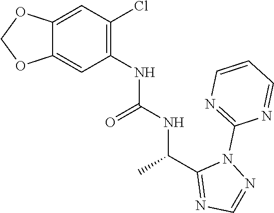 [SMILES: C[C@H](NC(=O)Nc1cc2c(cc1Cl)OCO2)c1ncnn1-c1ncccn1] | 0.96 | 388.29 | 3 | |
| P371 | 1-(5-bromo-4-fluoro-2-iodo-phenyl)- 3-[(1S)-1-(2-pyrimidin-2-yl-1,2,4- triazol-3-yl)ethyl]urea | 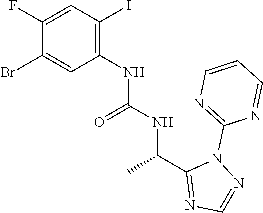 [SMILES: C[C@H](NC(=O)Nc1cc(Br)c(F)cc1I)c1ncnn1-c1ncccn1] | 1.27 | 532.21 | 3 | |
| P372 | 1-(2-methoxy-4,5-dimethyl-phenyl)- 3-[(1S)-1-(2-pyrimidin-2-yl-1,2,4- triazol-3-yl)ethyl]urea | 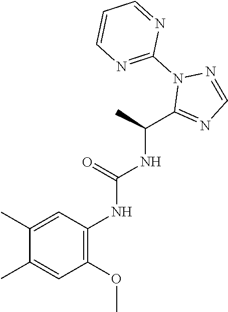 [SMILES: COc1cc(C)c(C)cc1NC(=O)N[C@@H](C)c1ncnn1-c1ncccn1] | 1.12 | 368.36 | 3 | |
| P373 | 1-(4-bromo-5-chloro-2-fluoro- phenyl)-3-[(1S)-1-(2-pyrimidin-2-yl- 1,2,4-triazol-3-yl)ethyl]urea | 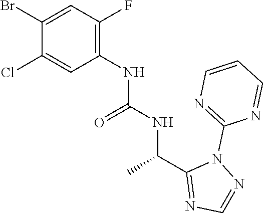 [SMILES: C[C@H](NC(=O)Nc1cc(Cl)c(Br)cc1F)c1ncnn1-c1ncccn1] | 1.32 | 440.21 | 3 | |
| P374 | methyl 4-(difluoromethoxy)-5- methoxy-2-[[(1S)-1-(2-pyrimidin-2- yl-1,2,4-triazol-3- yl)ethyl]carbamoylamino]benzoate | 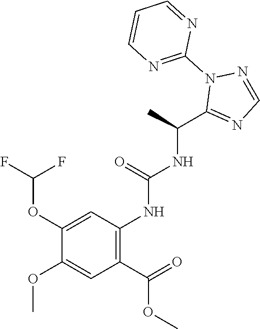 [SMILES: COC(=O)c1cc(OC)c(OC(F)F)cc1NC(=O)N[C@@H](C)c1ncnn1-c1ncccn1] | 1.21 | 464.37 | 3 | |
| P375 | methyl 4,5-dimethoxy-2-[[(1S)-1-(2- pyrimidin-2-yl-1,2,4-triazol-3- yl)ethyl]carbamoylamino]benzoate | 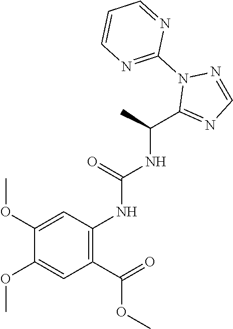 [SMILES: COC(=O)c1cc(OC)c(OC)cc1NC(=O)N[C@@H](C)c1ncnn1-c1ncccn1] | 1.03 | 428.36 | 3 | |
| P376 | 1-(4-chloro-5-fluoro-2-iodo-phenyl)- 3-[(1S)-1-(2-pyrimidin-2-yl-1,2,4- triazol-3-yl)ethyl]urea | 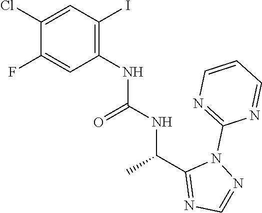 [SMILES: C[C@H](NC(=O)Nc1cc(F)c(Cl)cc1I)c1ncnn1-c1ncccn1] | 1.33 | 488.23 | 3 | |
| P377 | 1-(4-chloro-2-methoxy-5-methyl- phenyl)-3-[(1S)-1-(2-pyrimidin-2-yl- 1,2,4-triazol-3-yl)ethyl]urea | 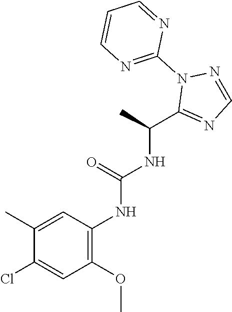 [SMILES: COc1cc(Cl)c(C)cc1NC(=O)N[C@@H](C)c1ncnn1-c1ncccn1] | 1.24 | 388.32 | 3 | |
| P378 | 1-(5-fluoro-4-methoxy-2-methyl- phenyl)-3-[(1S)-1-(2-pyrimidin-2-yl- 1,2,4-triazol-3-yl)ethyl]urea | 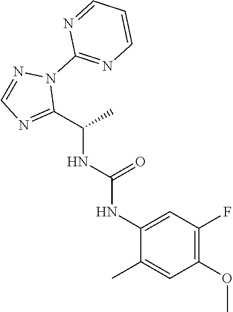 [SMILES: COc1cc(C)c(NC(=O)N[C@@H](C)c2ncnn2-c2ncccn2)cc1F] | 0.92 | 372.32 | 3 | |
| P379 | 1-(5-chloro-2,4-difluoro-phenyl)-3- [(1S)-1-(2-pyrimidin-2-yl-1,2,4- triazol-3-yl)ethyl]urea | 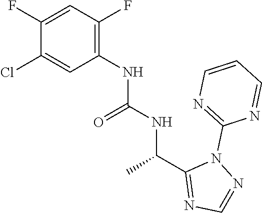 [SMILES: C[C@H](NC(=O)Nc1cc(Cl)c(F)cc1F)c1ncnn1-c1ncccn1] | 1.14 | 380.26 | 3 | |
| P380 | 1-(2,4-dichloro-5-fluoro-phenyl)-3- [(1S)-1-(2-pyrimidin-2-yl-1,2,4- triazol-3-yl)ethyl]urea | 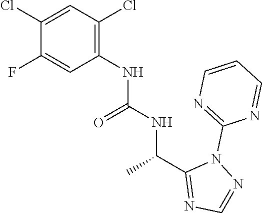 [SMILES: C[C@H](NC(=O)Nc1cc(F)c(Cl)cc1Cl)c1ncnn1-c1ncccn1] | 1.31 | 396.25 | 3 | |
| P381 | 1-(5-fluoro-3-methyl-2-oxo-1,3- benzoxazol-6-yl)-3-[(1S)-1-(2- pyrimidin-2-yl-1,2,4-triazol-3- yl)ethyl]urea | 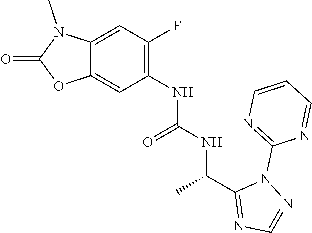 [SMILES: C[C@H](NC(=O)Nc1cc2oc(=O)n(C)c2cc1F)c1ncnn1-c1ncccn1] | 0.79 | 399.32 | 3 | |
| P382 | methyl 2-bromo-4-methyl-5-[[(1S)- 1-(2-pyrimidin-2-yl-1,2,4-triazol-3- yl)ethyl]carbamoylamino]benzoate | 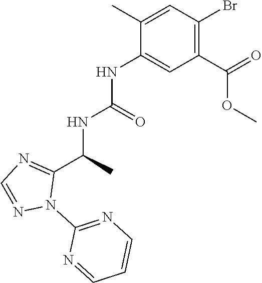 [SMILES: COC(=O)c1cc(NC(=O)N[C@@H](C)c2ncnn2-c2ncccn2)c(C)cc1Br] | 1.12 | 460.28 | 3 | |
| P383 | 1-(4,5-difluoro-2-methoxy-phenyl)- 3-[(1S)-1-(2-pyrimidin-2-yl-1,2,4- triazol-3-yl)ethyl]urea | 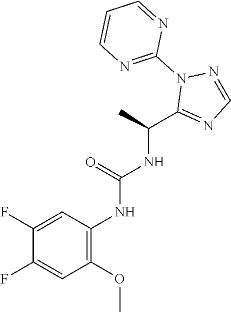 [SMILES: COc1cc(F)c(F)cc1NC(=O)N[C@@H](C)c1ncnn1-c1ncccn1] | 1.08 | 376.31 | 3 | |
| P384 | 1-(2-chloro-4-methoxy-5-methyl- phenyl)-3-[(1S)-1-(2-pyrimidin-2-yl- 1,2,4-triazol-3-yl)ethyl]urea | 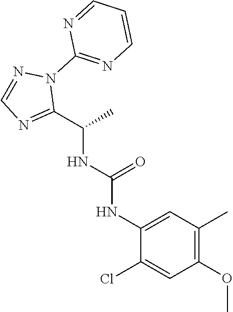 [SMILES: COc1cc(Cl)c(NC(=O)N[C@@H](C)c2ncnn2-c2ncccn2)cc1C] | 1.1 | 388.32 | 3 | |
| P385 | 1-(2-chloro-4-fluoro-5-methyl- phenyl)-3-[(1S)-1-(2-pyrimidin-2-yl- 1,2,4-triazol-3-yl)ethyl]urea | 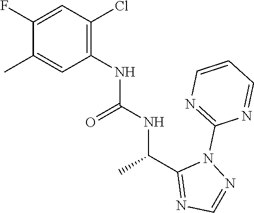 [SMILES: Cc1cc(NC(=O)N[C@@H](C)c2ncnn2-c2ncccn2)c(Cl)cc1F] | 1.15 | 376.28 | 3 | |
| P386 | 1-(4-bromo-2-ethoxy-5-fluoro- phenyl)-3-[(1S)-1-(2-pyrimidin-2-yl- 1,2,4-triazol-3-yl)ethyl]urea | 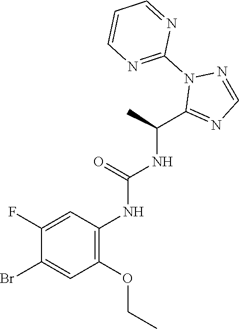 [SMILES: CCOc1cc(Br)c(F)cc1NC(=O)N[C@@H](C)c1ncnn1-c1ncccn1] | 1.37 | 450.28 | 3 | |
| P387 | 1-(5-chloro-2,4-dimethoxy-phenyl)- 3-[(1S)-1-(2-pyrimidin-2-yl-1,2,4- triazol-3-yl)ethyl]urea | 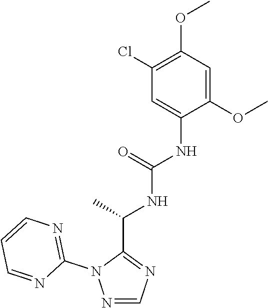 [SMILES: COc1cc(OC)c(NC(=O)N[C@@H](C)c2ncnn2-c2ncccn2)cc1Cl] | 1.04 | 404.32 | 3 | |
| P388 | 1-(5-bromo-2-chloro-4-fluoro- phenyl)-3-[(1S)-1-(2-pyrimidin-2-yl- 1,2,4-triazol-3-yl)ethyl]urea | 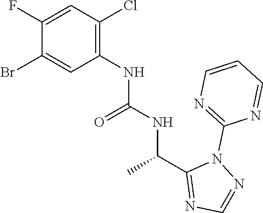 [SMILES: C[C@H](NC(=O)Nc1cc(Br)c(F)cc1Cl)c1ncnn1-c1ncccn1] | 1.26 | 440.21 | 3 | |
| P389 | 1-[2,5-dichloro-4- (difluoromethoxy)phenyl]-3-[(1S)-1- (2-pyrimidin-2-yl-1,2,4-triazol-3- yl)ethyl]urea | 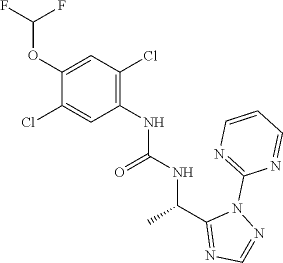 [SMILES: C[C@H](NC(=O)Nc1cc(Cl)c(OC(F)F)cc1Cl)c1ncnn1-c1ncccn1] | 1.3 | 444.26 | 3 | |
| P390 | 1-[4-bromo-2-(difluoromethoxy)-5- fluoro-phenyl]-3-[(1S)-1-(2- pyrimidin-2-yl-1,2,4-triazol-3- yl)ethyl]urea | 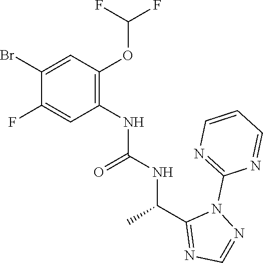 [SMILES: C[C@H](NC(=O)Nc1cc(F)c(Br)cc1OC(F)F)c1ncnn1-c1ncccn1] | 1.34 | 472.26 | 3 | |
| P391 | 1-(5-cyano-2,4-difluoro-phenyl)-3- [(1S)-1-(2-pyrimidin-2-yl-1,2,4- triazol-3-yl)ethyl]urea | 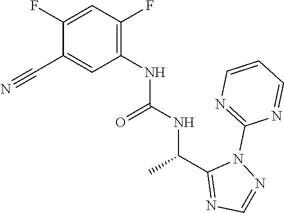 [SMILES: C[C@H](NC(=O)Nc1cc(C#N)c(F)cc1F)c1ncnn1-c1ncccn1] | 0.95 | 371.29 | 3 | |
| P392 | N-[2,4-difluoro-5-[[(1S)-1-(2- pyrimidin-2-yl-1,2,4-triazol-3- yl)ethyl]carbamoylamino]phenyl]acet- amide | 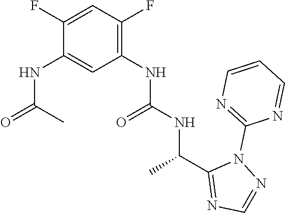 [SMILES: CC(=O)Nc1cc(NC(=O)N[C@@H](C)c2ncnn2-c2ncccn2)c(F)cc1F] | 0.7 | 403.32 | 3 | |
| P393 | 1-[4-chloro-2-fluoro-5- (trifluoromethyl)phenyl]-3-[(1S)-1- (2-pyrimidin-2-yl-1,2,4-triazol-3- yl)ethyl]urea | 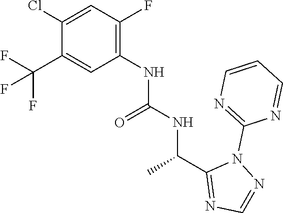 [SMILES: C[C@H](NC(=O)Nc1cc(C(F)(F)F)c(Cl)cc1F)c1ncnn1-c1ncccn1] | 1.37 | 430.25 | 3 | |
| P394 | methyl 4-bromo-2-fluoro-5-[[(1S)-1- (2-pyrimidin-2-yl-1,2,4-triazol-3- yl)ethyl]carbamoylamino]benzoate | 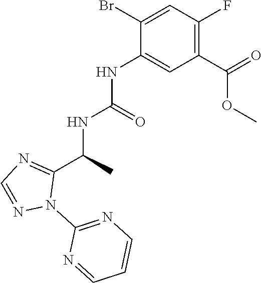 [SMILES: COC(=O)c1cc(NC(=O)N[C@@H](C)c2ncnn2-c2ncccn2)c(Br)cc1F] | 1.08 | 464.24 | 3 | |
| P395 | 2-[5-[(1S)-1-[[2,4-dichloro-5- (trifluoromethyl)phenyl]carbamoyl- amino]ethyl]-1,2,4-triazol-1-yl]-N- methyl-thiazole-5-carboxamide | 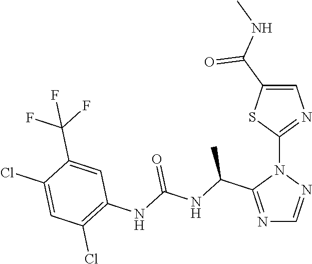 [SMILES: CNC(=O)c1cnc(-n2ncnc2[C@H](C)NC(=O)Nc2cc(C(F)(F)F)c(Cl)cc2Cl)s1] | 1.18 | 508.0 | 1 | 193 - 195 |
| P396 | 2-[5-[(1S)-1-[[2,4-dichloro-5-(1,1,2,2- tetrafluoroethoxy)phenyl]carbamoyl- amino]ethyl]-1,2,4-triazol-1-yl]-N,N- dimethyl-thiazole-5-carboxamide | 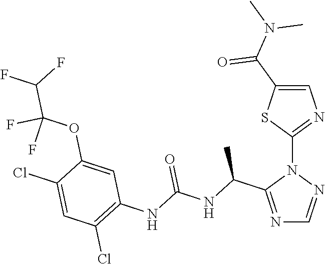 [SMILES: C[C@H](NC(=O)Nc1cc(OC(F)(F)C(F)F)c(Cl)cc1Cl)c1ncnn1-c1ncc(C(=O)N(C)C)s1] | 1.19 | 569.0 | 1 | 143 - 145 |
| P397 | 3-[4-chloro-6-(trifluoromethyl)-3- pyridyl]-1-methyl-1-[(1S)-1-(2- pyrimidin-2-yl-1,2,4-triazol-3- yl)ethyl]urea | 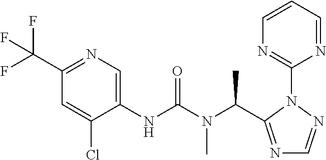 [SMILES: C[C@@H](c1ncnn1-c1ncccn1)N(C)C(=O)Nc1cnc(C(F)(F)F)cc1Cl] | 0.83 | 427.2 | 2 | |
| P398 | 1-[4-iodo-6-(trifluoromethyl)-3- pyridyl]-3-[(1S)-1-(2-pyrimidin-2-yl- 1,2,4-triazol-3-yl)ethyl]urea | 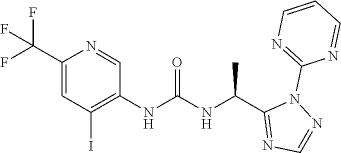 [SMILES: C[C@H](NC(=O)Nc1cnc(C(F)(F)F)cc1I)c1ncnn1-c1ncccn1] | 0.79 | 505.0 | 2 | 183 - 186 |

TABLE Q
Examples of intermediates:

| | | | Rt | [M + H]+ | | MP |
|---|---|---|---|---|---|---|
| Entry | IUPAC name | STRUCTURE | (min) | (measured) | Method | ° C. |
| I-1 | phenyl N-[1-(2-pyrimidin-2-yl-1,2,4-triazol- 3-yl)ethyl]carbamate | 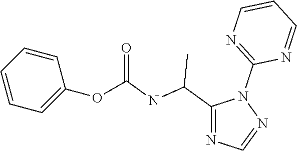 [SMILES: CC(NC(=O)Oc1ccccc1)c1ncnn1-c1ncccn1] | 0.93 | 311.5 | 1 | |
| I-2 | 1,5-dichloro-2-nitro-4-(1,1,2,2- tetrafluoroethoxy)benzene | 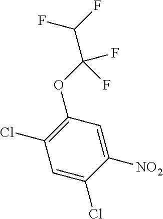 [SMILES: O=[N+]([O-])c1cc(OC(F)(F)C(F)F)c(Cl)cc1Cl] | 1.22 | | 2 | |
| I-3 | 2,4-dichloro-5-(1,1,2,2- tetrafluoroethoxy)aniline | 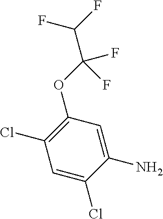 [SMILES: Nc1cc(OC(F)(F)C(F)F)c(Cl)cc1Cl] | 1.06 | 278 | 2 | |
| I-4 | methyl (2S)-2-[[2,4-dichloro-5-(1,1,2,2- tetrafluoroethoxy)phenyl]carbamoylamino] propanoate | 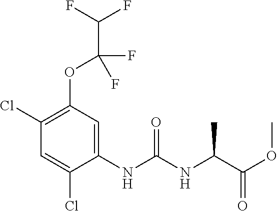 [SMILES: COC(=O)[C@H](C)NC(=O)Nc1cc(OC(F)(F)C(F)F)c(Cl)cc1Cl] | 1.04 | 407 | 2 | |
| I-5 | methyl (2S)-2-[3-[2,4-dichloro-5-(1,1,2,2- tetrafluoroethoxy)phenyl]-2-oxo- imidazolidin-1-yl]propanoate | 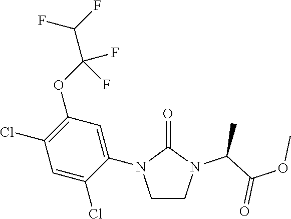 [SMILES: COC(=O)[C@H](C)N1CCN(c2cc(OC(F)(F)C(F)F)c(Cl)cc2Cl)C1=O] | 1.05 | 433 | 2 | |
| I-6 | (2S)-2-[3-[2,4-dichloro-5-(1,1,2,2- tetrafluoroethoxy)phenyl]-2-oxo- imidazolidin-1-yl]propanamide | 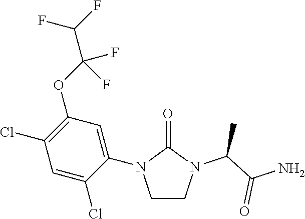 [SMILES: C[C@@H](C(N)=O)N1CCN(c2cc(OC(F)(F)C(F)F)c(Cl)cc2Cl)C1=O] | 0.91 | 418 | 2 | |
| I-7 | methyl (2S)-2-[[[2,4-dichloro-5-(1,1,2,2- tetrafluoroethoxy)phenyl]-methyl- carbamoyl]-methyl-amino]propanoate | 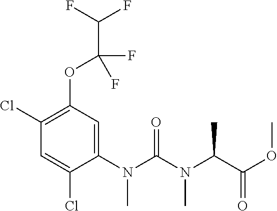 [SMILES: COC(=O)[C@H](C)N(C)C(=O)N(C)c1cc(OC(F)(F)C(F)F)c(Cl)cc1Cl] | 1.10 | 435.1 | 2 | |
| I-8 | (2S)-2-[[[2,4-dichloro-5-(1,1,2,2- tetrafluoroethoxy)phenyl]-methyl- carbamoyl]-methyl-amino]propanamide | 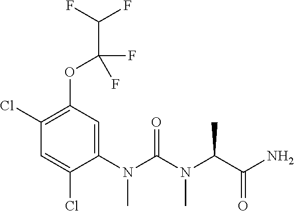 [SMILES: C[C@@H](C(N)=O)N(C)C(=O)N(C)c1cc(OC(F)(F)C(F)F)c(Cl)cc1Cl] | 0.95 | 419 | 2 | |
| I-9 | methyl (2S)-2-[3-[2,4-dichloro-5-(1,1,2,2- tetrafluoroethoxy)phenyl]-2-oxo- hexahydropyrimidin-1-yl]propanoate | 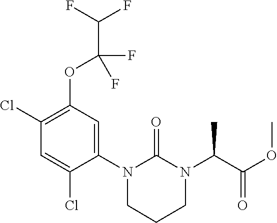 [SMILES: COC(=O)[C@H](C)N1CCCN(c2cc(OC(F)(F)C(F)F)c(Cl)cc2Cl)C1=O] | 1.06 | 447 | 2 | |
| I-10 | (2S)-2-[3-[2,4-dichloro-5-(1,1,2,2- tetrafluoroethoxy)phenyl]-2-oxo- hexahydropyrimidin-1-yl]propenamide | 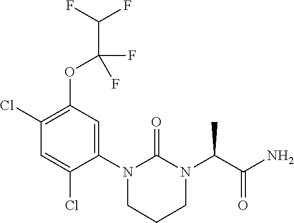 [SMILES: C[C@@H](C(N)=O)N1CCCN(c2cc(OC(F)(F)C(F)F)c(Cl)cc2Cl)C1=O] | 0.92 | 431 | 2 | |
| I-11 | methyl (2S)-2-[5-[2,4-dichloro-5-(1,1,2,2- tetrafluoroethoxy)phenyl]-4-oxo-1,3,5- oxadiazinan-3-yl]propanoate | 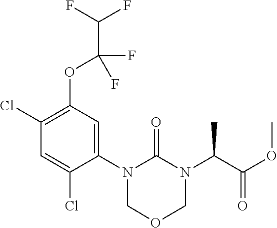 [SMILES: COC(=O)[C@H](C)N1COCN(c2cc(OC(F)(F)C(F)F)c(Cl)cc2Cl)C1=O] | 1.02 | 449 | 2 | |
| I-12 | (2S)-2-[5-[2,4-dichloro-5-(1,1,2,2- tetrafluoroethoxy)phenyl]-4-oxo-1,3,5- oxadiazinan-3-yl]propanamide | 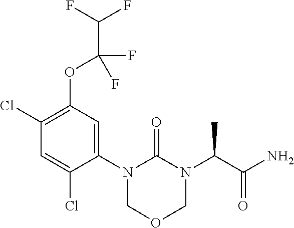 [SMILES: C[C@@H](C(N)=O)N1COCN(c2cc(OC(F)(F)C(F)F)c(Cl)cc2Cl)C1=O] | 0.90 | 433 | 2 | |
| I-13 | methyl (2S)-2-[[4-chloro-6- (trifluoromethyl)-3- pyridyl]carbamoylamino]propanoate | 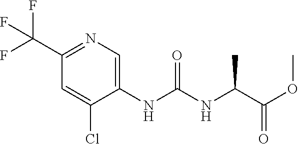 [SMILES: COC(=O)[C@H](C)NC(=O)Nc1cnc(C(F)(F)F)cc1Cl] | 0.87 | 326.2 | 2 | |
| I-14 | 2-(2,4-dichloro-5-nitro-phenoxy)pyrimidine | 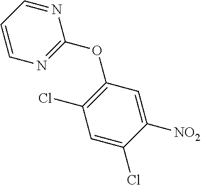 [SMILES: O=[N+]([O-])c1cc(Oc2ncccn2)c(Cl)cc1Cl] | 0.96 | 285.9 | 2 | |
| I-15 | 2,4-dichloro-5-pyrimidin-2-yloxy-aniline | 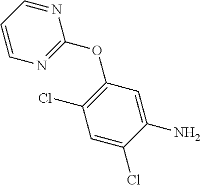 [SMILES: Nc1cc(Oc2ncccn2)c(Cl)cc1Cl] | 0.84 | 255.9 | 2 | |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of a compound of formula I with an active substance are preferred (the abbreviation “TX” means “one compound selected from the compounds defined in Tables A-1 to A-243, Tables B-1 to B-81, Table X and Table P, preferably in Table X and Table P”):
an adjuvant selected from the group of substances consisting of petroleum oils (alternative name) (628)+TX;
an insect control active substance selected from abamectin+TX, acequinocyl+TX, acetamiprid+TX, acetoprole+TX, acrinathrin+TX, acynonapyr+TX, afidopyropen+TX, afoxolaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, S-bioallethrin+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2095470-94-1+TX, CAS number: 2377084-09-6+TX, CAS number: 1445683-71-5+TX, CAS number: 2408220-94-8+TX, CAS number: 2408220-91-5+TX, CAS number: 1365070-72-9+TX, CAS number: 2171099-09-3+TX, CAS number: 2396747-83-2+TX, CAS number: 2133042-31-4+TX, CAS number: 2133042-44-9+TX, CAS number: 1445684-82-1+TX, CAS number: 1445684-82-1+TX, CAS number: 1922957-45-6+TX, CAS number: 1922957-46-7+TX, CAS number: 1922957-47-8+TX, CAS number: 1922957-48-9+TX, CAS number: 2415706-16-8+TX, CAS number: 1594624-87-9+TX, CAS number: 1594637-65-6+TX, CAS number: 1594626-19-3+TX, CAS number: 1990457-52-7+TX, CAS number: 1990457-55-0+TX, CAS number: 1990457-57-2+TX, CAS number: 1990457-77-6+TX, CAS number: 1990457-66-3+TX, CAS number: 1990457-85-6+TX, CAS number: 2220132-55-6+TX, CAS number: 1255091-74-7+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cyclobutrifluram+TX, cycloprothrin+TX, cycloxaprid+TX, cyenopyrafen+TX, cyetpyrafen+TX, cyflumetofen+TX, cyfluthrin+TX, cyhalodiamide+TX, cyhalothrin+TX, cypermethrin+TX, cyphenothrin+TX, cyproflanilide+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin (or emamectin benzoate)+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenmezoditiaz+TX, fenitrothion+TX, fenobucarb+TX, fenothiocarb+TX, fenoxycarb+TX, fenpropathrin+TX, fenpyroximate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, fluchlordiniliprole+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupentiofenox+TX, flupyradifurone+TX, flupyrimin+TX, fluralaner+TX, fluvalinate+TX, fluxametamide+TX, fosthiazate+TX, gamma-cyhalothrin+TX, Gossyplure™+TX, guadipyr+TX, halofenozide+TX, halfenprox+TX, heptafluthrin+TX, hexythiazox+TX, hydramethylnon+TX, imicyafos+TX, imidacloprid+TX, imiprothrin+TX, indazapyroxamet+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-cyhalothrin+TX, lepimectin+TX, lotilaner+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nicofluprole+TX; nitenpyram+TX, nithiazine+TX, omethoate+TX, oxamyl+TX, oxazosulfyl+TX, parathion-ethyl+TX, permethrin+TX, phenothrin+TX, phosphocarb+TX, piperonylbutoxide+TX, pirimicarb+TX, pirimiphos-ethyl+TX, pirimiphos-methyl+TX, Polyhedrosis virus+TX, prallethrin+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyriminostrobin+TX, pyriprole+TX, pyriproxyfen+TX, resmethrin+TX, sarolaner+TX, selamectin+TX, silafluofen+TX, spinetoram+TX, spinosad+TX, spirodiclofen+TX, spiromesifen+TX, spiropidion+TX, spirotetramat+TX, spidoxamat+TX, sulfoxaflor+TX, tebufenozide+TX, tebufenpyrad+TX, tebupirimiphos+TX, tefluthrin+TX, temephos+TX, tetrachlorantraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tigolaner+TX, tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, triflumezopyrim+TX, tyclopyrazoflor+TX, zeta-cypermethrin+TX, Extract of seaweed and fermentation product derived from melasse+TX, Extract of seaweed and fermentation product derived from melasse comprising urea+TX, amino acids+TX, potassium and molybdenum and EDTA-chelated manganese+TX, Extract of seaweed and fermented plant products+TX, Extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, Granulovirus+TX, Harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.+TX;
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX;
an anthelmintic selected from the group of substances consisting of abamectin (1)+TX, crufomate (1011)+TX, cyclobutrifluram+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX;
an avicide selected from the group of substances consisting of chloralose (127)+TX, endrin (1122)+TX, fenthion (346)+TX, pyridin-4-amine (IUPAC name) (23) and strychnine (745)+TX;
a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX;
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri(alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX;
a soil sterilant selected from the group of substances consisting of iodomethane (IUPAC name) (542) and methyl bromide (537)+TX;
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX;
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B, (alternative name) (839)+TX, trimedlure B₂ (alternative name) (839)+TX, trimedlure C (alternative name) (839) and trunc-call (alternative name) [CCN]+TX;
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX;
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX;
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cyclobutrifluram+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX;
a nitrification inhibitor selected from the group of substances consisting of potassium ethylxanthate [CCN] and nitrapyrin (580)+TX;
a plant activator selected from the group of substances consisting of acibenzolar (6)+TX, acibenzolar-S-methyl (6)+TX, probenazole (658) and Reynoutria sachalinensis extract (alternative name) (720)+TX;
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (including alpha-bromadiolone)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX;
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S⁴²¹ (development code) (724)+TX, sesamex (1393)+TX, sesasmolin (1394) and sulfoxide (1406)+TX;
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX;
a virucide selected from the group of substances consisting of imanin (alternative name) [CCN] and ribavirin (alternative name) [CCN]+TX;
a wound protectant selected from the group of substances consisting of mercuric oxide (512)+TX, octhilinone (590) and thiophanate-methyl (802)+TX;
a biologically active substance selected from 1,1-bis(4-chlorophenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxafos+TX, benzyl benzoate+TX, bixafen+TX, brofenvalerate+TX, bromocyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chinomethionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin I+TX, cinerin II+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dinopenton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fenpyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11 E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure B, +TX, trimedlure B₂+TX, trimedlure C+TX, trunc-call+TX, 2-(octylthio)ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)ethane+TX, 1,2-dichloropropane with 1,3-dichloropropene+TX, 1-bromo-2-chloroethane+TX, 2,2,2-trichloro-1-(3,4-dichlorophenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethylcarbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, 0-5-dichloro-4-iodophenyl O-ethyl ethylphosphonothioate+TX, 0,0-diethyl 0-4-methyl-2-oxo-2H-chromen-7-yl phosphorothioate+TX, 0,0-diethyl 0-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothiophene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, anisiflupurin+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, sodium fluoroacetate+TX, thallium sulfate+TX, warfarin+TX, 2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S⁴²¹+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, chloroinconazide+TX, mercuric oxide+TX, thiophanate-methyl+TX, azaconazole+TX, bitertanol+TX, bromuconazole+TX, cyproconazole+TX, difenoconazole+TX, diniconazole+TX, epoxiconazole+TX, fenbuconazole+TX, fluquinconazole+TX, flusilazole+TX, flutriafol+TX, furametpyr+TX, hexaconazole+TX, imazalil+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, simeconazole+TX, tebuconazole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidin+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, metalaxyl-+TX, Rmetalaxyl+TX, ofurace+TX, oxadixyl+TX, carbendazim+TX, debacarb+TX, fuberidazole+TX, thiabendazole+TX, chlozolinate+TX, dichlozoline+TX, myclozoline+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim-methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb+TX, chlorothalonil+TX, cyflufenamid+TX, cymoxanil+TX, cyclobutrifluram+TX, diclocymet+TX, diclomezine+TX, dicloran+TX, diethofencarb+TX, dimethomorph+TX, flumorph+TX, dithianon+TX, ethaboxam+TX, etridiazole+TX, famoxadone+TX, fenamidone+TX, fenoxanil+TX, ferimzone+TX, fluazinam+TX, flumetylsulforim+TX, fluopicolide+TX, fluoxytioconazole+TX, flusulfamide+TX, fluxapyroxad+TX, fenhexamid+TX, fosetyl-aluminium+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo-4-fluorophenyl)-N-(2-chloro-6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, metarylpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide+TX, N-[2-[2-chloro-4-(trifluoromethyl)phenoxy]phenyl]-3-(difluoromethyl)-1-methyl-pyrazole-4-carboxamide+TX, benzothiostrobin+TX, phenamacril+TX, 5-amino-1,3,4-thiadiazole-2-thiol zinc salt (2:1)+TX, fluopyram+TX, flufenoxadiazam+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1, 1, 3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan-4-yl) pyridine-3-carboxamide+TX, α-(1,1-dimethylethyl)-α-[4′-(trifluoromethoxy) [1, 1′-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy] benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, seboctylamine+TX, N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-[(2-trifluoromethyl)oxetan-2-yl]phenyl]-N-methyl-formamidine+TX, N-ethyl-N′-[5-methoxy-2-methyl-4-[(2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1 S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[N-methoxy-C-methyl-carbonimidoyl]-4-[5-(trifluoromethyl)-1,2,4-oxadiazol-3-yl]benzamide+TX (these compounds may be prepared from the methods described in WO 2018/202428);
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone-formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C₉-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus;

Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®); pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC-LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX, 11 Tetradecatrienyl acetate+TX, (Z+TX,Z+TX,E)-7+TX,11+TX,13-Hexadecatrienal+TX, (E+TX,Z)-7+TX, 9-Dodecadien-1-yl acetate+TX, 2-Methyl-1-butanol+TX, Calcium acetate+TX, Scenturion®+TX, Biolure®+TX, Check-Mate®+TX, Lavandulyl senecioate,
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni—System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator,
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX; and
a safener, such as benoxacor+TX, cloquintocet (including cloquintocet-mexyl)+TX, cyprosulfamide+TX, dichlormid+TX, fenchlorazole (including fenchlorazole-ethyl)+TX, fenclorim+TX, fluxofenim+TX, furilazole+TX, isoxadifen (including isoxadifen-ethyl)+TX, mefenpyr (including mefenpyr-diethyl)+TX, metcamifen+TX and oxabetrinil+TX.

The references in brackets behind the active ingredients, e.g. [3878-19-1] refer to the Chemical Abstracts Registry number. The above-described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from the compounds defined in the Tables A-1 to A-243, Tables B-1 to B-81, Table X and Table P with active ingredients described above comprises a compound selected from one compound defined in the Tables A-1 to A-243, Tables B-1 to B-81, Table X and Table P, preferably defined in Tables X and Table P, and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 to 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from the compounds defined in the Tables A-1 to A-243, Tables B-1 to B-81, Table X and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of formula I of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means, in a preferred embodiment, true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula I. Further, it is hereby made available, a composition comprising a plant propagation material treated with a compound of formula 1.

Seed treatment comprises all suitable seed treatment techniques known in the art, such as seed dressing, seed coating, seed dusting, seed soaking and seed pelleting. The seed treatment application of the compound formula I can be carried out by any known methods, such as spraying or by dusting the seeds before sowing or during the sowing/planting of the seeds.

In each aspect and embodiment of the invention, “consisting essentially” and inflections thereof are a preferred embodiment of “comprising” and its inflections, and “consisting of” and inflections thereof are a preferred embodiment of “consisting essentially of” and its inflections.

The disclosure in the present application makes available each and every combination of embodiments disclosed herein.

It should be noted that the disclosure herein in respect of a compound of formula I applies equally in respect of a compound of each of formulae I*, I′a, Iaa, and Iab.

The compounds of the invention can be distinguished from other similar compounds by virtue of greater efficacy at low application rates and/or different pest control, which can be verified by the person skilled in the art using the experimental procedures, using lower concentrations if necessary, for example 10 ppm, 5 ppm, 2 ppm, 1 ppm or 0.2 ppm; or lower application rates, such as 300, 200 or 100, mg of Al per m². The greater efficacy can be observed by an increased safety profile (against non-target organisms above and below ground (such as fish, birds and bees), improved physico-chemical properties, or increased biodegradability).

### BIOLOGICAL EXAMPLES

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 24 ppm, 12.5 ppm, 6 ppm, 3 ppm, 1.5 ppm, 0.8 ppm or 0.2 ppm.

### Example B1: Chilo suppressalis (Striped Rice Stemborer)

24-well microtiter plates with artificial diet were treated with aqueous test solutions prepared from 10,000 ppm DMSO stock solutions by pipetting. After drying, the plates were infested with L2 larvae (6-8 per well). The samples were assessed for mortality, anti-feeding effect, and growth inhibition in comparison to untreated samples 6 days after infestation. Control of Chilo suppressalis by a test sample is given when at least one of the categories mortality, anti-feedant effect, and growth inhibition is higher than the untreated sample.

The following compounds resulted in at least 80% control in at least one of the three categories (mortality, anti-feedant effect, or growth inhibition) at an application rate of 200 ppm: P1, P4, P5, P6, P7, P9, P10, P13, P14, P15, P25, P30, P35, P43, P45, P46, P48, P50, P51, P55, P58, P70, P72, P76, P79, P103, P106, P107, P114, P116, P118, P127, P128, P129, P130, P131, P132, P133, P135, P136, P137, P138, P140, P170, P172, P173, P176, P177, P178, P181, P182, P192, P200, P202, P205, P210, P212, P214, P215, P217, P219, P220, P221, P223, P235, P236, P237, P238, P240, P241, P242, P243, P244, P245, P247, P248, P249, P250, P251, P253, P255, P256, P257, P258, P260, P261, P263, P264, P265, P270, P271, P272, P273, P274, P275, P276, P277, P278, P279, P280, P294, P295, P296, P297, P299, P300, P303, P311, P314, P316, P318, P322, P323, P328, P332, P339, P342, P343, P344, P348, P350, P351, P355, P360, P367, P370, P371, P373, P376, P378, P379, P380, P383, P385, P388, P390, P391, P393, P395, P396, P397.

### Example B2: Diabrotica balteata (Corn Root Worm)

Maize sprouts placed onto an agar layer in 24-well microtiter plates were treated with aqueous test solutions prepared from 10,000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% control in at least one of the two categories (mortality or growth inhibition) at an application rate of 200 ppm:

### P1, P4, P5, P6, P13, P14, P35, P43, P46, P48, P57, P58, P103, P114, P132, P133, P135, P136, P137, P140, P172, P176, P177, P178, P205, P214, P215, P219, P223, P236, P238, P246, P257, P258, P261, P269, P272, P276, P277, P278, P279, P280, P390.

### Example B3: Euschistus heros (Neotropical Brown Stink Bug)

Soybean leaves on agar in 24-well microtiter plates were sprayed with aqueous test solutions prepared from 10,000 ppm DMSO stock solutions. After drying the leaves were infested with N2 nymphs. The samples were assessed for mortality and growth inhibition in comparison to untreated samples 5 days after infestation.

The following compounds gave an effect of at least 80% control in at least one of the two categories (mortality or growth inhibition) at an application rate of 200 ppm:

### P1, P5, P18, P46, P55, P62, P72, P103, P172, P182, P235, P246, P255, P269, P273, P277, P280, P328, P348, P390, P391, P397.

### Example B4: Frankliniella occidentalis (Western Flower Thrips):Feeding/Contact Activity

Sunflower leaf discs were placed on agar in 24-well microtiter plates and sprayed with aqueous test solutions prepared from 10,000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm:

### P71, P103, P142, P199, P208, P219, P235, P280.

### Example B5: Myzus persicae (Green Peach Aphid):Feeding/Contact Activity

Sunflower leaf discs were placed onto agar in a 24-well microtiter plate and sprayed with aqueous test solutions prepared from 10,000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm:

### P17, P32, P46, P58, P62, P91, P103, P108, P132, P133, P135, P137, P145, P168, P172, P176, P181, P202, P204, P205, P207, P235, P244, P277, P278, P328, P348.

### Example B6: Myzus persicae (Green Peach Aphid). Systemic Activity

Roots of pea seedlings infested with an aphid population of mixed ages were placed directly into aqueous test solutions prepared from 10,000 DMSO stock solutions. The samples were assessed for mortality 6 days after placing seedlings into test solutions.

The following compounds resulted in at least 80% mortality at a test rate of 24 ppm:

### P103, P172, P235, P244, P273, P348, P397.

### Example B7: Myzus persicae (Green Peach Aphid). Intrinsic Activity

Test compounds prepared from 10,000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds gave an effect of at least 80% control at an application rate of 12.5 ppm:

P1, P5, P6, P18, P30, P35, P46, P48, P55, P57, P58, P72, P79, P99, P103, P122, P127, P130, P132, P133, P135, P137, P138, P172, P176, P177, P178, P179, P181, P182, P202, P205, P210, P214, P215, P219, P221, P231, P235, P236, P238, P241, P244, P250, P257, P269, P272, P273, P274, P276, P277, P278, P280, P304, P315, P316, P328, P332, P348, P355, P360, P380, P390, P391, P397.

### Example B8: Plutella xylostella (Diamond Back Moth)

24-well microtiter plates with artificial diet were treated with aqueous test solutions prepared from 10,000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation.

The following compounds gave an effect of at least 80% in at least one of the two categories (mortality or growth inhibition) at an application rate of 200 ppm:

P1, P4, P5, P6, P7, P9, P10, P11, P12, P13, P14, P25, P26, P33, P35, P43, P46, P48, P50, P51, P55, P57, P58, P70, P72, P76, P79, P103, P107, P114, P118, P127, P128, P129, P130, P132, P133, P135, P136, P137, P138, P140, P161, P170, P171, P172, P173, P175, P176, P177, P178, P182, P191, P200, P202, P203, P205, P209, P210, P211, P214, P215, P217, P219, P220, P221, P223, P228, P229, P231, P235, P236, P237, P238, P240, P241, P243, P244, P245, P247, P248, P249, P250, P251, P253, P254, P255, P256, P257, P258, P259, P260, P261, P263, P264, P265, P270, P271, P272, P273, P274, P275, P276, P277, P278, P279, P280, P288, P290, P299, P300, P304, P305, P314, P316, P351, P367, P368, P369, P376, P380, P388, P390, P395, P396, P397.

### Example B9: Spodoptera littoralis (Egyptian Cotton Leaf Worm)

Cotton leaf discs were placed onto agar in 24-well microtiter plates and sprayed with aqueous test solutions prepared from 10,000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control in at least one of the three categories (mortality, anti-feedant effect, or growth inhibition) at an application rate of 200 ppm:

P1, P3, P4, P5, P6, P7, P11, P13, P14, P15, P18, P26, P35, P43, P46, P48, P55, P57, P58, P70, P72, P76, P79, P95, P102, P103, P114, P118, P127, P129, P130, P131, P132, P133, P135, P137, P140, P172, P173, P176, P177, P178, P182, P191, P197, P202, P203, P205, P209, P214, P215, P219, P223, P236, P237, P238, P240, P241, P242, P243, P244, P246, P247, P248, P249, P250, P251, P253, P254, P255, P256, P257, P261, P262, P263, P264, P265, P269, P271, P272, P273, P276, P277, P278, P279, P280, P300, P314, P316, P332, P343, P348, P350, P351, P360, P367, P369, P370, P373, P380, P383, P387, P388, P390, P395, P396.

### Example B10: Spodoptera littoralis (Egyptian Cotton Leaf Worm)

Test compounds were applied by pipette from 10,000 ppm DMSO stock solutions into 24-well plates and mixed with agar. Lettuce seeds were placed onto the agar and the multi well plate was closed by another plate which also contained agar. After 7 days the compound was absorbed by the roots and the lettuce grew into the lid plate. The lettuce leaves were then cut off into the lid plate. Spodoptera eggs were pipetted through a plastic stencil onto a humid gel blotting paper and the lid plate was closed with it. The samples were assessed for mortality, anti-feedant effect and growth inhibition in comparison to untreated samples 6 days after infestation.

The following compounds gave an effect of at least 80% in at least one of the three categories (mortality, anti-feeding, or growth inhibition) at a test rate of 12.5 ppm:

### P5.

### Example B11: Myzus persicae (Green Peach Aphid)

Test compounds prepared from 10,000 ppm DMSO stock solutions were applied by a liquid handling robot into 96-well microtiter plates and mixed with a sucrose solution. Parafilm was stretched over the 96-well microtiter plate and a plastic stencil with 96 holes was placed onto the plate. Aphids were sieved into the wells directly onto the Parafilm. The infested plates were closed with a gel blotting card and a second plastic stencil and then turned upside down. The samples were assessed for mortality 5 days after infestation.

The following compounds resulted in at least 80% mortality at an application rate of 50 ppm:

### P5, P6, P46, P103.

### Example B12: Plutella xylostella (Diamondback Moth)

96-well microtiter plates containing artificial diet were treated with aqueous test solutions, prepared from 10,000 ppm DMSO stock solutions, by a liquid handling robot. After drying, eggs (-30 per well) were infested onto a netted lid which was suspended above the diet. The eggs hatch and L1 larvae move down to the diet. The samples were assessed for mortality 9 days after infestation.

The following compounds gave an effect of at least 80% average mortality at an application rate of 500 ppm:

### P4, P5, P6.

### Example B13: Tetranychus urticae (Two-Spotted Spider Mite):Feeding/Contact Activity

Bean leaf discs on agar in 24-well microtiter plates were sprayed with aqueous test solutions prepared from 10,000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm:

### P277.

Abbreviations used in synthesis schemes and preparatory examples
CAN acetonitrile
Boc t-butoxycarbonyl
DBU 1,8-diazabicyclo[5.4.0]undec-7-ene
DCM dichloromethane
DDQ 2,3-dichloro-5,6-dicyano-1,4-benzoquinone
DMSO dimethyl sulfoxide
DMSO-d6 deuterated dimethylsulfoxide
DPEN diphenylethylenediamine
Et₃N triethylamine
EtOAc ethyl acetate
MeCN acetonitrile
MeOH ethanol
Ms methanesulfonyl (mesyl)
n-Bu n-butyl
NHC N-heterocyclic carbene
NPhth phthalimide-1-yl
OMs mesylate group
OTf triflate group
OTs tosylate group
PdCl2dppf 1,1′-bis(diphenylphosphino)ferrocene]palladium(II) dichloride
TBME tert-butyl methyl ether
TEA triethylamine
TEMPO (2,2,6,6-tetramethylpiperidin-1-yl)oxidanyl
Tf trifluoromethanesulfonyl (triflyl)
TFA trifluoroacetic acid
THE tetrahydrofuran
Ts p-toluenesulfonyl (tosyl)
X-Phos 2-dicyclohexylphosphino-2′,4′,6′-triisopropylbiphenyl aq. aqueous
° C. degrees Celsius
equiv. equivalent
h hour(s)
LC/MS or LC-MS liquid chromatography mass spectrometry
M molar
MHz megahertz
min minutes
mp or M.P. melting point
NMR nuclear magnetic resonance
ppm parts per million
RT room temperature
Rt retention time
RBF round-bottom flask

1. A compound of the formula I 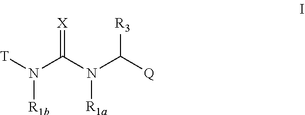
wherein: p
X is O or S; 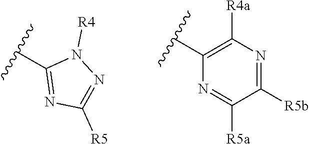
Q is Q^{a} or Q^{b};
T is phenyl, pyridine, pyrimidine, pyrazine, pyridazine or a five or nine membered heteroaromatic ring; or
T is phenyl, pyridine, pyrimidine, pyrazine, pyridazine or a five or nine membered heteroaromatic ring, each of which, independent of each other, is substituted with one to three substituents independently selected from R₂;
R₁ₐ and R_{1b} are independently selected from hydrogen, C₁-C₆alkyl, C₁-C₆alkyl substituted with one substituent independently selected from CN, C₁-C₃alkoxy, C(O)NH₂, C(O)OH, NO₂ and —Si(CH₃)₃, C₁-C₆haloalkyl, C₂-C₆alkenyl, C₂-C₆haloalkenyl, C₂-C₆alkynyl, C₂-C₆haloalkynyl, C₃-C₄cycloalkyl-C₁-C₂alkyl, C₃-C₄cycloalkyl-C₁-C₂alkyl substituted with 1 or 2 halo atoms, oxetan-3-yl-CH₂—, C₁-C₆alkylcarbonyl, C₁-C₆alkoxycarbonyl, phenyloxycarbonyl, benzyloxycarbonyl, benzyl and benzyl substituted with 1 to 3 substituents independently selected from halogen, C₁-C₆alkoxy and C₁-C₆haloalkyl; or
R₁ₐ and R_{1b} together is selected from —(CH₂)₂—, —(CH₂)₃—, —CH₂—O—CH₂—, —CH₂—S—CH₂—,
—CH₂—S(═O)—CH₂—, —CH₂—SO₂—CH₂— and —CH₂—N(R_{Y})—CH₂—, to form a 5- or 6-membered ring with the nitrogen atoms to which they are connected;
R_{1b} and T together with the nitrogen atom form a nine or ten membered bicyclic heterocyclic ring, which can be substituted with one to three substituents independently selected from C₁-C₃alkyl, C₁-C₃haloalkyl, C₁-C₃alkoxy, C₃-C₄cycloalkyl, halogen, hydroxyl, cyano and C₁-C₃haloalkoxy;
R₂ is independently selected from C₁-C₃alkyl, C₁-C₃haloalkyl, C₁-C₃haloalkylthio, C₁-C₃alkoxy, C₁-C₃haloalkoxy, halo, NO₂, SF₅, CN, C(O)NR₇R₈, C(O)OH, C(S)NH₂, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, C₃-C₆cycloalkyl, C₃-C₆cycloalkyl substituted with one to three substituents independently selected from Rₓ, C₃-C₆cycloalkylcarbonyl, phenyl, phenyl substituted with one to three substituents independently selected from Rₓ, heteroaryl, heteroaryl substituted with one to three substituents independently selected from Rₓ, OR₆, piperidin-2-on-1-yl, piperidin-2-on-1-yl substituted with one to two substituents independently selected from Rₓ, pyridin-2-on-1-yl, pyridin-2-on-1-yl substituted with one to two substituents independently selected from Rₓ, azetidin-1-yl, azetidin-1-yl substituted with one to two substituents independently selected from Rₓ, pyrrolidin-1-yl, pyrrolidin-1-yl substituted with one to two substituents independently selected from Rₓ, C₃-C₆cycloalkylC₁-C₄alkyl, C₃-C₆cycloalkylC₁-C₄alkyl substituted with one to two substituents independently selected from R_{z}, C₃-C₆cycloalkylC₁-C₃alkoxy, C₃-C₆cycloalkylC₁-C₃alkoxy substituted with one to two substituents independently selected from Rₓ, C₁-C₅cyanoalkyl, C₁-C₅cyanoalkoxy, C₁-C₄alkylsulfonyl, C₁-C₄alkylsulfonyl substituted with one to two substituents independently selected from Rₓ, C₁-C₄alkylsulfinyl, C₁-C₄alkylsulfinyl substituted with one to two substituents independently selected from Rₓ, C₃-C₆cycloalkylsulfanyl, C₃-C₆cycloalkylsulfinyl, C₃-C₆cycloalkylsulfonyl; a divalent group selected from —O—C₁-C₂haloalkanediyl-O—, —CH₂—C(CH₃)₂—O—, —CH₂—C(CH₃)₂—S—, —CH₂—C(CH₃)₂—SO₂—, wherein said divalent group is connected to T via two adjacent ring members of T; and eight to twelve membered spiroheterocyclic ring, which ring can be substituted by one or two substituents selected from halo, C₁-C₃alkyl, C₁-C₃haloalkyl, and C₁-C₃alkoxy; or
R₃ is C₁-C₃alkyl or C₁-C₃haloalkyl;
R₄ is pyridine, pyrimidine, pyrazine or pyridazine; or
R₄ is pyridine, pyrimidine, pyrazine or pyridazine each of which, independently of each other, is substituted with one to two substituents independently selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, NH₂C(O)—, NH₂C(S)—, (OH)N═C(NH₂)— and a 5-membered heteroaryl ring optionally substituted by 1 to 3 substituents independently selected from halogen, C₁-C₃alkyl, C₁-C₃haloalkyl, C₁-C₃alkoxy and C₁-C₃haloalkoxy;
R₄ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl; or
R₄ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl each of which, independently of each other, is substituted with one to two substituents independently selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, NH₂C(O)—, NH₂C(S)—, (OH)N═C(NH₂)— and a 5-membered heteroaryl ring optionally substituted by 1 to 3 substituents independently selected from halogen, C₁-C₃alkyl, C₁-C₃haloalkyl, C₁-C₃alkoxy and C₁-C₃haloalkoxy;
R₄ₐ is pyridine, pyrimidine, pyrazine, or pyridazine; or
R₄ₐ is pyridine, pyrimidine, pyrazine or pyridazine each of which, independently of each other, is substituted with one to three substituents independently selected from C₁-C₃alkyl, C₁-C₃haloalkyl, C₁-C₃alkoxy, C₃-C₄cycloalkyl, halogen, hydroxyl, cyano and C₁-C₃haloalkoxy; or
R₄ₐ is Y1, Y2, Y3 or Y4 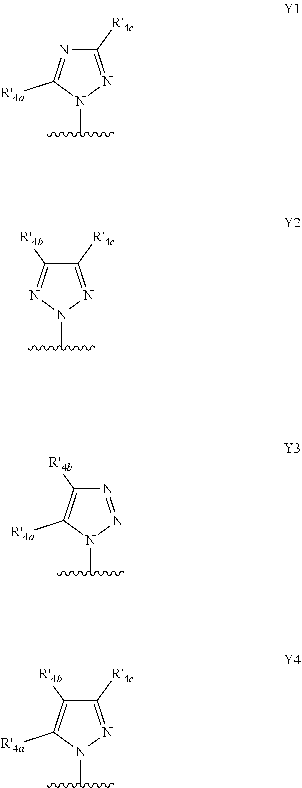
wherein, R′₄ₐ, R′_{4b} and R′_{4c}, independently of each other and independently of Y1 to Y4, are selected from hydrogen, halogen, CN, C₁-C₃alkyl, C₁-C₃haloalkyl, C₃-C₄cycloalkyl, C₁-C₃alkoxy and C₁-C₃haloalkoxy;
R₄ₐ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl; or
R₄ₐ is thiazol-2-yl, thiazol-4-yl, thiazol-5-yl, isothiazol-3-yl, isothiazol-4-yl, isothiazol-5-yl, 1,2,4-thiadiazol-3-yl, 1,3,4-thiadiazol-2-yl or 1,2,4-thiadiazol-5-yl each of which, independently of each other, is substituted with one to two substituents independently selected from C₁-C₃alkyl, C₁-C₃haloalkyl, C₁-C₃alkoxy, C₃-C₄cycloalkyl, halogen, hydroxyl, cyano, and C₁-C₃haloalkoxy; R₅ is hydrogen, C₁-C₃alkyl, C₁-C₃haloalkyl, C₃-C₄cycloalkyl, C₁-C₃alkoxy, C₃-C₄alkoxyC(O)—, (C₁-C₃alkoxy)₂CH—, halogen, CN, NH₂C(O), amino (i.e. NH₂), (C₁-C₃alkyl)amino, di(C₁-C₃alkyl)amino, hydroxy, C₃-C₄halocycloalkyl, C₃-C₄cyanocycloalkyl, C₂-C₆alkenyl, C₂-C₆haloalkenyl, C₂-C₆alkynyl, C₂-C₆haloalkynyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl, C₁-C₄alkylsulfonyl, C₁-C₃alkoxy-C₁-C₃alkyl, C₁-C₃alkoxy-C₁-C₃alkoxy-C₁-C₃alkyl, (C₁-C₃alkyl)sulfonylamino, (C₁-C₃alkyl)sulfonyl(C₁-C₃alkyl)amino, (C₁-C₃alkyl)NHC(O), (C₁-C₃alkyl)₂NC(O), (C₁-C₃cycloalkyl)NHC(O), (C₁-C₃cycloalkyl)(C₁-C₃alkyl)NC(O), (C₁-C₃alkyl)C(O)(C₁-C₃alkyl)N, (C₁-C₃alkyl)C(O)NH, (C₁-C₃alkyl)C(O), (C₁-C₃alkoxy)C(O), HC(O), diphenylmethanimine, C₁-C₃haloalkoxy, phenyl, or a 5-membered heteroaromatic ring; or
R₅ is phenyl substituted with one to three substituents selected from C₁-C₃alkyl, C₁-C₃haloalkyl, C₁-C₃alkoxy, C₃-C₄cycloalkyl, halogen, CN, and hydroxyl; or
R₅ is a 5-membered heteroaromatic ring substituted with one to three substituents selected from C₁-C₃alkyl, C₁-C₃haloalkyl, C₁-C₃alkoxy, C₃-C₄cycloalkyl, halogen, CN, and hydroxyl;
R₅ₐ and R_{5b} are, independently of each other, selected from hydrogen, halogen, CN, C₁-C₃alkyl, C₁-C₃haloalkyl, C₃-C₄cycloalkyl, C₁-C₃alkoxy and C₁-C₃haloalkoxy;
R₆ is phenyl, benzyl, heteroaryl, C₃-C₆cycloalkyl, or C₃-C₆cycloalkylC₁-C₃alkyl; or
R₆ is phenyl, benzyl, heteroaryl, C₃-C₆ cycloalkyl, or C₃-C₆cycloalkylC₁-C₃alkyl, each of which, independent of each other, is substituted with one to three substituents independently selected from Rₓ;
R₇ is hydrogen, C₁-C₃alkyl or C₃-C₆cycloalkyl; or
R₇ is C₁-C₃alkyl or C₃-C₆cycloalkyl, each of which, independent of each other, is substituted with one to three substituents independently selected from R_{z};
R₈ is hydrogen, C₁-C₃alkyl or C₃-C₆cycloalkyl; or
R₈ is C₁-C₃alkyl or C₃-C₆cycloalkyl, each of which, independent of each other, is substituted with one to three substituents independently selected from R_{z}; or
R₇ and R₈ together is —(CH₂)₂—O—(CH₂)₂— and form 6-membered ring with the nitrogen atom;
Rₓ is independently selected from halogen, C₁-C₃alkyl, C₁-C₃haloalkyl, C₁-C₃alkoxy, C₁-C₃haloalkoxy, NO₂, SF₅, CN, C(O)NH₂, C(S)NH₂, C₃-C₆cycloalkyl, C₁-C₄haloalkylsulfanyl, C₁-C₄haloalkylsulfinyl, C₁-C₄haloalkylsulfonyl, C₁-C₄alkylsulfanyl, C₁-C₄alkylsulfinyl and C₁-C₄alkylsulfonyl;
R_{Y} is C₁-C₄ alkyl, C₃-C₅ alkenyl, C₃-C₄ cycloalkyl, C₂-C₅ alkynyl, or benzyl;
R_{z} is selected from oxo, halogen, C₁-C₃ alkyl, C₁-C₃haloalkyl, C₁-C₃alkoxy, C₁-C₃haloalkoxy and CN; or an agrochemically acceptable salt, stereoisomer, enantiomer, tautomer, and N-oxide of the compound of formula I.

2. The compound according to claim 1, wherein X is oxygen.

3. The compound according to claim 1, wherein X is sulfur.

4. The compound according to claim 1, wherein R₃ is C₁-C₂alkyl or C₁-C₂haloalkyl.

5. The compound according to claim 1, wherein R₁ₐ and R_{1b} independent of each other, is hydrogen, methyl, ethyl, cyanomethyl, methoxymethyl, cyclopropyl-methyl, allyl, propargyl, benzyloxycarbonyl, or benzyl, or R₁ₐ and R_{1b} together is —CH₂—O—CH₂—.

6. The compound according to claim 1, wherein T is phenyl, pyridine, pyrimidine, pyrazine, pyridazine or a heteroaromatic ring selected from J-13, J-16, J-22, J-25, J-26, J-27, J-28, J-31, J-36, J-37, J-38, J-39, J-40, J-41, thieno[2,3-d]pyrimidinyl, [1,2,4]triazolo[1,5-a]pyrimidinyl, and indazolyl, which are, independently of each other, unsubstituted or substituted with one to three substituents independently selected from R₂.

7. The compound according to claim 1, wherein R₂, independent of each other, is selected from halogen, C₁-C₃alkyl, C₁-C₃haloalkyl, C₁-C₃alkoxy, C₁-C₃haloalkoxy, CN, C₃-C₄cycloalkyl, C₃-C₆cycloalkylcarbonyl, phenyl, heteroaryl selected from J-1 and J-41, each of C₃-C₄cycloalkyl, phenyl or heteroaryl, independent of each other, being substituted with one to three substituents Rₓ; OR₆, piperidin-2-one-1-yl, pyridin-2-one-1-yl, azetidin-1-yl optionally substituted with Rₓ, pyrrolidin-1-yl, C₃-C₆cycloalkylC₁-C₄alkyl substituted with one or two substituents R_{z}, C₃-C₆cycloalkylC₁-C₃alkoxy optionally substituted with Rₓ, C₁-C₅cyanoalkyl, C₁-C₅cyanoalkoxy, C₁-C₄alkylsulfanyl optionally substituted by one to three substituents Rₓ, C₁-C₄alkylsulfonyl optionally substituted by one to three substituents Rₓ, C₁-C₄alkylsulfinyl optionally substituted by one to three substituents Rₓ, C(O)NR₇R₈, C₁-C₄alkylsulfonylamino, aminosulfonyl, C₁-C₄alkylaminosulfonyl, di(C₁-C₄alkyl)aminosulfonyl, C₃-C₆cycloalkylaminosulfonyl, —O—C₁-C₂haloalkyl-O—, and 10 membered optionally substituted spiroheterocyclic ring.

8. The compound according to claim 1, wherein Q is selected from Q^{a}-1 to Q^{a}-16, and Q^{b}-1 to Q^{b}-13.

9. The compound according to claim 1, wherein R₄, (independently of Q^{a} in the instance Q is one of Q^{a}-1 to Q^{a}-16), is pyridine or pyrimidine, wherein the pyridine or pyrimidine, independently of each other, is optionally substituted with one substituent selected from C₁-C₃alkyl, C₁-C₃haloalkyl, C₁-C₃alkoxy, C₃-C₄cycloalkyl, halo, hydroxyl, CN, C₁-C₆haloalkoxy, C₂-C₆haloalkenyloxy, C₂-C₆haloalkynyloxy, C₃-C₄halocycloalkoxy, C₃-C₆cycloalkylC₁-C₄haloalkoxy, NH₂C(O)—, NH₂C(S)—, (OH)N═C(NH₂)—, J-13 optionally substituted by C₁-C₃haloalkyl, J-20 optionally substituted by C₁-C₃haloalkyl and 1H-tetrazol-5-yl.

10. The compound according to a claim 1, wherein R₄ₐ, (independently of Q^{b} in the instance Q is one of Q^{b}-1 to Q^{b}-13), is pyridine or pyrimidine, wherein the pyridine or pyrimidine is optionally substituted with one substituent selected from cyclopropyl, F, Cl, Br, CN, trifluoromethoxy, difluoromethoxy, 2,2-difluoroethoxy and 2,2,2-trifluoroethoxy, or R₄ₐ is selected from Y1 to Y4.

11. The compound according to claim 1 wherein the formula I is represented by 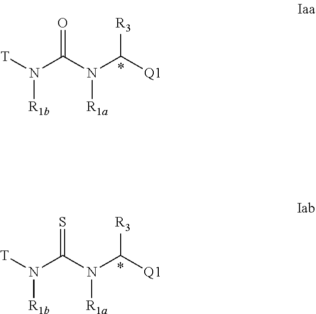
wherein T, R₁ₐ, R_{1b}, R₃ are as defined in claim 1, and Q₁ is as defined for Q in claim 1.

12. The compound according to claim 11 wherein Q1 is selected from Q^{aa} to Q^{ag} and Q^{ba} to Q^{bf}.

13. A composition comprising a compound as defined in claim 1, one or more auxiliaries and diluent, and optionally one or more other active ingredient.

14. A method
(i) of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound as defined in claim 1; or
(ii) for the protection of plant propagation material from the attack by insects, acarines, nematodes or molluscs, which comprises treating the propagation material or the site, where the propagation material is planted, with an effective amount of a compound as defined in claim 1; or
(iii) of controlling parasites in or on an animal in need thereof comprising administering an effective amount of a compound as defined in claim 1.

15. A plant propagation material, such as a seed, comprising, or treated with or adhered thereto, a compound as defined in claim 1.

16. A compound of the formula III 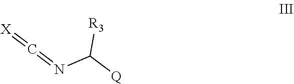
wherein X is oxygen or sulfur, and R₃ and Q are as defined in claim 1.

17. A compound of the formula IV 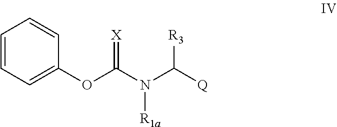
wherein X is oxygen or sulfur, and R₁ₐ and Q are as defined in claim 1.

18. A compound of the formula VII 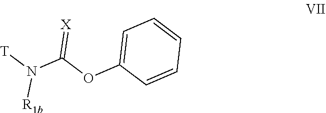
wherein X is oxygen or sulfur, and R_{1b} and T are as defined in claim 1, and R₂ when T is substituted with one to three substituents independently selected from R₂, is as defined in claim 1.

19. A compound of the formula XIII or a compound of the formula XVI 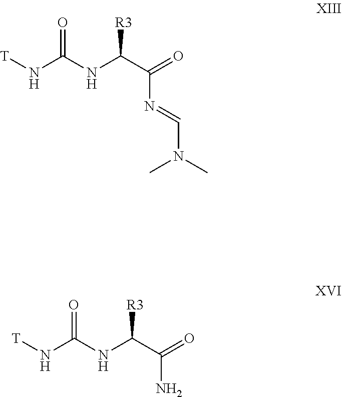
wherein T and R³ are as defined in claim 1, and R² when T is substituted with one to three substituents independently selected from R², is as defined in claim 1.
